SEQUENCE LISTING (9 sequences)

SEQ ID NO: 1 - protein, 9 aa, Artificial Sequence
  Synthetic polypeptide
LAGLIDADG

SEQ ID NO: 2 - DNA, 3301 bp, Artificial Sequence
  Synthetic polynucleotide
gtgcctcttgcagcagcctaacccagaagcaggggggaatcctgaatcgagctgagagggcttccccggttctcctgggaaccccatcggccccctgccagcacacacctgagcagcatcacaggacatggccccctcagccacctagctggggcccatctaggagtggcatcttttttggtgccctgaaggccagctctggaccttcccaggaaaagtgccagctcacagaactgcttgaccaaaggaccggctcttgagacatcccccaacccacctggcccccagctagggtgggggctccaggagactgagattagcctgccctctttggacagcagctccaggacagggcgggtgggctgaccacccaaaccccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattctaaaaaaaaaaaaaaaaaa

SEQ ID NO: 3 - protein, 462 aa, Artificial Sequence
  Synthetic polypeptide
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 4 - DNA, 20 bp, Artificial Sequence
  Synthetic polynucleotide
gatgtacgagagtgtagaag

SEQ ID NO: 5 - DNA, 20 bp, Artificial Sequence
  Synthetic polynucleotide
tatatccactaactggaagc

SEQ ID NO: 6 - DNA, 20 bp, Artificial Sequence
  Synthetic polynucleotide
gtccgtactccaccccgctc

SEQ ID NO: 7 - DNA, 20 bp, Artificial Sequence
  Synthetic polynucleotide
ccattgaggtgcccgccccc

SEQ ID NO: 8 - DNA, 20 bp, Artificial Sequence
  Synthetic polynucleotide
cttgtagatgcggggtagag

SEQ ID NO: 9 - DNA, 20 bp, Artificial Sequence
  Synthetic polynucleotide
tgatgatgcagttcttgtcc

(19) United States
(12) Patent Application Publication
(10) Pub. No.: US 2020/0390731 A1
(43) Pub. Date: Dec. 17, 2020

(54) MANIPULATION OF THE RETINOIC ACID SIGNALING PATHWAY

(71) Applicant: THE REGENTS OF THE UNIVERSITY OF CALIFORNIA, Oakland, CA (US)

(72) Inventors: Richard H. Kramer, Oakland, CA (US); Michel Telias, Emeryville, CA (US); Bristol Layne Denlinger, Alameda, CA (US); Zachary John Helft, Berkeley, CA (US)

(21) Appl. No.: 16/763,180

(22) PCT Filed: Nov. 16, 2018
(86) PCT No.: PCT/US18/61689

§ 371 Date: May 11, 2020

Related U.S. Application Data
(60) Provisional application No. 62/588,181, filed on Nov. 17, 2017.

(51) Int. Cl. A61K 31/192; A61P 27/02; C12N 9/22; C12N 15/113; C12N 15/86; A61K 9/00

(52) U.S. Cl. CPC A61K 31/192; A61P 27/02; A61K 9/0048; C12N 15/113; C12N 15/86; C12N 9/22

(57) ABSTRACT

Disclosed herein, inter alia, are compositions and methods for modulating the retinoic acid receptor signaling pathway and treating vision degeneration.

### STATEMENT AS TO RIGHTS TO INVENTIONS MADE UNDER FEDERALLY SPONSORED RESEARCH AND DEVELOPMENT

This invention was made with government support under grant nos. EY003176 and EY024334 awarded by the National Institutes of Health. The government has certain rights in the invention.

### CROSS-REFERENCES TO RELATED APPLICATIONS

This application claims the benefit of U.S. Provisional Application No. 62/588,181, filed Nov. 17, 2017, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 052103-512001WO_Sequence_Listing_ST25.txt, created Nov. 14, 2018, 10,280 bytes, machine format IBM-PC, MS Windows operating system, is hereby incorporated by reference.

### BACKGROUND

Light responses are initiated in rod and cone photoreceptors, processed by interneurons, and synaptically transmitted to retinal ganglion cells (RGCs), which generate action potentials that carry visual information to the brain. In mouse models of inherited retinal degeneration, the RGCs survive but exhibit electrophysiological remodeling, including heightened spontaneous activity that obscures responses to dim light. Understanding the biochemical pathway involved in the progressive degeneration of rod and cone photoreceptors and RGC electrophysiological remodeling is vital to unlock potential therapeutic targets for technologies aimed at restoring visual perception in diseases associated with vision loss, such as retinitis pigmentosa and age-related macular degeneration. Disclosed herein, inter alia, are solutions to these and other problems in the art.

### BRIEF SUMMARY

In an aspect is provided a method of treating vision degeneration, the method including administering to a subject in need thereof an effective amount of a retinoic acid receptor inhibitor.

In an aspect is provided a method for treating vision degeneration, the method including administering a virus or viral vector, wherein the virus or viral vector includes a nucleic acid sequence encoding a modified retinoic acid receptor or retinoid x receptor.

In an aspect is provided a method of inhibiting the activity of a retinoic acid receptor in a subject in need thereof, including contacting the retinoic acid receptor with a retinoic acid receptor inhibitor.

In an aspect is provided a method of treating vision degeneration, the method including administering to a subject in need thereof an effective amount of an inhibitor of the level of retinoic acid in the subject.

In an aspect is provided a retinoic acid receptor inhibitor, having the formula:

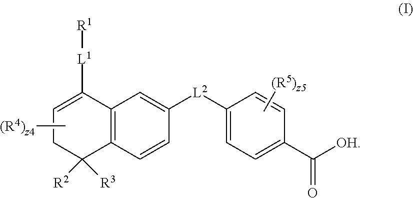

L¹ is a bond, —S(O)₂—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is —S(O)₂—, —NH—, —O—, —S—, —C(O)—, —C(O)NH—, —NHC(O)—, —NHC(O)NH—, —C(O)O—, —OC(O)—, —C(S)—, —C(S)NH—, —NHC(S)—, —NHC(S)NH—, —C(S)O—, —OC(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² and R³ are each independently hydrogen, substituted or unsubstituted alkyl, or substituted or unsubstituted heteroalkyl. R⁴ and R⁵ are each independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z4 is an integer from 0 to 3. The symbol z5 is an integer from 0 to 4.

### BRIEF DESCRIPTION OF THE DRAWINGS

FIGS. 1A-1B. Blocking RA signaling in degenerated retinas decreases dye permeability in RGCs. FIG. 1A: Representative images of YO-PRO-1 loading into RGCs in a WT retina (left), an rd1 retina injected with vehicle (middle) and an rd1 retina injected with the pan-RAR inhibitor BMS-493 (right). Ganglion cells (1) were included in the analysis while vascular associated cells (2) were excluded. FIG. 1B: Quantification of the fraction of cells in the GCL permeable to YO-PRO-1. ˜1.0 μl of solution was intravitreally injected 3-7 days prior to dye loading. Injections included: 1% DMSO in PBS (vehicle), DEAB 20 μM, Citral 50 μM and BMS-493 0.5 μM. Data are shown as the percentage of YO-PRO-1 positive cells in a field of view (counterstained with Nuclear I.D.). All values greater than 40% are represented visually at a single level for effective data visualization. Values are shown as mean %±SEM. *p<0.05, ***p<0.001, unpaired 2-tailed Student's T-tests.

FIGS. 2A-2F. Blocking RA signaling in degenerated retinas decreases photoswitch-mediated photosensitization and spontaneous activity in RGCs. FIG. 2A: Blocking RA signaling reduces rd1 photosensitization with QAQ. Representative raster plots and multi-electrode array (MEA) recordings of QAQ-mediated photosensitization of the rd1 retina, untreated (left) and after intravitreal injection with the RAR antagonist BMS-493 (right). Light responses were elicited by cycling between 380 nm (dark grey) and 500 nm (light grey) light. FIG. 2B: Quantification of FIG. 2A. RGC activity was recorded under synaptic isolation. For BMS-493 treatment, retinas were analyzed 3-7 days post-injection. QAQ was bath-loaded at 300 μM. rd1 n=4 retinas, rd1+BMS-493 n=8 retinas. Values represent the mean Photoswitch Index±SEM. **p<0.005, unpaired 2-tailed Student's t-test. FIG. 2C: Blocking RA signaling reduces rd1 photosensitization with BENAQ. Representative raster plots and MEA recordings of BENAQ-mediated photosensitization in the rd1 retina, untreated (left), and after an intravitreal injection of BMS-493 (right). Light responses were elicited by cycling between white light and darkness. FIG. 2D: Quantification of FIG. 2C. RGC activity was recorded under synaptic isolation. For BMS-493 treatment, retinas were analyzed 3-7 days post-injection. BENAQ was bath-loaded at 30 μM. rd1 n=7 retinas, rd1+BMS-493 n=8 retinas. Values represent the mean Photoswitch Index±SEM. *p<0.05, unpaired 2-tailed Student's t-test. FIG. 2E: Representative raster plots and MEA recordings of RGC spontaneous activity in darkness, in the untreated rd1 retina (left), and after an intravitreal injection of BMS-493 (right). FIG. 2F: Quantification of FIG. 2E. RGC activity was recorded in ACSF. For BMS-493 treatment, retinas were analyzed 3-7 days post-injection. rd1 n=10 retinas, rd1+BMS-493 n=16 retinas. Values represent the mean Firing Rate (Hz)±SEM. **p<0.005, unpaired 2-tailed Student's T-test.

FIGS. 3A-3D. Blocking RA signaling improves light sensitivity of the retina in vision-impaired rd10 mice. 6-week-old rd10 mice were injected with BMS-493 in one eye and received a vehicle injection in the other. FIG. 3A: MEA recordings of a single 50 ms light flash of 0.2 μW light shows a response in BMS-493 injected animals but not vehicle injected mice. (Top) Bar represents the light state presented to the retina showing the location of the flash. (Middle) Raster plots for each unit show reduced spontaneous activity and simultaneous action potential firing during the flash for BMS-493 injected animals. (Bottom) Averaged responses for all units. FIG. 3B: Averaged response over 9 light flash cycles shows robust light responses and reduced spontaneous activity in BMS-493 injected retinas but not vehicle. FIG. 3C: Comparing the light responses within an individual mouse between eyes reveals a significantly increased light response in every subject at 0.2 μW light intensity. Data are represented as mean±SEM. Paired t-test, n=5. FIG. 3D: Response curves within the same pieces of retina over a range of light intensities. BMS-493 injected retinas showed a leftward and upward shift of fitted sigmoidal curves as represented by the ratio of firing rate in the light:firing rate in the dark compared to vehicle. Data are represented as mean±SEM.

FIGS. 4A-4C. Activating RA signaling in WT retinas increases dye permeability in RGCs. FIG. 4A: Representative images of YO-PRO-1 loading into RGCs in an rd1 retina (left), and in WT retinas treated with vehicle (middle left), all-trans retinoic acid (ATRA; middle right), and ATRA+the P2X receptor antagonist TNP-ATP during dye loading (right). FIGS. 4B-4C: Quantification of the fraction of cells in the GCL permeable to YO-PRO-1. Treatments included in FIG. 4B: 1% DMSO in PBS (vehicle), ATRA 0.1 μM, Liarozole 100 μM and TNP-ATP 200 μM. Treatments included in FIG. 4C: ATRA 0.1 μM, BMS-493 0.5 retinaldehyde 1 μM, and DEAB 20 μM. All treatments were administered as ˜1.0 μl intravitreal injections, 3-7 days prior to dye loading, with the exception of TNP-ATP which was bath-applied ex-vivo. Data are shown as the percentage of YO-PRO-1 in a field of view (counterstained with Nuclear I.D., not shown). All values greater than 40% are represented visually at a single level for effective data visualization. Values are shown as mean % SEM. *p<0.05, ***p<0.001, ANOVA with Tukey HSD (in FIG. 4B) and unpaired 2-tailed Student's T-test (in FIG. 4B, FIG. 4C).

FIGS. 5A-5E. Inducing RA signaling in WT retinas mimics photosensitization with QAQ but not BENAQ. FIGS. 5A-5B: Representative raster plots of multi-electrode array (MEA) recordings of QAQ-mediated photosensitization of the WT retina, untreated (FIG. 5A) and after intravitreal injection with all-trans retinoic acid (ATRA) and the cyp26 antagonist liarozole (FIG. 5B). Light responses were elicited by cycling between 380 nm (dark grey) and 500 nm (light grey) light. FIGS. 5C-5D: Representative raster plots of MEA recordings of BENAQ-application to WT retinas, untreated (FIG. 5C), and after an intravitreal injection with ATRA and liarozole (FIG. 5D). Light responses were elicited by cycling between white light and darkness. FIG. 5E: Quantification of (FIGS. 5A-5D). RGC activity was recorded under synaptic isolation. All retinas were analyzed 3-7 days post-injection, with the exception of “6 wk ATRA+Liarozole”, analyzed 6 weeks post-injection. Data from rd1 are shown on the left. All eyes were intravitreally injected with a volume of ˜1.0 μl. Injections included: ATRA 0.1 μM and Liarozole 100 μM. TNP-ATP 100 μM and QAQ 300 μM were bath-loaded. The number of experiments carried out for each group was as follows: rd1 n=4, WT n=4, ATRA n=7, Liarozole n=4, ATRA+Liarozole n=8, ATRA+Liarozole+TNP-ATP n=5, 6 WK n=5. Values represent the mean Photoswitch Index±SEM. **p<0.005, unpaired 2-tailed Student's T-test.

FIGS. 6A-6C. A retinoic acid receptor-dependent genetically-encoded dual reporter. FIG. 6A: Schematic representation of the reporter sequences, including the constitutive expression of red fluorescence protein (RFP) under the cytomegalovirus promoter (CMV), and the retinoic-acid regulated expression of green fluorescence protein (GFP), obtained by inserting three repetitions of the retinoic acid response element (RARE) sequence upstream to the weak SV40 promoter, resulting in RA-regulated expression. FIG. 6B: Representative images of in-vitro transfection of human embryonic kidney (HEK) cells with the reporter. Cells were transfected using lipofectamine and 48 hrs later either vehicle alone (0.1% DMSO in PBS) or with ATRA (1 μM) were added to the culture medium for an additional 48 hrs. Images show strong RFP expression in both vehicle and ATRA treatment, but a significant increase in GFP only following ATRA treatment. FIG. 6C: Quantification of RFP and GFP fluorescence corresponding to (FIG. 6B). Data are shown as normalized values for RFP and GFP in cells treated with vehicle (0.1% DMSO in PBS) or with ATRA (1 The experiment was repeated 3 times. Values are shown as mean±SEM. n.s.: non-significant, ***p<0.001, unpaired 2-tailed Student's T-test.

FIGS. 7A-7C. Retinal degeneration is associated with increased retinoic acid in the inner retina. FIG. 7A: Representative images of flat-mounted whole retinas with the GCL facing up from WT (left, up) and rd1 (left, bottom) mice infected with the RA dual reporter virus at birth and analyzed at 2-3 months of age. Distribution of GFP values in RFP⁺ cells and mean F value for GFP±SEM, in WT (right, up) and rd1 (right, bottom) mice. In unlabeled naive rd1 and WT retina, we measured background fluorescence levels and established a threshold composed of the mean fluorescence value+2SD (vertical black line). Analysis was conducted on Z-stacks comprising the RGC and the IPL layers. Data were pulled from 6-8 retinas for each strain. Images correspond to 2D projections of Z-stacks using a spinning disk confocal microscope. FIG. 7B: Similar to FIG. 7A, carried out in WT (up) and s334ter (bottom) rats. Viral infection was carried out through intravitreal injections in 3-4 months old animals, and retinas were analyzed 2-3 weeks post-injection. Images correspond to 2D projections of Z-stacks using a laser-scanning confocal microscope. FIG. 7C: Representative images of DAPI and GFP staining in cross-sections obtained from an s334ter rat retina following infection with RARE double reporter virus (left). Dotted lines indicate the limits of the inner nuclear layer (INL), the inner plexiform layer (IPL), ganglion cell layer (GCL) and the two different sub-laminae, FIG. 7A and FIG. 7B. Quantification of GFP levels in FIG. 7A vs. FIG. 7B sub-lamina (OFF- and ON-RGCs stratification, respectively) was carried out using a GFP-specific antibody with no RFP cross-reaction. Data are shown as the normalized mean GFP fluorescence±SEM. n.s.: non-significant difference, unpaired 2-tailed Student's T-test.

FIGS. 8A-8B. ATRA does not cause retinal neuron degeneration. FIG. 8A: Representative images of a TUNEL assay carried out in order to assess whether intravitreal injections of retinoic acid trigger apoptosis in the inner retina. Insets emphasize the photoreceptor nuclear layer. Positive controls were obtained using DNAse, as per the manufacturer's instructions. Nuclear I.D. was used as counterstaining. Vehicle was PBS containing DMSO 0.1%. The final concentration of ATRA and Liarozole in the eye was 100 nM and 100 respectively. Eyes were collected 5-6 days post-injection. The experiment was repeated 2-3 times for each condition. FIG. 8B: Quantification of the fraction of WT RGCs loading YO-PRO-1 following intravitreal ATRA injection with a final concentration of 100 nM in the eye. Loading was evaluated 1 hr, 3, 7, 14, and 42 days after injection. Cell nuclei were counterstained with Nuclear I.D. Uninjected data is the same as that shown in FIGS. 3A-3D. All data are represented as mean %±SEM.

FIGS. 9A-9B. Intraocular injections of ATRA result in increased expression of RARβ in RGCs. Representative images (FIG. 9A) and quantification (FIG. 9B) of immunohistochemistry assays for the detection of retinoic acid receptor β (RARβ) relative levels in retinal cross-sections, 5-6 days following intravitreal injections of vehicle (PBS w/DMSO 0.1%) or all-trans retinoic acid (ATRA, 100 nM). A significant increase in RARβ was detected in the ganglion cell layer (GCL; white box). Background fluorescence levels were established in assays including secondary antibody only (not shown), and results are shown as the intensity of fluorescence in vehicle or ATRA treatments over background. Values are shown as mean±SEM, ***p<0.001, Student's T-test.

FIGS. 10A-10B. Spontaneous activity of WT RGCs following acute induction of RA signaling. FIG. 10A: Representative raster plots and MEA recordings of RGC spontaneous activity in darkness, in the WT retina, untreated (top), and after an intravitreal injection of ATRA+Liarozole (bottom). FIG. 10B: Quantification of FIG. 10A. RGC activity was recorded in ACSF. Retinas were analyzed 3-7 days post-injection. WT n=8 retinas, WT+ATRA+Liarozole n=16 retinas. Values represent the mean Firing Rate (Hz)±SEM; unpaired 2-tailed Student's T-test.

FIGS. 11A-11B. In-vitro ratiometric calibration of RA-dual reporter dose and time response. FIG. 11A: Ratiometric analysis of dose-dependent induction of GFP by ATRA. HEK-293 cells were transfected by Lipofectamin 2000 with the RAR reporter construct. Cells were treated with different doses of ATRA for 48 hrs and then fixed with paraformaldehyde. Images were separately analyzed for RFP and GFP fluorescence levels, and then the ratio of fluorescence was calculated. ATRA treatments also included 100 μM Liarozole to prevent ATRA degradation. Individual data points and mean±SEM values are shown. FIG. 11B: Ratiometric analysis of time-dependent induction of GFP by ATRA. Transfected HEK cells were treated with 1 μM ATRA+100 μM Liarozole, and fixed after 1, 2, 4, 12 and 24 hrs. Images were analyzed for RFP and GFP fluorescence levels. Individual data points and mean±SEM values are shown.

FIGS. 12A-12D. Retinal degeneration induces hyperactivity. FIG. 12A: MEA recordings of retinal light responses in retina of rd10 mice at ages P14, 28 and 60. FIG. 12B: Quantification of peak light responses (circle) and spontaneous activity in the dark (square) as a function of age in rd10 mice ex vivo retinal pieces. Values are mean±SEM, measured in 7-10 retinal rd10 pieces at each age, including P14, 21, 28, 35, 42 and 60. FIG. 12C: MEA recordings of spontaneous activity of synaptically-isolated RGCs in the dark in P60 rd1 retina and in a normal WT counterpart. FIG. 12D: Spontaneous firing in normal saline (first set of data) and after adding synaptic blockers (second set of data). To eliminate rod-mediated light responses, we promoted bleaching adaptation by exposing the isolated retinas to room light for 30 minutes before recording (Dowling, J., 1987). In all three strains synaptic blockade caused no change in spontaneous firing (n.s.: non-significant, WT: n=9, p=0.99; rd1: n=6, p=0.28; rd10: n=4, p=0.76; paired t-test). Spontaneous firing in rd1 and rd10 remained significantly higher than in WT, even after synaptic blockade. Values are mean±SEM.

FIGS. 13A-13B. Neurotransmitter receptor antagonist cocktail blocks chemical synaptic responses in RGCs. FIG. 13A: MEA recording of light responses in WT retina in saline before (left panel) or after (right panel) perfusion of a mixture of synaptic blockers, including: (in μM) 10 AP4, 40 DNQX, 30 APS, 10 SR-95531, 50 TPMPA, 10 strychnine, and 50 tubocurarine. Light responses disappeared in 9 out of 9 recordings. FIG. 13B: Patch-clamp recordings of spontaneous excitatory postsynaptic currents in an rd1-RGC neuron, voltage clamped to −60 mV (normal saline, left panel). After perfusion of saline including the synaptic blocker mixture (right panel), excitatory post-synaptic currents disappeared in 23 out of 23 cells recorded.

FIGS. 14A-14C. A genetically-encoded RAR-reporter shows increased RAR-signaling in-vivo. FIG. 14A: Left: fluorescence images of HEK293 cells expressing the RAR reporter. Cells were lipofectamine-transfected and 48 hrs later, either vehicle alone (0.1% DMSO in PBS) or with 1 μM all-trans retinoic-acid (ATRA) were added to the culture medium for an additional 48 hrs. Images show strong RFP expression after either vehicle or ATRA treatment, but a significant increase in GFP expression only following ATRA treatment. Right: quantification of RFP and GFP fluorescence, normalized to mean RFP fluorescence values in vehicle-treated cells. Values are shown as mean±SEM, 3 separate experiments were conducted in duplicate wells of HEK cells grown in serum-free media. n.s.: non-significant, ***p<0.001, 2-tailed t-test. FIGS. 14B-14C: The RAR reporter shows RA signaling in the ganglion cell layer (GCL) from WT and degenerated mouse retina (rd1, FIG. 14B), and WT and degenerated rat retina (s334ter, FIG. 14C). RA-dependent signaling was quantified in individual cells by measuring GFP fluorescence in RFP-expressing cells (mean gray value in arbitrary units, ‘a.u.’). Histograms show the distribution of GFP fluorescence in RFP-positive cells. Data for individual cell values were pooled from 8-10 retinas in 4-5 animals per strain. Each retina was divided into 3-4 pieces, and 4-6 fields of view were imaged and analyzed for each piece. Images correspond to 2D projections of Z-stacks from a spinning disk confocal microscope. Vertical line indicates the median value of GFP fluorescence for each strain. Animals were injected intravitreally at P30-45 with AAV2-RAR-reporter and analyzed ˜60-90 days later.

FIG. 15. Analysis of human transcriptome data for RA-responsive genes. Gene expression in a retinal sample from an RP patient compared to a sample from a control donor. Primary data were from Mullins et al., 2012 (36). Boxplots for RP/Ctrl are shown for the whole dataset (left) vs. from the subset consisting of 120 AmiGO-validated RA-responsive genes (right). RP/control for RA-responsive gene transcripts=3.03±0.71; RP/control for entire population of transcripts=1.44±0.014; p=0.0136, Mann-Whitney Rank Sum Test.

FIGS. 16A-16E. RAR activation induces hyperpermeability of degenerated retinas through P2X receptors. FIG. 16A: Images of Yo-Pro-1 labeling of RGCs in the GCL of WT retinas injected with vehicle or ATRA, and in rd1 retinas injected with vehicle, or RAR inhibitor BMS 493. Scale bar is 20 μm in length. FIG. 16B: Quantification of the fraction of cells labeled with Yo-Pro-1 for vehicle-injected (1 μL intra-vitreous, 1% DMSO in PBS, 3-7 days prior to dye loading assay) in WT and rd1 retinal pieces. Data are shown as the percentage of Yo-Pro-1 positive cells in a field of view (counterstained with Nuclear I.D., not shown). FIG. 16C: Quantification as in FIG. 16B, above. All experiments were conducted in WT retinas. Intravitreal injections included 0.1 μM all-trans retinoic acid (ATRA); 100 μM Liarozole (Cyp26 inhibitor); and 0.5 μM BMS-493 (pan-RAR inverse agonist). 200 μM TNP-ATP (P2X antagonist) was bath loaded. Black asterisks—compared to WT baseline (FIG. 16B, dotted line), boxed asterisks—ATRA vs. ATRA+Liarozole. FIG. 16D: Quantification as in FIG. 16B, above. All experiments were conducted in WT retinas. Intravitreal injections included 0.5 μM retinaldehyde (RAL); 20 μM N,N-diethylaminobenzaldehyde (DEAB, RALDH inhibitor). Black asterisks—compared to WT baseline (FIG. 16B, dotted line), boxed asterisks—RAL vs. RAL+DEAB. FIG. 16E: Quantification as in FIG. 16B, above. All experiments were conducted in rd1 retinas. Intravitreal injections included 20 μM DEAB, n=30; 50 μM Citral (RALDH inhibitor); 0.5 μM BMS 493. Black asterisks—compared to rd1 baseline (FIG. 16B, dotted line). FIGS. 16B-16E: *p<0.05, ***p<0.001, t-test and Mann-Whitney.

FIG. 17. Intravitreal injection of ATRA is non-toxic and reversible. Eyes were collected and stained 5-6 days post-injection. Injections were performed in 2 mice. For each mouse, one eye was injected and analyzed for each condition, and a total of 6 different fixed retinal sections were stained and analyzed for each treatment. % of fluorescin-labeled cells is shown for positive control, vehicle, ATRA and ATRA+Liarozole. Values are mean±SEM, *** p<0.001, n.s.—non-significant, Kruskal-Wallis.

FIG. 18. Quantification of the fraction of WT RGCs labeled with Yo-Pro-1 following intravitreal injection of 0.1 μM ATRA. Labeling was evaluated in WT retinas, 1 hr or 3, 7, 15, or 42 days after injection. Cell nuclei were counterstained with Nuclear I.D. All data are represented as mean±SEM. *p<0.05, ***p<0.001, t-test and Mann-Whitney Test.

FIGS. 19A-19E. Pharmacological activation of RAR is necessary and sufficient for degeneration-dependent chemical photosensitization. FIGS. 19A-19B: MEA recordings from QAQ-treated WT retina, without (FIG. 19A) or with (FIG. 19B) prior intravitreal injection of ATRA plus liarozole. Photoswitching was elicited by alternating between 380 nm (dark grey) and 500 nm (light grey) light. QAQ (300 μM) was applied onto the isolated retina for 30 minutes and then washed away. FIG. 19C: Quantification of FIGS. 19A-19B. Photosensitivity (Photoswitch Index, PI) induced by QAQ was measured in WT retinas. Recordings were obtained 3-7 days after ˜1 μL intravitreal injection, including 1% DMSO in PBS (vehicle control, ‘Ctrl.’, dotted line), 0.1 μM all-trans retinoic acid (ATRA), 100 μM Liarozole (Cyp26 inhibitor). Recordings were also obtained 6 weeks after injection of ATRA+Liarozole. 200 μM TNP-ATP (P2X antagonist) was bath loaded. **—Ctrl. vs ATRA+Liarozole, *—ATRA+Liarozole vs. ATRA+Liarozole+TNP-ATP. FIG. 19D: Blocking RAR reduces photosensitization of rd1 retinas. Retinas were obtained from eyes without (left) or with (right) BMS-493 (0.5 μM), injected into the vitreous at 3-7 days prior to retina isolation and recording. FIG. 19E: Quantification of FIG. 19D in rd1 retinas. Control (‘Ctrl.’, non-injected eyes), were compared to rd1 eyes injected with 0.5 μM BMS-493, 3-7 days prior to recordings. Values are shown as mean±SEM. *p<0.05, **p<0.01, Kruskal-Wallis and t-test.

FIGS. 20A-20B. Retinal expression of RAR_{DN}. FIG. 20A: Confocal fluorescent image of flat-mount retina in a P90 rd1 mouse injected with intravitreally with an AAV2 serotype of the RAR_{DN} virus (pAAV-hSyn1-RAR_{DN}—RFP-WPRE) at P30. The whole retinal flat-mount, with the ganglion cell layer (GCL) upwards, was imaged in a single frame using a 4× objective (low magnification, left) and with a 40× objective (high magnification, right), in 20-30 μm thick 3D Z-stacks through the GCL, that were flattened for 2D-renderization. FIG. 20B: Quantification of the fraction of RFP-positive cells in the GCL of rd1 mice intravitreally injected with AAV2-RAR_{DN} (as in FIG. 20A), and in P40-50 rd10 mice injected in the tail vein with an AAV9-RAR_{DN} at P2-3. Each retinal piece was imaged in 3-5 different fields. Total number of cells per field of view was established using Nuclear I.D. in naive retinas. All data are represented as mean±SEM.

FIGS. 21A-21B. Pharmacological or genetic inhibition of RAR reduces hyperactivity in the degenerated rd1 retina. FIG. 21A: MEA recordings of spontaneous activity in the dark, in a naive rd1 retina (left), and in a BMS 493-injected rd1 retina (right), both in saline. FIG. 21B: Quantification of spontaneous activity in rd1 control retinas (‘Ctrl.’, non-injected eyes), in rd1 retinas injected with 0.5 μM BMS 493 3-7 days prior to the experiment, and in rd1 retinas from P90 mice, injected at P30 with AAV2-RAR_{DN}. Values represent the mean Firing Rate (Hz)±SEM. *p<0.05, **p<0.01, one-way ANOVA.

FIGS. 22A-22B. Inhibition of RAR activation reduces hyperactivity of RGCs and boosts light responses in vision-impaired mice. FIG. 22A: Responses from 5 mice, comparing the light-elicited change in firing in the vehicle-injected (1% DMSO in PBS) and BMS-493-injected eye. Data is shown as mean±SEM, *p<0.05, Paired t-test. FIG. 22B: Intensity-response curves from the retinas of vehicle-injected and BMS-493-injected eyes. Solid line is a basis-spline fit to the data. Data are represented as the mean ratio of firing rate in the light/dark±SEM.

FIGS. 23A-23E. In vivo blockade of RAR increases innate and learned visual responses. FIG. 23A: Quantification of innate aversion to light in naive rd10 mice as compared to rd10 mice injected at P2-3 with RAR_{DN}. All mice were tested at P37-38. The graph shows the % of time spent by the mouse in the dark side of the chamber as a function of light intensity (darkness, ˜250 μW/cm², 2500 μW/cm²). Values are mean±SEM, *p<0.05, χ²-test. FIG. 23B: Individual traces for light responses (% time spent freezing) to darkness (0) and four different intensities of light (240, 550, 1400 and 2500 μW/cm²), using the learned light aversion behavioral paradigm. Untreated WT and rd10 mice were compared to rd10 mice injected at P2-3 with RAR_{DN}. All mice were tested at P33-35. FIG. 23C: Quantification of FIG. 23B. The graph shows % of time spent freezing as a function of light intensity. Values are mean±SEM, ***p<0.001, t-test (WT and rd10-RAR_{DN} vs. rd10). FIG. 23D: Probability of mouse displaying a response above threshold for the first light flash using the dimmest intensity (˜240 μW/cm²), in each strain, including WT, rd10 and rd10-RAR_{DN}. For each individual trace in FIG. 23B, the slope of the response was measured, and threshold was set as a slope that is ≥+0.05. FIG. 23E: Gel electrophoresis (left) and quantification (right) of semi-quantitative reverse-transcription PCR assay carried out on RNA extracted from retinas of rd10-RAR_{DN} and naive rd10 mice. n=3 for each, all mice were P40 at the day of dissection and RNA purification. ntc—non-template control; Rho—rhodopsin; RARα—retinoic acid receptor alpha; RAM—retinoic acid receptor beta; Cyp26—cytochrome P450 26A1; RFP—red fluorescent protein (mStrawberry). β-Actin was used as a housekeeping gene. Quantification of relative transcript levels for gene expression was normalized to β-actin. For each subtype, rd10 is shown first and rd10-RAR_{DN} is shown second. Values are mean±SEM, *p<0.05, **p<0.001, t-test.

### DETAILED DESCRIPTION

Retinoic acid is the initiator of retinal ganglion cell hyperexcitability in inherited degenerative blinding disease, presenting new therapeutic targets for improving or restoring light-sensitivity in the vision-impaired. Light responses are initiated in rod and cone photoreceptors, processed by interneurons, and synaptically transmitted to retinal ganglion cells (RGCs), which generate action potentials that carry visual information to the brain. In mouse models of inherited retinal degeneration, the RGCs survive but exhibit electrophysiological remodeling, including heightened spontaneous activity that obscures responses to dim light. Herein we disclose that retinoic acid (RA), a developmental morphogen, is the signal that triggers electrophysiological remodeling. Blocking RA signaling reduces RGC remodeling and unmasks light responses in degenerating retinas, enhancing RA signaling mimics remodeling in healthy retinas, and a genetically-encoded fluorescent reporter verifies that RA signaling is actually increased during degeneration. Identification of RA as the initiator of remodeling presents a new therapeutic opportunity for boosting low-level vision and enhancing the effectiveness of visual prosthetic technologies during degenerative blindness.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl, or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

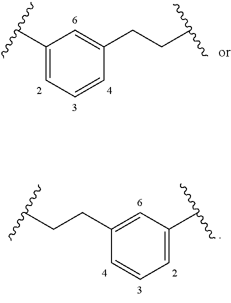 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CHzI, —CN, —OH, —NHz, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHF₂, —OCH₂C₁, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted aryl ene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted aryl ene, and/or substituted heteroarylene) is substituted with at least one size-limited substituent group, wherein if the substituted moiety is substituted with a plurality of size-limited sub stituent groups, each size-limited sub stituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog or derivative is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g., glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

The term “aptamer” as provided herein refers to oligonucleotides (e.g., short oligonucleotides or deoxyribonucleotides), that bind (e.g., with high affinity and specificity) to proteins, peptides, and small molecules. Aptamers may be RNA. Aptamers may have secondary or tertiary structure and, thus, may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990). In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Anti-cancer agents (e.g., chemotherapy drugs, toxins, and siRNAs) may be successfully delivered to cancer cells in vitro using aptamers.

“Nucleic acid” refers to deoxyribonucleotides or derivatives thereof or nucleotide analogs thereof, ribonucleotides or derivatives thereof or nucleotide analogs thereof, and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof (e.g., derivatives, analogs), including the nucleotide analogs described below. Examples of nucleic acids (e.g., polynucleotides) contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms above (e.g., nucleic acid, DNA, RNA) also encompass nucleic acids including known nucleotide analogs or modified backbone residues or linkages, which are synthetic, naturally occurring, and non-naturally occurring, which may have similar binding properties as the reference nucleic acid, and which may be metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. In embodiments, nucleic acid analogs include peptide nucleic acids (PNA), 2′-O-methyl (2′-OMe), or 2′-O-methyoxyethyl 92′-OMOE). Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both. In embodiments, DNA includes one or more nucleotide analogs. In embodiments, RNA includes one or more nucleotide analogs. In embodiments, DNA does not include one or more nucleotide analogs. In embodiments, RNA does not include one or more nucleotide analogs.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g., DNA, RNA, polymer of nucleotide analogs) that is capable of binding to a target nucleic acid (e.g., an mRNA translatable into a protein) and reducing transcription of the target nucleic acid (e.g., mRNA from DNA) or reducing the translation of the target nucleic acid (e.g., mRNA) or altering transcript splicing (e.g., single stranded morpholino oligo).

As used herein the term “nucleic acid molecule” refers to a covalently linked sequence of nucleotides or bases or nucleotide derivatives or analogs (e.g., ribonucleotides for RNA and deoxyribonucleotides for DNA but also include DNA/RNA hybrids where the DNA is in separate strands or in the same strands) in which the 3′ position of the pentose of one nucleotide is joined by a phosphodiester linkage to the 5′ position of the pentose of the next nucleotide or modified backbone residues or linkages. A nucleic acid molecule may be single- or double-stranded or partially double-stranded. A nucleic acid molecule may appear in linear or circularized form in a supercoiled or relaxed formation with blunt or sticky ends and may contain “nicks.” Nucleic acid molecules may be composed of completely complementary single strands or of partially complementary single strands forming at least one mismatch of bases. Nucleic acid molecules may further comprise two self-complementary sequences that may form a double-stranded stem region, optionally separated at one end by a loop sequence. The two regions of nucleic acid molecules which comprise the double-stranded stem region are substantially complementary to each other, resulting in self-hybridization. However, the stem can include one or more mismatches, insertions or deletions. As described above, nucleic acid molecules may include chemically, enzymatically, or metabolically modified forms of nucleic acid molecules or combinations thereof. Chemically synthesized nucleic acid molecules may refer to nucleic acids typically less than or equal to 150 nucleotides long (e.g., between 5 and 150, between 10 and 100, between 15 and 50 nucleotides in length) whereas enzymatically synthesized nucleic acid molecules may encompass smaller as well as larger nucleic acid molecules as described elsewhere in the application. Enzymatic synthesis of nucleic acid molecules may include stepwise processes using enzymes such as polymerases, ligases, exonucleases, endonucleases or the like or a combination thereof. The terms “genome editing” or “gene editing” as provided herein refer to stepwise processes involving enzymes such as polymerases, ligases, exonucleases, endonucleases or the like or a combinations thereof. For example, gene editing may include processes where a nucleic acid molecule is cleaved, nucleotides at the cleavage site or in close vicinity thereto are excised, new nucleotides are newly synthesized and the cleaved strands are ligated.

The term nucleic acid molecule also refers to short nucleic acid molecules, often referred to as, for example, “primers” or “probes.” Primers are often referred to as single stranded starter nucleic acid molecules for enzymatic assembly reactions whereas probes may be typically used to detect at least partially complementary nucleic acid molecules. A nucleic acid molecule has a “5′-terminus” and a “3′-terminus” because nucleic acid molecule phosphodiester linkages (or modified linkages, for example, phosphodiester derivatives) occur between the 5′ carbon and 3′ carbon of the pentose ring of the substituent mononucleotides. The end of a nucleic acid molecule at which a new linkage would be to a 5′ carbon is its 5′ terminal nucleotide. The end of a nucleic acid molecule at which a new linkage would be to a 3′ carbon is its 3′ terminal nucleotide. A terminal nucleotide or base, as used herein, is the nucleotide at the end position of the 3′- or 5′-terminus. A nucleic acid molecule sequence, even if internal to a larger nucleic acid molecule (e.g., a sequence region within a nucleic acid molecule), also can be said to have 5′- and 3′-ends.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is uridine or thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

A “vector” as used herein is a nucleic acid molecule that can be used as a vehicle to transfer genetic material into a cell. A vector can be a plasmid, a virus or bacteriophage, a cosmid or an artificial chromosome such as, e.g., yeast artificial chromosomes (YACs), bacterial artificial chromosomes (BAC) or other sequences which are able to replicate or be replicated in vitro or in a host cell, or to convey a desired nucleic acid segment to a desired location within a host cell. In embodiments a vector refers to a DNA molecule harboring at least one origin of replication, a multiple cloning site (MCS) and one or more selection markers. A vector is typically composed of a backbone region and at least one insert or transgene region or a region designed for insertion of a DNA fragment or transgene such as a MCS. The backbone region often contains an origin of replication for propagation in at least one host and one or more selection markers. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment which do not require the use of recombination, transpositions or restriction enzymes (such as, but not limited to, uracil N glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. In embodiments, a vector contains additional features. Such additional features may include natural or synthetic promoters, genetic markers, antibiotic resistance cassettes or selection markers (e.g., toxins such as ccdB or tse2), epitopes or tags for detection, manipulation or purification (e.g., V5 epitope, c-myc, hemagglutinin (HA), FLAG™, polyhistidine (His), glutathione-S-transferase (GST), maltose binding protein (MBP)), scaffold attachment regions (SARs) or reporter genes (e.g., green fluorescent protein (GFP), red fluorescence protein (RFP), luciferase, β-galactosidase, etc.). In embodiments, vectors are used to isolate, multiply or express inserted DNA fragments in a target host. A vector can for example be a cloning vector, an expression vector, a functional vector, a capture vector, a co-expression vector (for expression of more than one open reading frame), a viral vector or an episome (i.e., a nucleic acid capable of extrachromosomal replication), etc.

An “expression vector” is designed for expression of a transgene and generally harbors at least one promoter sequence that drives expression of the transgene. Expression as used herein refers to transcription of a transgene or transcription and translation of an open reading frame and can occur in a cell-free environment such as a cell-free expression system or in a host cell. In embodiments, expression of an open reading frame or a gene results in the production of a polypeptide or protein. An expression vector is typically designed to contain one or more regulatory sequences such as enhancer, promoter and terminator regions that control expression of the inserted transgene. Suitable expression vectors include, without limitation, plasmids and viral vectors. Vectors and expression systems for various applications are available from commercial suppliers such as Novagen (Madison, Wis.), Clontech (Palo Alto, Calif.), Stratagene (La Jolla, Calif.), and Life Technologies Corp. (Carlsbad, Calif.). In embodiments an expression vector is engineered for expression of a TAL effector fusion.

A “viral vector” generally relates to a genetically-engineered noninfectious virus containing modified viral nucleic acid sequences. In embodiments, a viral vector contains at least one viral promoter and is designed for insertion of one or more transgenes or DNA fragments. In embodiments a viral vector is delivered to a target host together with a helper virus providing packaging or other functions. In embodiments viral vectors are used to stably integrate transgenes into the genome of a host cell. A viral vector may be used for delivery and/or expression of transgenes.

Viral vectors may be derived from bacteriophage, baculoviruses, tobacco mosaic virus, vaccinia virus, retrovirus (avian leukosis-sarcoma, mammalian C-type, B-type viruses, D type viruses, HTLV-BLV group, lentivirus, spumavirus), adenovirus, parvovirus (e.g., adenoassociated viruses), coronavirus, negative strand RNA viruses such as orthomyxovirus (e.g., influenza virus) or sendai virus, rhabdovirus (e.g., rabies and vesicular stomatitis virus), paramyxovirus (e.g., measles and Sendai), positive strand RNA viruses such as picornavirus and alphavirus (such as Semliki Forest virus), and double-stranded DNA viruses including adenovirus, herpes virus (e.g., Herpes Simplex virus types 1 and 2, Epstein-Barr virus, cytomegalovirus), and poxvirus (e.g., vaccinia, fowlpox and canarypox). Other viruses include without limitation Norwalk virus, togavirus, flavivirus, reoviruses, papovavirus, hepadnavirus, and hepatitis virus. For example common viral vectors used for gene delivery are lentiviral vectors based on their relatively large packaging capacity, reduced immunogenicity and their ability to stably transduce with high efficiency a large range of different cell types. Such lentiviral vectors can be “integrative” (i.e., able to integrate into the genome of a target cell) or “non-integrative” (i.e., not integrated into a target cell genome). Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The terms “transfection,” “transduction,” “transfecting,” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

For specific proteins described herein (e.g., Cas9, Argonaute), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Thus, a “CRISPR associated protein 9,” “Cas9,” or “Cas9 protein” as referred to herein includes any of the recombinant or naturally-occurring forms of the Cas9 endonuclease or variants or homologs thereof that maintain Cas9 activity (e.g., within at least 50%, 80%, 90%, 95%, 96%, 97%, 98%, 99%, or 100% activity compared to Cas9) (e.g., endonuclease enzyme activity). In some aspects, the variants or homologs have at least 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase.” In embodiments, Cas9 binds a CRISPR (clustered regularly interspaced short palindromic repeats) nucleic acid sequence. In embodiments, the CRISPR nucleic acid sequence is a prokaryotic nucleic acid sequence. Examples of Cas9 proteins useful for the invention provided herein include without limitation, cas9 mutant proteins such as, HiFi Cas9 as described by Kleinstiver, Benjamin P., et al. (“High-fidelity CRISPR-Cas9 nucleases with no detectable genome-wide off-target effects.” Nature (2016). PubMed PMID: 26735016); Cas9 proteins binding modified PAMs and orthologous Cas9 proteins such as CRISPR from Prevotella and Francisella 1(Cpfl). Any of the mutant Cas9 forms commonly known and described in the art may be used for the methods and compositions provided herein. Non-limiting examples of mutant Cas9 proteins contemplated for the methods and compositions provided herein are described in Slaymaker, Ian M., et al. (“Rationally engineered Cas9 nucleases with improved specificity.” Science (2015): aad5227. PubMed PMID: 26628643) and Kleinstiver, Benjamin P., et al. (“High-fidelity CRISPR-Cas9 nucleases with no detectable genome-wide off-target effects.” Nature (2016). PubMed PMID: 26735016) which are incorporated by reference in their entirety and for all purposes.

The term “CRISPR,” “Clustered regularly interspaced short palindrome repeats” or the like refer, in the usual and customary sense, to segments of DNA (e.g., prokaryotic DNA) containing short repetitions of base sequences. Each repetition is typically followed by short segments of spacer DNA, as known in the art, from previous exposures to an infectious agent, e.g., a bacteriophage virus or plasmid. The term “CRISPR/Cas system” or the like refers, in the usual and customary sense, to a prokaryotic immune system that confers resistance to foreign genetic elements such as those present within plasmids and phages, providing a form of acquired immunity. As known in the art, CRISPR associated proteins (Cas) use the CRISPR spacers to recognize and cut these exogenous genetic elements. Accordingly, delivery of the Cas9 nuclease and appropriate guide RNAs (e.g., nucleic acid sequences described herein) into a cell can result in scission of the genome of the cell at a desired location, allowing existing genes to be removed and/or new genes or fragments thereof to be added. CRISPR/Cas system typically include a guide RNA (gRNA) designed to associate with a CRISPR-associated endonuclease (e.g., Cas9) and which includes a target nucleotide sequence that targets (e.g., binds) the genomic sequence to be modified and a CRISPR-associated endonuclease (e.g., Cas9) that makes the DNA double-strand break.

As used herein, “CRISPR complex” refers to the CRISPR proteins and nucleic acid (e.g., RNA) that associate with each other to form an aggregate that has functional activity. An example of a CRISPR complex is a wild type Cas9 (sometimes referred to as Csn1) protein that is bound to a guide RNA specific for a target locus.

As used herein, “CRISPR protein” refers to a protein comprising a nucleic acid (e.g., RNA) binding domain nucleic acid and an effector domain (e.g., Cas9, such as Streptococcus pyogenes Cas9, or CPF1 (cleavage and polyadenylation factor 1)). The nucleic acid binding domains interact with a first nucleic acid molecules either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

In some embodiments, CRISPR complexes may generate double-stranded breaks or may have a combined action for the generation of double-stranded breaks. For example, mutations may be introduced into CRISPR components that prevent CRISPR complexes from making double-stranded breaks but still allow for these complexes to nick DNA. Mutations have been identified in Cas9 proteins that allow for the preparation of Cas9 proteins that nick DNA rather than making double-stranded cuts.

CRISPR systems that may be used vary greatly. These systems will generally have the functional activities of a being able to form complex comprising a protein and a first nucleic acid where the complex recognizes a second nucleic acid. CRISPR systems can be a type I, a type II, or a type III system. Non-limiting examples of suitable CRISPR proteins include Cas3, Cas4, Cas5, Cas5e (or CasD), Cash, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the CRISPR protein (e.g., Cas9) is derived from a type II CRISPR system. In some embodiments, the CRISPR system is designed to act as an oligonucleotide (e.g., DNA or RNA) guided endonuclease derived from a Cas9 protein. The Cas9 protein for this and other functions set out herein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

The term “guide RNA” or “gRNA” as provided herein refers, in the usual and customary sense, to a ribonucleotide sequence capable of binding a nucleoprotein, thereby forming ribonucleoprotein complex. In embodiments, the guide RNA includes one or more RNA molecules. In embodiments, the gRNA includes a nucleotide sequence complementary to a target site. The complementary nucleotide sequence may mediate binding of the ribonucleoprotein complex to the target site thereby providing the sequence specificity of the ribonucleoprotein complex. Thus, in embodiments, the guide RNA is complementary to a target nucleic acid. In embodiments, the guide RNA binds a target nucleic acid sequence. In embodiments, the guide RNA is complementary to a CRISPR nucleic acid sequence. In embodiments, the complement of the guide RNA has a sequence identity of about 50%, 55%, 60%, 65%, 70%, 75%, 80%, 85%, 90%, 95%, 96%, 97%, 98%, 99%, or 100% to a target nucleic acid. In embodiments, the complement of the guide RNA has a sequence identity of at least 50%, 55%, 60%, 65%, 70%, 75%, 80%, 85%, 90%, 95%, 96%, 97%, 98%, 99%, or 100% to a target nucleic acid. In embodiments, a target nucleic acid sequence as provided herein is a nucleic acid sequence expressed by a cell. In embodiments, the target nucleic acid sequence is an exogenous nucleic acid sequence. In embodiments, the target nucleic acid sequence is an endogenous nucleic acid sequence. In embodiments, the target nucleic acid sequence forms part of a cellular gene (i.e., is a fragment thereof). Thus, in embodiments, the guide RNA is complementary to a cellular gene or fragment thereof (e.g., retinoic acid receptor or a fragment thereof or gene or a complement thereof). In embodiments, the guide RNA binds a cellular gene sequence or a fragment thereof (e.g., retinoic acid receptor gene or a fragment thereof, or a complement thereof). In embodiments, a guide RNA is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a complementary sequence to the target gene or target nucleic acid (e.g., retinoic acid receptor or a fragment thereof, or a complement thereof). In embodiments, the guide RNA binds a cellular gene sequence or a fragment thereof (e.g., retinoic acid receptor gene or a fragment thereof, or a complement thereof) adjacent to a PAM sequence. In embodiments, the target gene or target nucleic acid (e.g., retinoic acid receptor gene or a fragment thereof, or a complement thereof) is adjacent to a PAM sequence.

The term “protospacer adjacent motif” or “PAM” as provided herein refers, in the usual and customary sense, to a 2 to 8 base pair nucleic acid (e.g., DNA) sequence immediately following the nucleic acid (e.g., DNA) sequence targeted by the Cas9 nuclease in the CRISPR bacterial adaptive immune sustem. In embodiments, the PAM is required for a Cas nuclease to cut. In embodiments, the PAM sequence is 1 to 10 nucleotides downstream from the cut site. In embodiments, the PAM sequence is 3 to 4 nucleotides downstream from the cut site. In embodiments, the PAM sequence is the sequence chosen from the group (read from 5′ to 3′): NGG, NGA, TTTN, TTTV, YTN, NGRRT, NGRRN, NNNNGATT, NNNNRYAC, NNAGAAW, or NAAAAC, wherein N is any nucleobase; V is guanine, cytosine or adenine; R is guanine or adenine; Y is cytosine or thymine; and W is adenine or thymine.

In embodiments, the guide RNA is a single-stranded ribonucleic acid. In embodiments, the guide RNA is about 10, 20, 30, 40, 50, 60, 70, 80, 90, 100 or more nucleic acid residues in length. In embodiments, the guide RNA is from about 10 to about 30 nucleic acid residues in length. In embodiments, the guide RNA is about 20 nucleic acid residues in length. In embodiments, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the guide RNA is from 10 to 15, 10 to 20, 10 to 30, 10 to 40, or 10 to 50 residues in length. In embodiments, the guide RNA is from 19 to 23 residues in length.

The term “Argonaute (AGO) protein,” “NgAgo,” “Natronobacterium gregoryi Argonaute,” or “N. gregoryi SP2 Argonaute” as referred to herein includes any of the recombinant or naturally-occurring forms of the NgAgo or variants or homologs thereof that maintain NgAgo endonuclease enzyme activity (e.g., within at least 50%, 80%, 90%, 95%, 96%, 97%, 98%, 99% or 100% activity compared to wild type NgAgo). In embodiments, the variants or homologs have at least 70%, 75%, 80%, 85%, 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring NgAgo protein. In embodiments, the NgAgo protein is substantially identical to the protein identified by the National Center for Biotechnology Information (NCBI) protein identifier AFZ73749.1 or a variant or homolog having at least 70%, 75%, 80%, 85%, 90%, 95%, 96%, 97%, 98%, 99%, or 100% amino acid sequence identity thereto. In embodiments, Argonaute proteins can also include nuclease domains (i.e., DNase or RNase domains), additional DNA binding domains, helicase domains, protein-protein interaction domains, dimerization domains, as well as other domains.

The term “Transcription Activator-Like Effector Nuclease (TALEN)” is used in accordance with its plain ordinary meaning and refers to enzymes engineered to excise a specific portion of a nucleic acid. TALEN systems typically include transcription activator-like (TAL) effectors of plant pathogenic Xanothomonas spp fused to a FokI nuclease. Genomic targeting specificity is accomplished through customization of the polymorphic amino acid repeats in the TAL effectors.

As used herein “TAL effector” or “TAL effector protein” as provided herein refers to a protein including more than one TAL repeat and capable of binding to nucleic acid in a sequence specific manner. In embodiments, TAL effector protein includes at least six (e.g., at least 8, at least 10, at least 12, at least 15, at least 17, from about 6 to about 25, from about 6 to about 35, from about 8 to about 25, from about 10 to about 25, from about 12 to about 25, from about 8 to about 22, from about 10 to about 22, from about 12 to about 22, from about 6 to about 20, from about 8 to about 20, from about 10 to about 22, from about 12 to about 20, from about 6 to about 18, from about 10 to about 18, from about 12 to about 18, etc.) TAL repeats. In embodiments, the TAL effector protein includes 18 or 24 or 17.5 or 23.5 TAL nucleic acid binding cassettes. In embodiments, the TAL effector protein includes 15.5, 16.5, 18.5, 19.5, 20.5, 21.5, 22.5 or 24.5 TAL nucleic acid binding cassettes. A TAL effector protein includes at least one polypeptide region which flanks the region containing the TAL repeats. In embodiments, flanking regions are present at the amino and/or the carboxyl termini of the TAL repeats.

The term “zinc-finger nuclease” is used in accordance with its plain ordinary meaning and refers to a protein comprising a polypeptide having nucleic acid (e.g., DNA) binding domains that are stabilized by zinc. The individual DNA binding domains are typically referred to as “fingers,” such that a zinc-finger protein or polypeptide has at least one finger, more typically two fingers, or three fingers, or even four or five fingers, to at least six or more fingers. In some embodiments, a zinc-finger nuclease will contain three or four zinc fingers. Each finger typically binds from two to four base pairs of DNA. Each finger usually comprises an about 30 amino acids zinc-chelating, DNA-binding region (see, e.g., U.S. Pat. Publ. No. 2012/0329067 A1, the disclosure of which is incorporated herein by reference). Zinc-finger nuclease refers to enzymes engineered to excise a specific portion of a nucleic acid by fusing a zinc finger DNA-binding domain to a DNA-cleavage domain. The DNA binding domain includes two-finger modules, each of which recognize a unique sequence of DNA, and are fused to create a zinc-finger protein. The DNA-cleaving domain includes the nuclease domain of FokI. The first (DNA-binding domain) and second (DNA-cleavage domain) domains are fused, thereby creating a complex that cleaves double-stranded DNA at a target genomic location defined by the zinc-finger protein.

The term “meganuclease” or “homing meganuclease” is used in accordance with its plain ordinary meaning and refers to endodeoxyribonucleases characterized by a large recognition site (double-stranded DNA sequences of 12 to 40 base pairs). Meganucleases are molecular DNA scissors that can be used to replace, eliminate or modify sequences in a highly targeted way. By modifying their recognition sequence through protein engineering, the targeted sequence can be changed.

The term “homing endonuclease” is used in accordance with its plain ordinary meaning and refers to a class of meganucleases encoded either as freestanding genes within introns, as fusions with host proteins, or as self-splicing protein introns. Endonucleases are enzymes that cleave the phosphodiester bond within a polynucleotide chain. In embodiments, the homing endonuclease is a LAGLIDADG endonuclease. In embodiments, the homing endonuclease has one LAGLIDADG (SEQ ID NO:1) structural motif. In embodiments, the homing endonuclease has two LAGLIDADG (SEQ ID NO:1) structural motifs.

As used herein, the term “homologous recombination” refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbial. Mal. Biol. Rev. 63:349-404 (1999)). In the method of the present invention, homologous recombination is enabled by the presence of said first and said second flanking element being placed upstream (5′) and downstream (3′), respectively, of said donor DNA sequence each of which being homologous to a continuous DNA sequence within said target sequence (e.g., retinoic acid receptor).

As used herein, the term “non-homologous end joining” (NHEJ) refers to cellular processes that join the two ends of double-strand breaks (DSBs) through a process largely independent of homology. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. NHEJ (non-homologous end-joining) pathways join the two ends of a DSB through a process largely independent of homology. Depending on the specific sequences and chemical modifications generated at the DSB, NHEJ may be precise or mutagenic (Lieber M R., The mechanism of double-strand DNA break repair by the nonhomologous DNA end-joining pathway. Annu Rev Biochem 79:181-211).

As used herein, the term “homologous recombination system” or “HR system” refers components of systems set out herein that maybe used to alter cells by homologous recombination. In particular, zinc-finger nucleases, TAL effector nucleases, CRISPR endonucleases, homing endonucleases, and Argonaute editing systems.

As used herein, the term “nucleic acid cutting entity” refers to a single molecule or a complex of molecules that has nucleic acid cutting activity (e.g., double-stranded nucleic acid cutting activity). Exemplary nucleic acid cutting entities include Argonuate complexes, zinc-finger proteins, transcription activator-like effectors (TALEs), CRISPR complexes, and homing endonucleases or meganucleases. In embodiments, nucleic acid cutting entities will have an activity that allows them to be nuclear localized (e.g., will contain nuclear localization signals (NLS)).

As used herein, the term “gene modulating reagents” encompasses “gene editing reagents” and “gene modulating nucleic acids.” At least one or more gene modulating reagents may be selected from the group including but not limited to: a CRISPR complex, a TAL effector nuclease, a zinc finger nuclease, a meganuclease, a homing endonuclease, an antisense nucleic acid, or an siRNA.

The term “gene editing reagents” refers to agents designed to cut intracellular DNA at the target locus or alter cells by homologous recombination. At least one or more gene editing reagents may be selected from the group including but not limited to: a CRISPR complex, a TAL effector nuclease, a zinc finger nuclease, a meganuclease, or a homing endonuclease. The term “gene modulating nucleic acids” refers to agents designed to reduce or inhibit expression of a gene or target gene. At least one or more gene modulating nucleic acids may be selected from the group including but not limited to: antisense nucleic acid or siRNA.

As used herein, the term “double-stranded break site” refers to a location in a nucleic acid molecule where a double-stranded break occurs. In embodiments, this will be generated by the nicking of the nucleic acid molecule at two close locations (e.g., within from about 3 to about 50 base pairs, from about 5 to about 50 base pairs, from about 10 to about 50 base pairs, from about 15 to about 50 base pairs, from about 20 to about 50 base pairs, from about 3 to about 40 base pairs, from about 5 to about 40 base pairs, from about 10 to about 40 base pairs, from about 15 to about 40 base pairs, from about 20 to about 40 base pairs, etc.). Typically, nicks may be further apart in nucleic acid regions that contain higher AT content, as compared to nucleic acid regions that contain higher GC content.

As used herein, the term “matched termini” refers to termini of nucleic acid molecules that share sequence identity of greater than 90%. A matched terminus of a double-strand break at a target locus may be double-stranded or single-stranded. A matched terminus of a donor nucleic acid molecule will generally be single-stranded.

An “antisense nucleic acid” as referred to herein is a nucleic acid (e.g., DNA or RNA molecule or derivative thereof or analog thereof) that is complementary to at least a portion of a specific target nucleic acid (e.g., an mRNA translatable into a protein) and is capable of reducing transcription of the target nucleic acid (e.g., mRNA from DNA) or reducing the translation of the target nucleic acid (e.g., mRNA) or altering transcript splicing (e.g., single stranded morpholino oligo). See, e.g., Weintraub, Scientific American, 262:40 (1990). Typically, synthetic antisense nucleic acids (e.g., oligonucleotides) are generally from 15 to 25 bases in length. Thus, antisense nucleic acids are capable of hybridizing to (e.g., selectively hybridizing to) a target nucleic acid (e.g., target mRNA). In embodiments, the antisense nucleic acid hybridizes to the target nucleic acid sequence (e.g., mRNA) under stringent hybridization conditions. In embodiments, the antisense nucleic acid hybridizes to the target nucleic acid (e.g., mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides, or a nucleotide analog described herein. Antisense nucleic acids include, for example, siRNA, microRNA and the like. In embodiments, an antisense nucleic acid is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, an antisense nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, an antisense nucleic acid is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, an antisense nucleic acids is from 10 to 15, 10 to 20, 10 to 30, 10 to 40, or 10 to 50 residues in length. In embodiments, an antisense nucleic acid is from 19 to 23 residues in length. In embodiments, an antisense nucleic acid is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical to a complementary sequence to the target gene or target nucleic acid (e.g., retinoic acid receptor or a fragment thereof).

The phrase “stringent hybridization conditions” refers to conditions under which a first nucleic acid will hybridize to its target subsequence, typically in a complex mixture of nucleic acid, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). In embodiments, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). In embodiments, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). In embodiments, stringent conditions may also be achieved with the addition of destabilizing agents such as formamide. In embodiments, for selective or specific hybridization, a positive signal is at least two times background, optionally 10 times background hybridization. In embodiments, exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. In embodiments, such washes can be performed for 5, 15, 30, 60, 120, or more minutes. Exemplary “moderately stringent hybridization conditions” may include a hybridization in a buffer of 40% formamide, 1 M NaCl, 1% SDS at 37° C., and a wash in 1×SSC at 45° C. Such washes can be performed for 5, 15, 30, 60, 120, or more minutes. In embodiments, a positive hybridization is at least twice background. Those of ordinary skill will readily recognize that alternative hybridization and wash conditions can be utilized to provide conditions of similar stringency.

An “siRNA,” “small interfering RNA,” “small RNA,” or “RNAi” as provided herein, refers to a nucleic acid that forms a double stranded RNA, which double stranded RNA (e.g., including nucleotide analog(s)) has the ability to reduce or inhibit expression of a gene or target gene when present in the same cell as the gene or target gene. The complementary portions of the nucleic acid that hybridize to form the double stranded molecule typically have substantial or complete identity. In one embodiment, an siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. In embodiments, an siRNA is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical to a complementary sequence to the target gene or target nucleic acid (e.g., retinoic acid receptor).

A “retinoic acid receptor inhibitor” refers to an agent (e.g., nucleic acid, protein, antibody, or compound) capable of detectably decreasing the level or the activity (e.g., level of activity of RAR protein or level of RAR activity in a cell, organ, tissue, subject, or vessel or level of RAR protein in a cell, organ, tissue, subject, or vessel or level of an RAR transcript in a cell, organ, tissue, subject, or vessel) of a retinoic acid receptor (RAR) when compared to a control, such as the absence of the inhibitor, or an agent with known inactivity. In embodiments, the retinoic acid receptor inhibitor is a compound, an aptamer, an antibody, a gene modulating reagent (e.g., CRISPR complex, TAL effector nuclease, zinc-finger nuclease, meganuclease, homing endonuclease, antisense nucleic acid, or siRNA), as disclosed herein, that reduces the level of activity (e.g., in a cell, of the protein, in an organism, in an organ, in a retinal ganglion cell) of retinoic acid receptor (RAR) when compared to a control, such as absence of the inhibitor or a compound, an aptamer, an antibody, or a gene modulating reagent (e.g., CRISPR complex, TAL effector nuclease, zinc-finger nuclease, meganuclease, homing endonuclease, antisense nucleic acid, or siRNA), with known inactivity. In embodiments, the retinoic acid receptor inhibitor is a compound (e.g., a compound described herein). In embodiments, the retinoic acid receptor inhibitor is an aptamer. In embodiments, the retinoic acid receptor inhibitor is an antibody. In embodiments, the retinoic acid receptor inhibitor is a gene modulating reagent. In embodiments, the retinoic acid receptor inhibitor is a CRISPR complex. In embodiments, the retinoic acid receptor inhibitor is a TAL effector nuclease. In embodiments, the retinoic acid receptor inhibitor is a zinc-finger nuclease. In embodiments, the retinoic acid receptor inhibitor is a meganuclease. In embodiments, the retinoic acid receptor inhibitor is a homing endonuclease. In embodiments, the retinoic acid receptor inhibitor is an antisense nucleic acid. In embodiments, the retinoic acid receptor inhibitor is an siRNA. In embodiments, the retinoic acid receptor inhibitor is an RAR antagonist. In embodiments, the RAR antagonist inhibits the binding of a nuclear receptor coactivator to the retinoic acid receptor. In embodiments, the retinoic acid receptor inhibitor is an RAR inverse agonist. In embodiments, the RAR inhibitor is an inhibitor described in Germain et al. Pharmacological reviews, 58(4), 712-725; 2006, which is incorporated herein by reference in its entirety. In embodiments, the retinoic acid receptor inhibitor is

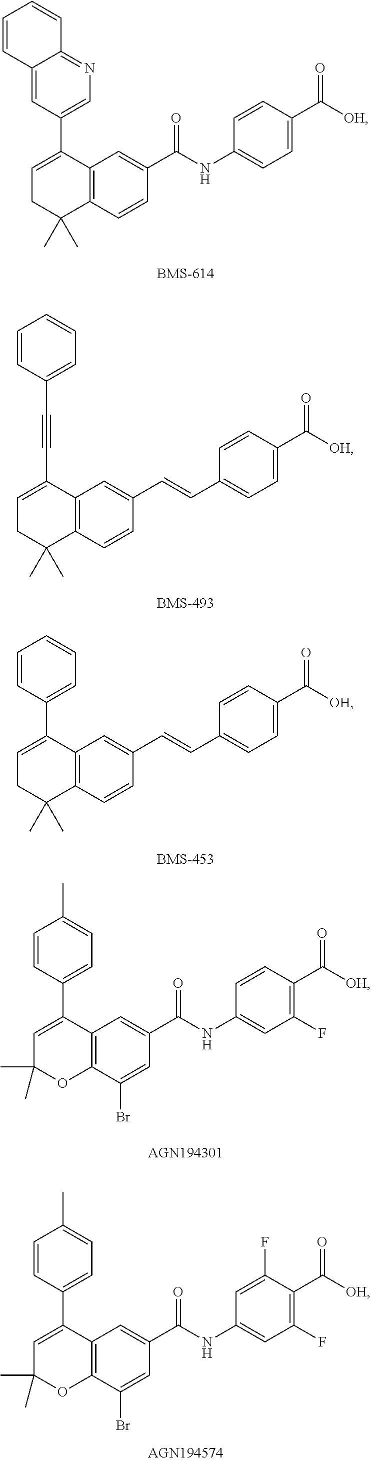 [SMILES: CC1(C)CC=C(c2cnc3ccccc3c2)c2cc(C(=O)Nc3ccc(C(=O)O)cc3)ccc21 ; CC1(C)CC=C(C#Cc2ccccc2)c2cc(/C=C/c3ccc(C(=O)O)cc3)ccc21 ; CC1(C)CC=C(c2ccccc2)c2cc(/C=C/c3ccc(C(=O)O)cc3)ccc21 ; Cc1ccc(C2=CC(C)(C)Oc3c(Br)cc(C(=O)Nc4ccc(C(=O)O)c(F)c4)cc32)cc1 ; Cc1ccc(C2=CC(C)(C)Oc3c(Br)cc(C(=O)Nc4cc(F)c(C(=O)O)c(F)c4)cc32)cc1]

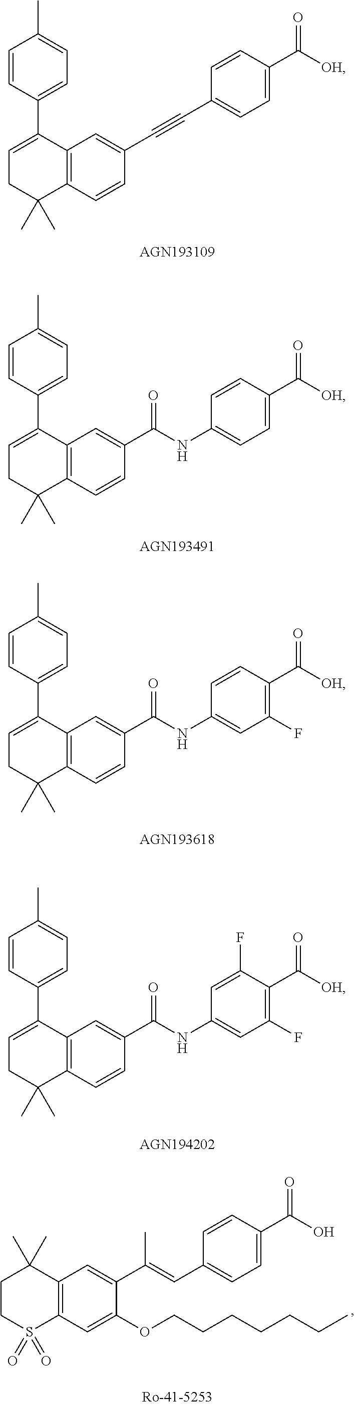 [SMILES: Cc1ccc(C2=CCC(C)(C)c3ccc(C#Cc4ccc(C(=O)O)cc4)cc32)cc1 ; Cc1ccc(C2=CCC(C)(C)c3ccc(C(=O)Nc4ccc(C(=O)O)cc4)cc32)cc1 ; Cc1ccc(C2=CCC(C)(C)c3ccc(C(=O)Nc4ccc(C(=O)O)c(F)c4)cc32)cc1 ; Cc1ccc(C2=CCC(C)(C)c3ccc(C(=O)Nc4cc(F)c(C(=O)O)c(F)c4)cc32)cc1 ; CCCCCCCOc1cc2c(cc1/C(C)=C/c1ccc(C(=O)O)cc1)C(C)(C)CCS2(=O)=O]

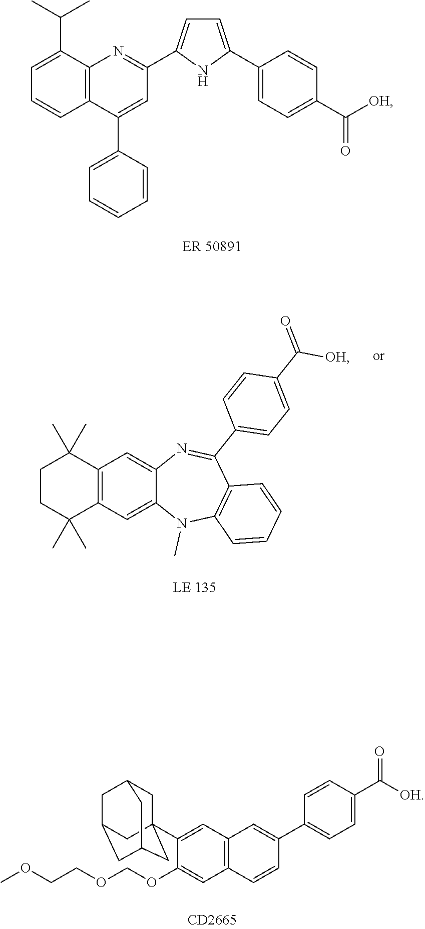 [SMILES: CC(C)c1cccc2c(-c3ccccc3)cc(-c3ccc(-c4ccc(C(=O)O)cc4)[nH]3)nc12 ; CN1c2cc3c(cc2N=C(c2ccc(C(=O)O)cc2)c2ccccc21)C(C)(C)CCC3(C)C ; "or" ; COCCOCOc1cc2ccc(-c3ccc(C(=O)O)cc3)cc2cc1C12CC3CC(CC(C3)C1)C2]

The terms “reverse agonist” or “inverse agonist” are used interchangeably and are used with their commonly understood meaning in the field of pharmacology, wherein an inverse agonist refers to an agent (e.g., a compound described herein) that binds to the same receptor as an agonist (e.g., retinoic acid receptor) but induces a pharmacological response opposite to that agonist (e.g., reduces the activity of retinoic acid receptor (RAR) when compared to a control, such as absence of the compound or a compound with known inactivity. In embodiments, the RAR inverse agonist increases the binding of a nuclear receptor corepressor to the retinoic acid receptor. The inverse agonist can decrease expression or activity at least 10%, 20%, 30%, 40%, 50%, 60%, 70%, 80%, 90%, 95%, 96%, 97%, 98%, or 99% in comparison to a control in the absence of the inverse agonist. In certain instances, expression or activity is 1.5-fold, 2-fold, 3-fold, 4-fold, 5-fold, 10-fold or lower than the expression or activity in the absence of the inverse agonist.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The terms “RAR” and “retinoic acid receptor” refer to a protein (including homologs, isoforms, and functional fragments thereof) which behave as ligand-activated transcription regulators. In embodiments, the retinoic acid receptor is RARα, RARβ, or RARγ. The term includes any recombinant or naturally-occurring form of RAR (e.g., RARα, RARβ, or RARγ) variants thereof that maintain RAR activity (e.g., within at least 30%, 40%, 50%, 60%, 70%, 80%, 90%, 95%, or 100% activity compared to wildtype RAR). In embodiments, the RARα protein encoded by the RARA gene has the amino acid sequence set forth in or corresponding to Entrez 5914, UniProt P10276, UniProt Q619R7, RefSeq (mRNA) NM_000964.3 (SEQ ID NO:2), RefSeq (mRNA) NM_001024809, RefSeq (mRNA) NM_001033603, RefSeq (mRNA) NM_001145301, RefSeq (mRNA) NM_001145302, RefSeq (protein) NP_000955.1 (SEQ ID NO:3), RefSeq (protein) NP_001019980, RefSeq (protein) NP_001138773, or RefSeq (protein) NP_001138774. In embodiments, the RARα protein has the following nucleic acid sequence:

(SEQ ID NO: 2)
GTGCCTCTTGCAGCAGCCTAACCCAGAAGCAGGGGGGAATCCTGAA
TCGAGCTGAGAGGGCTTCCCCGGTTCTCCTGGGAACCCCATCGGCC
CCCTGCCAGCACACACCTGAGCAGCATCACAGGACATGGCCCCCTC
AGCCACCTAGCTGGGGCCCATCTAGGAGTGGCATCTTTTTTGGTGC
CCTGAAGGCCAGCTCTGGACCTTCCCAGGAAAAGTGCCAGCTCACA
GAACTGCTTGACCAAAGGACCGGCTCTTGAGACATCCCCCAACCCA
CCTGGCCCCCAGCTAGGGTGGGGGCTCCAGGAGACTGAGATTAGCC
TGCCCTCTTTGGACAGCAGCTCCAGGACAGGGCGGGTGGGCTGACC
ACCCAAACCCCATCTGGGCCCAGGCCCCATGCCCCGAGGAGGGGTG
GTCTGAAGCCCACCAGAGCCCCCTGCCAGACTGTCTGCCTCCCTTC
TGACTGTGGCCGCTTGGCATGGCCAGCAACAGCAGCTCCTGCCCGA
CACCTGGGGGCGGGCACCTCAATGGGTACCCGGTGCCTCCCTACGC
CTTCTTCTTCCCCCCTATGCTGGGTGGACTCTCCCCGCCAGGCGCT
CTGACCACTCTCCAGCACCAGCTTCCAGTTAGTGGATATAGCACAC
CATCCCCAGCCACCATTGAGACCCAGAGCAGCAGTTCTGAAGAGAT
AGTGCCCAGCCCTCCCTCGCCACCCCCTCTACCCCGCATCTACAAG
CCTTGCTTTGTCTGTCAGGACAAGTCCTCAGGCTACCACTATGGGG
TCAGCGCCTGTGAGGGCTGCAAGGGCTTCTTCCGCCGCAGCATCCA
GAAGAACATGGTGTACACGTGTCACCGGGACAAGAACTGCATCATC
AACAAGGTGACCCGGAACCGCTGCCAGTACTGCCGACTGCAGAAGT
GCTTTGAAGTGGGCATGTCCAAGGAGTCTGTGAGAAACGACCGAAA
CAAGAAGAAGAAGGAGGTGCCCAAGCCCGAGTGCTCTGAGAGCTAC
ACGCTGACGCCGGAGGTGGGGGAGCTCATTGAGAAGGTGCGCAAAG
CGCACCAGGAAACCTTCCCTGCCCTCTGCCAGCTGGGCAAATACAC
TACGAACAACAGCTCAGAACAACGTGTCTCTCTGGACATTGACCTC
TGGGACAAGTTCAGTGAACTCTCCACCAAGTGCATCATTAAGACTG
TGGAGTTCGCCAAGCAGCTGCCCGGCTTCACCACCCTCACCATCGC
CGACCAGATCACCCTCCTCAAGGCTGCCTGCCTGGACATCCTGATC
CTGCGGATCTGCACGCGGTACACGCCCGAGCAGGACACCATGACCT
TCTCGGACGGGCTGACCCTGAACCGGACCCAGATGCACAACGCTGG
CTTCGGCCCCCTCACCGACCTGGTCTTTGCCTTCGCCAACCAGCTG
CTGCCCCTGGAGATGGATGATGCGGAGACGGGGCTGCTCAGCGCCA
TCTGCCTCATCTGCGGAGACCGCCAGGACCTGGAGCAGCCGGACCG
GGTGGACATGCTGCAGGAGCCGCTGCTGGAGGCGCTAAAGGTCTAC
GTGCGGAAGCGGAGGCCCAGCCGCCCCCACATGTTCCCCAAGATGC
TAATGAAGATTACTGACCTGCGAAGCATCAGCGCCAAGGGGGCTGA
GCGGGTGATCACGCTGAAGATGGAGATCCCGGGCTCCATGCCGCCT
CTCATCCAGGAAATGTTGGAGAACTCAGAGGGCCTGGACACTCTGA
GCGGACAGCCGGGGGGTGGGGGGCGGGACGGGGGTGGCCTGGCCCC
CCCGCCAGGCAGCTGTAGCCCCAGCCTCAGCCCCAGCTCCAACAGA
AGCAGCCCGGCCACCCACTCCCCGTGACCGCCCACGCCACATGGAC
ACAGCCCTCGCCCTCCGCCCCGGCTTTTCTCTGCCTTTCTACCGAC
CATGTGACCCCGCACCAGCCCTGCCCCCACCTGCCCTCCCGGGCAG
TACTGGGGACCTTCCCTGGGGGACGGGGAGGGAGGAGGCAGCGACT
CCTTGGACAGAGGCCTGGGCCCTCAGTGGACTGCCTGCTCCCACAG
CCTGGGCTGACGTCAGAGGCCGAGGCCAGGAACTGAGTGAGGCCCC
TGGTCCTGGGTCTCAGGATGGGTCCTGGGGGCCTCGTGTTCATCAA
GACACCCCTCTGCCCAGCTCACCACATCTTCATCACCAGCAAACGC
CAGGACTTGGCTCCCCCATCCTCAGAACTCACAAGCCATTGCTCCC
CAGCTGGGGAACCTCAACCTCCCCCCTGCCTCGGTTGGTGACAGAG
GGGGTGGGACAGGGGCGGGGGGTTCCCCCTGTACATACCCTGCCAT
ACCAACCCCAGGTATTAATTCTCGCTGGTTTTGTTTTTATTTTAAT
TTTTTTGTTTTGATTTTTTTAATAAGAATTTTCATTTTAAGCACAT
TTATACTGAAGGAATTTGTGCTGTGTATTGGGGGGAGCTGGATCCA
GAGCTGGAGGGGGTGGGTCCGGGGGAGGGAGTGGCTCGGAAGGGGC
CCCCACTCTCCTTTCATGTCCCTGTGCCCCCCAGTTCTCCTCCTCA
GCCTTTTCCTCCTCAGTTTTCTCTTTAAAACTGTGAAGTACTAACT
TTCCAAGGCCTGCCTTCCCCTCCCTCCCACTGGAGAAGCCGCCAGC
CCCTTTCTCCCTCTGCCTGACCACTGGGTGTGGACGGTGTGGGGCA
GCCCTGAAAGGACAGGCTCCTGGCCTTGGCACTTGCCTGCACCCAC
CATGAGGCATGGAGCAGGGCAGAGCAAGGGCCCCGGGACAGAGTTT
TCCCAGACCTGGCTCCTCGGCAGAGCTGCCTCCCGTCAGGGCCCAC
ATCATCTAGGCTCCCCAGCCCCCACTGTGAAGGGGCTGGCCAGGGG
CCCGAGCTGCCCCCACCCCCGGCCTCAGCCACCAGCACCCCCATAG
GGCCCCCAGACACCACACACATGCGCGTGCGCACACACACAAACAC
ACACACACTGGACAGTAGATGGGCCGACACACACTTGGCCCGAGTT
CCTCCATTTCCCTGGCCTGCCCCCCACCCCCAACCTGTCCCACCCC
CGTGCCCCCTCCTTACCCCGCAGGACGGGCCTACAGGGGGGTCTCC
CCTCACCCCTGCACCCCCAGCTGGGGGAGCTGGCTCTGCCCCGACC
TCCTTCACCAGGGGTTGGGGCCCCTTCCCCTGGAGCCCGTGGGTGC
ACCTGTTACTGTTGGGCTTTCCACTGAGATCTACTGGATAAAGAAT
AAAGTTCTATTTATTCTAAAAAAAAAAAAAAAAAA.

In embodiments, the RARα protein has the following amino acid sequence:

(SEQ ID NO: 3)
MASNSSSCPTPGGGHLNGYPVPPYAFFFPPMLGGLSPPGALTTLQH
QLPVSGYSTPSPATIETQSSSSEEIVPSPPSPPPLPRIYKPCFVCQ
DKSSGYHYGVSACEGCKGFERRSIQKNMVYTCHRDKNCIINKVTRN
RCQYCRLQKCFEVGMSKESVRNDRNKKKKEVPKPECSESYTLTPEV
GELIEKVRKAHQETFPALCQLGKYTTNNSSEQRVSLDIDLWDKFSE
LSTKCIIKTVEFAKQLPGFTTLTIADQITLLKAACLDILILRICTR
YTPEQDTMTFSDGLTLNRTQMHNAGFGPLTDLVFAFANQLLPLEMD
DAETGLLSAICLICGDRQDLEQPDRVDMLQEPLLEALKVYVRKRRP
SRPHMFPKMLMKITDLRSISAKGAERVITLKMEIPGSMPPLIQEML
ENSEGLDTLSGQPGGGGRDGGGLAPPPGSCSPSLSPSSNRSSPATH
SP.

In embodiments, the RARβ protein encoded by the RARB gene has the amino acid sequence set forth in or corresponding to Entrez 5915, UniProt P10826, UniProt Q5QHG3, RefSeq (mRNA) NM_000965, RefSeq (mRNA) NM_001290216, RefSeq (mRNA) NM_001290217, RefSeq (mRNA) NM_001290266, RefSeq (mRNA) NM_001290276, RefSeq (protein) NP_000956, RefSeq (protein) NP_001277145, RefSeq (protein) NP_001277146, RefSeq (protein) NP_001277195, or RefSeq (protein) NP_001277205. In embodiments, the RARγ protein encoded by the RARG gene has the amino acid sequence set forth in or corresponding to Entrez 5916, UniProt P13631, RefSeq (mRNA) NM_000966, RefSeq (mRNA) NM_001042728, RefSeq (mRNA) NM_001243730, RefSeq (mRNA) NM_001243731, RefSeq (mRNA) NM_001243732, RefSeq (protein) NP_000957, RefSeq (protein) NP_001036193, RefSeq (protein) NP_001230659, RefSeq (protein) NP_001230660, or RefSeq (protein) NP_001230661. In embodiments, the RAR is a human RAR. Members of the RAR family (e.g., RARα, RARβ, or RARγ) bind to specific DNA elements as a heterodimer with a retinoid X receptor (RXR).

For each RAR subtype (e.g., RARα, RARβ, or RARγ), several isoforms exist which differ primarily in their N-terminal region. There are two major isoforms for RARα (α1 and β2) and for RARγ (γ1 and γ2) and four major isoforms for RARβ (β1, β2, β3, and β4). In embodiments, RAR refers to RARα (e.g., α1 and α2). In embodiments, RAR refers to RARβ (e.g., β1, β2, β3, and β4). In embodiments, RAR refers to RARγ (γ1 and γ2). RARs typically heterodimerize with the three retinoid X receptors, RXRα, RXRβ, or RXRγ, which then act as ligand-dependent transcriptional regulators.

The terms “RXR” and “retinoid X receptor” refer to a protein (including homologs, isoforms, and functional fragments thereof) which behave as ligand-activated transcription regulators. In embodiments, the retinoid X receptor is RXRα, RXRβ, or RXRγ. The term includes any recombinant or naturally-occurring form of RXR (e.g., RXRα, RXRβ, or RXRγ) variants thereof that maintain RXR activity (e.g., within at least 30%, 40%, 50%, 60%, 70%, 80%, 90%, 95%, or 100% activity compared to wildtype RXR). In embodiments, the RXRα protein encoded by the RXRA gene has the amino acid sequence set forth in or corresponding to Entrez 6256, UniProt P19793, UniProt F1D8Q5, RefSeq (mRNA) NM_002957, RefSeq (mRNA) NM_001291921, RefSeq (protein) NP_002948, or RefSeq (protein) NP_001278850. In embodiments, the RXRβ protein encoded by the RXRB gene has the amino acid sequence set forth in or corresponding to Entrez 6257, UniProt P28702, UniProt Q5STP9, RefSeq (mRNA) NM_021976, RefSeq (mRNA) NM_001270401, RefSeq (protein) NP_068811, or RefSeq (protein) NP_001257330. In embodiments, the RXRy protein encoded by the RXRG gene has the amino acid sequence set forth in or corresponding to Entrez 6258, UniProt P48443, UniProt B6ZGT6, RefSeq (mRNA) NM_006917, RefSeq (mRNA) NM_001256571, RefSeq (protein) NP_008848, or RefSeq (protein) NP_001243500. In embodiments, the RXR is a human RXR.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. In some embodiments, a vision loss associated disease modulator is a compound that reduces the severity of one or more symptoms of a disease associated with vision loss.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., vision loss or vision degeneration is associated with photoreceptor degenerative diseases, including retinitis pigmentosa, Leber's congenital amaurosis, Usher's syndrome, Bardet-Biedl syndrome, Stargardt disease, age-related macular degeneration, cone dystrophy, or rod-cone dystrophy) means that the disease (e.g., vision loss, vision degeneration, photoreceptor degenerative diseases, including retinitis pigmentosa, Leber's congenital amaurosis, Usher's syndrome, Bardet-Biedl syndrome, Stargardt disease, age-related macular degeneration, cone dystrophy, or rod-cone dystrophy) is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components. For example, binding of a RAR with an agent (e.g., retinoic acid receptor inhibitor or compound described herein) as described herein may reduce the level of a product of the RAR catalyzed reaction or the level of a downstream derivative of the product or binding may reduce the interactions between the RAR protein or an RAR reaction product and downstream effectors or signaling pathway components, resulting in changes in expression, protein activity, cell growth, proliferation, or survival.

The terms “disease” or “condition” refer to a state of being or health status of a patient or subject capable of being treated with the compounds or methods provided herein. The disease may be a photoreceptor degenerative disease. The disease may be retinitis pigmentosa. The disease may be Leber's congenital amaurosis. The disease may be Usher's syndrome. The disease may be Bardet-Biedl syndrome. The disease may be Stargardt disease. The disease may be age-related macular degeneration. The disease may be cone dystrophy. The disease may be a rod-cone dystrophy.

The terms “treating” or “treatment” refers to any indicia of success in the therapy or amelioration of an injury, disease, pathology or condition (e.g., vision regeneration), including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. The treatment or amelioration of symptoms can be based on visual performance as measured by electrophysiological method such as electroretinogram (ERG) or visual evoked potential recording (VEP) and psychophysical parameters including visual threshold, contrast sensitivity, visual acuity, or flicker fusion rate.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms (e.g., vision degeneration) or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms (e.g., vision degeneration) in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist or inverse agonist required to decrease the activity of an enzyme relative to the absence of the antagonist or inverse agonist. A “function disrupting amount,” as used herein, refers to the amount of antagonist or inverse agonist required to disrupt the function of an enzyme or protein relative to the absence of the antagonist or inverse agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” is meant that a composition described herein is administered at the same time, just prior to, or just after the administration of one or more additional therapies.

The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The terms “vision loss” and “vision degeneration” are used interchangeably and refer to their common ordinary meaning, namely impairment of vision, for example, as a result of degeneration of rod and/or cone photoreceptors. Visual degeneration is typically diagnosed via an eye exam. In embodiments, vision loss is characterized as a reduction in overall vision (e.g., a 1-99% reduction in overall vision). In embodiments, vision loss refers to complete blindness. In embodiments, vision loss is characterized as blurred or no vision in the center of the visual field. In embodiments vision loss is measured by electrophysiological method such as electroretinogram (ERG) or visual evoked potential recording (VEP) and/or psychophysical parameters including visual threshold, contrast sensitivity, visual acuity, or flicker fusion. In embodiments, symptoms of vision degeneration include night blindness or nyctalopia; tunnel vision, loss of peripheral vision, latticework vision; photopsia (e.g., blinking/shimmering lights), photophobia (e.g., aversion to glare), development of bone spicules in the fundus, loss of central vision, slow adjustment from dark to light environments and vice versa, blurring of vision, poor color separation, and/or the loss of the mid-peripheral visual field. In embodiments, vision degeneration is associated with Usher syndrome, Alport's syndone, Kearns-Sayre syndrome, abetalipoproteinemia, McLeod syndrome, Bardet-Biedl syndrome, neurosyphilis, toxoplasmosis, or Refsum's disease. In embodiments, vision loss is not complete blindness. In embodiments, vision degeneration is associated with Retinitis Pigmentosa, Cone Dystrophy, Rod Distrophy, Rod-cone Distrophy, Cone-Rod Distrophy, Bardet-Biedl syndrome, Leber congenital amaurosis, macular degeneration, age-related macular degeneration, Senior-Loken syndrome with retinitis pigmentosa or LCA, Joubert syndrome with retinitis pigmentosa, Alström syndrome with CRD, Meckel syndrome, retinitis pigmentosa in ciliopathies, Usher syndrome, Bietti crystalline corneoretinal dystrophy, Stargardt's Disease, Abetalipoproteinaemia, Refsum disease, Zellweger syndrome, Oguchi disease, Stargardt disease, fundus flavimaculatus, Bothnia dystrophy, retinitis punctata albescens, Newfoundland CRD, vitreoretinochoroidopathy, bestrophinopathy, Doyne honeycomb retinal degeneration (Malattia Leventinese), retinoschisis, Sorsby's fundus dystrophy, vitreoretinopathy in Stickler syndrome, digenic exudative vitreoretinopathy, retinopathy of prematurity, familial exudative vitreoretinopathy, Wagner disease, erosive vitreoretinopathy, gyrate atrophy, Hallervorden-Spatz syndrome, spinocerebellar ataxia with macular dystrophy, Goldmann-Favre syndrome, Sveinsson chorioretinal atrophy, Kearns-Sayre syndrome, Leigh syndrome, Leber hereditary optic neuropathy, pigmented paravenous chorioretinal atrophy, maculopathy in pseudoxanthoma elasticum, Choroideremia, Batten disease with retinitis pigmentosa, Jalili syndrome, Alagille syndrome, microphthalmos, or retinal disease syndrome.

The term “nuclear receptor corepressor” is used in accordance with its plain ordinary meaning and refers to transcriptional coregulatory proteins which contains multiple nuclear receptor interacting domains that typically decrease and/or silence gene expression. Non-limiting examples include nuclear receptor co-repressor 1 (NCOR1) (e.g., Entrez 9611, UniProt O75376, RefSeq (mRNA) NM_001190438.1, RefSeq (mRNA) NM_001190440.1, RefSeq (mRNA) NM_006311.3, RefSeq (protein) NP_001177367.1, RefSeq (protein) NP_001177369.1, or RefSeq (protein) NP_006302.2) and nuclear receptor co-repressor 2 (NCOR2) also referred to herein as silencing mediator of retinoic acid and thyroid hormone receptor (SMRT) (e.g., Entrez 9612, UniProt Q9Y618, RefSeq (mRNA) NM_006312.5, RefSeq (mRNA) NM_001077261.3, RefSeq (mRNA) NM_001206654.1, RefSeq (protein) NP_001070729.2, RefSeq (protein) NP_001193583.1, or RefSeq (protein) NP_006303.4).

The term “nuclear receptor coactivator” is used in accordance with its plain ordinary meaning and refers to transcriptional coregulatory proteins which contains multiple nuclear receptor interacting domains that typically increase gene expression by binding to an activator (e.g., transcription factor) which contains a DNA binding domain. Non-limiting examples include nuclear receptor coactivator 1 (NCOA1) (e.g., Entrez 8648, UniProt Q15788, RefSeq (mRNA) NM_003743.4, RefSeq (mRNA) NM_147223.2, RefSeq (mRNA) NM_147233.2, RefSeq (protein) NP_003734.3, RefSeq (protein) NP_671756.1, or RefSeq (protein) NP_671766.1) and nuclear receptor coactivator 2 (NCOA2) also referred to herein as glucocorticoid receptor-interacting protein 1 (GRIP1), steroid receptor coactivator-2 (SRC-2), or transcriptional mediators/intermediary factor 2 (TIF2) (e.g., Entrez 10499, UniProt Q15596, RefSeq (mRNA) NM_006540.3, RefSeq (mRNA) NM_001321703.1, RefSeq (mRNA) NM_001321707.1, RefSeq (mRNA) NM_001321711.1, RefSeq (mRNA) NM_001321712.1, RefSeq (protein) NP_001308632.1, RefSeq (protein) NP_001308636.1, RefSeq (protein) NP_001308640.1, RefSeq (protein) NP_001308641.1, or RefSeq (protein) NP_001308642.1).

The terms “ATP-gated P2X receptor cation channel” or “P2X receptor” refer to membrane receptors consisting of cation-permeable ligand-gated ion channels that open in response to the binding of extracellular adenosine 5′triphosphate (ATP). Genes coding for P2X subunits include, for example, P2RX1 (e.g., Entrez 5023, UniProt P51575, RefSeq (mRNA) NM_002558, or RefSeq (protein) NP_002549), P2RX2 (e.g., Entrez 22953, UniProt Q9UBL9, RefSeq (mRNA) NM_001282164, RefSeq (mRNA) NM_001282165, RefSeq (mRNA) NM_012226, RefSeq (mRNA) NM_016318, RefSeq (mRNA) NM_170682, RefSeq (protein) NP_001269093, RefSeq (protein) NP_001269094, RefSeq (protein) NP_036358, RefSeq (protein) NP_057402, or RefSeq (protein) NP_733782), P2RX3 (e.g., Entrez 5024, UniProt P56373, RefSeq (mRNA) NM_002559, or RefSeq (protein) NP_002550), P2RX4 (e.g., Entrez 5025, UniProt Q99571, RefSeq (mRNA) NM_001256796, RefSeq (mRNA) NM_001261397, RefSeq (mRNA) NM_001261398, RefSeq (mRNA) NM_002560, RefSeq (mRNA) NM_175567, RefSeq (protein) NP_001243725, RefSeq (protein) NP_001248326, RefSeq (protein) NP_001248327, or RefSeq (protein) NP_002551), P2RX5 (e.g., Entrez 5026, UniProt Q93086, RefSeq (mRNA) NM_175081, RefSeq (mRNA) NM_001204519, RefSeq (mRNA) NM_001204520, RefSeq (mRNA) NM_002561, RefSeq (mRNA) NM_175080, RefSeq (protein) NP_001191448, RefSeq (protein) NP_001191449, RefSeq (protein) NP_002552, or RefSeq (protein) NP_778255), P2RX6 (e.g., Entrez 9127, UniProt 015547, RefSeq (mRNA) NM_001159554, RefSeq (mRNA) NM_005446, RefSeq (mRNA) NM_001349874, RefSeq (mRNA) NM_001349875, RefSeq (mRNA) NM_001349876, RefSeq (protein) NP_001153026, RefSeq (protein) NP_005437, RefSeq (protein) NP_001336803, RefSeq (protein) NP_001336804, or RefSeq (protein) NP_001336805), or P2RX7 (e.g., Entrez 5027, UniProt Q99572, RefSeq (mRNA) NM_002562, RefSeq (mRNA) NM_177427, or RefSeq (protein) NP_002553).

The terms “HCN channel” or “hyperpolarization-activated cyclic nucleotide-gated channel” as used herein refer to nonselective ligand-gated cation channels in the plasma membranes. In embodiments, the HCN channel is the HCN1 channel, HCN2 channel, HCN3 channel, or the HCN4 channel. In embodiments, the HCN channel is the HCN1 channel (e.g., Entrez 348980, UniProt 060741, RefSeq (mRNA) NM_021072.3, or RefSeq (protein NP_066550.2)). In embodiments, the HCN channel is the HCN2 channel (e.g., Entrez 610, UniProt Q9UL51, RefSeq (mRNA) NM_001194.3, or RefSeq (protein NP_001185.3)). In embodiments, the HCN channel is the HCN3 channel (e.g., Entrez 57657, UniProt Q9P1Z3, RefSeq (mRNA) NM_020897.2, or RefSeq (protein NP_065984.1)). In embodiments, the HCN channel is the HCN4 channel (e.g., Entrez 10021, UniProt Q9Y3Q4, RefSeq (mRNA) NM_005477.2, or RefSeq (protein NP_005468.1)).

The term light sensitivity, in the context of light sensitivity of retinal ganglion cells, refers to the ability of retinal ganglion cells to detect light and transmit visual information in the form of an action potential. Light sensitivity may be measured by electrophysiological method such as electroretinogram (ERG) or visual evoked potential recording (VEP) and psychophysical parameters including visual threshold, contrast sensitivity, visual acuity, or flicker fusion rate.

The term hyperexcitability, in the context of hyperexcitability of retinal ganglion cells, refers to the spontaneous transmission of action potentials of retinal ganglion cells. Blindness occurs in the face of sustained hyperactivity among retinal ganglion cells. In embodiments, retinal ganglion cells experiencing hyperexcitability begin firing spontaneously in darkness at rates many times greater than normal. In embodiments, hyperactivity refers to a higher rate of spontaneous firing in darkness. In embodiments, hyperexcitability does not include light-evoked activity. Hyperexcitability may be measured according to the techniques put forth in Stasheff, S. F. Journal of neurophysiology, 99(3), 1408-1421 (2008); and Stasheff, S. F. et al. Journal of neurophysiology, 105(6), 3002-3009 (2011), which are incorporated herein by reference in their entirety for all purposes.

The term hyperpermeability is used in accordance with its plain ordinary meaning. In embodiments, a cell having hyperpermeability is a cell having greater permeability (e.g., to ions, cations, anions, sodium ion, calcium ion, chloride ion, small molecules) than in a normal non-diseased state of the same cell.

The term retinaldehyde dehydrogenase inhibitor, as used herein, refers to an agent (e.g., compound) which reduces the level or activity of retinaldehyde dehydrogenase relative to a control (e.g., the absence of the inhibitor). In embodiments, the retinaldehyde dehydrogenase inhibitor reduces the level of retinoic acid. Non-limiting examples of retinaldehyde dehydrogenase inhibitors include ampal, benomyl, citral, chloral hydrate, coprine, cyanamide, diadzin, CVT-10216, DEAB, DPAB, dislfiram, gossypol, molinate, nitroglycerin, and pargyline. Additional details and mechanistic insight into retinaldehyde dehydrogenase inhibitors may be found in Koppaka et al. Pharmacol Rev. 2012 July; 64(3): 520-539, which is incorporated herein by reference in its entirety for all purposes.

### II. Compounds

In an aspect is provided a retinoic acid receptor inhibitor, having the formula:

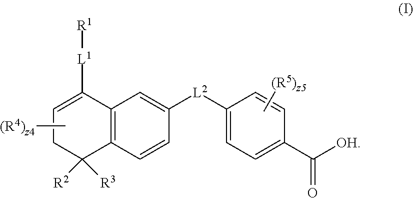

L¹ is a bond, —S(O)₂—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is —S(O)₂—, —NH—, —O—, —S—, —C(O)—, —C(O)NH—, —NHC(O)—, —NHC(O)NH—, —C(O)O—, —OC(O)—, —C(S)—, —C(S)NH—, —NHC(S)—, —NHC(S)NH—, —C(S)O—, —OC(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² and R³ are each independently hydrogen, or substituted or unsubstituted alkyl, or substituted or unsubstituted heteroalkyl; R² and R³ may optionally be joined to form a substituted or unsubstituted cycloalkyl, substituted or unsubstituted heterocycloalkyl, substituted or unsubstituted aryl, or substituted or unsubstituted heteroaryl. R⁴ and R⁵ are each independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol z4 is an integer from 0 to 3. The symbol z5 is an integer from 0 to 4.

In embodiments, L¹ is a bond, —S(O)₂—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered).

In embodiments, L¹ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene.

In embodiments, L¹ is unsubstituted alkylene, unsubstituted heteroalkylene, unsubstituted cycloalkylene, unsubstituted heterocycloalkylene, unsubstituted arylene, or unsubstituted heteroarylene.

In embodiments, L¹ is substituted (e.g., substituted with a substituent group, a size-limited substituent group, or lower substituent group) or unsubstituted alkylene. In embodiments, L¹ is substituted (e.g., substituted with a substituent group, a size-limited substituent group, or lower substituent group) alkylene. In embodiments, L¹ is unsubstituted alkylene. In embodiments, L¹ is substituted or unsubstituted alkylene (e.g., C₁-C₈, C₁-C₆, C₁-C₄, or C₁-C₂). In embodiments, L¹ is substituted alkylene (e.g., C₁-C₈, C₁-C₆, C₁-C₄, or C₁-C₂). In embodiments, L¹ is unsubstituted alkylene (e.g., C₁-C₈, C₁-C₆, C₁-C₄, or C₁-C₂).

In embodiments, L¹ is substituted (e.g., substituted with a substituent group, a size-limited substituent group, or lower substituent group) or unsubstituted heteroalkylene. In embodiments, L¹ is substituted (e.g., substituted with a substituent group, a size-limited substituent group, or lower substituent group) heteroalkylene. In embodiments, L¹ is unsubstituted heteroalkylene. In embodiments, L¹ is substituted or unsubstituted heteroalkylene (e.g., 2 to 8 membered, 2 to 6 membered, 4 to 6 membered, 2 to 3 membered, or 4 to 5 membered). In embodiments, L¹ is substituted heteroalkylene (e.g., 2 to 8 membered, 2 to 6 membered, 4 to 6 membered, 2 to 3 membered, or 4 to 5 membered). In embodiments, L¹ is an unsubstituted heteroalkylene (e.g., 2 to 8 membered, 2 to 6 membered, 4 to 6 membered, 2 to 3 membered, or 4 to 5 membered).

In embodiments, L¹ is substituted (e.g., substituted with a substituent group, a size-limited substituent group, or lower substituent group) or unsubstituted cycloalkylene. In embodiments, L¹ is substituted (e.g., substituted with a substituent group, a size-limited substituent group, or lower substituent group) cycloalkylene. In embodiments, L¹ is an unsubstituted cycloalkylene. In embodiments, L¹ is substituted or unsubstituted cycloalkylene (e.g., C₃-C₈, C₃-C₆, C₄-C₆, or C₅-C₆). In embodiments, L¹ is substituted cycloalkylene (e.g., C₃-C₈, C₃-C₆, C₄-C₆, or C₅-C₆). In embodiments, L¹ is unsubstituted cycloalkylene (e.g., C₃-C₈, C₃-C₆, C₄-C₆, or C₅-C₆).

In embodiments, L¹ is substituted (e.g., substituted with a substituent group, a size-limited substituent group, or lower substituent group) or unsubstituted heterocycloalkylene. In embodiments, L¹ is substituted (e.g., substituted with a substituent group, a size-limited substituent group, or lower substituent group) heterocycloalkylene. In embodiments, L¹ is an unsubstituted heterocycloalkylene. In embodiments, L¹ is substituted or unsubstituted heterocycloalkylene (e.g., 3 to 8 membered, 3 to 6 membered, 4 to 6 membered, 4 to 5 membered, or 5 to 6 membered). In embodiments, L¹ is substituted heterocycloalkylene (e.g., 3 to 8 membered, 3 to 6 membered, 4 to 6 membered, 4 to 5 membered, or 5 to 6 membered). In embodiments, L¹ an unsubstituted heterocycloalkylene (e.g., 3 to 8 membered, 3 to 6 membered, 4 to 6 membered, 4 to 5 membered, or 5 to 6 membered).

In embodiments, L¹ is substituted (e.g., substituted with a substituent group, a size-limited substituent group, or lower substituent group) or unsubstituted arylene. In embodiments, L¹ is substituted (e.g., substituted with a substituent group, a size-limited substituent group, or lower substituent group) arylene. In embodiments, L¹ is an unsubstituted arylene. In embodiments, L¹ is substituted or unsubstituted arylene (e.g., C₆-C₁₀ or phenylene). In embodiments, L¹ is substituted arylene (e.g., C₆-C₁₀ or phenylene). In embodiments, L¹ is an unsubstituted arylene (e.g., C₆-C₁₀ or phenylene).

In embodiments, L¹ is substituted (e.g., substituted with a substituent group, a size-limited substituent group, or lower substituent group) or unsubstituted heteroarylene. In embodiments, L¹ is substituted (e.g., substituted with a substituent group, a size-limited substituent group, or lower substituent group) heteroarylene. In embodiments, L¹ is an unsubstituted heteroarylene. In embodiments, L¹ is substituted or unsubstituted heteroarylene (e.g., 5 to 10 membered, 5 to 9 membered, or 5 to 6 membered). In embodiments, L¹ is substituted heteroarylene (e.g., 5 to 10 membered, 5 to 9 membered, or 5 to 6 membered). In embodiments, L¹ is an unsubstituted heteroarylene (e.g., 5 to 10 membered, 5 to 9 membered, or 5 to 6 membered).

In embodiments, -L¹-R¹ has the formula:

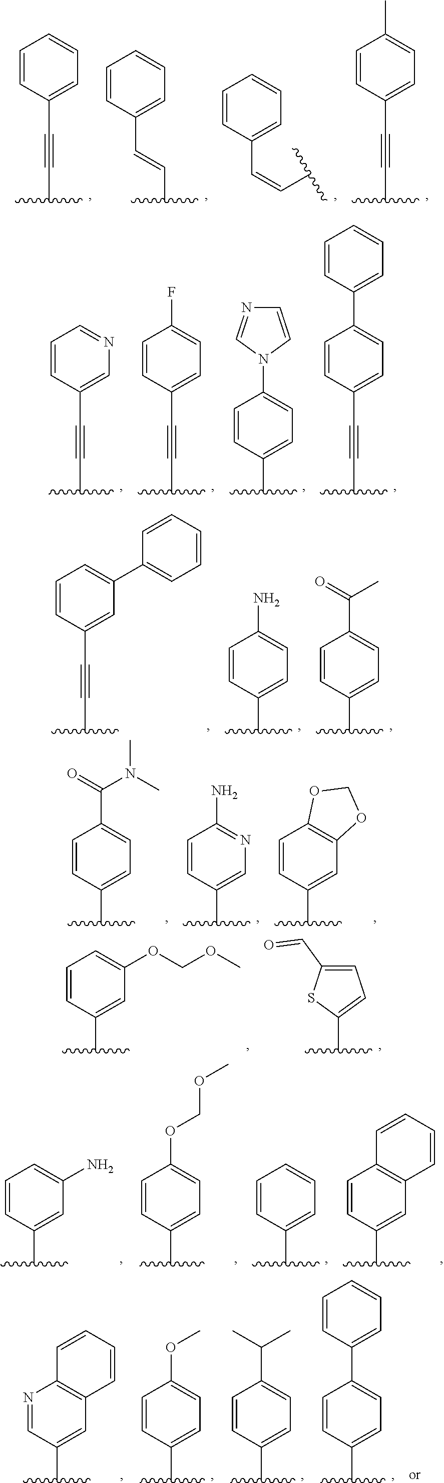

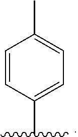 [SMILES: *c1ccc(C)cc1]

In embodiments, -L¹-R¹ has the formula

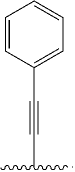 [SMILES: *C#Cc1ccccc1]

In embodiments, -L¹-R¹ has the formula

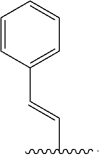 [SMILES: */C=C/c1ccccc1]

In embodiments, -L¹-R¹ has the formula

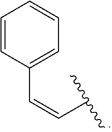 [SMILES: */C=C\c1ccccc1]

In embodiments, -L¹-R¹ has the formula

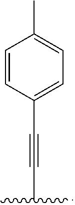 [SMILES: *C#Cc1ccc(C)cc1]

In embodiments, -L¹-R¹ has the formula

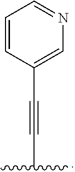 [SMILES: *C#Cc1cccnc1]

In embodiments, -L¹-R¹ has the formula

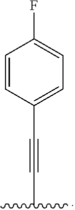 [SMILES: *C#Cc1ccc(F)cc1]

In embodiments, -L¹-R¹ has the formula

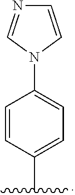 [SMILES: *c1ccc(-n2ccnc2)cc1]

In embodiments, -L¹-R¹ has the formula

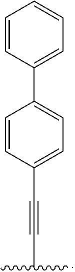 [SMILES: *C#Cc1ccc(-c2ccccc2)cc1]

In embodiments, -L¹-R¹ has the formula

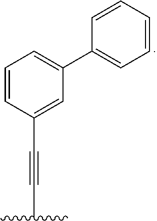 [SMILES: *C#Cc1cccc(-c2ccccc2)c1]

In embodiments, -L¹-R¹ has the formula

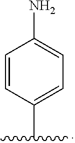 [SMILES: *c1ccc(N)cc1]

In embodiments, -L¹-R¹ has the formula

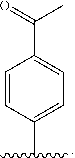 [SMILES: *c1ccc(C(C)=O)cc1]

In embodiments, -L¹-R¹ has the formula

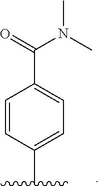 [SMILES: *c1ccc(C(=O)N(C)C)cc1]

In embodiments, -L¹-R¹ has the formula

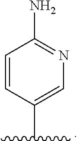 [SMILES: *c1ccc(N)nc1]

In embodiments, -L¹-R¹ has the formula

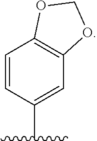 [SMILES: *c1ccc2c(c1)OCO2]

In embodiments, -L¹-R¹ has the formula

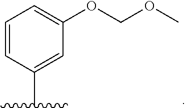 [SMILES: *c1cccc(OCOC)c1]

In embodiments, -L¹-R¹ has the formula

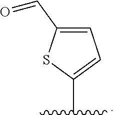 [SMILES: *c1ccc(C=O)s1]

In embodiments, -L¹-R¹ has the formula

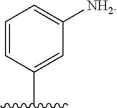 [SMILES: *c1cccc(N)c1]

In embodiments, -L¹-R¹ has the formula

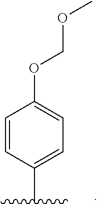 [SMILES: *c1ccc(OCOC)cc1]

In embodiments, -L¹-R¹ has the formula

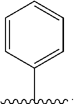 [SMILES: *c1ccccc1]

In embodiments, -L¹-R¹ has the formula

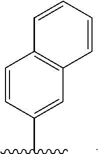 [SMILES: *c1ccc2ccccc2c1]

In embodiments, -L¹-R¹ has the formula

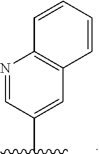 [SMILES: *c1cnc2ccccc2c1]

In embodiments, -L¹-R¹ has the formula

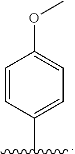 [SMILES: *c1ccc(OC)cc1]

In embodiments, -L¹-R¹ has the formula

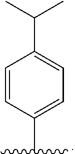 [SMILES: *c1ccc(C(C)C)cc1]

In embodiments, -L¹-R¹ has the formula

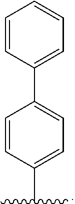 [SMILES: *c1ccc(-c2ccccc2)cc1]

In embodiments, -L¹-R¹ has the formula

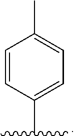 [SMILES: *c1ccc(C)cc1]

In embodiments, L² is —S(O)₂—, —NH—, —O—, —S—, —C(O)—, —C(O)NH—, —NHC(O)—, —NHC(O)NH—, —C(O)O—, —OC(O)—, —C(S)—, —C(S)NH—, —NHC(S)—, —NHC(S)NH—, —C(S)O—, —OC(S)—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered).

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene.

In embodiments, L² is unsubstituted alkylene, unsubstituted heteroalkylene, unsubstituted cycloalkylene, unsubstituted heterocycloalkylene, unsubstituted arylene, or unsubstituted heteroarylene.

In embodiments, L² is substituted (e.g., substituted with a substituent group, a size-limited substituent group, or lower substituent group) or unsubstituted alkylene. In embodiments, L² is substituted (e.g., substituted with a substituent group, a size-limited substituent group, or lower substituent group) alkylene. In embodiments, L² is unsubstituted alkylene. In embodiments, L² is substituted or unsubstituted alkylene (e.g., C₁-C₈, C₁-C₆, C₁-C₄, or C₁-C₂). In embodiments, L² is substituted alkylene (e.g., C₁-C₈, C₁-C₆, C₁-C₄, or C₁-C₂). In embodiments, L² is unsubstituted alkylene (e.g., C₁-C₈, C₁-C₆, C₁-C₄, or C₁-C₂).

In embodiments, L² is substituted (e.g., substituted with a substituent group, a size-limited substituent group, or lower substituent group) or unsubstituted heteroalkylene. In embodiments, L² is substituted (e.g., substituted with a substituent group, a size-limited substituent group, or lower substituent group) heteroalkylene. In embodiments, L² is unsubstituted heteroalkylene. In embodiments, L² is substituted or unsubstituted heteroalkylene (e.g., 2 to 8 membered, 2 to 6 membered, 4 to 6 membered, 2 to 3 membered, or 4 to 5 membered). In embodiments, L² is substituted heteroalkylene (e.g., 2 to 8 membered, 2 to 6 membered, 4 to 6 membered, 2 to 3 membered, or 4 to 5 membered). In embodiments, L² is an unsubstituted heteroalkylene (e.g., 2 to 8 membered, 2 to 6 membered, 4 to 6 membered, 2 to 3 membered, or 4 to 5 membered).

In embodiments, L² is substituted (e.g., substituted with a substituent group, a size-limited substituent group, or lower substituent group) or unsubstituted cycloalkylene. In embodiments, L² is substituted (e.g., substituted with a substituent group, a size-limited substituent group, or lower substituent group) cycloalkylene. In embodiments, L² is an unsubstituted cycloalkylene. In embodiments, L² is substituted or unsubstituted cycloalkylene (e.g., C₃-C₈, C₃-C₆, C₄-C₆, or C₅-C₆). In embodiments, L² is substituted cycloalkylene (e.g., C₃-C₈, C₃-C₆, C₄-C₆, or C₅-C₆). In embodiments, L² is unsubstituted cycloalkylene (e.g., C₃-C₈, C₃-C₆, C₄-C₆, or C₅-C₆).

In embodiments, L² is substituted (e.g., substituted with a substituent group, a size-limited substituent group, or lower substituent group) or unsubstituted heterocycloalkylene. In embodiments, L² is substituted (e.g., substituted with a substituent group, a size-limited substituent group, or lower substituent group) heterocycloalkylene. In embodiments, L² is an unsubstituted heterocycloalkylene. In embodiments, L² is substituted or unsubstituted heterocycloalkylene (e.g., 3 to 8 membered, 3 to 6 membered, 4 to 6 membered, 4 to 5 membered, or 5 to 6 membered). In embodiments, L² is substituted heterocycloalkylene (e.g., 3 to 8 membered, 3 to 6 membered, 4 to 6 membered, 4 to 5 membered, or 5 to 6 membered). In embodiments, L² an unsubstituted heterocycloalkylene (e.g., 3 to 8 membered, 3 to 6 membered, 4 to 6 membered, 4 to 5 membered, or 5 to 6 membered).

In embodiments, L² is substituted (e.g., substituted with a substituent group, a size-limited substituent group, or lower substituent group) or unsubstituted arylene. In embodiments, L² is substituted (e.g., substituted with a substituent group, a size-limited substituent group, or lower substituent group) arylene. In embodiments, L² is an unsubstituted arylene. In embodiments, L² is substituted or unsubstituted arylene (e.g., C₆-C₁₀ or phenylene). In embodiments, L² is substituted arylene (e.g., C₆-C₁₀ or phenylene). In embodiments, L² is an unsubstituted arylene (e.g., C₆-C₁₀ or phenylene).

In embodiments, L² is substituted (e.g., substituted with a substituent group, a size-limited substituent group, or lower substituent group) or unsubstituted heteroarylene. In embodiments, L² is substituted (e.g., substituted with a substituent group, a size-limited substituent group, or lower substituent group) heteroarylene. In embodiments, L² is an unsubstituted heteroarylene. In embodiments, L² is substituted or unsubstituted heteroarylene (e.g., 5 to 10 membered, 5 to 9 membered, or 5 to 6 membered). In embodiments, L² is substituted heteroarylene (e.g., 5 to 10 membered, 5 to 9 membered, or 5 to 6 membered). In embodiments, L² is an unsubstituted heteroarylene (e.g., 5 to 10 membered, 5 to 9 membered, or 5 to 6 membered).

In embodiments, L² is

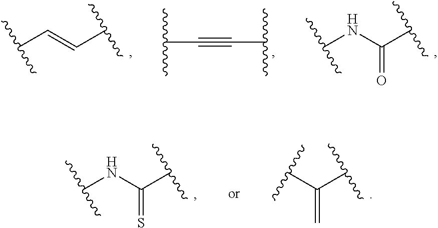

In embodiments, L² is

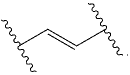 [SMILES: */C=C/*]

In embodiments, L² is

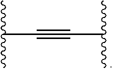 [SMILES: *C#C*]

In embodiments, L² is

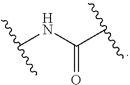 [SMILES: *NC(*)=O]

In embodiments, L² is

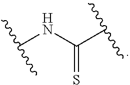 [SMILES: *NC(*)=S]

In embodiments, L² is

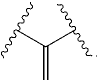 [SMILES: *C(*)=C]

In embodiments, R¹ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R¹ is unsubstituted alkyl, unsubstituted heteroalkyl, unsubstituted cycloalkyl, unsubstituted heterocycloalkyl, unsubstituted aryl, or unsubstituted heteroaryl.

In embodiments, R¹ is substituted (e.g., substituted with a substituent group, a size-limited substituent group, or lower substituent group) or unsubstituted alkyl. In embodiments, R¹ is substituted (e.g., substituted with a substituent group, a size-limited substituent group, or lower substituent group) alkyl. In embodiments, R¹ is unsubstituted alkyl. In embodiments, R¹ is substituted or unsubstituted alkyl (e.g., C₁-C₈, C₁-C₆, C₁-C₄, or C₁-C₂). In embodiments, R¹ is substituted alkyl (e.g., C₁-C₈, C₁-C₆, C₁-C₄, or C₁-C₂). In embodiments, R¹ is unsubstituted alkyl (e.g., C₁-C₈, C₁-C₆, C₁-C₄, or C₁-C₂).

In embodiments, R¹ is substituted (e.g., substituted with a substituent group, a size-limited substituent group, or lower substituent group) or unsubstituted heteroalkyl. In embodiments, R¹ is substituted (e.g., substituted with a substituent group, a size-limited substituent group, or lower substituent group) heteroalkyl. In embodiments, R¹ is unsubstituted heteroalkyl. In embodiments, R¹ is substituted or unsubstituted heteroalkyl (e.g., 2 to 8 membered, 2 to 6 membered, 4 to 6 membered, 2 to 3 membered, or 4 to 5 membered). In embodiments, R¹ is substituted heteroalkyl (e.g., 2 to 8 membered, 2 to 6 membered, 4 to 6 membered, 2 to 3 membered, or 4 to 5 membered). In embodiments, R¹ is an unsubstituted heteroalkyl (e.g., 2 to 8 membered, 2 to 6 membered, 4 to 6 membered, 2 to 3 membered, or 4 to 5 membered).

In embodiments, R¹ is substituted (e.g., substituted with a substituent group, a size-limited substituent group, or lower substituent group) or unsubstituted cycloalkyl. In embodiments, R¹ is substituted (e.g., substituted with a substituent group, a size-limited substituent group, or lower substituent group) cycloalkyl. In embodiments, R¹ is an unsubstituted cycloalkyl. In embodiments, R¹ is substituted or unsubstituted cycloalkyl (e.g., C₃-C₈, C₃-C₆, C₄-C₆, or C₅-C₆). In embodiments, R¹ is substituted cycloalkyl (e.g., C₃-C₈, C₃-C₆, C₄-C₆, or C₅-C₆). In embodiments, R¹ is unsubstituted cycloalkyl (e.g., C₃-C₈, C₃-C₆, C₄-C₆, or C₅-C₆).

In embodiments, R¹ is substituted (e.g., substituted with a substituent group, a size-limited substituent group, or lower substituent group) or unsubstituted heterocycloalkyl. In embodiments, R¹ is substituted (e.g., substituted with a substituent group, a size-limited substituent group, or lower substituent group) heterocycloalkyl. In embodiments, R¹ is an unsubstituted heterocycloalkyl. In embodiments, R¹ is substituted or unsubstituted heterocycloalkyl (e.g., 3 to 8 membered, 3 to 6 membered, 4 to 6 membered, 4 to 5 membered, or 5 to 6 membered). In embodiments, R¹ is substituted heterocycloalkyl (e.g., 3 to 8 membered, 3 to 6 membered, 4 to 6 membered, 4 to 5 membered, or 5 to 6 membered). In embodiments, R¹ an unsubstituted heterocycloalkyl (e.g., 3 to 8 membered, 3 to 6 membered, 4 to 6 membered, 4 to 5 membered, or 5 to 6 membered).

In embodiments, R¹ is substituted (e.g., substituted with a substituent group, a size-limited substituent group, or lower substituent group) or unsubstituted aryl. In embodiments, R¹ is substituted (e.g., substituted with a substituent group, a size-limited substituent group, or lower substituent group) aryl. In embodiments, R¹ is an unsubstituted aryl. In embodiments, R¹ is substituted or unsubstituted aryl (e.g., C₆-C₁₀ or phenyl). In embodiments, R¹ is substituted aryl (e.g., C₆-C₁₀ or phenyl). In embodiments, R¹ is an unsubstituted aryl (e.g., C₆-C₁₀ or phenyl).

In embodiments, R¹ is substituted (e.g., substituted with a substituent group, a size-limited substituent group, or lower substituent group) or unsubstituted heteroaryl. In embodiments, R¹ is substituted (e.g., substituted with a substituent group, a size-limited substituent group, or lower substituent group) heteroaryl. In embodiments, R¹ is an unsubstituted heteroaryl. In embodiments, R¹ is substituted or unsubstituted heteroaryl (e.g., 5 to 10 membered, 5 to 9 membered, or 5 to 6 membered). In embodiments, R¹ is substituted heteroaryl (e.g., 5 to 10 membered, 5 to 9 membered, or 5 to 6 membered). In embodiments, R¹ is an unsubstituted heteroaryl (e.g., 5 to 10 membered, 5 to 9 membered, or 5 to 6 membered).

In embodiments, R² is hydrogen, substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R² is substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, R² is substituted (e.g., substituted with a substituent group, a size-limited substituent group, or lower substituent group) or unsubstituted alkyl, or substituted (e.g., substituted with a substituent group, a size-limited substituent group, or lower substituent group) or unsubstituted heteroalkyl.

In embodiments, R² is unsubstituted alkyl or unsubstituted heteroalkyl.

In embodiments, R² is substituted or unsubstituted methyl. In embodiments, R² is substituted or unsubstituted C₂ alkyl. In embodiments, R² is substituted or unsubstituted C₃ alkyl. In embodiments, R² is substituted or unsubstituted C₄ alkyl. In embodiments, R² is substituted or unsubstituted C₅ alkyl. In embodiments, R² is substituted or unsubstituted C₆ alkyl. In embodiments, R² is substituted or unsubstituted C₇ alkyl. In embodiments, R² is substituted or unsubstituted C₈ alkyl. In embodiments, R² is substituted methyl. In embodiments, R² is substituted C₂ alkyl. In embodiments, R² is substituted C₃ alkyl. In embodiments, R² is substituted C₄ alkyl. In embodiments, R² is substituted C₅ alkyl. In embodiments, R² is substituted C₆ alkyl. In embodiments, R² is substituted C₇ alkyl. In embodiments, R² is substituted C₈ alkyl. In embodiments, R² is an unsubstituted methyl. In embodiments, R² is an unsubstituted C₂ alkyl. In embodiments, R² is an unsubstituted C₃ alkyl. In embodiments, R² is an unsubstituted C₄ alkyl. In embodiments, R² is an unsubstituted C₅ alkyl. In embodiments, R² is an unsubstituted C₆ alkyl. In embodiments, R² is an unsubstituted C₇ alkyl. In embodiments, R² is an unsubstituted C₈ alkyl.

In embodiments, R² is substituted (e.g., substituted with a substituent group, a size-limited substituent group, or lower substituent group) or unsubstituted alkyl. In embodiments, R² is substituted (e.g., substituted with a substituent group, a size-limited substituent group, or lower substituent group) alkyl. In embodiments, R² is unsubstituted alkyl. In embodiments, R² is substituted or unsubstituted alkyl (e.g., C₁-C₈, C₁-C₆, C₁-C₄, or C₁-C₂). In embodiments, R² is substituted alkyl (e.g., C₁-C₈, C₁-C₆, C₁-C₄, or C₁-C₂). In embodiments, R² is unsubstituted alkyl (e.g., C₁-C₈, C₁-C₆, C₁-C₄, or C₁-C₂).

In embodiments, R² is substituted (e.g., substituted with a substituent group, a size-limited substituent group, or lower substituent group) or unsubstituted heteroalkyl. In embodiments, R² is substituted (e.g., substituted with a substituent group, a size-limited substituent group, or lower substituent group) heteroalkyl. In embodiments, R² is unsubstituted heteroalkyl. In embodiments, R² is substituted or unsubstituted heteroalkyl (e.g., 2 to 8 membered, 2 to 6 membered, 4 to 6 membered, 2 to 3 membered, or 4 to 5 membered). In embodiments, R² is substituted heteroalkyl (e.g., 2 to 8 membered, 2 to 6 membered, 4 to 6 membered, 2 to 3 membered, or 4 to 5 membered). In embodiments, R² is an unsubstituted heteroalkyl (e.g., 2 to 8 membered, 2 to 6 membered, 4 to 6 membered, 2 to 3 membered, or 4 to 5 membered).

In embodiments, R³ is hydrogen, substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R³ is substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, R³ is substituted (e.g., substituted with a substituent group, a size-limited substituent group, or lower substituent group) or unsubstituted alkyl, or substituted (e.g., substituted with a substituent group, a size-limited substituent group, or lower substituent group) or unsubstituted heteroalkyl.

In embodiments, R³ is unsubstituted alkyl or unsubstituted heteroalkyl.

In embodiments, R³ is substituted or unsubstituted methyl. In embodiments, R³ is substituted or unsubstituted C₂ alkyl. In embodiments, R³ is substituted or unsubstituted C₃ alkyl. In embodiments, R³ is substituted or unsubstituted C₄ alkyl. In embodiments, R³ is substituted or unsubstituted C₅ alkyl. In embodiments, R³ is substituted or unsubstituted C₆ alkyl. In embodiments, R³ is substituted or unsubstituted C₇ alkyl. In embodiments, R³ is substituted or unsubstituted C₈ alkyl. In embodiments, R³ is substituted methyl. In embodiments, R³ is substituted C₂ alkyl. In embodiments, R³ is substituted C₃ alkyl. In embodiments, R³ is substituted C₄ alkyl. In embodiments, R³ is substituted C₅ alkyl. In embodiments, R³ is substituted C₆ alkyl. In embodiments, R³ is substituted C₇ alkyl. In embodiments, R³ is substituted C₈ alkyl. In embodiments, R³ is an unsubstituted methyl. In embodiments, R³ is an unsubstituted C₂ alkyl. In embodiments, R³ is an unsubstituted C₃ alkyl. In embodiments, R³ is an unsubstituted C₄ alkyl. In embodiments, R³ is an unsubstituted C₅ alkyl. In embodiments, R³ is an unsubstituted C₆ alkyl. In embodiments, R³ is an unsubstituted C₇ alkyl. In embodiments, R³ is an unsubstituted C₈ alkyl.

In embodiments, R³ is substituted (e.g., substituted with a substituent group, a size-limited substituent group, or lower substituent group) or unsubstituted alkyl. In embodiments, R³ is substituted (e.g., substituted with a substituent group, a size-limited substituent group, or lower substituent group) alkyl. In embodiments, R³ is unsubstituted alkyl. In embodiments, R³ is substituted or unsubstituted alkyl (e.g., C₁-C₈, C₁-C₆, C₁-C₄, or C₁-C₂). In embodiments, R³ is substituted alkyl (e.g., C₁-C₈, C₁-C₆, C₁-C₄, or C₁-C₂). In embodiments, R³ is unsubstituted alkyl (e.g., C₁-C₈, C₁-C₆, C₁-C₄, or C₁-C₂).

In embodiments, R³ is substituted (e.g., substituted with a substituent group, a size-limited substituent group, or lower substituent group) or unsubstituted heteroalkyl. In embodiments, R³ is substituted (e.g., substituted with a substituent group, a size-limited substituent group, or lower substituent group) heteroalkyl. In embodiments, R³ is unsubstituted heteroalkyl. In embodiments, R³ is substituted or unsubstituted heteroalkyl (e.g., 2 to 8 membered, 2 to 6 membered, 4 to 6 membered, 2 to 3 membered, or 4 to 5 membered). In embodiments, R³ is substituted heteroalkyl (e.g., 2 to 8 membered, 2 to 6 membered, 4 to 6 membered, 2 to 3 membered, or 4 to 5 membered). In embodiments, R³ is an unsubstituted heteroalkyl (e.g., 2 to 8 membered, 2 to 6 membered, 4 to 6 membered, 2 to 3 membered, or 4 to 5 membered).

In embodiments, R⁴ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁴ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R⁴ is unsubstituted alkyl, unsubstituted heteroalkyl, unsubstituted cycloalkyl, unsubstituted heterocycloalkyl, unsubstituted aryl, or unsubstituted heteroaryl.

In embodiments, R⁴ is substituted (e.g., substituted with a substituent group, a size-limited substituent group, or lower substituent group) or unsubstituted alkyl. In embodiments, R⁴ is substituted (e.g., substituted with a substituent group, a size-limited substituent group, or lower substituent group) alkyl. In embodiments, R⁴ is unsubstituted alkyl. In embodiments, R⁴ is substituted or unsubstituted alkyl (e.g., C₁-C₈, C₁-C₆, C₁-C₄, or C₁-C₂). In embodiments, R⁴ is substituted alkyl (e.g., C₁-C₈, C₁-C₆, C₁-C₄, or C₁-C₂). In embodiments, R⁴ is unsubstituted alkyl (e.g., C₁-C₈, C₁-C₆, C₁-C₄, or C₁-C₂).

In embodiments, R⁴ is substituted (e.g., substituted with a substituent group, a size-limited substituent group, or lower substituent group) or unsubstituted heteroalkyl. In embodiments, R⁴ is substituted (e.g., substituted with a substituent group, a size-limited substituent group, or lower substituent group) heteroalkyl. In embodiments, R⁴ is unsubstituted heteroalkyl. In embodiments, R⁴ is substituted or unsubstituted heteroalkyl (e.g., 2 to 8 membered, 2 to 6 membered, 4 to 6 membered, 2 to 3 membered, or 4 to 5 membered). In embodiments, R⁴ is substituted heteroalkyl (e.g., 2 to 8 membered, 2 to 6 membered, 4 to 6 membered, 2 to 3 membered, or 4 to 5 membered). In embodiments, R⁴ is an unsubstituted heteroalkyl (e.g., 2 to 8 membered, 2 to 6 membered, 4 to 6 membered, 2 to 3 membered, or 4 to 5 membered).

In embodiments, R⁴ is substituted (e.g., substituted with a substituent group, a size-limited substituent group, or lower substituent group) or unsubstituted cycloalkyl. In embodiments, R⁴ is substituted (e.g., substituted with a substituent group, a size-limited substituent group, or lower substituent group) cycloalkyl. In embodiments, R⁴ is an unsubstituted cycloalkyl. In embodiments, R⁴ is substituted or unsubstituted cycloalkyl (e.g., C₃-C₈, C₃-C₆, C₄-C₆, or C₅-C₆). In embodiments, R⁴ is substituted cycloalkyl (e.g., C₃-C₈, C₃-C₆, C₄-C₆, or C₅-C₆). In embodiments, R⁴ is unsubstituted cycloalkyl (e.g., C₃-C₈, C₃-C₆, C₄-C₆, or C₅-C₆).

In embodiments, R⁴ is substituted (e.g., substituted with a substituent group, a size-limited substituent group, or lower substituent group) or unsubstituted heterocycloalkyl. In embodiments, R⁴ is substituted (e.g., substituted with a substituent group, a size-limited substituent group, or lower substituent group) heterocycloalkyl. In embodiments, R⁴ is an unsubstituted heterocycloalkyl. In embodiments, R⁴ is substituted or unsubstituted heterocycloalkyl (e.g., 3 to 8 membered, 3 to 6 membered, 4 to 6 membered, 4 to 5 membered, or 5 to 6 membered). In embodiments, R⁴ is substituted heterocycloalkyl (e.g., 3 to 8 membered, 3 to 6 membered, 4 to 6 membered, 4 to 5 membered, or 5 to 6 membered). In embodiments, R⁴ is unsubstituted heterocycloalkyl (e.g., 3 to 8 membered, 3 to 6 membered, 4 to 6 membered, 4 to 5 membered, or 5 to 6 membered).

In embodiments, R⁴ is substituted (e.g., substituted with a substituent group, a size-limited substituent group, or lower substituent group) or unsubstituted aryl. In embodiments, R⁴ is substituted (e.g., substituted with a substituent group, a size-limited substituent group, or lower substituent group) aryl. In embodiments, R⁴ is an unsubstituted aryl. In embodiments, R⁴ is substituted or unsubstituted aryl (e.g., C₆-C₁₀ or phenyl). In embodiments, R⁴ is substituted aryl (e.g., C₆-C₁₀ or phenyl). In embodiments, R⁴ is an unsubstituted aryl (e.g., C₆-C₁₀ or phenyl).

In embodiments, R⁴ is substituted (e.g., substituted with a substituent group, a size-limited substituent group, or lower substituent group) or unsubstituted heteroaryl. In embodiments, R⁴ is substituted (e.g., substituted with a substituent group, a size-limited substituent group, or lower substituent group) heteroaryl. In embodiments, R⁴ is an unsubstituted heteroaryl. In embodiments, R⁴ is substituted or unsubstituted heteroaryl (e.g., 5 to 10 membered, 5 to 9 membered, or 5 to 6 membered). In embodiments, R⁴ is substituted heteroaryl (e.g., 5 to 10 membered, 5 to 9 membered, or 5 to 6 membered). In embodiments, R⁴ is an unsubstituted heteroaryl (e.g., 5 to 10 membered, 5 to 9 membered, or 5 to 6 membered).

In embodiments, R⁵ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁵ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁵ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R⁵ is unsubstituted alkyl, unsubstituted heteroalkyl, unsubstituted cycloalkyl, unsubstituted heterocycloalkyl, unsubstituted aryl, or unsubstituted heteroaryl.

In embodiments, R⁵ is substituted (e.g., substituted with a substituent group, a size-limited substituent group, or lower substituent group) or unsubstituted alkyl. In embodiments, R⁵ is substituted (e.g., substituted with a substituent group, a size-limited substituent group, or lower substituent group) alkyl. In embodiments, R⁵ is unsubstituted alkyl. In embodiments, R⁵ is substituted or unsubstituted alkyl (e.g., C₁-C₈, C₁-C₆, C₁-C₄, or C₁-C₂). In embodiments, R⁵ is substituted alkyl (e.g., C₁-C₈, C₁-C₆, C₁-C₄, or C₁-C₂). In embodiments, R⁵ is unsubstituted alkyl (e.g., C₁-C₈, C₁-C₆, C₁-C₄, or C₁-C₂).

In embodiments, R⁵ is substituted (e.g., substituted with a substituent group, a size-limited substituent group, or lower substituent group) or unsubstituted heteroalkyl. In embodiments, R⁵ is substituted (e.g., substituted with a substituent group, a size-limited substituent group, or lower substituent group) heteroalkyl. In embodiments, R⁵ is unsubstituted heteroalkyl. In embodiments, R⁵ is substituted or unsubstituted heteroalkyl (e.g., 2 to 8 membered, 2 to 6 membered, 4 to 6 membered, 2 to 3 membered, or 4 to 5 membered). In embodiments, R⁵ is substituted heteroalkyl (e.g., 2 to 8 membered, 2 to 6 membered, 4 to 6 membered, 2 to 3 membered, or 4 to 5 membered). In embodiments, R⁵ is an unsubstituted heteroalkyl (e.g., 2 to 8 membered, 2 to 6 membered, 4 to 6 membered, 2 to 3 membered, or 4 to 5 membered).

In embodiments, R⁵ is substituted (e.g., substituted with a substituent group, a size-limited substituent group, or lower substituent group) or unsubstituted cycloalkyl. In embodiments, R⁵ is substituted (e.g., substituted with a substituent group, a size-limited substituent group, or lower substituent group) cycloalkyl. In embodiments, R⁵ is an unsubstituted cycloalkyl. In embodiments, R⁵ is substituted or unsubstituted cycloalkyl (e.g., C₃-C₈, C₃-C₆, C₄-C₆, or C₅-C₆). In embodiments, R⁵ is substituted cycloalkyl (e.g., C₃-C₈, C₃-C₆, C₄-C₆, or C₅-C₆). In embodiments, R⁵ is unsubstituted cycloalkyl (e.g., C₃-C₈, C₃-C₆, C₄-C₆, or C₅-C₆).

In embodiments, R⁵ is substituted (e.g., substituted with a substituent group, a size-limited substituent group, or lower substituent group) or unsubstituted heterocycloalkyl. In embodiments, R⁵ is substituted (e.g., substituted with a substituent group, a size-limited substituent group, or lower substituent group) heterocycloalkyl. In embodiments, R⁵ is an unsubstituted heterocycloalkyl. In embodiments, R⁵ is substituted or unsubstituted heterocycloalkyl (e.g., 3 to 8 membered, 3 to 6 membered, 4 to 6 membered, 4 to 5 membered, or 5 to 6 membered). In embodiments, R⁵ is substituted heterocycloalkyl (e.g., 3 to 8 membered, 3 to 6 membered, 4 to 6 membered, 4 to 5 membered, or 5 to 6 membered). In embodiments, R⁵ an unsubstituted heterocycloalkyl (e.g., 3 to 8 membered, 3 to 6 membered, 4 to 6 membered, 4 to 5 membered, or 5 to 6 membered).

In embodiments, R⁵ is substituted (e.g., substituted with a substituent group, a size-limited substituent group, or lower substituent group) or unsubstituted aryl. In embodiments, R⁵ is substituted (e.g., substituted with a substituent group, a size-limited substituent group, or lower substituent group) aryl. In embodiments, R⁵ is an unsubstituted aryl. In embodiments, R⁵ is substituted or unsubstituted aryl (e.g., C₆-C₁₀ or phenyl). In embodiments, R⁵ is substituted aryl (e.g., C₆-C₁₀ or phenyl). In embodiments, R⁵ is an unsubstituted aryl (e.g., C₆-C₁₀ or phenyl).

In embodiments, R⁵ is substituted (e.g., substituted with a substituent group, a size-limited substituent group, or lower substituent group) or unsubstituted heteroaryl. In embodiments, R⁵ is substituted (e.g., substituted with a substituent group, a size-limited substituent group, or lower substituent group) heteroaryl. In embodiments, R⁵ is an unsubstituted heteroaryl. In embodiments, R⁵ is substituted or unsubstituted heteroaryl (e.g., 5 to 10 membered, 5 to 9 membered, or 5 to 6 membered). In embodiments, R⁵ is substituted heteroaryl (e.g., 5 to 10 membered, 5 to 9 membered, or 5 to 6 membered). In embodiments, R⁵ is an unsubstituted heteroaryl (e.g., 5 to 10 membered, 5 to 9 membered, or 5 to 6 membered).

In embodiments, z4 is 0. In embodiments, z4 is 1. In embodiments, z4 is 2. In embodiments, z4 is 3. In embodiments, z5 is 0. In embodiments, z5 is 1. In embodiments, z5 is 2. In embodiments, z5 is 3. In embodiments, z5 is 4.

In embodiments, the retinoic acid receptor inhibitor is a compound described in US 2001/0003780 A1, US 2002/0048580 A1, U.S. Pat. No. 6,713,515, US 2002/0090352 A1, U.S. Pat. No. 5,618,839, WO 98/46228, US 2014/0187504 A1, or Germain et al. Pharmacol Rev 58:712-725, 2006, which are incorporated herein by reference in their entirety for all purposes.

In embodiments, the retinoic acid receptor inhibitor is

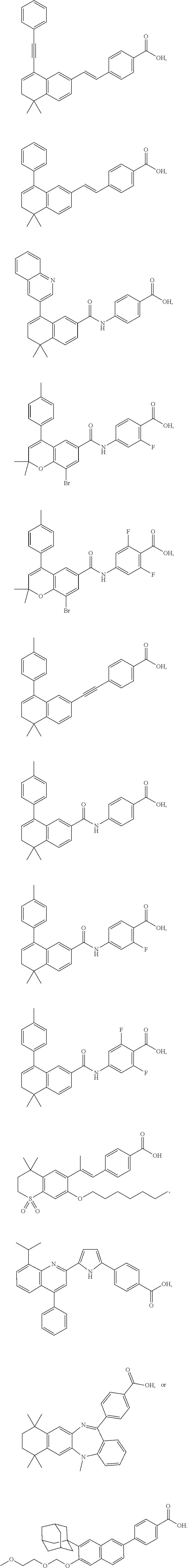

In embodiments, the retinoic acid receptor inhibitor is not

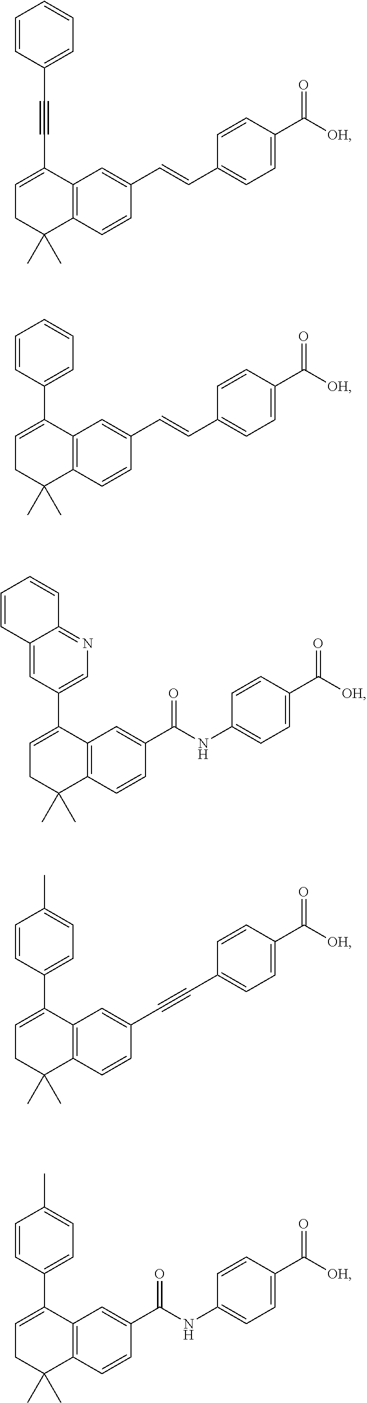 [SMILES: CC1(C)CC=C(C#Cc2ccccc2)c2cc(/C=C/c3ccc(C(=O)O)cc3)ccc21 ; CC1(C)CC=C(c2ccccc2)c2cc(/C=C/c3ccc(C(=O)O)cc3)ccc21 ; CC1(C)CC=C(c2cnc3ccccc3c2)c2cc(C(=O)Nc3ccc(C(=O)O)cc3)ccc21 ; Cc1ccc(C2=CCC(C)(C)c3ccc(C#Cc4ccc(C(=O)O)cc4)cc32)cc1 ; Cc1ccc(C2=CCC(C)(C)c3ccc(C(=O)Nc4ccc(C(=O)O)cc4)cc32)cc1]

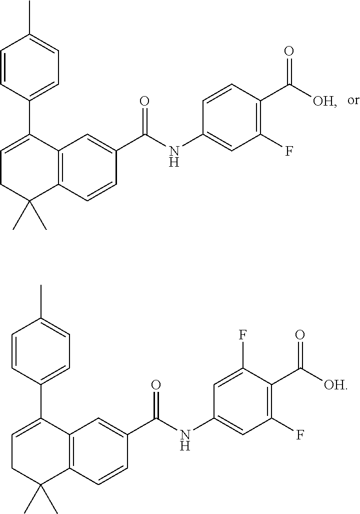 [SMILES: Cc1ccc(C2=CCC(C)(C)c3ccc(C(=O)Nc4ccc(C(=O)O)c(F)c4)cc32)cc1 ; "or" ; Cc1ccc(C2=CCC(C)(C)c3ccc(C(=O)Nc4cc(F)c(C(=O)O)c(F)c4)cc32)cc1]

In an aspect is provided a retinoic acid receptor inhibitor (e.g., a compound or retinoic acid receptor inhibitor described herein, a gene modulating reagent (e.g., CRISPR complex, TAL effector nuclease, zinc-finger nuclease, homing endonuclease, meganuclease, antisense nucleic acid, or siRNA)), and a pharmaceutically acceptable excipient. In embodiments, the retinoic acid receptor inhibitor is a compound described herein. In embodiments, the pharmaceutical composition includes an effective amount of the retinoic acid receptor inhibitor. In embodiments, the pharmaceutical composition includes a therapeutically effective amount of the retinoic acid receptor inhibitor. In embodiments, the retinoic acid receptor inhibitor is a compound (e.g., described herein). In embodiments, the retinoic acid receptor inhibitor is a gene modulating reagent (e.g., described herein). In embodiments, the gene modulating reagent target gene or target nucleic acid is a DNA or RNA sequence corresponding to the sequence NM_000964.3 or a fragment thereof, or a complement thereof. In embodiments, the gene modulating reagent target gene or target nucleic acid is a DNA or RNA sequence corresponding to the sequence NM_000965.4 or a fragment thereof, or a complement thereof. In embodiments, the gene modulating reagent target gene or target nucleic acid is a DNA or RNA sequence corresponding to the sequence NM_000966.5 or a fragment thereof, or a complement thereof. In embodiments, the retinoic acid receptor inhibitor is a CRISPR complex (e.g., described herein). In embodiments, the CRISPR complex target gene or target nucleic acid is a DNA sequence corresponding to the sequence NM_000964.3 or a fragment thereof, or a complement thereof. In embodiments, the CRISPR complex target gene or target nucleic acid is a DNA sequence corresponding to the sequence NM_000965.4 or a fragment thereof, or a complement thereof. In embodiments, the CRISPR complex target gene or target nucleic acid is a DNA sequence corresponding to the sequence NM_000966.5 or a fragment thereof, or a complement thereof. In embodiments, the retinoic acid receptor inhibitor is a TAL effector nuclease (e.g., described herein). In embodiments, the TAL effector nuclease target gene or target nucleic acid is a DNA or RNA sequence corresponding to the sequence NM_000964.3 or a fragment thereof, or a complement thereof. In embodiments, the TAL effector nuclease target gene or target nucleic acid is a DNA or RNA sequence corresponding to the sequence NM_000965.4 or a fragment thereof, or a complement thereof. In embodiments, the TAL effector nuclease target gene or target nucleic acid is a DNA or RNA sequence corresponding to the sequence NM_000966.5 or a fragment thereof, or a complement thereof. In embodiments, the retinoic acid receptor inhibitor is a zinc-finger nuclease (e.g., described herein). In embodiments, the zinc-finger nuclease target gene or target nucleic acid is a DNA or RNA sequence corresponding to the sequence NM_000964.3 or a fragment thereof, or a complement thereof. In embodiments, the zinc-finger nuclease target gene or target nucleic acid is a DNA or RNA sequence corresponding to the sequence NM_000965.4 or a fragment thereof, or a complement thereof. In embodiments, the zinc-finger nuclease target gene or target nucleic acid is a DNA or RNA sequence corresponding to the sequence NM_000966.5 or a fragment thereof, or a complement thereof. In embodiments, the retinoic acid receptor inhibitor is a homing endonuclease (e.g., described herein). In embodiments, the homing endonuclease target gene or target nucleic acid is a DNA or RNA sequence corresponding to the sequence NM_000964.3 or a fragment thereof, or a complement thereof. In embodiments, the homing endonuclease target gene or target nucleic acid is a DNA or RNA sequence corresponding to the sequence NM_000965.4 or a fragment thereof, or a complement thereof. In embodiments, the homing endonuclease target gene or target nucleic acid is a DNA or RNA sequence corresponding to the sequence NM_000966.5 or a fragment thereof, or a complement thereof. In embodiments, the retinoic acid receptor inhibitor is a meganuclease (e.g., described herein). In embodiments, the meganuclease target gene or target nucleic acid is a DNA or RNA sequence corresponding to the sequence NM_000964.3 or a fragment thereof, or a complement thereof. In embodiments, the meganuclease target gene or target nucleic acid is a DNA or RNA sequence corresponding to the sequence NM_000965.4 or a fragment thereof, or a complement thereof. In embodiments, the meganuclease target gene or target nucleic acid is a DNA or RNA sequence corresponding to the sequence NM_000966.5 or a fragment thereof, or a complement thereof. In embodiments, the retinoic acid receptor inhibitor is an antisense nucleic acid (e.g., described herein). In embodiments, the antisense nucleic acid target gene or target nucleic acid is an RNA sequence corresponding to the sequence NM_000964.3 or a fragment thereof, or a complement thereof. In embodiments, the antisense nucleic acid target gene or target nucleic acid is an RNA sequence corresponding to the sequence NM_000965.4 or a fragment thereof, or a complement thereof. In embodiments, the antisense nucleic acid target gene or target nucleic acid is an RNA sequence corresponding to the sequence NM_000966.5 or a fragment thereof, or a complement thereof. In embodiments, the retinoic acid receptor inhibitor is an siRNA (e.g., described herein). In embodiments, the siRNA target gene or target nucleic acid is an RNA sequence corresponding to the sequence NM_000964.3 or a fragment thereof, or a complement thereof. In embodiments, the siRNA target gene or target nucleic acid is an RNA sequence corresponding to the sequence NM_000965.4 or a fragment thereof, or a complement thereof. In embodiments, the siRNA target gene or target nucleic acid is an RNA sequence corresponding to the sequence NM_000966.5 or a fragment thereof, or a complement thereof.

### III. Pharmaceutical Compositions

In an aspect is provided a pharmaceutical composition including a compound (e.g., a compound or retinoic acid receptor inhibitor described herein), pharmaceutical salt thereof, or a prodrug thereof, as described herein and a pharmaceutically acceptable excipient. In embodiments, the retinoic acid receptor inhibitor is a compound described herein.

In embodiments, the pharmaceutical composition includes an effective amount of the compound. In embodiments, the pharmaceutical composition includes a therapeutically effective amount of the compound. In embodiments, the pharmaceutical composition includes an effective amount of the retinoic acid receptor inhibitor. In embodiments, the pharmaceutical composition includes a therapeutically effective amount of the retinoic acid receptor inhibitor. In embodiments, the pharmaceutical composition includes a second agent (e.g., retinoic acid metabolism-blocking agent, or retinaldehyde dehydrogenase inhibitor, such as for example diethylaminobenzaldehyde, citral, or disulfiram). In embodiments of the pharmaceutical compositions, the pharmaceutical composition includes a second agent in a therapeutically effective amount.

The pharmaceutical compositions may include optical isomers, diastereomers, or pharmaceutically acceptable salts of the modulators disclosed herein. The compound included in the pharmaceutical composition may be covalently attached to a carrier moiety. Alternatively, the compound included in the pharmaceutical composition is not covalently linked to a carrier moiety.

In an aspect is provided a pharmaceutical composition including a retinoic acid receptor inhibitor (e.g., a compound or retinoic acid receptor inhibitor described herein, a gene modulating reagent (e.g., CRISPR complex, TAL effector nuclease, zinc-finger nuclease, homing endonuclease, meganuclease, antisense nucleic acid, or siRNA)), and a pharmaceutically acceptable excipient. In embodiments, the retinoic acid receptor inhibitor is a compound described herein. In embodiments, the pharmaceutical composition includes an effective amount of the retinoic acid receptor inhibitor. In embodiments, the pharmaceutical composition includes a therapeutically effective amount of the retinoic acid receptor inhibitor. In embodiments, the retinoic acid receptor inhibitor is a compound (e.g., described herein). In embodiments, the retinoic acid receptor inhibitor is a gene modulating reagent (e.g., described herein). In embodiments, the gene modulating reagent target gene or target nucleic acid is a DNA or RNA sequence corresponding to the sequence NM_000964.3 or a fragment thereof, or a complement thereof. In embodiments, the gene modulating reagent target gene or target nucleic acid is a DNA or RNA sequence corresponding to the sequence NM_000965.4 or a fragment thereof, or a complement thereof. In embodiments, the gene modulating reagent target gene or target nucleic acid is a DNA or RNA sequence corresponding to the sequence NM_000966.5 or a fragment thereof, or a complement thereof. In embodiments, the retinoic acid receptor inhibitor is a CRISPR complex (e.g., described herein). In embodiments, the CRISPR complex target gene or target nucleic acid is a DNA sequence corresponding to the sequence NM_000964.3 or a fragment thereof, or a complement thereof. In embodiments, the CRISPR complex target gene or target nucleic acid is a DNA sequence corresponding to the sequence NM_000965.4 or a fragment thereof, or a complement thereof. In embodiments, the CRISPR complex target gene or target nucleic acid is a DNA sequence corresponding to the sequence NM_000966.5 or a fragment thereof, or a complement thereof. In embodiments, the retinoic acid receptor inhibitor is a TAL effector nuclease (e.g., described herein). In embodiments, the TAL effector nuclease target gene or target nucleic acid is a DNA or RNA sequence corresponding to the sequence NM_000964.3 or a fragment thereof, or a complement thereof. In embodiments, the TAL effector nuclease target gene or target nucleic acid is a DNA or RNA sequence corresponding to the sequence NM_000965.4 or a fragment thereof, or a complement thereof. In embodiments, the TAL effector nuclease target gene or target nucleic acid is a DNA or RNA sequence corresponding to the sequence NM_000966.5 or a fragment thereof, or a complement thereof. In embodiments, the retinoic acid receptor inhibitor is a zinc-finger nuclease (e.g., described herein). In embodiments, the zinc-finger nuclease target gene or target nucleic acid is a DNA or RNA sequence corresponding to the sequence NM_000964.3 or a fragment thereof, or a complement thereof. In embodiments, the zinc-finger nuclease target gene or target nucleic acid is a DNA or RNA sequence corresponding to the sequence NM_000965.4 or a fragment thereof, or a complement thereof. In embodiments, the zinc-finger nuclease target gene or target nucleic acid is a DNA or RNA sequence corresponding to the sequence NM_000966.5 or a fragment thereof, or a complement thereof. In embodiments, the retinoic acid receptor inhibitor is a homing endonuclease (e.g., described herein). In embodiments, the homing endonuclease target gene or target nucleic acid is a DNA or RNA sequence corresponding to the sequence NM_000964.3 or a fragment thereof, or a complement thereof. In embodiments, the homing endonuclease target gene or target nucleic acid is a DNA or RNA sequence corresponding to the sequence NM_000965.4 or a fragment thereof, or a complement thereof. In embodiments, the homing endonuclease target gene or target nucleic acid is a DNA or RNA sequence corresponding to the sequence NM_000966.5 or a fragment thereof, or a complement thereof. In embodiments, the retinoic acid receptor inhibitor is a meganuclease (e.g., described herein). In embodiments, the meganuclease target gene or target nucleic acid is a DNA or RNA sequence corresponding to the sequence NM_000964.3 or a fragment thereof, or a complement thereof. In embodiments, the meganuclease target gene or target nucleic acid is a DNA or RNA sequence corresponding to the sequence NM_000965.4 or a fragment thereof, or a complement thereof. In embodiments, the meganuclease target gene or target nucleic acid is a DNA or RNA sequence corresponding to the sequence NM_000966.5 or a fragment thereof, or a complement thereof. In embodiments, the retinoic acid receptor inhibitor is an antisense nucleic acid (e.g., described herein). In embodiments, the antisense nucleic acid target gene or target nucleic acid is an RNA sequence corresponding to the sequence NM_000964.3 or a fragment thereof, or a complement thereof. In embodiments, the antisense nucleic acid target gene or target nucleic acid is an RNA sequence corresponding to the sequence NM_000965.4 or a fragment thereof, or a complement thereof. In embodiments, the antisense nucleic acid target gene or target nucleic acid is an RNA sequence corresponding to the sequence NM_000966.5 or a fragment thereof, or a complement thereof. In embodiments, the retinoic acid receptor inhibitor is an siRNA (e.g., described herein). In embodiments, the siRNA target gene or target nucleic acid is an RNA sequence corresponding to the sequence NM_000964.3 or a fragment thereof, or a complement thereof. In embodiments, the siRNA target gene or target nucleic acid is an RNA sequence corresponding to the sequence NM_000965.4 or a fragment thereof, or a complement thereof. In embodiments, the siRNA target gene or target nucleic acid is an RNA sequence corresponding to the sequence NM_000966.5 or a fragment thereof, or a complement thereof. In embodiments, the retinoic acid receptor inhibitor is an aptamer (e.g., described herein). In embodiments, the aptamer target gene or target nucleic acid is an RNA sequence corresponding to the sequence NM_000964.3 or a fragment thereof, or a complement thereof. In embodiments, the aptamer target gene or target nucleic acid is an RNA sequence corresponding to the sequence NM_000965.4 or a fragment thereof, or a complement thereof. In embodiments, the aptamer target gene or target nucleic acid is an RNA sequence corresponding to the sequence NM_000966.5 or a fragment thereof, or a complement thereof.

### IV. Methods of Use

In an aspect is provided a method of treating vision degeneration, the method including administering to a subject in need thereof an effective amount of a retinoic acid receptor inhibitor. In embodiments, the retinoic acid receptor inhibitor is a compound, an aptamer, an antibody, a gene modulating reagent (e.g., CRISPR complex, TAL effector nuclease, zinc-finger nuclease, homing endonuclease, antisense nucleic acid, or siRNA) as disclosed herein, that reduces the level of activity of retinoic acid receptor (RAR) when compared to a control, such as absence of the inhibitor or a compound, an aptamer, an antibody, a gene modulating reagent (e.g., CRISPR complex, TAL effector nuclease, zinc-finger nuclease, homing endonuclease, antisense nucleic acid, or siRNA) with known inactivity. In embodiments, the retinoic acid receptor inhibitor is a compound (e.g., a compound described herein). In embodiments, the retinoic acid receptor inhibitor is an aptamer. In embodiments, the retinoic acid receptor inhibitor is an antibody. In embodiments the retinoic acid receptor inhibitor is a gene modulating reagent. In embodiments the retinoic acid receptor inhibitor is a CRISPR complex. In embodiments the retinoic acid receptor inhibitor is a TAL effector nuclease. In embodiments the retinoic acid receptor inhibitor is a zinc-finger nuclease. In embodiments the retinoic acid receptor inhibitor is a homing endonuclease. In embodiments the retinoic acid receptor inhibitor is an antisense nucleic acid. In embodiments the retinoic acid receptor inhibitor is a siRNA. In embodiments, the retinoic acid receptor inhibitor is a RAR antagonist. In embodiments, the RAR antagonist is BMS-453. In embodiments, the RAR antagonist is BMS-493. In embodiments, the RAR antagonist is BMS-614. In embodiments, the RAR antagonist is AGN 193109. In embodiments, the RAR antagonist is AGN 193491. In embodiments, the RAR antagonist is AGN 193618. In embodiments, the RAR antagonist is AGN 194202. In embodiments, the RAR antagonist is AGN 194301. In embodiments, the RAR antagonist is AGN 194574. In embodiments, the RAR antagonist is Ro 41-5253. In embodiments, the RAR antagonist is ER 50891. In embodiments, the RAR antagonist is CD 2665. In embodiments, the RAR antagonist is LE 135. In embodiments, the RAR antagonist inhibits the binding of a nuclear receptor coactivator to the retinoic acid receptor. In embodiments, the retinoic acid receptor inhibitor is an RAR inverse agonist. In embodiments, the RAR inverse agonist is BMS-493. In embodiments, the retinoic acid receptor inhibitor reduces the level of retinoic acid receptor (e.g., compared to control, for example absence of the retinoic acid receptor inhibitor). In embodiments, the retinoic acid receptor inhibitor reduces the level of retinoic acid receptor (e.g., compared to control, for example absence of the retinoic acid receptor inhibitor) in a cell. In embodiments, the retinoic acid receptor inhibitor reduces the level of retinoic acid receptor (e.g., compared to control, for example absence of the retinoic acid receptor inhibitor) in a subject. In embodiments, the retinoic acid receptor inhibitor reduces the level of retinoic acid receptor (e.g., compared to control, for example absence of the retinoic acid receptor inhibitor) in a nerve cell. In embodiments, the retinoic acid receptor inhibitor reduces the level of retinoic acid receptor (e.g., compared to control, for example absence of the retinoic acid receptor inhibitor) in a retinal ganglion cell. In embodiments, the retinoic acid receptor inhibitor reduces the level of a component of a signaling pathway including a retinoic acid receptor (e.g., compared to control, for example absence of the retinoic acid receptor inhibitor). In embodiments, the retinoic acid receptor inhibitor reduces the level of activity of a signaling pathway including a retinoic acid receptor (e.g., compared to control, for example absence of the retinoic acid receptor inhibitor).

In embodiments, the retinoic acid receptor inhibitor is an RAR antagonist. In embodiments, the retinoic acid receptor inhibitor (e.g., RAR antagonist) decreases expression or activity by at least 10%, 20%, 30%, 40%, 50%, 60%, 70%, 80%, 90%, 95%, 96%, 97%, 98%, or 99% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 10% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 20% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 30% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 40% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 50% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 60% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 70% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 80% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 90% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 95% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 96% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 97% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 98% in comparison to a control (e.g., absence of the retinoic acid receptor inhibitor (e.g., RAR antagonist)). In embodiments, the retinoic acid receptor inhibitor (e.g., RAR antagonist) decreases expression or activity by at least 99% in comparison to a control (e.g., absence of the retinoic acid receptor inhibitor (e.g., RAR antagonist)).

In embodiments, expression or activity is at least 1.5-fold, 2-fold, 3-fold, 4-fold, 5-fold, or 10-fold lower than the expression or activity in the absence of the retinoic acid receptor inhibitor (e.g., RAR antagonist). In embodiments, expression or activity is at least 1.5-fold lower than the expression or activity in the absence of the retinoic acid receptor inhibitor (e.g., RAR antagonist). In embodiments, expression or activity is at least 2-fold lower than the expression or activity in the absence of the retinoic acid receptor inhibitor (e.g., RAR antagonist). In embodiments, expression or activity is at least 3-fold lower than the expression or activity in the absence of the retinoic acid receptor inhibitor (e.g., RAR antagonist). In embodiments, expression or activity is at least 4-fold lower than the expression or activity in the absence of the retinoic acid receptor inhibitor (e.g., RAR antagonist). In embodiments, expression or activity is at least 5-fold lower than the expression or activity in the absence of the retinoic acid receptor inhibitor (e.g., RAR antagonist). In embodiments, expression or activity is at least 10-fold lower than the expression or activity in the absence of the retinoic acid receptor inhibitor (e.g., RAR antagonist). In embodiments, the retinoic acid receptor inhibitor (e.g., RAR antagonist) inhibits the binding of a nuclear receptor coactivator (e.g., NCOA1 or NCOA2) to the retinoic acid receptor. In embodiments, the retinoic acid receptor inhibitor (e.g., RAR antagonist) inhibits the binding of NCOA1 to the retinoic acid receptor. In embodiments, the retinoic acid receptor inhibitor (e.g., RAR antagonist) inhibits the binding of NCOA2 to the retinoic acid receptor. In embodiments, the retinoic acid receptor inhibitor (e.g., RAR antagonist) inhibits the binding of GRIP1 to the retinoic acid receptor. In embodiments, the retinoic acid receptor inhibitor (e.g., RAR antagonist) inhibits the binding of SRC-2 to the retinoic acid receptor. In embodiments, the retinoic acid receptor inhibitor (e.g., RAR antagonist) inhibits the binding of TIF2 to the retinoic acid receptor.

In embodiments, the retinoic acid receptor inhibitor is an RAR inverse agonist. In embodiments, the RAR inverse agonist decreases expression or activity by at least 10%, 20%, 30%, 40%, 50%, 60%, 70%, 80%, 90%, 95%, 96%, 97%, 98%, or 99% in comparison to a control (e.g., absence of the RAR inverse agonist). In embodiments, the RAR inverse agonist decreases expression or activity by at least 10% in comparison to a control (e.g., absence of the RAR inverse agonist). In embodiments, the RAR inverse agonist decreases expression or activity by at least 20% in comparison to a control (e.g., absence of the RAR inverse agonist). In embodiments, the RAR inverse agonist decreases expression or activity by at least 30% in comparison to a control (e.g., absence of the RAR inverse agonist). In embodiments, the RAR inverse agonist decreases expression or activity by at least 40% in comparison to a control (e.g., absence of the RAR inverse agonist). In embodiments, the RAR inverse agonist decreases expression or activity by at least 50% in comparison to a control (e.g., absence of the RAR inverse agonist). In embodiments, the RAR inverse agonist decreases expression or activity by at least 60% in comparison to a control (e.g., absence of the RAR inverse agonist). In embodiments, the RAR inverse agonist decreases expression or activity by at least 70% in comparison to a control (e.g., absence of the RAR inverse agonist). In embodiments, the RAR inverse agonist decreases expression or activity by at least 80% in comparison to a control (e.g., absence of the RAR inverse agonist). In embodiments, the RAR inverse agonist decreases expression or activity by at least 90% in comparison to a control (e.g., absence of the RAR inverse agonist). In embodiments, the RAR inverse agonist decreases expression or activity by at least 95% in comparison to a control (e.g., absence of the RAR inverse agonist). In embodiments, the RAR inverse agonist decreases expression or activity by at least 96% in comparison to a control (e.g., absence of the RAR inverse agonist). In embodiments, the RAR inverse agonist decreases expression or activity by at least 97% in comparison to a control (e.g., absence of the RAR inverse agonist). In embodiments, the RAR inverse agonist decreases expression or activity by at least 98% in comparison to a control (e.g., absence of the RAR inverse agonist). In embodiments, the RAR inverse agonist decreases expression or activity by at least 99% in comparison to a control (e.g., absence of the RAR inverse agonist).

In embodiments, expression or activity is at least 1.5-fold, 2-fold, 3-fold, 4-fold, 5-fold, or 10-fold lower than the expression or activity in the absence of the RAR inverse agonist. In embodiments, expression or activity is at least 1.5-fold lower than the expression or activity in the absence of the RAR inverse agonist. In embodiments, expression or activity is at least 2-fold lower than the expression or activity in the absence of the RAR inverse agonist. In embodiments, expression or activity is at least 3-fold lower than the expression or activity in the absence of the RAR inverse agonist. In embodiments, expression or activity is at least 4-fold lower than the expression or activity in the absence of the RAR inverse agonist. In embodiments, expression or activity is at least 5-fold lower than the expression or activity in the absence of the RAR inverse agonist. In embodiments, expression or activity is at least 10-fold lower than the expression or activity in the absence of the RAR inverse agonist. In embodiments, the RAR inverse agonist increases the binding of NCOR1 to the retinoic acid receptor. In embodiments, the RAR inverse agonist increases the binding of NCOR2 to the retinoic acid receptor. In embodiments, the RAR inverse agonist increases the binding of SMRT to the retinoic acid receptor.

In embodiments, the gene modulating reagent includes gene editing reagents and gene modulating nucleic acids. In embodiments, the gene modulating reagent is a CRISPR complex, a TAL effector nuclease, a zinc-finger nuclease, a meganuclease, a homing endonuclease, an antisense nucleic acid, or an siRNA. In embodiments, the gene modulating reagent is a CRISPR complex. In embodiments, the gene modulating reagent is a TAL effector nuclease. In embodiments, the gene modulating reagent is a zinc-finger nuclease. In embodiments, the gene modulating reagent is a meganuclease. In embodiments, the gene modulating reagent is a homing endonuclease. In embodiments, the gene modulating reagent is an antisense nucleic acid. In embodiments, the gene modulating reagent is an siRNA. In embodiments, the gene editing reagent is a CRISPR complex, a TAL effector nuclease, a zinc finger nuclease, a meganuclease, or a homing endonuclease. In embodiments, the gene editing reagent is a CRISPR complex. In embodiments, the gene editing reagent is a TAL effector nuclease. In embodiments, the gene editing reagent is a zinc-finger nuclease. In embodiments, the gene editing reagent is a meganuclease. In embodiments, the gene editing reagent is a homing endonuclease. In embodiments, the gene modulating nucleic acid includes an antisense nucleic acid or an siRNA. In embodiments, the gene modulating nucleic acid is an antisense nucleic acid. In embodiments, the gene modulating nucleic acid is an siRNA.

In embodiments, the gene modulating reagent is capable of modifying the nucleic acid sequence of the retinoic acid receptor (e.g., RARα, SEQ ID NO:2, RARβ, or RARγ). In embodiments, the gene modulating reagent is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the gene modulating reagent is capable of modifying the nucleic acid sequence of the retinoid x receptor. In embodiments, the gene modulating reagent is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid-retinoid x receptor heterodimer.

In embodiments, the CRISPR complex is capable of modifying the nucleic acid sequence of the retinoic acid receptor (e.g., RARα, SEQ ID NO:2, RARβ, or RARγ). In embodiments, the CRISPR complex is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the CRISPR complex is capable of modifying the nucleic acid sequence of the retinoid x receptor. In embodiments, the CRISPR complex is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid-retinoid x receptor heterodimer. In embodiments, the CRISPR complex includes a guide RNA and a Cas9 protein.

In embodiments, the guide RNA is complementary to a target nucleic acid. In embodiments, the guide RNA binds a target nucleic acid sequence. In embodiments, the guide RNA is complementary to a CRISPR nucleic acid sequence. In embodiments, the complement of the guide RNA has a sequence identity of about 50%, 55%, 60%, 65%, 70%, 75%, 80%, 85%, 90%, 95%, 96%, 97%, 98%, 99%, or 100% to a CRISPR nucleic acid sequence. In embodiments, the complement of the guide RNA has a sequence identity of about 50% to a CRISPR nucleic acid sequence. In embodiments, the complement of the guide RNA has a sequence identity of about 55% to a CRISPR nucleic acid sequence. In embodiments, the complement of the guide RNA has a sequence identity of about 60% to a CRISPR nucleic acid sequence. In embodiments, the complement of the guide RNA has a sequence identity of about 65% to a CRISPR nucleic acid sequence. In embodiments, the complement of the guide RNA has a sequence identity of about 70% to a CRISPR nucleic acid sequence. In embodiments, the complement of the guide RNA has a sequence identity of about 75% to a CRISPR nucleic acid sequence. In embodiments, the complement of the guide RNA has a sequence identity of about 80% to a CRISPR nucleic acid sequence. In embodiments, the complement of the guide RNA has a sequence identity of about 85% to a CRISPR nucleic acid sequence. In embodiments, the complement of the guide RNA has a sequence identity of about 90% to a CRISPR nucleic acid sequence. In embodiments, the complement of the guide RNA has a sequence identity of about 95% to a CRISPR nucleic acid sequence. In embodiments, the complement of the guide RNA has a sequence identity of about 96% to a CRISPR nucleic acid sequence. In embodiments, the complement of the guide RNA has a sequence identity of about 97% to a CRISPR nucleic acid sequence. In embodiments, the complement of the guide RNA has a sequence identity of about 98% to a CRISPR nucleic acid sequence. In embodiments, the complement of the guide RNA has a sequence identity of about 99% to a CRISPR nucleic acid sequence. In embodiments, the complement of the guide RNA has a sequence identity of about 100% to a CRISPR nucleic acid sequence. In embodiments, the complement of the guide RNA has a sequence identity of at least 50%, 55%, 60%, 65%, 70%, 75%, 80%, 85%, 90%, 95%, 96%, 97%, 98%, 99%, or 100% to a target nucleic acid. In embodiments, a CRISPR nucleic acid sequence as provided herein is a nucleic acid sequence expressed by a cell. In embodiments, the CRISPR nucleic acid sequence is an exogenous nucleic acid sequence. In embodiments, the CRISPR nucleic acid sequence is an endogenous nucleic acid sequence. In embodiments, the CRISPR nucleic acid sequence forms part of a cellular gene. In embodiments, the CRISPR nucleic acid sequence is adjacent to a PAM sequence. In embodiments, the PAM sequence is the sequence chosen from the group (read from 5′ to 3′): NGG, NGA, TTTN, TTTV, YTN, NGRRT, NGRRN, NNNNGATT, NNNNRYAC, NNAGAAW, or NAAAAC, wherein N is any nucleobase; V is guanine, cytosine or adenine; R is guanine or adenine; Y is cytosine or thymine; and W is adenine or thymine.

In embodiments, the guide RNA is complementary to a cellular gene or fragment thereof (e.g., retinoic acid receptor gene or a complement thereof). In embodiments, the guide RNA binds a cellular gene sequence (e.g., retinoic acid receptor gene or a fragment thereof, or a complement thereof). In embodiments, a guide RNA is at least 60%, 65%, 70%, 75%, 80%, 85%, 90%, 95%, 96%, 97%, 98%, 99%, or 100% identical to a complementary sequence to the target gene or target nucleic acid (e.g., retinoic acid receptor gene or a fragment thereof, or a complement thereof). In embodiments, a guide RNA is at least 60% identical to a complementary sequence to the target gene or target nucleic acid (e.g., retinoic acid receptor gene or a fragment thereof, or a complement thereof). In embodiments, a guide RNA is at least 65% identical to a complementary sequence to the target gene or target nucleic acid (e.g., retinoic acid receptor gene or a fragment thereof, or a complement thereof). In embodiments, a guide RNA is at least 70% identical to a complementary sequence to the target gene or target nucleic acid (e.g., retinoic acid receptor gene or a fragment thereof, or a complement thereof). In embodiments, a guide RNA is at least 75% identical to a complementary sequence to the target gene or target nucleic acid (e.g., retinoic acid receptor gene or a fragment thereof, or a complement thereof). In embodiments, a guide RNA is at least 80% identical to a complementary sequence to the target gene or target nucleic acid (e.g., retinoic acid receptor gene or a fragment thereof, or a complement thereof). In embodiments, a guide RNA is at least 85% identical to a complementary sequence to the target gene or target nucleic acid (e.g., retinoic acid receptor gene or a fragment thereof, or a complement thereof). In embodiments, a guide RNA is at least 90% identical to a complementary sequence to the target gene or target nucleic acid (e.g., retinoic acid receptor gene or a fragment thereof, or a complement thereof). In embodiments, a guide RNA is at least 95% identical to a complementary sequence to the target gene or target nucleic acid (e.g., retinoic acid receptor gene or a fragment thereof, or a complement thereof). In embodiments, a guide RNA is at least 96% identical to a complementary sequence to the target gene or target nucleic acid (e.g., retinoic acid receptor gene or a fragment thereof, or a complement thereof). In embodiments, a guide RNA is at least 97% identical to a complementary sequence to the target gene or target nucleic acid (e.g., retinoic acid receptor gene or a fragment thereof, or a complement thereof). In embodiments, a guide RNA is at least 98% identical to a complementary sequence to the target gene or target nucleic acid (e.g., retinoic acid receptor gene or a fragment thereof, or a complement thereof). In embodiments, a guide RNA is at least 99% identical to a complementary sequence to the target gene or target nucleic acid (e.g., retinoic acid receptor gene or a fragment thereof, or a complement thereof). In embodiments, a guide RNA is at least 100% identical to a complementary sequence to the target gene or target nucleic acid (e.g., retinoic acid receptor gene or a fragment thereof, or a complement thereof). In embodiments, a guide RNA includes one or more nucleotide analogs (e.g., nucleotide analog(s) described herein). In embodiments, target gene or target nucleic acid (e.g., retinoic acid receptor gene or a fragment thereof, or a complement thereof) is adjacent to a PAM sequence. In embodiments, the target gene or target nucleic acid (e.g., retinoic acid receptor gene or a fragment thereof, or a complement thereof) is adjacent to a PAM sequence. In embodiments, the PAM sequence is the sequence chosen from the group (read from 5′ to 3′): NGG, NGA, TTTN, TTTV, YTN, NGRRT, NGRRN, NNNNGATT, NNNNRYAC, NNAGAAW, or NAAAAC, wherein N is any nucleobase; V is guanine, cytosine or adenine; R is guanine or adenine; Y is cytosine or thymine; and W is adenine or thymine.

In embodiments, a guide RNA is at least 90% identical to a complementary sequence to the target gene or target nucleic acid (e.g., retinoic acid receptor gene or a fragment thereof, or a complement thereof), or a complementary sequence to the nucleic acid sequence upstream or downstream of the target gene or target nucleic acid (e.g., retinoic acid receptor gene or a fragment thereof, or a complement thereof) transcription start site. In embodiments, a guide RNA is at least 90% identical to a complementary sequence to the target gene or target nucleic acid (e.g., retinoic acid receptor gene or a fragment thereof, or a complement thereof) or nucleic acid sequence within 100 nucleotides upstream of the retinoic acid receptor transcription start site. In embodiments, a guide RNA is at least 90% identical to a complementary sequence to the target gene or target nucleic acid (e.g., retinoic acid receptor gene or a fragment thereof, or a complement thereof) or nucleic acid sequence within 100 nucleotides downstream of the retinoic acid receptor transcription start site. In embodiments, a guide RNA is at least 95% identical to a complementary sequence to the target gene or target nucleic acid (e.g., retinoic acid receptor gene or a fragment thereof, or a complement thereof), or a complementary sequence to the nucleic acid sequence upstream or downstream of the target gene or target nucleic acid (e.g., retinoic acid receptor gene or a fragment thereof, or a complement thereof) transcription start site. In embodiments, a guide RNA is at least 95% identical to a complementary sequence to the target gene or target nucleic acid (e.g., retinoic acid receptor gene or a fragment thereof, or a complement thereof) or nucleic acid sequence within 100 nucleotides upstream of the retinoic acid receptor transcription start site. In embodiments, a guide RNA is at least 95% identical to a complementary sequence to the target gene or target nucleic acid (e.g., retinoic acid receptor gene or a fragment thereof, or a complement thereof) or nucleic acid sequence within 100 nucleotides downstream of the retinoic acid receptor transcription start site. In embodiments, a guide RNA is at least 100% identical to a complementary sequence to the target gene or target nucleic acid (e.g., retinoic acid receptor gene or a fragment thereof, or a complement thereof), or a complementary sequence to the nucleic acid sequence upstream or downstream of the target gene or target nucleic acid (e.g., retinoic acid receptor gene or a fragment thereof, or a complement thereof) transcription start site. In embodiments, a guide RNA is at least 100% identical to a complementary sequence to the target gene or target nucleic acid (e.g., retinoic acid receptor gene or a fragment thereof, or a complement thereof) or nucleic acid sequence within 100 nucleotides upstream of the retinoic acid receptor transcription start site. In embodiments, a guide RNA is at least 100% identical to a complementary sequence to the target gene or target nucleic acid (e.g., retinoic acid receptor gene or a fragment thereof, or a complement thereof) or nucleic acid sequence within 100 nucleotides downstream of the retinoic acid receptor transcription start site. In embodiments, target gene or target nucleic acid (e.g., retinoic acid receptor gene or a fragment thereof, or a complement thereof) is adjacent to a PAM sequence. In embodiments, the target gene or target nucleic acid (e.g., retinoic acid receptor gene or a fragment thereof, or a complement thereof) is adjacent to a PAM sequence. In embodiments, the PAM sequence is the sequence chosen from the group (read from 5′ to 3′): NGG, NGA, TTTN, TTTV, YTN, NGRRT, NGRRN, NNNNGATT, NNNNRYAC, NNAGAAW, or NAAAAC, wherein N is any nucleobase; V is guanine, cytosine or adenine; R is guanine or adenine; Y is cytosine or thymine; and W is adenine or thymine.

In embodiments, the guide RNA is a single-stranded ribonucleic acid. In embodiments, the guide RNA is about 10, 20, 30, 40, 50, 60, 70, 80, 90, 100 or more nucleic acid residues in length. In embodiments, the guide RNA is from about 10 to about 30 nucleic acid residues in length. In embodiments, the guide RNA is about 20 nucleic acid residues in length. In embodiments, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the guide RNA is from 10 to 15, 10 to 20, 10 to 30, 10 to 40, or 10 to 50 residues in length. In embodiments, the guide RNA is from 19 to 23 residues in length.

In embodiments, the guide RNA includes a nucleic acid sequence from 10 to 50 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 10 to 30 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 19 to 23 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 10 to 50 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 10 to 30 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 19 to 23 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 10 to 50 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 10 to 30 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site. In embodiments, the guide RNA includes a nucleic acid sequence from 19 to 23 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site.

In embodiments, the guide RNA includes a nucleic acid sequence from 10 to 50 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 10 to 30 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 19 to 23 nucleotides in length and at least 90% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 10 to 50 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 10 to 30 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 19 to 23 nucleotides in length and at least 95% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 10 to 50 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 10 to 30 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site. In embodiments, the guide RNA includes a nucleic acid sequence from 19 to 23 nucleotides in length and at least 100% identical to an RNA sequence of a retinoic acid receptor or a fragment thereof, or a complement thereof, or an RNA sequence or a fragment thereof, or a complement thereof corresponding to a nucleic acid sequence within 100 nucleotides upstream or downstream of the retinoic acid receptor transcription start site.

In embodiments, the guide RNA includes a nucleic acid sequence from 10 to 50 nucleotides in length and at least 90% identical to an RNA sequence of a retinoic acid receptor or a fragment thereof, or a complement thereof. In embodiments, the guide RNA includes a nucleic acid sequence from 10 to 30 nucleotides in length and at least 90% identical to an RNA sequence of a retinoic acid receptor or a fragment thereof, or a complement thereof. In embodiments, the guide RNA includes a nucleic acid sequence from 19 to 23 nucleotides in length and at least 90% identical to an RNA sequence of a retinoic acid receptor or a fragment thereof, or a complement thereof. In embodiments, the guide RNA includes a nucleic acid sequence from 10 to 50 nucleotides in length and at least 95% identical to an RNA sequence of a retinoic acid receptor or a fragment thereof, or a complement thereof. In embodiments, the guide RNA includes a nucleic acid sequence from 10 to 30 nucleotides in length and at least 95% identical to an RNA sequence of a retinoic acid receptor or a fragment thereof, or a complement thereof. In embodiments, the guide RNA includes a nucleic acid sequence from 19 to 23 nucleotides in length and at least 95% identical to an RNA sequence of a retinoic acid receptor or a fragment thereof, or a complement thereof. In embodiments, the guide RNA includes a nucleic acid sequence from 10 to 50 nucleotides in length and at least 100% identical to an RNA sequence of a retinoic acid receptor or a fragment thereof, or a complement thereof. In embodiments, the guide RNA includes a nucleic acid sequence from 10 to 30 nucleotides in length and at least 100% identical to an RNA sequence of a retinoic acid receptor or a fragment thereof, or a complement thereof. In embodiments, the guide RNA includes a nucleic acid sequence from 19 to 23 nucleotides in length and at least 100% identical to an RNA sequence of a retinoic acid receptor or a fragment thereof, or a complement thereof.

In embodiments, the guide RNA includes a nucleic acid sequence complementary to the sequence GATGTACGAGAGTGTAGAAG (SEQ ID NO:4). In embodiments, the guide RNA includes a nucleic acid sequence complementary to the sequence TATATCCACTAACTGGAAGC (SEQ ID NO:5). In embodiments, the guide RNA includes a nucleic acid sequence complementary to the sequence GTCCGTACTCCACCCCGCTC (SEQ ID NO:6). In embodiments, the guide RNA includes a nucleic acid sequence complementary to the sequence CCATTGAGGTGCCCGCCCCC (SEQ ID NO:7). In embodiments, the guide RNA includes a nucleic acid sequence complementary to the sequence CTTGTAGATGCGGGGTAGAG (SEQ ID NO:8). In embodiments, the guide RNA includes a nucleic acid sequence complementary to the sequence TGATGATGCAGTTCTTGTCC (SEQ ID NO:9).

In embodiments, the guide RNA includes a nucleic acid sequence corresponding to the sequence GATGTACGAGAGTGTAGAAG (SEQ ID NO:4). In embodiments, the guide RNA includes a nucleic acid sequence corresponding to the sequence TATATCCACTAACTGGAAGC (SEQ ID NO:5). In embodiments, the guide RNA includes a nucleic acid sequence corresponding to the sequence GTCCGTACTCCACCCCGCTC (SEQ ID NO:6). In embodiments, the guide RNA includes a nucleic acid sequence corresponding to the sequence CCATTGAGGTGCCCGCCCCC (SEQ ID NO:7). In embodiments, the guide RNA includes a nucleic acid sequence corresponding to the sequence CTTGTAGATGCGGGGTAGAG (SEQ ID NO:8). In embodiments, the guide RNA includes a nucleic acid sequence corresponding to the sequence TGATGATGCAGTTCTTGTCC (SEQ ID NO:9).

In embodiments, the TAL effector nuclease is capable of modifying the nucleic acid sequence of the retinoic acid receptor (e.g., RARα, SEQ ID NO:2, RARβ, or RARγ). In embodiments, the TAL effector nuclease is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the TAL effector nuclease is capable of modifying the nucleic acid sequence of the retinoid x receptor. In embodiments, the TAL effector nuclease is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid receptor-retinoid x receptor heterodimer.

In embodiments, TAL effector protein includes at least 6 TAL repeats. In embodiments, TAL effector protein includes at least 8 TAL repeats. In embodiments, TAL effector protein includes at least 10 TAL repeats. In embodiments, TAL effector protein includes at least 12 TAL repeats. In embodiments, TAL effector protein includes at least 15 TAL repeats. In embodiments, TAL effector protein includes at least 17 TAL repeats. In embodiments, TAL effector protein includes from about 6 to about 25 TAL repeats. In embodiments, TAL effector protein includes from about 6 to about 35 TAL repeats. In embodiments, TAL effector protein includes from about 8 to about 25 TAL repeats. In embodiments, TAL effector protein includes at least 10 to about 25 TAL repeats. In embodiments, TAL effector protein includes from about 12 to about 25 TAL repeats. In embodiments, TAL effector protein includes from about 8 to about 22 TAL repeats. In embodiments, TAL effector protein includes from about 10 to about 22 TAL repeats. In embodiments, TAL effector protein includes from about 12 to about 22 TAL repeats. In embodiments, TAL effector protein includes from about 6 to about 20 TAL repeats. In embodiments, TAL effector protein includes from about 8 to about 20 TAL repeats. In embodiments, TAL effector protein includes from about 10 to about 22 TAL repeats. In embodiments, TAL effector protein includes from about 12 to about 20 TAL repeats. In embodiments, TAL effector protein includes from about 6 to about 18 TAL repeats. In embodiments, TAL effector protein includes from about 10 to about 18 TAL repeats. In embodiments, TAL effector protein includes from about 12 to about 18 TAL repeats. In embodiments, the TAL effector protein includes 18 or 24 or 17.5 or 23.5 TAL nucleic acid binding cassettes. In embodiments, the TAL effector protein includes 15.5, 16.5, 18.5, 19.5, 20.5, 21.5, 22.5 or 24.5 TAL nucleic acid binding cassettes. In embodiments, a TAL effector protein includes at least one polypeptide region which flanks the region containing the TAL repeats. In embodiments, flanking regions are present at the amino and/or the carboxyl termini of the TAL repeats.

In embodiments, the zinc-finger nuclease is capable of modifying the nucleic acid sequence of the retinoic acid receptor (e.g., RARα, SEQ ID NO:2, RARβ, or RARγ). In embodiments, the zinc-finger nuclease is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the zinc-finger nuclease is capable of modifying the nucleic acid sequence of the retinoid x receptor. In embodiments, the zinc-finger nuclease is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid receptor-retinoid x receptor heterodimer. In embodiments, a zinc-finger protein has at least one finger. In embodiments, a zinc-finger protein has at least two fingers. In embodiments, a zinc-finger protein has at least three fingers. In embodiments, a zinc-finger protein has at least four fingers. In embodiments, a zinc-finger protein has at least five fingers. In embodiments, a zinc-finger protein has at least six fingers.

In embodiments, the meganuclease is capable of modifying the nucleic acid sequence of the retinoic acid receptor (e.g., RARα, SEQ ID NO:2, RARβ, or RARγ). In embodiments, the meganuclease is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the megauclease is capable of modifying the nucleic acid sequence of the retinoid x receptor. In embodiments, the meganuclease is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid-retinoid x receptor heterodimer.

In embodiments, the homing endonuclease is capable of modifying the nucleic acid sequence of the retinoic acid receptor (e.g., RARα, SEQ ID NO:2, RARβ, or RARγ). In embodiments, the homing endonuclease is capable of modifying the nucleic acid sequence of the retinoic acid receptor (e.g., RARα, SEQ ID NO:2, RARβ, or RARγ) such that the modification to the nucleic acid sequence of the retinoic acid receptor reduces the activity of the retinoic acid receptor (e.g., the activity of the retinoic acid receptor protein). In embodiments, the homing endonuclease is capable of modifying the nucleic acid sequence of the retinoid x receptor. In embodiments, the homing endonuclease is capable of modifying the nucleic acid sequence of the retinoid x receptor such that the modification to the nucleic acid sequence of the retinoid x receptor reduces the activity of the retinoic acid receptor (e.g., the activity of the retinoic acid receptor protein) or the retinoic acid-retinoid x receptor heterodimer.

In embodiments, the antisense nucleic acid is capable of modifying the level of expression of the retinoic acid receptor (e.g., RARα, SEQ ID NO:2, SEQ ID NO:3, RARβ, or RARγ). In embodiments, the antisense nucleic acid is capable of modifying the level of expression of the retinoic acid receptor (e.g., RARα, SEQ ID NO:2, SEQ ID NO:3, RARβ, or RARγ) such that the modification reduces the activity of the retinoic acid receptor (e.g., the level of activity of the retinoic acid receptor protein in a cell, organ, subject, or other vessel). In embodiments, the antisense nucleic acid is capable of modifying the level of expression of the nucleic acid sequence of the retinoid x receptor. In embodiments, the antisense nucleic acid is capable of modifying the level of expression of the nucleic acid sequence of the retinoid x receptor such that the modification reduces the activity of the retinoic acid receptor (e.g., the level of activity of the retinoic acid receptor protein in a cell, organ, subject, or other vessel) or the retinoic acid receptor-retinoid x receptor heterodimer.

In embodiments, an antisense nucleic acid is about 5, 10, 15, 20, 25, 30, 35, 40, 45, 50, 55, 60, 65, 70, 75, 80, 85, 90, 95, 100 or more nucleic acid residues or sugar residues in length. In embodiments, an antisense nucleic acid is 5, 10, 15, 20, 25, 30, 35, 40, 45, 50, 55, 60, 65, 70, 75, 80, 85, 90, 95, 100 or more nucleic acid residues or sugar residues in length. In embodiments, an antisense nucleic acid is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, an antisense nucleic acids is from 10 to 15, 10 to 20, 10 to 30, 10 to 40, or 10 to 50 residues in length. In embodiments, an antisense nucleic acid is from 19 to 23 residues in length.

In embodiments, an antisense nucleic acid is at least 60%, 65%, 70%, 75%, 80%, 85%, 90%, 95%, 96%, 97%, 98%, 99%, or 100% identical to a complementary sequence to the target gene or target nucleic acid (e.g., retinoic acid receptor gene or a fragment thereof). In embodiments, an antisense nucleic acid is at least 60% identical to a complementary sequence to the target gene or target nucleic acid (e.g., retinoic acid receptor gene or a fragment thereof). In embodiments, an antisense nucleic acid is at least 65% identical to a complementary sequence to the target gene or target nucleic acid (e.g., retinoic acid receptor gene or a fragment thereof). In embodiments, an antisense nucleic acid is at least 70% identical to a complementary sequence to the target gene or target nucleic acid (e.g., retinoic acid receptor gene or a fragment thereof). In embodiments, an antisense nucleic acid is at least 75% identical to a complementary sequence to the target gene or target nucleic acid (e.g., retinoic acid receptor gene or a fragment thereof). In embodiments, an antisense nucleic acid is at least 80% identical to a complementary sequence to the target gene or target nucleic acid (e.g., retinoic acid receptor gene or a fragment thereof). In embodiments, an antisense nucleic acid is at least 85% identical to a complementary sequence to the target gene or target nucleic acid (e.g., retinoic acid receptor gene or a fragment thereof). In embodiments, an antisense nucleic acid is at least 90% identical to a complementary sequence to the target gene or target nucleic acid (e.g., retinoic acid receptor gene or a fragment thereof). In embodiments, an antisense nucleic acid is at least 95% identical to a complementary sequence to the target gene or target nucleic acid (e.g., retinoic acid receptor gene or a fragment thereof). In embodiments, an antisense nucleic acid is at least 96% identical to a complementary sequence to the target gene or target nucleic acid (e.g., retinoic acid receptor gene or a fragment thereof). In embodiments, an antisense nucleic acid is at least 97% identical to a complementary sequence to the target gene or target nucleic acid (e.g., retinoic acid receptor gene or a fragment thereof). In embodiments, an antisense nucleic acid is at least 98% identical to a complementary sequence to the target gene or target nucleic acid (e.g., retinoic acid receptor gene or a fragment thereof). In embodiments, an antisense nucleic acid is at least 99% identical to a complementary sequence to the target gene or target nucleic acid (e.g., retinoic acid receptor gene or a fragment thereof). In embodiments, an antisense nucleic acid is at least 100% identical to a complementary sequence to the target gene or target nucleic acid (e.g., retinoic acid receptor gene or a fragment thereof). In embodiments, the antisense nucleic acid is DNA (e.g., including one or more nucleotide analogs). In embodiments, the antisense nucleic acid is RNA (e.g., including one or more nucleotide analogs).

In embodiments, an antisense nucleic acid is at least 90% identical to a complementary sequence to the target gene or target nucleic acid sequence (e.g., retinoic acid receptor gene or a fragment thereof), or a complementary sequence to the nucleic acid sequence (e.g., retinoic acid receptor gene or a fragment thereof) upstream or downstream of the target gene or target nucleic acid (e.g., retinoic acid receptor) transcription start site. In embodiments, an antisense nucleic acid is at least 90% identical to a complementary sequence to the target gene or target nucleic acid sequence (e.g., retinoic acid receptor gene or a fragment thereof) within 100 nucleotides upstream of the target gene or target nucleic acid (e.g., retinoic acid receptor) transcription start site. In embodiments, an antisense nucleic acid is at least 90% identical to a complementary sequence to the target gene or target nucleic acid sequence (e.g., retinoic acid receptor gene or a fragment thereof) within 100 nucleotides downstream of the target gene or target nucleic acid (e.g., retinoic acid receptor) transcription start site. In embodiments, an antisense nucleic acid is at least 95% identical to a complementary sequence to the target gene or target nucleic acid sequence (e.g., retinoic acid receptor gene or a fragment thereof) upstream or downstream of the target gene or target nucleic acid (e.g., retinoic acid receptor) transcription start site. In embodiments, an antisense nucleic acid is at least 95% identical to a complementary sequence to the target gene or target nucleic acid sequence (e.g., retinoic acid receptor gene or a fragment thereof) within 100 nucleotides upstream of the target gene or target nucleic acid (e.g., retinoic acid receptor) transcription start site. In embodiments, an antisense nucleic acid is at least 95% identical to a complementary sequence to the target gene or target nucleic acid sequence (e.g., retinoic acid receptor gene or a fragment thereof) within 100 nucleotides downstream of the target gene or target nucleic acid (e.g., retinoic acid receptor) transcription start site. In embodiments, an antisense nucleic acid is at least 100% identical to a complementary sequence to the target gene or target nucleic acid sequence (e.g., retinoic acid receptor gene or a fragment thereof) upstream or downstream of the target gene or target nucleic acid (e.g., retinoic acid receptor) transcription start site. In embodiments, an antisense nucleic acid is at least 100% identical to a complementary sequence to the target gene or target nucleic acid sequence (e.g., retinoic acid receptor gene or a fragment thereof) within 100 nucleotides upstream of the target gene or target nucleic acid (e.g., retinoic acid receptor) transcription start site. In embodiments, an antisense nucleic acid is at least 100% identical to a complementary sequence to the target gene or target nucleic acid sequence (e.g., retinoic acid receptor gene or a fragment thereof) within 100 nucleotides downstream of the target gene or target nucleic acid (e.g., retinoic acid receptor) transcription start site. In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid includes a nucleic acid sequence from 10 to 50 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 10 to 30 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 19 to 23 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 10 to 50 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 10 to 30 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 19 to 23 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 10 to 50 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 10 to 30 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid includes a nucleic acid sequence from 19 to 23 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid is a nucleic acid sequence from 10 to 50 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 10 to 30 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 19 to 23 nucleotides in length and at least 90% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 10 to 50 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 10 to 30 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 19 to 23 nucleotides in length and at least 95% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 10 to 50 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 10 to 30 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is a nucleic acid sequence from 19 to 23 nucleotides in length and at least 100% identical to a complementary sequence to the target gene or target nucleic acid sequence (e.g., retinoic acid receptor gene or a fragment thereof) or a nucleic acid sequence upstream or downstream of the target gene or target nucleic acid (e.g., retinoic acid receptor) transcription start site. In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid includes a nucleic acid sequence from 10 to 50 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 10 to 30 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 19 to 23 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 10 to 50 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 10 to 30 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 19 to 23 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 10 to 50 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 10 to 30 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid includes a nucleic acid sequence from 19 to 23 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid is a nucleic acid sequence from 10 to 50 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 10 to 30 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 19 to 23 nucleotides in length and at least 90% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 10 to 50 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 10 to 30 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 19 to 23 nucleotides in length and at least 95% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 10 to 50 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 10 to 30 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is a nucleic acid sequence from 19 to 23 nucleotides in length and at least 100% identical to a complementary sequence to the target gene or target nucleic acid sequence (e.g., retinoic acid receptor gene or a fragment thereof) or a nucleic acid sequence within 100 nucleotides upstream or downstream of the target gene or target nucleic acid (e.g., retinoic acid receptor) transcription start site. In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid includes a nucleic acid sequence from 10 to 50 nucleotides in length and at least 90% identical to a complementary sequence to the target gene or target nucleic acid sequence (e.g., retinoic acid receptor gene or a fragment thereof). In embodiments, the antisense nucleic acid includes a nucleic acid sequence from 10 to 30 nucleotides in length and at least 90% identical to a complementary sequence to the target gene or target nucleic acid sequence (e.g., retinoic acid receptor gene or a fragment thereof). In embodiments, the antisense nucleic acid includes a nucleic acid sequence from 19 to 23 nucleotides in length and at least 90% identical to a complementary sequence to the target gene or target nucleic acid sequence (e.g., retinoic acid receptor gene or a fragment thereof). In embodiments, the antisense nucleic acid includes a nucleic acid sequence from 10 to 50 nucleotides in length and at least 95% identical to a complementary sequence to the target gene or target nucleic acid sequence (e.g., retinoic acid receptor gene or a fragment thereof). In embodiments, the antisense nucleic acid includes a nucleic acid sequence from 10 to 30 nucleotides in length and at least 95% identical to a complementary sequence to the target gene or target nucleic acid sequence (e.g., retinoic acid receptor gene or a fragment thereof). In embodiments, the antisense nucleic acid includes a nucleic acid sequence from 19 to 23 nucleotides in length and at least 95% identical to a complementary sequence to the target gene or target nucleic acid sequence (e.g., retinoic acid receptor gene or a fragment thereof). In embodiments, the antisense nucleic acid includes a nucleic acid sequence from 10 to 50 nucleotides in length and at least 100% identical to a complementary sequence to the target gene or target nucleic acid sequence (e.g., retinoic acid receptor gene or a fragment thereof). In embodiments, the antisense nucleic acid includes a nucleic acid sequence from 10 to 30 nucleotides in length and at least 100% identical to a complementary sequence to the target gene or target nucleic acid sequence (e.g., retinoic acid receptor gene or a fragment thereof). In embodiments, the antisense nucleic acid includes a nucleic acid sequence from 19 to 23 nucleotides in length and at least 100% identical to a complementary sequence to the target gene or target nucleic acid sequence (e.g., retinoic acid receptor gene or a fragment thereof). In embodiments, the antisense nucleic acid is DNA (e.g., including one or more nucleotide analogs). In embodiments, the antisense nucleic acid is RNA (e.g., including one or more nucleotide analogs).

In embodiments, the antisense nucleic acid is a nucleic acid sequence from 10 to 50 nucleotides in length and at least 90% identical to a complementary sequence to the target gene or target nucleic acid sequence (e.g., retinoic acid receptor gene or a fragment thereof). In embodiments, the antisense nucleic acid is a nucleic acid sequence from 10 to 30 nucleotides in length and at least 90% identical to a complementary sequence to the target gene or target nucleic acid sequence (e.g., retinoic acid receptor gene or a fragment thereof). In embodiments, the antisense nucleic acid is a nucleic acid sequence from 19 to 23 nucleotides in length and at least 90% identical to a complementary sequence to the target gene or target nucleic acid sequence (e.g., retinoic acid receptor gene or a fragment thereof). In embodiments, the antisense nucleic acid is a nucleic acid sequence from 10 to 50 nucleotides in length and at least 95% identical to a complementary sequence to the target gene or target nucleic acid sequence (e.g., retinoic acid receptor gene or a fragment thereof). In embodiments, the antisense nucleic acid is a nucleic acid sequence from 10 to 30 nucleotides in length and at least 95% identical to a complementary sequence to the target gene or target nucleic acid sequence (e.g., retinoic acid receptor gene or a fragment thereof). In embodiments, the antisense nucleic acid is a nucleic acid sequence from 19 to 23 nucleotides in length and at least 95% identical to a complementary sequence to the target gene or target nucleic acid sequence (e.g., retinoic acid receptor gene or a fragment thereof). In embodiments, the antisense nucleic acid is a nucleic acid sequence from 10 to 50 nucleotides in length and at least 100% identical to a complementary sequence to the target gene or target nucleic acid sequence (e.g., retinoic acid receptor gene or a fragment thereof). In embodiments, the antisense nucleic acid is a nucleic acid sequence from 10 to 30 nucleotides in length and at least 100% identical to a complementary sequence to the target gene or target nucleic acid sequence (e.g., retinoic acid receptor gene or a fragment thereof). In embodiments, the antisense nucleic acid is a nucleic acid sequence from 19 to 23 nucleotides in length and at least 100% identical to a complementary sequence to the target gene or target nucleic acid sequence (e.g., retinoic acid receptor gene or a fragment thereof). In embodiments, the antisense nucleic acid is DNA (e.g., including one or more nucleotide analogs). In embodiments, the antisense nucleic acid is RNA (e.g., including one or more nucleotide analogs).

In embodiments, the siRNA is capable of modifying the level of expression of the retinoic acid receptor (e.g., RARα, SEQ ID NO:2, SEQ ID NO:3, RARβ, or RARγ). In embodiments, the siRNA is capable of modifying the level of expression of the retinoic acid receptor (e.g., RARα, SEQ ID NO:2, SEQ ID NO:3, RARβ, or RARγ) such that the modification reduces the activity of the retinoic acid receptor (e.g., the level of activity of the retinoic acid receptor protein in a cell, organ, subject, or other vessel). In embodiments, the siRNA is capable of modifying the level of expression of the retinoid x receptor. In embodiments, the siRNA is capable of modifying the level of expression of the retinoid x receptor such that the modification reduces the activity of the retinoic acid receptor (e.g., the level of activity of the retinoic acid receptor protein in a cell, organ, subject, or other vessel) or the retinoic acid receptor-retinoid x receptor heterodimer.

In embodiments, the siRNA is from about 20 to about 30 nucleotides in length. In embodiments, the siRNA is from about 20 to about 25 nucleotides in length. In embodiments, the siRNA is from about 24 to about 29 nucleotides in length. In embodiments, the siRNA is 20, 21, 22, 23, 24, 25, 26, 27, 28, 29, or 30 nucleotides in length. In embodiments, the siRNA is at least 60%, 65%, 70%, 75%, 80%, 85%, 90%, 95%, 96%, 97%, 98%, 99%, or 100% identical to a complementary sequence to the target gene or target nucleic acid (e.g., retinoic acid receptor gene or a fragment thereof). In embodiments, the siRNA is at least 60% identical to a complementary sequence to the target gene or target nucleic acid (e.g., retinoic acid receptor gene or a fragment thereof). In embodiments, the siRNA is at least 65% identical to a complementary sequence to the target gene or target nucleic acid (e.g., retinoic acid receptor gene or a fragment thereof). In embodiments, the siRNA is at least 70% identical to a complementary sequence to the target gene or target nucleic acid (e.g., retinoic acid receptor gene or a fragment thereof). In embodiments, the siRNA is at least 75% identical to a complementary sequence to the target gene or target nucleic acid (e.g., retinoic acid receptor gene or a fragment thereof). In embodiments, the siRNA is at least 80% identical to a complementary sequence to the target gene or target nucleic acid (e.g., retinoic acid receptor gene or a fragment thereof). In embodiments, the siRNA is at least 85% identical to a complementary sequence to the target gene or target nucleic acid (e.g., retinoic acid receptor gene or a fragment thereof). In embodiments, the siRNA is at least 90% identical to a complementary sequence to the target gene or target nucleic acid (e.g., retinoic acid receptor gene or a fragment thereof). In embodiments, the siRNA is at least 95% identical to a complementary sequence to the target gene or target nucleic acid (e.g., retinoic acid receptor gene or a fragment thereof). In embodiments, the siRNA is at least 96% identical to a complementary sequence to the target gene or target nucleic acid (e.g., retinoic acid receptor gene or a fragment thereof). In embodiments, the siRNA is at least 97% identical to a complementary sequence to the target gene or target nucleic acid (e.g., retinoic acid receptor gene or a fragment thereof). In embodiments, the siRNA is at least 98% identical to a complementary sequence to the target gene or target nucleic acid (e.g., retinoic acid receptor gene or a fragment thereof). In embodiments, the siRNA is at least 99% identical to a complementary sequence to the target gene or target nucleic acid (e.g., retinoic acid receptor gene or a fragment thereof). In embodiments, the siRNA is at least 100% identical to a complementary sequence to the target gene or target nucleic acid (e.g., retinoic acid receptor gene or a fragment thereof).

In embodiments, an siRNA is at least 90% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, an siRNA is at least 90% identical to a complementary sequence to the target gene or target nucleic acid (e.g., retinoic acid receptor gene or a fragment thereof), or a complementary sequence to the nucleic acid sequence within 100 nucleotides upstream of the target gene or target nucleic acid (e.g., retinoic acid receptor) transcription start site. In embodiments, an siRNA is at least 90% identical to a complementary sequence to the target gene or target nucleic acid (e.g., retinoic acid receptor gene or a fragment thereof), or a complementary sequence to the nucleic acid sequence within 100 nucleotides downstream of the target gene or target nucleic acid (e.g., retinoic acid receptor) transcription start site. In embodiments, an siRNA is at least 95% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, an siRNA is at least 95% identical to a complementary sequence to the target gene or target nucleic acid (e.g., retinoic acid receptor gene or a fragment thereof), or a complementary sequence to the nucleic acid sequence within 100 nucleotides upstream of the target gene or target nucleic acid (e.g., retinoic acid receptor) transcription start site. In embodiments, an siRNA is at least 95% identical to a complementary sequence to the target gene or target nucleic acid (e.g., retinoic acid receptor gene or a fragment thereof), or a complementary sequence to the nucleic acid sequence within 100 nucleotides downstream of the target gene or target nucleic acid (e.g., retinoic acid receptor) transcription start site. In embodiments, an siRNA is at least 100% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, an siRNA is at least 100% identical to a complementary sequence to the target gene or target nucleic acid (e.g., retinoic acid receptor gene or a fragment thereof), or a complementary sequence to the nucleic acid sequence within 100 nucleotides upstream of the target gene or target nucleic acid (e.g., retinoic acid receptor) transcription start site. In embodiments, an siRNA is at least 100% identical to a complementary sequence to the target gene or target nucleic acid (e.g., retinoic acid receptor gene or or a fragment thereof), or a complementary sequence to the nucleic acid sequence within 100 nucleotides downstream of the target gene or target nucleic acid (e.g., retinoic acid receptor) transcription start site.

In embodiments, the siRNA includes a nucleic acid sequence from 20 to 30 nucleotides in length and at least 90% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA includes a nucleic acid sequence from 24 to 29 nucleotides in length and at least 90% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA includes a nucleic acid sequence from 20 to 30 nucleotides in length and at least 95% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA includes a nucleic acid sequence from 24 to 29 nucleotides in length and at least 95% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA includes a nucleic acid sequence from 20 to 30 nucleotides in length and at least 100% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA includes a nucleic acid sequence from 24 to 29 nucleotides in length and at least 100% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site.

In embodiments, the siRNA is a nucleic acid sequence from 20 to 30 nucleotides in length and at least 90% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA is a nucleic acid sequence from 24 to 29 nucleotides in length and at least 90% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA is a nucleic acid sequence from 20 to 30 nucleotides in length and at least 95% identical to a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA is a nucleic acid sequence from 24 to 29 nucleotides in length and at least 95% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA is a nucleic acid sequence from 20 to 30 nucleotides in length and at least 100% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site. In embodiments, the siRNA is a nucleic acid sequence from 24 to 29 nucleotides in length and at least 100% identical a complementary sequence to the target gene or target nucleic acid (e.g., retinoic acid receptor gene or a fragment thereof), or a complementary sequence to the nucleic acid sequence upstream or downstream of the target gene or target nucleic acid (e.g., retinoic acid receptor) transcription start site.

In embodiments, the siRNA includes a nucleic acid sequence from 20 to 30 nucleotides in length and at least 90% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA includes a nucleic acid sequence from 24 to 29 nucleotides in length and at least 90% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA includes a nucleic acid sequence from 20 to 30 nucleotides in length and at least 95% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA includes a nucleic acid sequence from 24 to 29 nucleotides in length and at least 95% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA includes a nucleic acid sequence from 20 to 30 nucleotides in length and at least 100% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA includes a nucleic acid sequence from 24 to 29 nucleotides in length and at least 100% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site.

In embodiments, the siRNA is a nucleic acid sequence from 20 to 30 nucleotides in length and at least 90% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA is a nucleic acid sequence from 24 to 29 nucleotides in length and at least 90% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA is a nucleic acid sequence from 20 to 30 nucleotides in length and at least 95% identical to a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA is a nucleic acid sequence from 24 to 29 nucleotides in length and at least 95% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA is a nucleic acid sequence from 20 to 30 nucleotides in length and at least 100% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site. In embodiments, the siRNA is a nucleic acid sequence from 24 to 29 nucleotides in length and at least 100% identical a complementary sequence to the target gene or target nucleic acid (e.g., retinoic acid receptor gene or a fragment thereof), or a complementary sequence to the nucleic acid sequence within 100 nucleotides upstream or downstream of the target gene or target nucleic acid (e.g., retinoic acid receptor) transcription start site.

In embodiments, the siRNA includes a nucleic acid sequence from 20 to 30 nucleotides in length and at least 90% identical to a complementary sequence to the target gene or target nucleic acid (e.g., retinoic acid receptor gene or a fragment thereof). In embodiments, the siRNA includes a nucleic acid sequence from 24 to 29 nucleotides in length and at least 90% identical to a complementary sequence to the target gene or target nucleic acid (e.g., retinoic acid receptor gene or a fragment thereof). In embodiments, the siRNA includes a nucleic acid sequence from 20 to 30 nucleotides in length and at least 95% identical to a complementary sequence to the target gene or target nucleic acid (e.g., retinoic acid receptor gene or a fragment thereof). In embodiments, the siRNA includes a nucleic acid sequence from 24 to 29 nucleotides in length and at least 95% identical a complementary sequence to the target gene or target nucleic acid (e.g., retinoic acid receptor gene or a fragment thereof). In embodiments, the siRNA includes a nucleic acid sequence from 20 to 30 nucleotides in length and at least 100% identical a complementary sequence to the target gene or target nucleic acid (e.g., retinoic acid receptor gene or a fragment thereof). In embodiments, the siRNA includes a nucleic acid sequence from 24 to 29 nucleotides in length and at least 100% identical a complementary sequence to the target gene or target nucleic acid (e.g., retinoic acid receptor gene or a fragment thereof).

In embodiments, the siRNA is a nucleic acid sequence from 20 to 30 nucleotides in length and at least 90% identical to a complementary sequence to the target gene or target nucleic acid (e.g., retinoic acid receptor gene or a fragment thereof). In embodiments, the siRNA is a nucleic acid sequence from 24 to 29 nucleotides in length and at least 90% identical to a complementary sequence to the target gene or target nucleic acid (e.g., retinoic acid receptor gene or a fragment thereof). In embodiments, the siRNA is a nucleic acid sequence from 20 to 30 nucleotides in length and at least 95% identical to a complementary sequence to the target gene or target nucleic acid (e.g., retinoic acid receptor gene or a fragment thereof). In embodiments, the siRNA is a nucleic acid sequence from 24 to 29 nucleotides in length and at least 95% identical a complementary sequence to the target gene or target nucleic acid (e.g., retinoic acid receptor gene or a fragment thereof). In embodiments, the siRNA is a nucleic acid sequence from 20 to 30 nucleotides in length and at least 100% identical a complementary sequence to the target gene or target nucleic acid (e.g., retinoic acid receptor gene or a fragment thereof). In embodiments, the siRNA is a nucleic acid sequence from 24 to 29 nucleotides in length and at least 100% identical a complementary sequence to the target gene or target nucleic acid (e.g., retinoic acid receptor gene or a fragment thereof).

In embodiments, the gene modulating reagent is capable of modifying the nucleic acid sequence of the retinaldehyde dehydrogenase (RALDH). In embodiments, the gene modulating reagent is capable of modifying the nucleic acid sequence of the RALDH such that the modification to the nucleic acid sequence of the RALDH reduces the activity of the RALDH (e.g., the activity of the RALDH protein).

In embodiments, the CRISPR complex is capable of modifying the nucleic acid sequence of the retinaldehyde dehydrogenase (RALDH). In embodiments, the CRISPR complex is capable of modifying the nucleic acid sequence of the RALDH such that the modification to the nucleic acid sequence of the RALDH reduces the activity of the RALDH (e.g., the activity of the RALDH protein or the level of activity of the RALDH).

In embodiments, the TAL effector nuclease is capable of modifying the nucleic acid sequence of the retinaldehyde dehydrogenase (RALDH). In embodiments, the TAL effector nuclease is capable of modifying the nucleic acid sequence of the RALDH such that the modification to the nucleic acid sequence of the RALDH reduces the activity of the RALDH (e.g., the activity of the RALDH protein or the level of activity of the RALDH).

In embodiments, the zinc-finger nuclease is capable of modifying the nucleic acid sequence of the retinaldehyde dehydrogenase (RALDH). In embodiments, the zinc-finger nuclease is capable of modifying the nucleic acid sequence of the RALDH such that the modification to the nucleic acid sequence of the RALDH reduces the activity of the RALDH (e.g., the activity of the RALDH protein or the level of activity of the RALDH).

In embodiments, the meganuclease is capable of modifying the nucleic acid sequence of the retinaldehyde dehydrogenase (RALDH). In embodiments, the meganuclease is capable of modifying the nucleic acid sequence of the RALDH such that the modification to the nucleic acid sequence of the RALDH reduces the activity of the RALDH (e.g., the activity of the RALDH protein or the level of activity of the RALDH).

In embodiments, the homing endonuclease is capable of modifying the nucleic acid sequence of the retinaldehyde dehydrogenase (RALDH). In embodiments, the homing endonuclease is capable of modifying the nucleic acid sequence of the RALDH such that the modification to the nucleic acid sequence of the RALDH reduces the activity of the RALDH (e.g., the activity of the RALDH protein or the level of activity of the RALDH).

In embodiments, the antisense nucleic acid is capable of modifying the level of expression of the retinaldehyde dehydrogenase (RALDH). In embodiments, the antisense nucleic acid is capable of modifying the level of expression of the RALDH such that the modification reduces the activity of the RALDH (e.g., the level of activity of the RALDH protein in a cell, organ, subject, or other vessel).

In embodiments, the siRNA is capable of modifying the level of expression of the retinaldehyde dehydrogenase (RALDH). In embodiments, the siRNA is capable of modifying the level of expression of the RALDH such that the modification reduces the activity of the RALDH (e.g., the level of activity of the RALDH protein in a cell, organ, subject, or other vessel).

In embodiments, the method includes administering an expression vector encoding the gene modulating reagent. In embodiments, the method includes administering an expression vector encoding for the components included in a CRISPR complex, a TAL effector nuclease, a zinc-finger nuclease, a meganuclease, a homing endonuclease, an antisense nucleic acid, or an siRNA. In embodiments, the method includes administering an expression vector encoding for the components included in a CRISPR complex. In embodiments, the method includes administering an expression vector encoding for a TAL effector nuclease. In embodiments, the method includes administering an expression vector encoding for a zinc-finger nuclease. In embodiments, the method includes administering an expression vector encoding for a meganuclease. In embodiments, the method includes administering an expression vector encoding for a homing endonuclease. In embodiments, the method includes administering an expression vector encoding for an antisense nucleic acid. In embodiments, the method includes administering an expression vector encoding for an siRNA. In embodiments, the expression vector is a viral vector. In embodiments, the viral vector is an adenovirus vector, adeno-associated virus vector, or a lentiviral vector. In embodiments, the viral vector is an adenovirus vector. In embodiments, the viral vector is an adeno-associated virus vector. In embodiments, the viral vector is a lentiviral vector. In embodiments, the method includes administering an expression vector encoding for one or more of the components of a CRISPR complex.

In embodiments, the method includes administering a virus or viral vector, wherein the virus or viral vector includes a nucleic acid sequence. In embodiments, the nucleic acid sequence encodes a modified retinoic acid receptor. In embodiments, the modified retinoic acid receptor is a dominant negative form of the retinoic acid receptor. A dominant negative RAR is a retinoic acid receptor wherein the natural function is disrupted, for example wherein retinoic acid-mediated release is prevented. For example, in human RARα (e.g., SEQ ID NO:3), truncating the protein at amino acid 403 leads to a dominant negative form that competes against the endogenous unaltered receptor (e.g., wild-type RAR), resulting in the suppression of RAR-induced genes (see additional details in Damm K. et al., PNAS Apr. 1, 1993 vol. 90 no. 7 2989-29933; Novitch B. G. et al., Neuron. 2003 Sep. 25; 40(1):81-95; which are incorporated herein by reference in their entirety). In embodiments, the virus is an adenovirus, an Adeno-associated virus (AAV), or a lentivirus. In embodiments, the virus is an adenovirus. In embodiments, the virus is an Adeno-associated virus (AAV). In embodiments, the virus is a lentivirus. In embodiments, the viral vector is an adenovirus vector, an Adeno-associated virus (AAV) vector, or a lentiviral vector. In embodiments, the viral vector is an adenovirus vector. In embodiments, the viral vector is an Adeno-associated virus (AAV) vector. In embodiments, the viral vector is a lentiviral vector. In embodiments, the method includes administering a virus or viral vector, wherein the virus or viral vector includes a nucleic acid sequence encoding a modified retinoid x receptor.

In embodiments, the viral vector includes a virus engineered by directed evolution (e.g., to augment gene delivery and/or reduce immunogenicity). In embodiments, the viral vector includes a virus with altered coat proteins to augment gene delivery and reduce immunogenicity. In embodiments, the viral vector includes a virus engineered to be replication-incompetent. In embodiments, the viral vector includes a hybrid virus derived from multiple parent viral types. In embodiments, the viral vector includes a virus described in Planul, A., Dalkara, D. “Vectors and Gene Delivery to the Retina.” Annu. Rev. Vis. Sci. (2017) 3: 121-140, which is incorporated herein by reference in its entirety for all purposes.

In an aspect is provided a method for treating vision degeneration, the method including administering a virus or viral vector, wherein the virus or viral vector includes a nucleic acid sequence encoding a modified retinoic acid receptor or retinoid x receptor. In embodiments, the modified retinoic acid receptor is a dominant negative form of the retinoic acid receptor.

In an aspect is provided a method for treating vision degeneration, the method including administering a virus or viral vector, wherein the virus or viral vector includes a nucleic acid sequence encoding a modified retinaldehyde dehydrogenase. In embodiments, the modified retinaldehyde dehydrogenase is not capable of converting retinaldehyde to retinoic acid.

In embodiments, light sensitivity of retinal ganglion cells in the subject is increased, relative to a control (e.g., retinal ganglion cells in a subject not being administered an effective amount of a retinoic acid receptor inhibitor).

In embodiments, hyperexcitability of retinal ganglion cells in the subject is inhibited relative to a control (e.g., retinal ganglion cells in a subject not being administered an effective amount of a retinoic acid receptor inhibitor). In embodiments, increases in the number, activity, or cellular distribution of hyperpolarization-activated cyclic nucleotide-gated channel in retinal ganglion cells are reduced. In embodiments, increases in the number of hyperpolarization-activated cyclic nucleotide-gated channel in retinal ganglion cells are reduced (e.g., compared to control, absence of the RAR inhibitor). In embodiments, there is no increase in the number of hyperpolarization-activated cyclic nucleotide-gated channels in retinal ganglion cells. In embodiments, increases in the activity of hyperpolarization-activated cyclic nucleotide-gated channel in retinal ganglion cells are reduced (e.g., compared to control, absence of the RAR inhibitor). In embodiments, there is no increase in the activity of hyperpolarization-activated cyclic nucleotide-gated channels in retinal ganglion cells. In embodiments, increases in the cellular distribution of hyperpolarization-activated cyclic nucleotide-gated channel in retinal ganglion cells are reduced (e.g., compared to control, absence of the RAR inhibitor). In embodiments, there is no increase in the cellular distribution of hyperpolarization-activated cyclic nucleotide-gated channels in retinal ganglion cells.

In embodiments, the vision degeneration is associated with retinitis pigmentosa, age-related macular degeneration, cone dystrophy, rod-cone dystrophy, Leber's congenital amarurosis, Usher's syndrome, Bardet-Biedl-syndrome, or Stargardt disease. In embodiments, the vision degeneration is associated with retinitis pigmentosa. In embodiments, the vision degeneration is associated with age-related macular degeneration. In embodiments, the vision degeneration is associated with cone dystrophy. In embodiments, the vision degeneration is associated with rod-cone dystrophy. In embodiments, the vision degeneration is associated with Leber's congenital amarurosis. In embodiments, the vision degeneration is associated with Usher's syndrome. In embodiments, the vision degeneration is associated with Bardet-Biedl-syndrome. In embodiments, the vision degeneration is associated with Stargardt disease. In embodiments, the vision degeneration is associated with a photoreceptor degenerative disease.

In embodiments, vision degeneration is associated with Retinitis Pigmentosa, Cone Dystrophy, Rod Distrophy, Rod-cone Distrophy, Cone-Rod Distrophy, Bardet-Biedl syndrome, Leber congenital amaurosis, macular degeneration, age-related macular degeneration, Senior-Loken syndrome with retinitis pigmentosa or LCA, Joubert syndrome with retinitis pigmentosa, Alström syndrome with CRD, Meckel syndrome, retinitis pigmentosa in ciliopathies, Usher syndrome, Bietti crystalline corneoretinal dystrophy, Stargardt's Disease, Abetalipoproteinaemia, Refsum disease, Zellweger syndrome, Oguchi disease, Stargardt disease, fundus flavimaculatus, Bothnia dystrophy, retinitis punctata albescens, Newfoundland CRD, vitreoretinochoroidopathy, bestrophinopathy, Doyne honeycomb retinal degeneration (Malattia Leventinese), retinoschisis, Sorsby's fundus dystrophy, vitreoretinopathy in Stickler syndrome, digenic exudative vitreoretinopathy, retinopathy of prematurity, familial exudative vitreoretinopathy, Wagner disease, erosive vitreoretinopathy, gyrate atrophy, Hallervorden-Spatz syndrome, spinocerebellar ataxia with macular dystrophy, Goldmann-Favre syndrome, Sveinsson chorioretinal atrophy, Kearns-Sayre syndrome, Leigh syndrome, Leber hereditary optic neuropathy, pigmented paravenous chorioretinal atrophy, maculopathy in pseudoxanthoma elasticum, Choroideremia, Batten disease with retinitis pigmentosa, Jalili syndrome, Alagille syndrome, microphthalmos, or retinal disease syndrome.

In embodiments, the method further includes administering an effective amount of a RAR agonist. In embodiments, the RAR agonist is all-trans retinoic acid (ATRA) or its isomer, 13-cis retinoic acid. In embodiments, the method further includes administering an effective amount of a retinoic acid metabolism-blocking agent (e.g., liarozole). In embodiments, the RAR agonist is administered after discontinuation of the administration of a retinoic acid receptor inhibitor.

In embodiments, the method further includes administering an effective amount of a photoswitch. In embodiments the photoswitch is an azobenzene photoswitch. In embodiments, the photoswitch is a photoswitch described in U.S. Pat. Nos. 8,114,843, 8,309,350, 9,097,707, US 20070128662, US 20120190094, US 20130137113, US 20150224193, which are incorporated herein by reference in their entirety for all purposes.

In an aspect is provided a method of increasing the light sensitivity of retinal ganglion cells in a subject in need thereof, the method including administering an effective amount of an RAR agonist and a photoswitch. In embodiments, the RAR agonist is all-trans retinoic acid (ATRA) or its isomer, 13-cis retinoic acid. In embodiments, the method further includes administering an effective amount of a retinoic acid metabolism-blocking agent (e.g., liarozole). In embodiments, the method further includes administering an effective amount of a photoswitch. In embodiments the photoswitch is an azobenzene photoswitch. In embodiments, the photoswitch is azobenzene-quaternary ammonium (AAQ), quaternary ammonium-azobenzene-quaternary ammonium (QAQ), diethylamine-azobenzene-quaternary ammonium (DENAQ), benzylethylamino-azobenzene-quaternary ammonium (BENAQ), or phenyl-ethyl aniline azobenzene quaternary ammonium (PhENAQ). In embodiments, the photoswitch is a photoswitch described in Mourot, Alexandre et al. “Tuning Photochromic Ion Channel Blockers.” ACS Chemical Neuroscience 2.9 (2011): 536-543, Tochitsky et al. Scientific Reports 7, Article number: 45487 (2017); or Joseph P. Nemargut, III, Scott Greenwald, Lauren Rotkis, Richard H. Kramer, Dirk Trauner, Russell N. Van Gelder; Restoring Photosensitivity In Blind Mice With Small Molecular Photoswitch Phenyl-ethyl Aniline Azobenzene Quaternary Ammonium. Invest. Ophthalmol. Vis. Sci. 2012; 53(14):3639; which are incorporated herein by reference in their entirety for all purposes.

In an aspect is provided a method of inhibiting the activity of a retinoic acid receptor in a subject in need thereof, including contacting the retinoic acid receptor with a retinoic acid receptor inhibitor. In embodiments, the retinoic acid receptor inhibitor is an RAR antagonist. In embodiments, the RAR antagonist is BMS-453. In embodiments, the RAR antagonist is BMS-493. In embodiments, the RAR antagonist is BMS-614. In embodiments, the RAR antagonist is AGN 193109. In embodiments, the RAR antagonist is AGN 193491. In embodiments, the RAR antagonist is AGN 193618. In embodiments, the RAR antagonist is AGN 194202. In embodiments, the RAR antagonist is AGN 194301. In embodiments, the RAR antagonist is AGN 194574. In embodiments, the RAR antagonist is Ro 41-5253. In embodiments, the RAR antagonist is ER 50891. In embodiments, the RAR antagonist is CD 2665. In embodiments, the RAR antagonist is LE 135. In embodiments, the RAR antagonist inhibits the binding of a nuclear receptor coactivator to the retinoic acid receptor. In embodiments, the retinoic acid receptor inhibitor is an RAR inverse agonist. In embodiments, the RAR inverse agonist is BMS-493. In embodiments, the RAR antagonist inhibits the binding of a nuclear receptor coactivator (e.g., NCOA1 or NCOA2) to the retinoic acid receptor. In embodiments, the RAR inverse agonist increases the binding of a nuclear receptor corepressor (e.g., corepressor proteins NCoR or SMRT and associated factors such as histone deacetylases (HDACs) or DNA-methyl transferases) to the retinoic acid receptor.

In an aspect is provided a method of reducing the level of activity of the retinoic acid receptor, the method including contacting a cell including the retinoic acid receptor with a retinoic acid receptor inhibitor. In embodiments, the retinoic acid receptor inhibitor is a compound, an aptamer, an antibody, or a gene modulating reagent (e.g., CRISPR complex, TAL effector nuclease, zinc-finger nuclease, meganuclease, homing endonuclease, antisense nucleic acid, or siRNA) as disclosed herein.

In embodiments, the retinoic acid receptor contacts a retinoid x receptor. In embodiments, the retinoic acid receptor inhibitor contacts the retinoid x receptor.

In embodiments, the retinoic acid receptor is RARα (e.g., SEQ ID NO:3). In embodiments, the retinoic acid receptor is RARβ. In embodiments, the retinoic acid receptor is RARγ. In embodiments, the retinoic acid receptor is RARα (e.g., SEQ ID NO:3) and RARβ. In embodiments, the retinoic acid receptor is RARα (e.g., SEQ ID NO:3) and RARγ. In embodiments, the retinoic acid receptor is RARβ and RARγ. In embodiments, the retinoic acid receptor is not RARα (e.g., SEQ ID NO:3). In embodiments, the retinoic acid receptor is not RARβ. In embodiments, the retinoic acid receptor is not RARγ.

In embodiments, the retinoid X receptor is RXRα. In embodiments, the retinoid X receptor is RXRβ. In embodiments, the retinoid X receptor is RXRγ. In embodiments, the retinoid X receptor is RXRα and RXRβ. In embodiments, the retinoid X receptor is RXRα and RXRγ. In embodiments, the retinoid X receptor is RXRβ and RXRγ. In embodiments, the retinoid X receptor is not RXRα. In embodiments, the retinoid X receptor is not RXRβ. In embodiments, the retinoid X receptor is not RXRγ.

In an aspect is provided a method of inhibiting the activity of a P2X receptor in a subject in need thereof, the method including contacting the P2X receptor with a P2X receptor inhibitor. In embodiments, the P2X receptor inhibitor is TNP-ATP. In embodiments, the method of inhibiting the activity of a P2X receptor includes administering a retinoic acid receptor inhibitor. In embodiments, the retinoic acid receptor inhibitor is a compound, an aptamer, an antibody, a gene modulating reagent (e.g., CRISPR complex, TAL effector nuclease, zinc-finger nuclease, meganuclease, homing endonuclease, antisense nucleic acid, or siRNA) as disclosed herein, that reduces the activity (or the level of activity in a cell, tissue, organ, or subject) of a P2X receptor when compared to a control, such as absence of the inhibitor or a compound, an aptamer, an antibody, a gene modulating reagent (e.g., CRISPR complex, TAL effector nuclease, zinc-finger nuclease, meganuclease, homing endonuclease, antisense nucleic acid, or siRNA) with known inactivity.

In an aspect is provided a method of inhibiting the activity of a HCN channel in a subject in need thereof, the method including administering a retinoic acid receptor inhibitor. In embodiments, the retinoic acid receptor inhibitor is a compound, an aptamer, an antibody, a gene modulating reagent (e.g., CRISPR complex, TAL effector nuclease, zinc-finger nuclease, meganuclease, homing endonuclease, antisense nucleic acid, or siRNA) as disclosed herein, that reduces the activity (or the level of activity in a cell, tissue, organ, or subject) of an HCN channel when compared to a control, such as absence of the inhibitor or a compound, an aptamer, an antibody, a gene modulating reagent (e.g., CRISPR complex, TAL effector nuclease, zinc-finger nuclease, meganuclease, homing endonuclease, antisense nucleic acid, or siRNA) with known inactivity.

In embodiments, the retinoic acid receptor inhibitor (e.g., a compound, an aptamer, an antibody, or a gene modulating reagent as described herein) is administered topically to the eye. In embodiments, the retinoic acid receptor inhibitor (e.g., a compound, an aptamer, an antibody, or a gene modulating reagent as described herein) is administered by intraocular, subconjunctival, intravitreal, retrobulbar, or intracameral administration. In embodiments, the retinoic acid receptor inhibitor (e.g., a compound, an aptamer, an antibody, or a gene modulating reagent as described herein) is administered by intravitreal administration. In embodiments, the retinoic acid receptor (e.g., a compound, an aptamer, an antibody, or a gene modulating reagent as described herein) inhibitor is administered via oral administration, topical contact, intravenous, parenteral, intraperitoneal, intramuscular, intralesional, intrathecal, intranasal or subcutaneous administration. Parenteral administration includes, e.g., intravenous, intramuscular, intra-arteriole, intradermal, subcutaneous, intraperitoneal, intraventricular, and intracranial. In embodiments, the retinoic acid receptor inhibitor (e.g., a compound, an aptamer, an antibody, or a gene modulating reagent as described herein) is administered systemically (e.g., intraveneously). In embodiments, the retinoic acid receptor inhibitor (e.g., a compound, an aptamer, an antibody, or a gene modulating reagent as described herein) is administered by intravenous administration. In embodiments, the retinoic acid receptor inhibitor (e.g., a compound, an aptamer, an antibody, or a gene modulating reagent as described herein) is administered orally. In embodiments, the retinaldehyde dehydrogenase inhibitor (e.g., a compound, an aptamer, an antibody, or a gene modulating reagent as described herein) is administered topically to the eye. In embodiments, the retinaldehyde dehydrogenase inhibitor (e.g., a compound, an aptamer, an antibody, or a gene modulating reagent as described herein) is administered by intraocular, subconjunctival, intravitreal, retrobulbar, or intracameral administration. In embodiments, the retinoic acid receptor inhibitor (e.g., a compound, an aptamer, an antibody, or a gene modulating reagent as described herein) is administered via oral administration, topical contact, intravenous, parenteral, intraperitoneal, intramuscular, intralesional, intrathecal, intranasal or subcutaneous administration. Parenteral administration includes, e.g., intravenous, intramuscular, intra-arteriole, intradermal, subcutaneous, intraperitoneal, intraventricular, and intracranial. In embodiments, the retinaldehyde dehydrogenase inhibitor (e.g., a compound, an aptamer, an antibody, or a gene modulating reagent as described herein) inhibitor is administered systemically (e.g., intraveneously). In embodiments, the retinaldehyde dehydrogenase inhibitor (e.g., a compound, an aptamer, an antibody, or a gene modulating reagent as described herein) is administered orally.

In an aspect is provided a method of treating vision degeneration, the method including administering to a subject in need thereof an effective amount of an inhibitor of the level of retinoic acid in the subject (e.g., an agent which reduces the level of retinoic acid in the subject relative to a control). In embodiments, the inhibitor is a retinaldehyde dehydrogenase inhibitor. In embodiments, the retinaldehyde dehydrogenase inhibitor is diethylaminobenzaldehyde, citral, or disulfiram.

In embodiments, the level of retinoic acid is reduced by administering phytanic acid, docosahexaenoic acid, or a combination thereof as described in Lampen, A., Meyer, S., & Nau, H. (2001) Biochimica et Biophysica Acta (BBA)-Gene Structure and Expression, 1521(1), 97-106; which is incorporated herein by reference in its entirety for all purposes.

In embodiments, the vision degeneration is associated with a reduction in cone cells. In embodiments, the vision degeneration is associated with a reduction in rod cells. In embodiments, the vision degeneration is associated with a reduction in cones. In embodiments, the vision degeneration is associated with a reduction in rods.

In an aspect is provided a method of treating vision degeneration, the method including administering to a subject in need thereof an effective amount of an inhibitor of the level of retinoic acid receptor in the subject (e.g., an agent which reduces the level of retinoic acid receptor in the subject relative to a control).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. Embodiments

Embodiment P1. A method of treating vision degeneration, said method comprising administering to a subject in need thereof an effective amount of a retinoic acid receptor inhibitor.

Embodiment P2. The method of embodiment P1, wherein the retinoic acid receptor inhibitor is an RAR antagonist.

Embodiment P3. The method of embodiment P2, wherein the RAR antagonist inhibits the binding of a nuclear receptor coactivator to the retinoic acid receptor.

Embodiment P4. The method of embodiment P1, wherein the retinoic acid receptor inhibitor is an RAR inverse agonist.

Embodiment P5. The method of embodiment P4, wherein the RAR inverse agonist increases the binding of a nuclear receptor corepressor to the retinoic acid receptor.

Embodiment P6. The method of one of embodiments P1 to P5, wherein light sensitivity of retinal ganglion cells in the subject is increased.

Embodiment P7. The method of one of embodiments P1 to P5, wherein hyperexcitability of retinal ganglion cells in the subject is inhibited.

Embodiment P8. The method of one of embodiments P1 to P5, wherein increases in the number, activity, or cellular distribution of hyperpolarization-activated cyclic nucleotide-gated channel in retinal ganglion cells are reduced.

Embodiment P9. The method of one of embodiments P1 to P8, wherein the vision degeneration is associated with retinitis pigmentosa, age-related macular degeneration, cone dystrophy, rod-cone dystrophy, Leber's congenital amarurosis, Usher's syndrome, Bardet-Biedl-syndrome, or Stargardt disease.

Embodiment P10. A method of inhibiting the activity of a retinoic acid receptor in a subject in need thereof, comprising contacting the retinoic acid receptor with a retinoic acid receptor inhibitor.

Embodiment P11. The method of embodiment P10, wherein the retinoic acid receptor inhibitor is an RAR antagonist.

Embodiment P12. The method of embodiment P11, wherein the RAR antagonist inhibits the binding of a nuclear receptor coactivator to the retinoic acid receptor.

Embodiment P13. The method of embodiment P10, wherein the retinoic acid receptor inhibitor is an RAR inverse agonist.

Embodiment P14. The method of embodiment P13, wherein the RAR inverse agonist increases the binding of a nuclear receptor corepressor to the retinoic acid receptor.

Embodiment P15. The method of one of embodiments P10 to P14, wherein the retinoic acid receptor contacts a retinoid x receptor.

Embodiment P16. The method of embodiment P15, wherein the retinoic acid receptor inhibitor contacts the retinoid x receptor.

Embodiment P17. The method of one of embodiments P1 to P16, wherein the retinoic acid receptor is RARα.

Embodiment P18. The method of one of embodiments P1 to P17, wherein the retinoic acid receptor inhibitor has the formula:

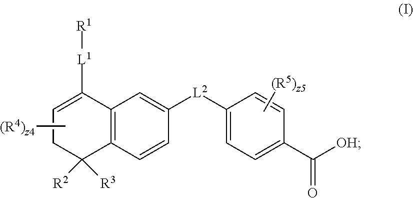

wherein
L¹ is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —S(O)₂—, —NH—, —O—, —S—, —C(O)—, —C(O)NH—, —NHC(O)—, —NHC(O)NH—, —NHC(O)NH—, —C(O)O—, —OC(O)—, —C(S)—, —C(S)NH—, —NHC(S)—, —NHC(S)NH—, —NHC(S)NH—, —C(S)O—, —OC(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are each independently hydrogen, or substituted or unsubstituted alkyl, or substituted or unsubstituted heteroalkyl;
R⁴ and R⁵ are each independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z4 is an integer from 0 to 3; and
z5 is an integer from 0 to 4.

Embodiment P19. The method of embodiment P18, wherein L² is

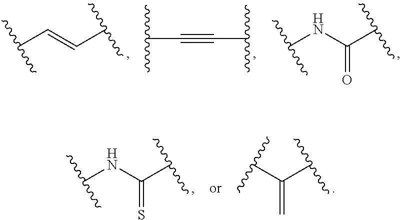

Embodiment P20. The method of one of embodiments P18 or P19, wherein -L¹-R¹ has the formula:

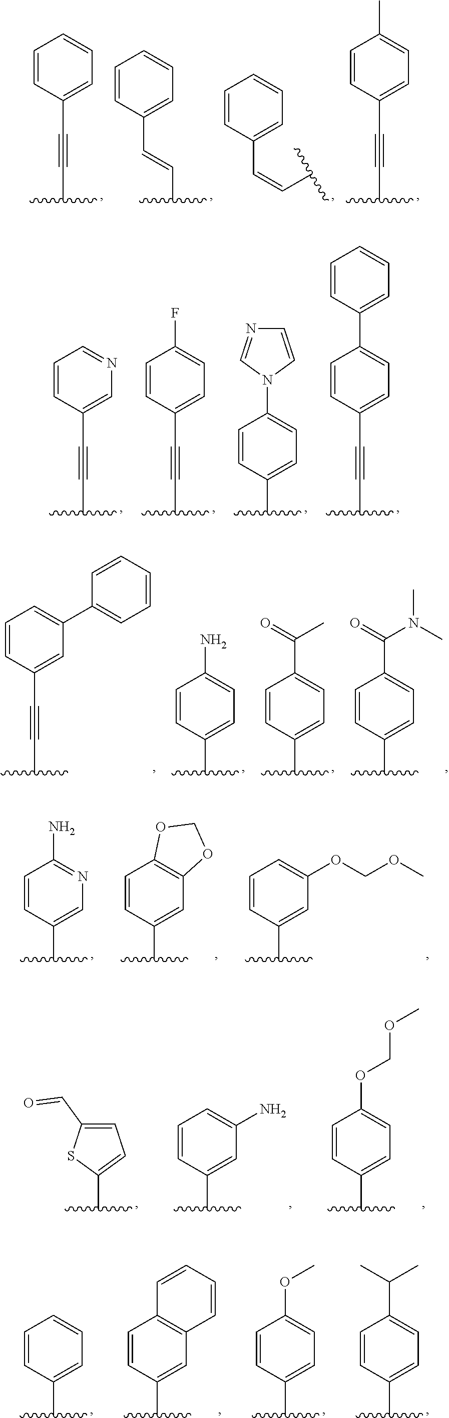

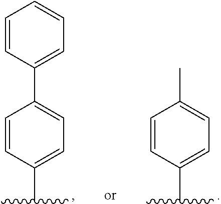

Embodiment P21. The method of embodiment P18, wherein the retinoic acid receptor inhibitor is

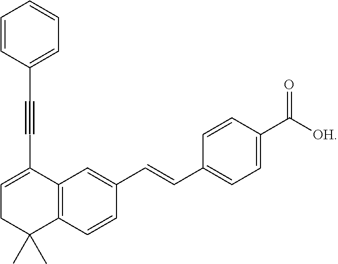 [SMILES: CC1(C)CC=C(C#Cc2ccccc2)c2cc(/C=C/c3ccc(C(=O)O)cc3)ccc21]

Embodiment P22. The method of one of embodiments P1 to P23, wherein the retinoic acid receptor inhibitor is administered topically to the eye.

Embodiment P23. The method of one of embodiments P1 to P23, wherein the retinoic acid receptor inhibitor is administered by intraocular, subconjunctival, intravitreal, retrobulbar, or intracameral administration.

Embodiment P24. A method of treating vision degeneration, said method comprising administering to a subject in need thereof an effective amount of an inhibitor of the level of retinoic acid in the subject.

Embodiment P25. The method of embodiment P24, wherein the inhibitor is a retinaldehyde dehydrogenase inhibitor.

Embodiment P26. The method of embodiment P25, wherein the retinaldehyde dehydrogenase inhibitor is diethylaminobenzaldehyde, citral, or disulfiram.

Embodiment P27. The method of one of embodiments P1 to P26, wherein the vision degeneration is associated with a reduction in cone cells.

Embodiment P28. The method of one of embodiments P1 to P26, wherein the vision degeneration is associated with a reduction in rod cells.

### VI. Additional Embodiments

Embodiment 1. A method of treating vision degeneration, said method comprising administering to a subject in need thereof an effective amount of a retinoic acid receptor inhibitor.

Embodiment 2. The method of embodiment 1, wherein the retinoic acid receptor inhibitor is an RAR antagonist.

Embodiment 3. The method of embodiment 2, wherein the RAR antagonist inhibits the binding of a nuclear receptor coactivator to the retinoic acid receptor.

Embodiment 4. The method of embodiment 1, wherein the retinoic acid receptor inhibitor is an RAR inverse agonist.

Embodiment 5. The method of embodiment 4, wherein the RAR inverse agonist increases the binding of a nuclear receptor corepressor to the retinoic acid receptor.

Embodiment 6. The method of one of embodiments 1 to 5, wherein light sensitivity of retinal ganglion cells in the subject is increased.

Embodiment 7. The method of one of embodiments 1 to 5, wherein hyperexcitability of retinal ganglion cells in the subject is inhibited.

Embodiment 8. The method of one of embodiments 1 to 5, wherein increases in the number, activity, or cellular distribution of hyperpolarization-activated cyclic nucleotide-gated channel in retinal ganglion cells are reduced.

Embodiment 9. The method of one of embodiments 1 to 8, wherein the vision degeneration is associated with retinitis pigmentosa, age-related macular degeneration, cone dystrophy, rod-cone dystrophy, Leber's congenital amarurosis, Usher's syndrome, Bardet-Biedl-syndrome, or Stargardt disease.

Embodiment 10. A method of inhibiting the activity of a retinoic acid receptor in a subject in need thereof, comprising contacting the retinoic acid receptor with a retinoic acid receptor inhibitor.

Embodiment 11. The method of embodiment 10, wherein the retinoic acid receptor inhibitor is an RAR antagonist.

Embodiment 12. The method of embodiment 11, wherein the RAR antagonist inhibits the binding of a nuclear receptor coactivator to the retinoic acid receptor.

Embodiment 13. The method of embodiment 10, wherein the retinoic acid receptor inhibitor is an RAR inverse agonist.

Embodiment 14. The method of embodiment 13, wherein the RAR inverse agonist increases the binding of a nuclear receptor corepressor to the retinoic acid receptor.

Embodiment 15. The method of one of embodiments 10 to 14, wherein the retinoic acid receptor contacts a retinoid x receptor.

Embodiment 16. The method of embodiment 15, wherein the retinoic acid receptor inhibitor contacts the retinoid x receptor.

Embodiment 17. The method of one of embodiments 1 to 16, wherein the retinoic acid receptor is RARα.

Embodiment 18. The method of one of embodiments 1 to 17, wherein the retinoic acid receptor inhibitor has the formula:

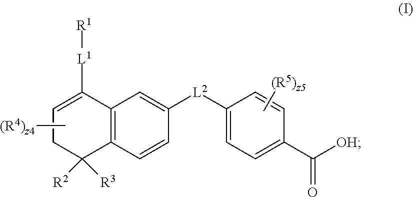

wherein
L¹ is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —S(O)₂—, —NH—, —O—, —S—, —C(O)—, —C(O)NH—, —NHC(O)—, —NHC(O)NH—, —NHC(O)NH—, —C(O)O—, —OC(O)—, —C(S)—, —C(S)NH—, —NHC(S)—, —NHC(S)NH—, —NHC(S)NH—, —C(S)O—, —OC(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is halogen, —CCl₃, —CF₃, —CI₃, —CHCl₂, —CHBr₂, —CHF₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are each independently hydrogen, or substituted or unsubstituted alkyl, or substituted or unsubstituted heteroalkyl;
R⁴ and R⁵ are each independently halogen, —CCl₃, —CF₃, —CI₃, —CHCl₂, —CHBr₂, —CHF₂, —CHI₂, —CH₂Cl, —CH₂Br, —CH₂F, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z4 is an integer from 0 to 3; and
z5 is an integer from 0 to 4.

Embodiment 19. The method of embodiment 18, wherein L² is

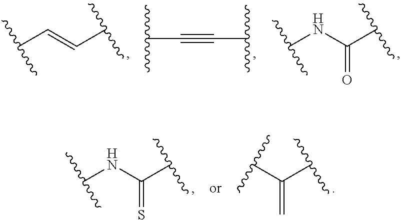

Embodiment 20. The method of one of embodiments 18 or 19, wherein -L¹⁻-R¹— has the formula:

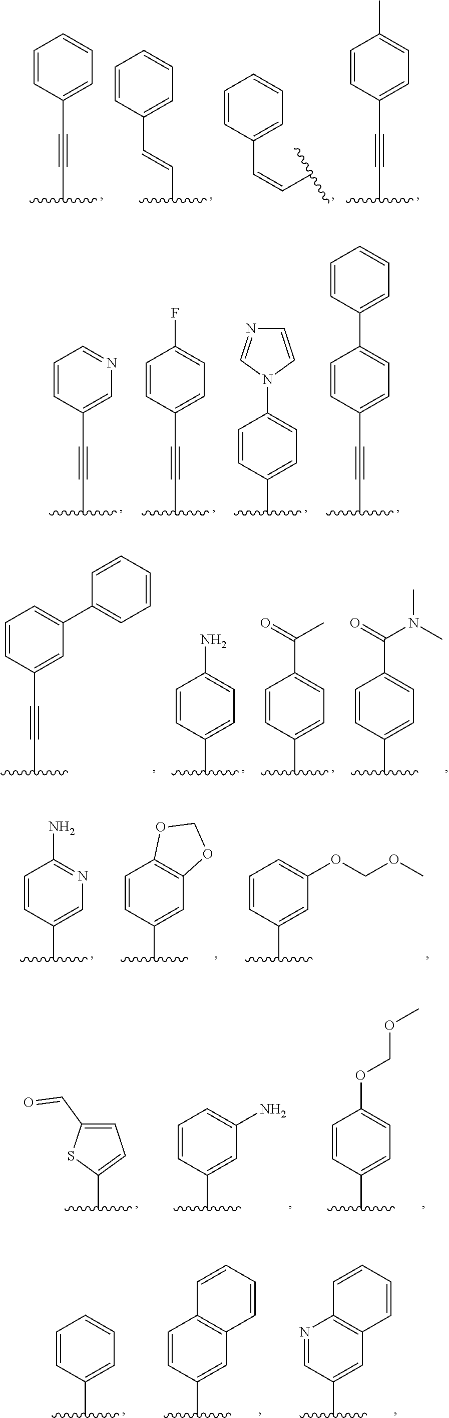

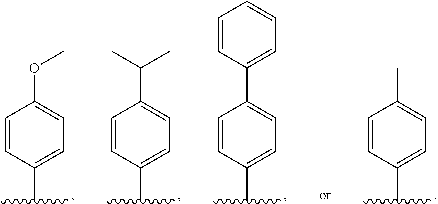

Embodiment 21. The method of one of embodiments 1 to 17, wherein the retinoic acid receptor inhibitor is

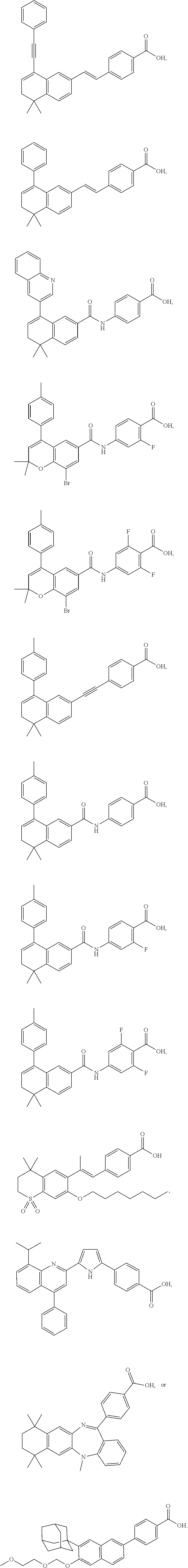

Embodiment 22. The method of embodiment 21, wherein the retinoic acid receptor inhibitor is

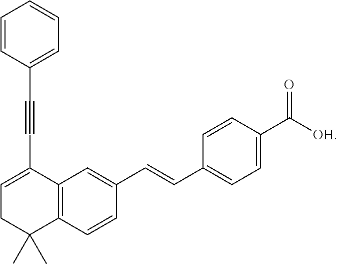 [SMILES: CC1(C)CC=C(C#Cc2ccccc2)c2cc(/C=C/c3ccc(C(=O)O)cc3)ccc21]

Embodiment 23. The method of embodiment 1, wherein the retinoic acid receptor inhibitor comprises a nucleic acid.

Embodiment 24. The method of embodiment 23, wherein the retinoic acid receptor inhibitor is a nucleic acid.

Embodiment 25. The method of one of embodiments 1 to 24, wherein the retinoic acid receptor inhibitor comprises a gene modulating reagent.

Embodiment 26. The method of embodiment 25, wherein the gene modulating reagent is a gene editing reagent or a gene modulating nucleic acid.

Embodiment 27. The method of embodiment 26, wherein the gene editing reagent is a CRISPR complex, a TAL effector nuclease, a zinc-finger nuclease, a meganuclease, or a homing endonuclease.

Embodiment 28. The method of embodiment 27, wherein the CRISPR complex comprises a guide RNA and a Cas9 nuclease.

Embodiment 29. The method of embodiment 28, wherein the guide RNA comprises a nucleic acid sequence at least 80% identical to an RNA sequence of a retinoic acid receptor or a fragment thereof or a complement thereof.

Embodiment 30. The method of embodiment 28, wherein the guide RNA comprises a nucleic acid sequence identical to an RNA sequence of a retinoic acid receptor or a fragment thereof, or a complement thereof.

Embodiment 31. The method of one of embodiments 29 to 30, wherein the guide RNA comprises a nucleic acid sequence from 10 to 30 nucleotides in length.

Embodiment 32. The method of embodiment 28, wherein the guide RNA comprises a nucleic acid sequence at least 80% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site.

Embodiment 33. The method of embodiment 28, wherein the guide RNA comprises a nucleic acid sequence from 10 to 30 nucleotides in length and at least 80% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site.

Embodiment 34. The method of embodiment 26, wherein the gene modulating nucleic acid is an antisense nucleic acid or an siRNA.

Embodiment 35. The method of embodiment 34, wherein the antisense nucleic acid comprises a nucleic acid sequence at least 80% identical to a nucleic acid sequence complementary to an RNA sequence of a retinoic acid receptor or a fragment thereof.

Embodiment 36. The method of embodiment 34, wherein the antisense nucleic acid comprises a nucleic acid sequence complementary to an RNA sequence of a retinoic acid receptor or a fragment thereof.

Embodiment 37. The method of embodiment 34, wherein the antisense nucleic acid comprises a nucleic acid sequence at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

Embodiment 38. The method of one of embodiments 35 to 37, wherein the antisense nucleic acid comprises a nucleic acid sequence from 10 to 50 nucleotides in length.

Embodiment 39. The method of embodiment 34, wherein the antisense nucleic acid comprises a nucleic acid sequence from 10 to 50 nucleotides in length and at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

Embodiment 40. The method of embodiment 34, wherein the siRNA comprises a nucleic acid sequence at least 80% identical to a nucleic acid sequence complementary to an RNA sequence of a retinoic acid receptor or a fragment thereof.

Embodiment 41. The method of embodiment 34, wherein the siRNA comprises a nucleic acid sequence identical to an RNA sequence complementary to an RNA sequence of a retinoic acid receptor or a fragment thereof.

Embodiment 42. The method of embodiment 34, wherein the siRNA comprises a nucleic acid sequence at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

Embodiment 43. The method of one of embodiments 40 to 42, wherein the siRNA comprises a nucleic acid sequence from 20 to 30 nucleotides in length.

Embodiment 44. The method of embodiment 34, wherein the siRNA comprises a nucleic acid sequence from 20 to 30 nucleotides in length and at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

Embodiment 45. The method of embodiment 23, wherein the retinoic acid receptor inhibitor comprises an expression vector.

Embodiment 46. The method of embodiment 45, wherein the expression vector is a viral vector.

Embodiment 47. The method of embodiment 45, wherein the expression vector is an adenovirus vector, adeno-associated virus vector, or a lentiviral vector.

Embodiment 48. The method of one of embodiments 45 to 47, wherein the expression vector is capable of expressing a dominant negative retinoic acid receptor protein.

Embodiment 49. The method of embodiment 48, wherein the dominant negative retinoic acid receptor protein is a truncated retinoic acid receptor compared to the wildtype retinoic acid receptor protein.

Embodiment 50. A method of treating vision degeneration, said method comprising administering to a subject in need thereof an effective amount of an inhibitor of the level of retinoic acid in the subject.

Embodiment 51. The method of embodiment 50, wherein the inhibitor is a retinaldehyde dehydrogenase inhibitor.

Embodiment 52. The method of embodiment 51, wherein the retinaldehyde dehydrogenase inhibitor is diethylaminobenzaldehyde, citral, or disulfiram.

Embodiment 53. The method of one of embodiments 1 to 52, wherein the retinoic acid receptor inhibitor is administered topically to the eye.

Embodiment 54. The method of one of embodiments 1 to 52, wherein the retinoic acid receptor inhibitor is administered by intraocular, subconjunctival, intravitreal, retrobulbar, or intracameral administration.

Embodiment 55. The method of one of embodiments 1 to 52, wherein the retinoic acid receptor inhibitor is administered by intravitreal or intravenous administration.

Embodiment 56. The method of one of embodiments 1 to 52, wherein the retinoic acid receptor inhibitor is administered by intravitreal administration.

Embodiment 57. The method of one of embodiments 1 to 52, wherein the vision degeneration is associated with a reduction in cone cells.

Embodiment 58. The method of one of embodiments 1 to 52, wherein the vision degeneration is associated with a reduction in rod cells.

### EXAMPLES

### Example 1: Retinoic Acid Mediates Electrophysiological Remodeling During Retinal Degeneration, Making the Retinoic Acid Receptor a Drug Target for Enhancing Vision

Retinitis pigmentosa (RP) and age-related macular degeneration (AMD) are blinding diseases caused by the progressive degeneration of rod and cone photoreceptors. Nevertheless, downstream retinal neurons survive and the axons of retinal ganglion cells (RGCs) remain intact, maintaining synaptic connectivity with the brain (1,2). The integrity of RGCs is the foundation for several technologies aimed at restoring visual perception in RP and AMD. Electronic implants (3), optogenetic tools (4), and optopharmacological tools (5) can either directly or indirectly impart artificial light responses onto RGCs and restore light-elicited behavioral responses mediated by visual circuits in the brain.

Even though downstream retinal neurons remain alive, they show gradual changes in morphology in both human RP patients and animal models of RP. In the rd1 mouse model, new dendritic branches appear and cell body position begins to change months after the photoreceptors die (6-8). In contrast, biochemical and physiological changes in retinal neurons start soon after photoreceptor death and may exacerbate vision loss. Within weeks, membrane receptors for ATP (P2X receptors) are up-regulated and become chronically active, resulting in increased membrane permeability. A type of ion channel that underlies spontaneous firing (the HCN channel), is up-regulated, causing RGCs to become hyperexcitable (9-11). The combination of increased membrane permeability and hyperexcitability allows azobenzene photoswitches to cross the plasma membrane into the cytoplasm and bind to the intracellular side of voltage-gated ion channels to confer light-dependent firing on RGCs in degenerated retinas (11).

What biochemical signal informs RGCs that rods and cones are degenerating? Perhaps rod and cone death leads to a decrease in a light-dependent-synaptic signal, such as glutamate, which might act as a suppressor of remodeling in healthy retina. Inconsistent with this idea, mice with mutations that eliminate phototransduction without causing degeneration show no remodeling (12). Rods synthesize and release trophic factors, including rod-derived cone viability factor (RdCVF, 13), another possible suppressor of RGC remodeling. However, the receptor for RdCVF is undetected in the inner retina (14), making this possibility unlikely.

An alternative scenario is that rod and cone death results in an increase in an inducer of remodeling. Retinal pigment epithelium (RPE)-derived retinoids, normally sequestered by photoreceptor outer segment opsins (15), may gain access to the inner retina after photoreceptor degeneration. Retinoic acid (RA), a molecule derived from the visual chromophore retinaldehyde, is a transcriptional regulator that plays crucial roles in embryonic development (16). RA has also been implicated as a neural signal in adulthood, mediating synaptic plasticity in the cortex and hippocampus during learning (17-20) and triggering dendritic growth in the outer retina after light-induced damage (21). Here we examine whether RA is the trigger of degeneration-dependent remodeling of RGCs in rodent models of hereditary blindness. Treatments that interfere with RA production or signaling should disrupt or prevent RGC remodeling, testing whether RA is necessary. Treatments that enhance RA signaling should mimic remodeling in RGCs, testing whether RA is sufficient. Lastly, an RA-selective transcription reporter could reveal whether elevated RA signaling is actually occurring during degenerative disease, validating its role as the initiator of electrophysiological remodeling.

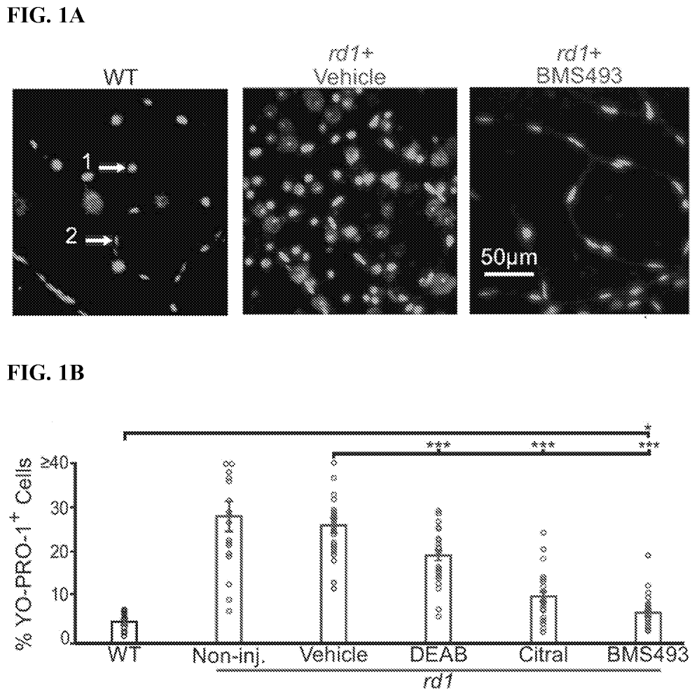

Blocking RA signaling prevents pathophysiological remodeling of RGCs in degenerated retina. When photoreceptors degenerate, RGCs become more permeant to fluorescent dyes, such as the DNA-binding dye YO-PRO-1. Increased permeability is mediated by up-regulation and hyperactivity of P2X receptors (11). We found that the fraction of RGC nuclei labeled with YO-PRO-1 was 10-fold greater in rd1 retina than in WT retina (FIGS. 1A-1B), consistent with heightened permeability (rd1=28.97±3.54%; WT=3.74±0.66%, n=12 retinal samples each, p<0.001). However, intravitreal injection of BMS-493, a retinoic acid receptor (RAR) antagonist, reduced YO-PRO-1 labeling in rd1 mice to near WT levels (BMS-493=6.13±1.43%; measured 3-7 days after injection). There was no difference in YO-PRO-1 labeling in rd1 mice between vehicle-injected and uninjected controls (n=30,18 respectively, p=0.56).

We next tested whether blocking RA synthesis can inhibit remodeling by injecting inhibitors of retinaldehyde dehydrogenase (RALDH), the enzyme that converts retinaldehyde to RA. Intravitreal injection of diethylaminobenzaldehyde (DEAB; 20 uM) or citral (50 uM) reduced, but did not completely eliminate YO-PRO-1 labeling measured 3-7 days after injection (DEAB=19.77±2.32%, n=30, and citral=9.87±2.39%, n=24; p<0.001). Blocking the receptor for RA is more effective than acutely blocking synthesis of RA, which would spare any RA that was present before drug treatment (22).

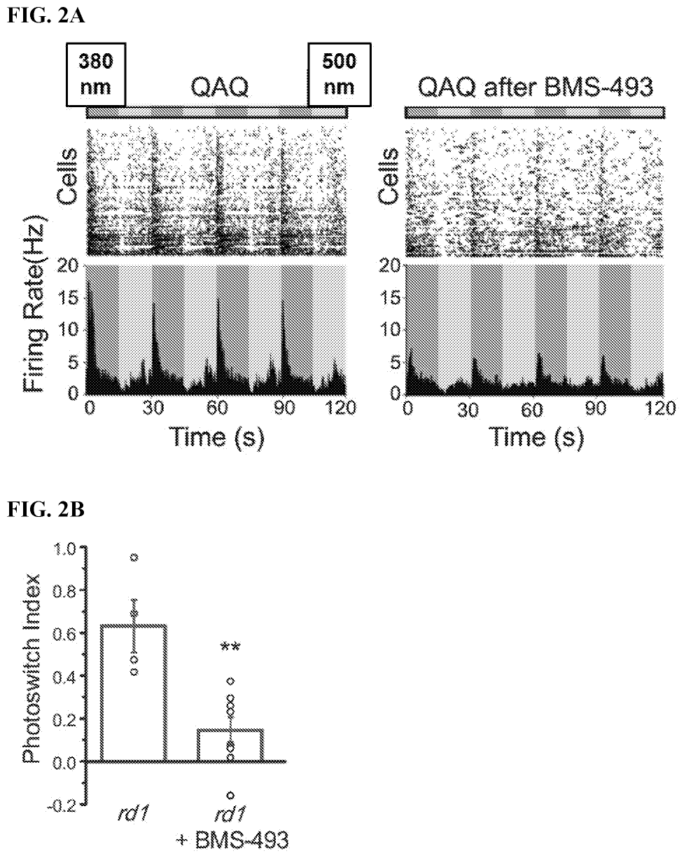

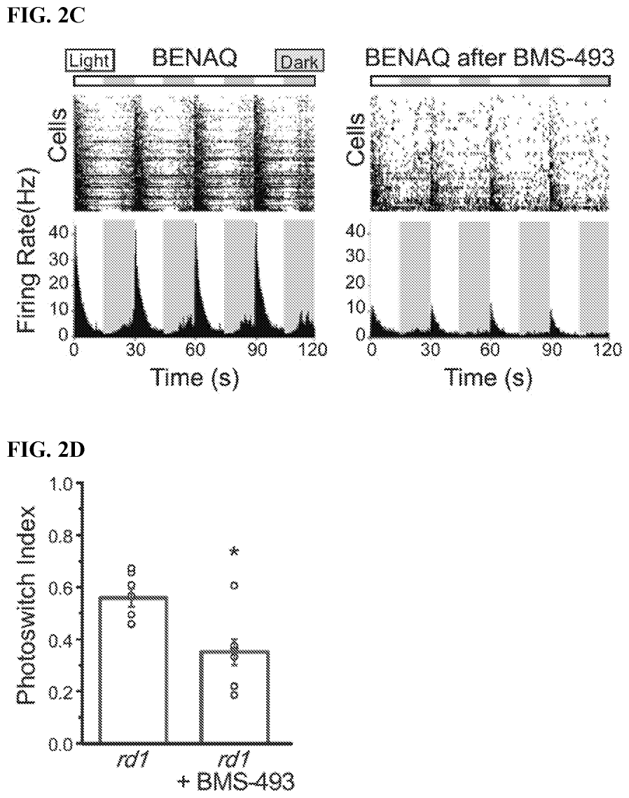

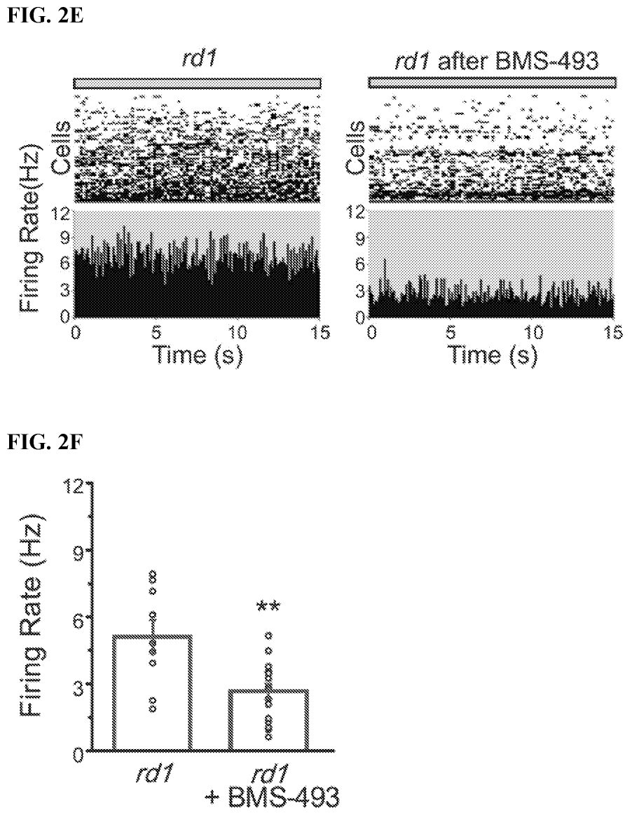

Heightened membrane permeability is also necessary for degeneration-dependent photosensitization of RGCs by azobenzene photoswitches (11). These compounds bestow light-sensitive action potential firing on RGCs from rd1 retina, but have no effect on RGCs from WT retina (12). We measured photoswitching in isolated rd1 retina with a multi-electrode array (MEA). BMS-493 injection reduced photosensitization elicited by two different photoswitches, QAQ and BENAQ, which act on different ion channels and respond to different wavelengths of light (FIG. 2A, FIG. 2C). For both photoswitches, the Photoswitch Index (PI), was significantly reduced in rd1 retina treated with BMS-493 (PI_{QAQ}=0.15±0.06, n=8; PI_{BENAQ}=0.35±0.05, n=7) as compared to untreated rd1 retina (PI_{QAQ}=0.63±0.12, n=4, p=0.002; PI_{BENAQ}=0.56±0.03, n=7, p_{QAQ}=0.002, p_{BENAQ}=0.005; FIG. 2B, FIG. 2D).

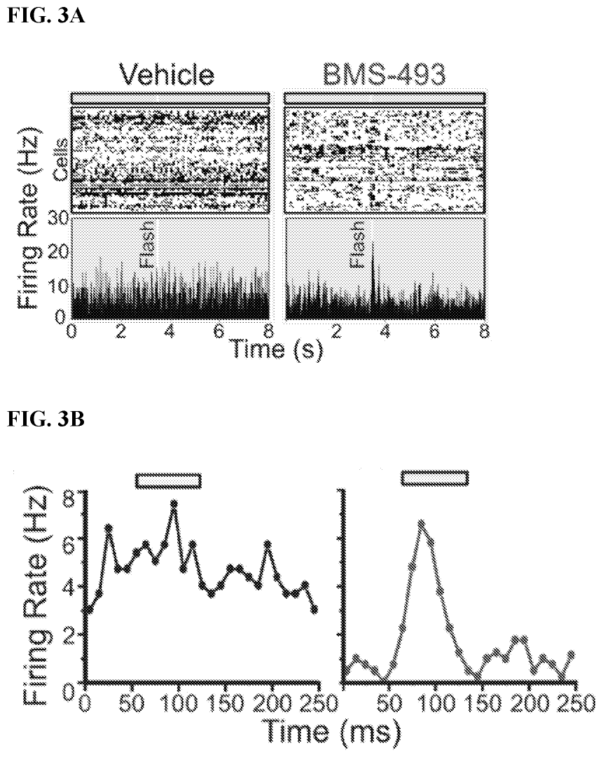

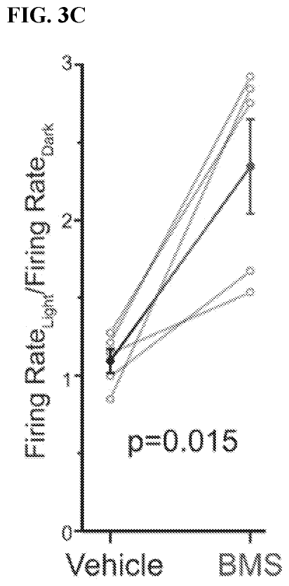

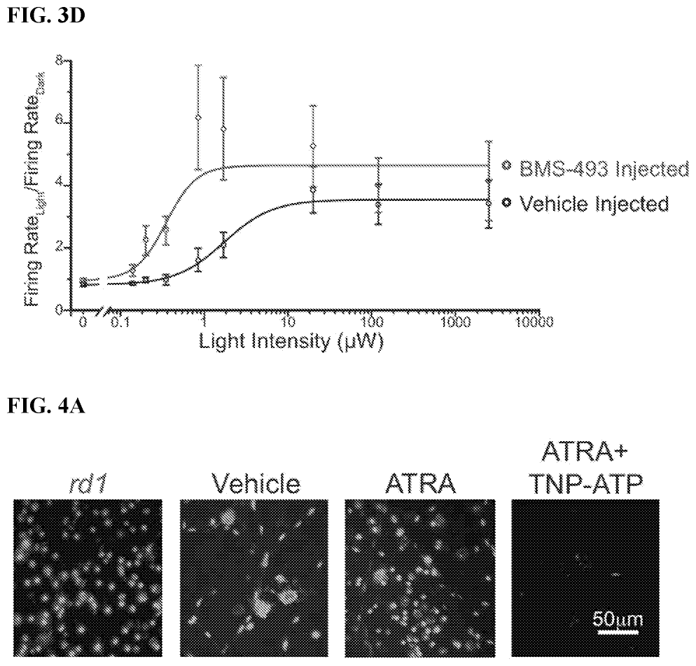

Photoreceptor degeneration leads to RGC hyperexcitability, manifest as an increase in the frequency of spontaneous action potential firing in darkness (9,10). Our results suggest that RGC hyperexcitability, like other aspects of remodeling, is also dependent on RA signaling. Within 3-7 days following a single intravitreal injection, BMS-493 led to lower spontaneous firing of rd1 RGCs (FIG. 2E) from 5.13±0.74 Hz (n=10), to 2.67±0.33 Hz (n=16, p=0.002) (FIG. 2E, FIG. 2F). In most patients with RP, at least some photoreceptors persist for years during the progression of retinal degeneration. During the period when their retinas are incompletely degenerated, high background firing of RGCs could obscure light responses, particularly to low-intensity stimuli. By reducing spontaneous firing, blockers of RA signaling might augment light responses and enhance visual performance. To test this idea we used the rd10 strain of mice, whose photoreceptors degenerate more slowly than rd1 mice. At 6 weeks, when their retinas were incompletely degenerated, we injected one eye with BMS-493 and the other with vehicle. At 3-7 days post-injection, we evaluated retinal sensitivity with light flashes of varying intensity. BMS-493-treated retinas showed a transient increase in RGC firing in response to a flash of dim light, whereas vehicle-treated retinas showed no RGC response to the same flash (Kruskal-Wallis ANOVA, Dunn's post-hoc p=0.0263, FIG. 3A, FIG. 3B). The emergence of the light response was associated with a decrease in the background firing rate in darkness. A BMS-493-augmented light response was observed in all 5 animals tested, comparing between drug-injected and vehicle-injected eyes (paired t-test, p=0.015, n=5, FIG. 3C). Measuring the response over a variety of intensities revealed a leftward shift in the midpoint of the intensity vs. response curve and an increase in the peak response (BMS-493 150=0.35 μW, Max=4.77; Vehicle I₅₀=1.73 μW, Max=3.68). Moreover, response threshold was lower for BMS-493-injected than for vehicle-injected eyes (0.2 μW vs. 0.85 μW; Kruskal-Wallis ANOVA with Dunn's post hoc, p_{BMS-493}=0.033, p_{vehicle}=0.005). Hence, inhibiting RA signaling improves the light response of RGCs in partially degenerated retinas.

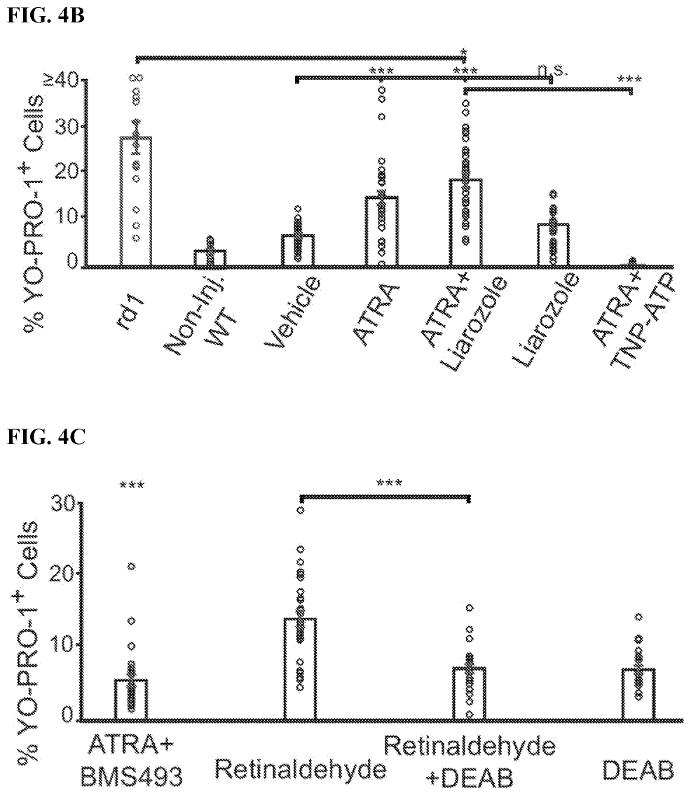

Activating RA signaling in WT retina mimics pathophysiological remodeling. To test whether RA is sufficient for triggering remodeling, we intravitreally injected all-trans retinoic acid (ATRA), the most photostable RA isomer. At 3-7 days after injection, we found increased YO-PRO-1 labeling as compared to vehicle-injected controls (FIG. 4A). The P2X antagonist TNP-ATP (23) prevented dye labeling, indicating the P2X receptor is required, just as in degenerating retinas. YO-PRO-1 labeling was increased 2-fold with ATRA (FIG. 4B), but did not change significantly with liarozole alone, which inhibits RA degradation (24) (Uninjected=3.74±0.66%, n=12; Vehicle=7.21±0.80%, n=36; ATRA=15.80±3.10%, n=34, p<0.001, liarozole: 8.66±1.36%, n=30 p=0.078). However, ATRA plus liarozole had a synergistic effect, increasing labeling by almost 3-fold (ATRA+Liarozole=19.79±2.85%, n=35, p<0.0001). Even though dye-labeling was increased, it was somewhat lower than in rd1 mice (28.98±6.93%, n=18, p=0.022) whose RGCs may be chronically exposed to elevated RA from early in life when photoreceptors begin to degenerate (i.e. within 14 days after birth). Treatment with TNP-ATP abolished RGC dye-labeling induced by ATRA (0.39±0.31%, n=9, p<0.001), consistent with mediation by P2X receptors. The effect of ATRA was blocked by co-injection of BMS-493, which prevents signaling through RAR (FIG. 4C; 5.74±1.54%, n=28, p<0.001). Taken together, these results indicate that RA-mediated signaling is sufficient to induce high permeability via P2X channels in WT RGCs, mimicking degeneration-dependent changes in rd1 RGCs.

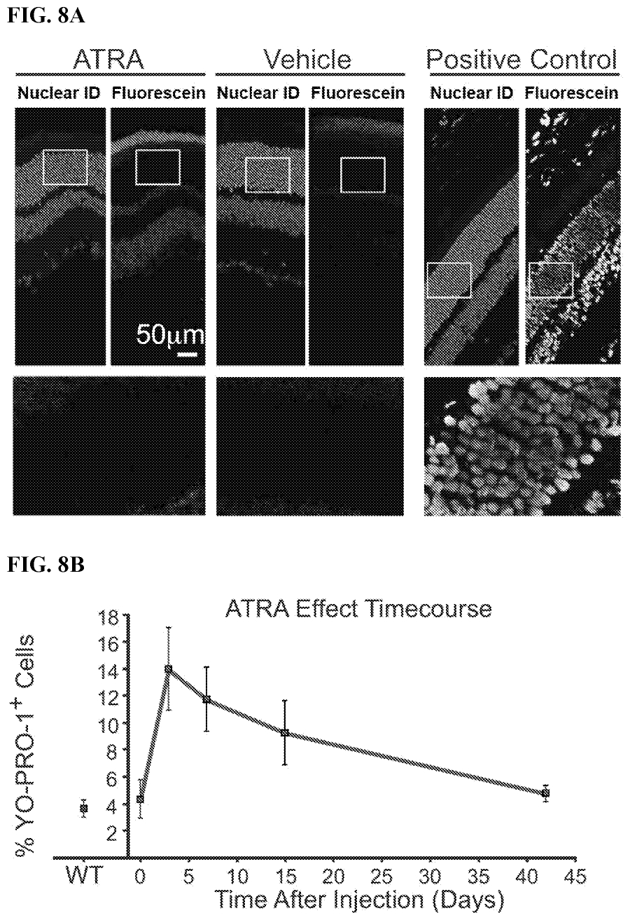

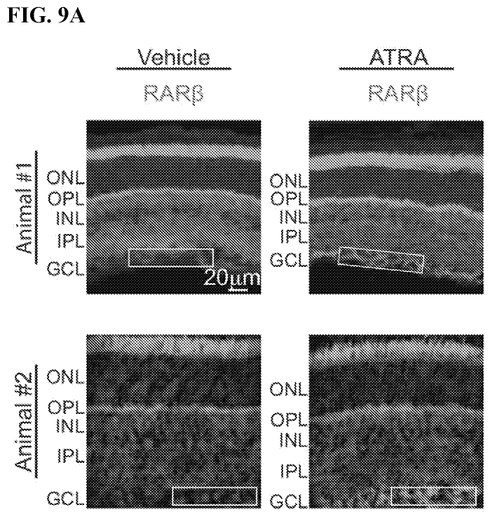

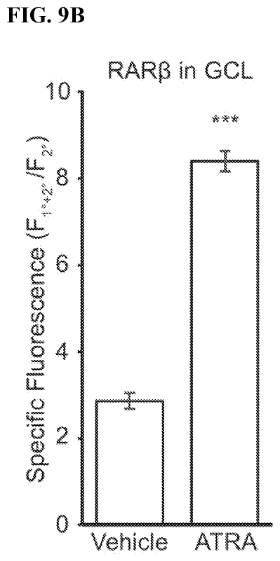

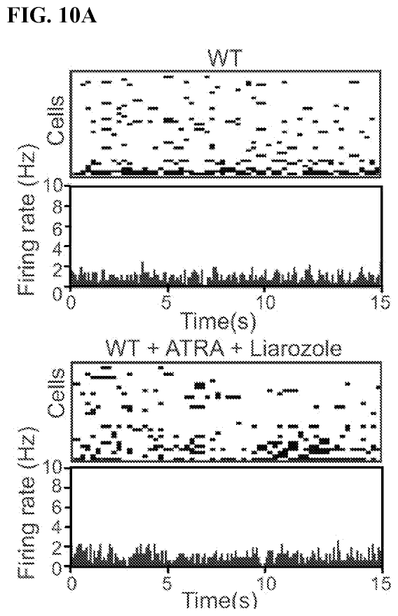

We considered a possible alternative explanation for these results. If ATRA were toxic, it might induce rod and cone cell death and thereby indirectly trigger RGC remodeling. However, a TUNEL assay showed no cell death after ATRA (FIG. 8A). Moreover, YO-PRO-1 loading induced by ATRA disappeared 6 weeks after injection, inconsistent with irreversible loss of photoreceptors (FIG. 8B). In contrast, immunolabeling of RGCs showed upregulation of the β-subunit of RAR (25), a gene product indicative of canonical RA transcriptional activation (FIGS. 9A-9B). These findings indicate that RA acts directly on RGCs to induce gene transcription.

RA is produced through dehydrogenation of retinaldehyde by RALDH (26), which is expressed in RPE and some retinal neurons (27). Supplying the WT retina with exogenous retinaldehyde could enhance production of RA, perhaps mimicking remodeling. We injected retinaldehyde at 3-7 days before imaging YO-PRO-1 fluorescence (FIG. 4C). Retinaldehyde increased YO-PRO-1 labeling (14.6±2.29%, n=28, p<0.001), nearly as much as ATRA itself (p=0.550). Co-injecting retinaldehyde with the RALDH inhibitor DEAB (28) resulted in no enhancement of YO-PRO-1 labeling (7.65±1.34%, n=23, p=0.583). Injection of DEAB alone caused no change in YO-PRO1 labeling in WT retina (7.47±1.21%, n=21; p=0.728). These results suggest that in WT retinas, the supply of retinaldehyde is rate-limiting for the production of RA, which is sufficient to trigger RGC remodeling.

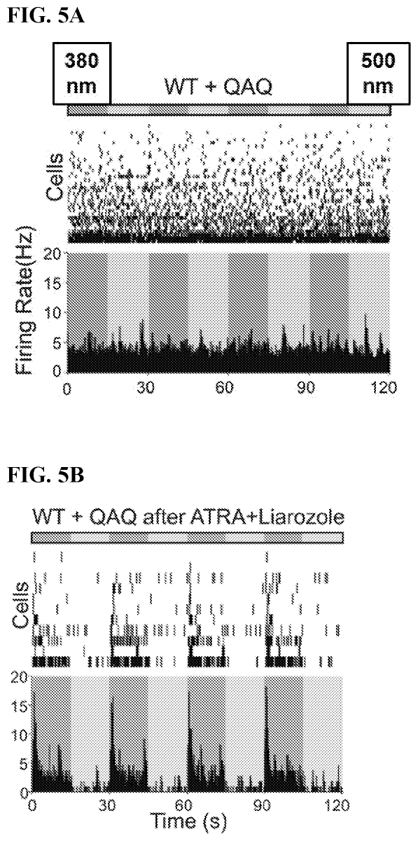

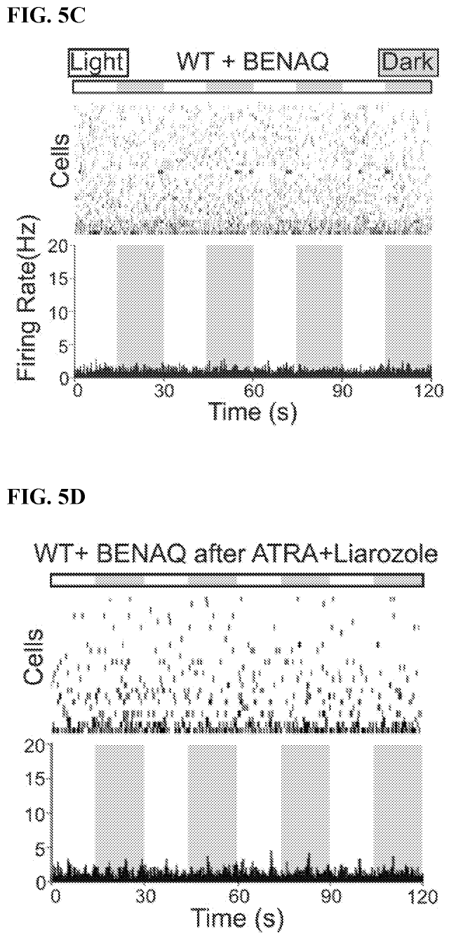

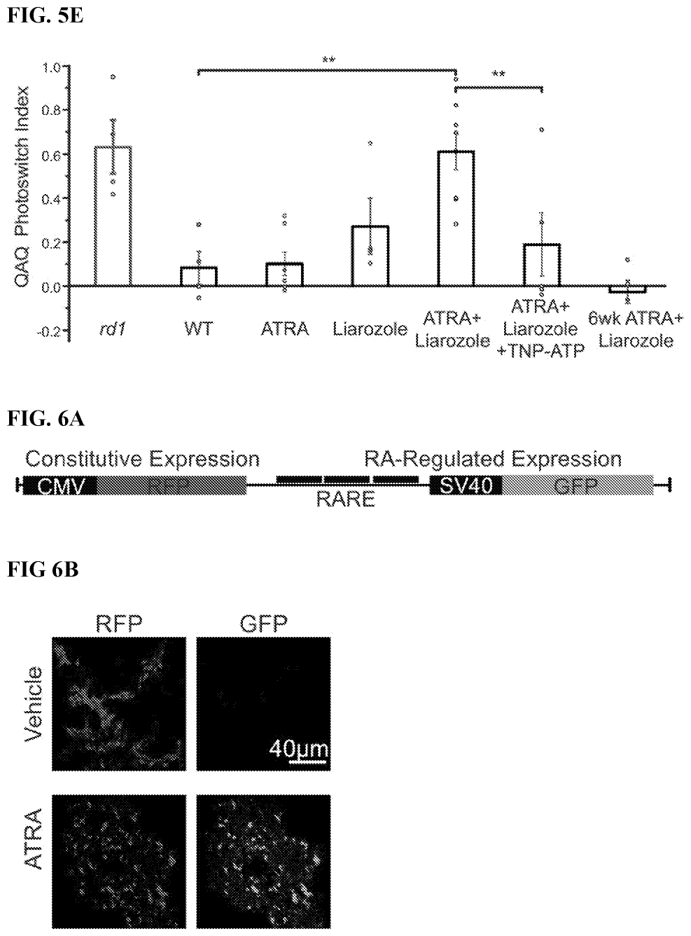

Activating RA signaling in WT retina enables chemical photosensitization of RGCs. Azobenzene photoswitches impart light-sensitivity on RGCs in rd1 mice but fail to photosensitize RGCs in WT mice (11,12). To test whether RA can enable photosensitization in WT RGCs, we used photoswitches that have different spectral sensitivities and target different types of ion channels. Both QAQ and BENAQ require active P2X receptors to permeate into RGCs, but whereas QAQ acts primarily on voltage-gated Na⁺ channels, BENAQ acts primarily on HCN channels. Treatment with ATRA plus liarozole enabled QAQ, which photoisomerizes between trans and cis with 380 or 500 nm light, to elicit light-dependent firing (FIG. 5A, FIG. 5B), similar to QAQ photosensitization in untreated rd1 (PI_{QAQ}=0.61±0.08, n=8, p=0.002,). Neither ATRA (PI=0.1±0.05, n=7) nor liarozole alone (PI_{QAQ}=0.27±0.13, n=4) enabled significant QAQ photosensitization. The P2X receptor antagonist TNP-ATP blocked QAQ photosensitization induced by ATRA plus liarozole (PI_{QAQ}=0.19±0.14, n=5, p=0.018) as is the case in rd1 mice. The effect of ATRA plus liarozole wore off within 6 weeks after intravitreal injection (PI_{QAQ}=−0.03±0.05, n=5), consistent with reversible enhancement of RA signaling. These features, including synergy between ATRA and liarozole, block by P2X receptor antagonists, and reversibility weeks after injection, mirror the effects of RA signaling on YO-PRO-1 labeling, consistent with a common mechanism.

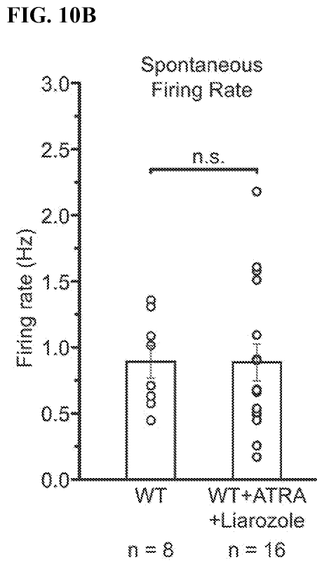

We next tested photosensitization by BENAQ. Unlike QAQ, BENAQ-elicited firing occurs in white light, and ceases abruptly in darkness (FIG. 2C). We observed no BENAQ photosensitization in WT retina after injection of ATRA plus liarozole (PI_{BENAQ}=−0.08±0.1, n=4) (FIG. 5C, FIG. 5D), suggesting insufficient up-regulation of HCN channels. We also found no increase in the spontaneous firing rate of RGCs following ATRA plus liarozole injection (FIGS. 10A-10B). Overall, short-term activation of RA signaling in WT enables QAQ photosensitization, but it does not enable BENAQ photosensitization or hyperexcitability, which may require the longer-term RA exposure that occurs during retinal degeneration.

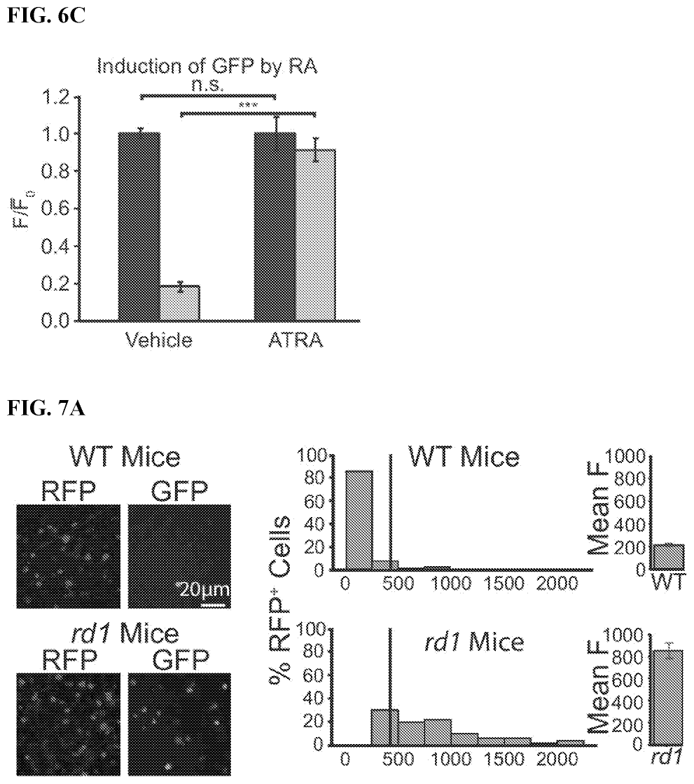

Detecting increased RA signaling in degenerated retina with an RAR reporter. If RGC remodeling is mediated by RA signaling through RAR, then there should be an increase in RA-dependent transcription. Free RA can be directly detected in intact tissue with a fluorescent probe (29) and RA-mediated transcription can be detected in tissue homogenates with a lac-Z-based reporter (30). However, verifying that RA mediates remodeling and identifying which cells are impacted by RA is best accomplished by visualization and quantification of RA-elicited transcription in intact retina. To achieve this, we have developed an RAR reporter that employs two fluorescent proteins for ratiometric measurement of RAR-dependent transcriptional activation. Multiple RA response elements (RAREs) are inserted upstream of a weak promoter (SV40) to drive GFP expression in response to activated RAR. A constitutive promoter (CMV) drives expression of red fluorescent protein (RFP) to report transfection or transduction efficiency (FIG. 6A).

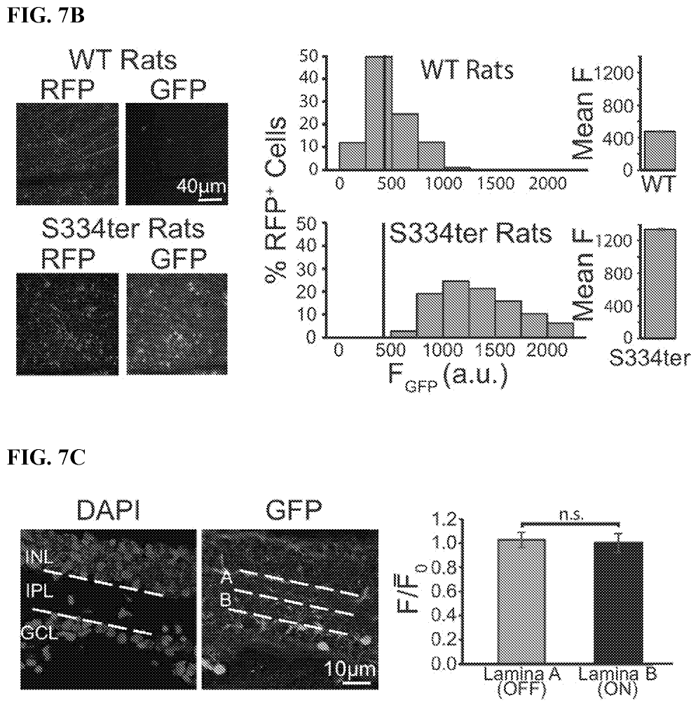

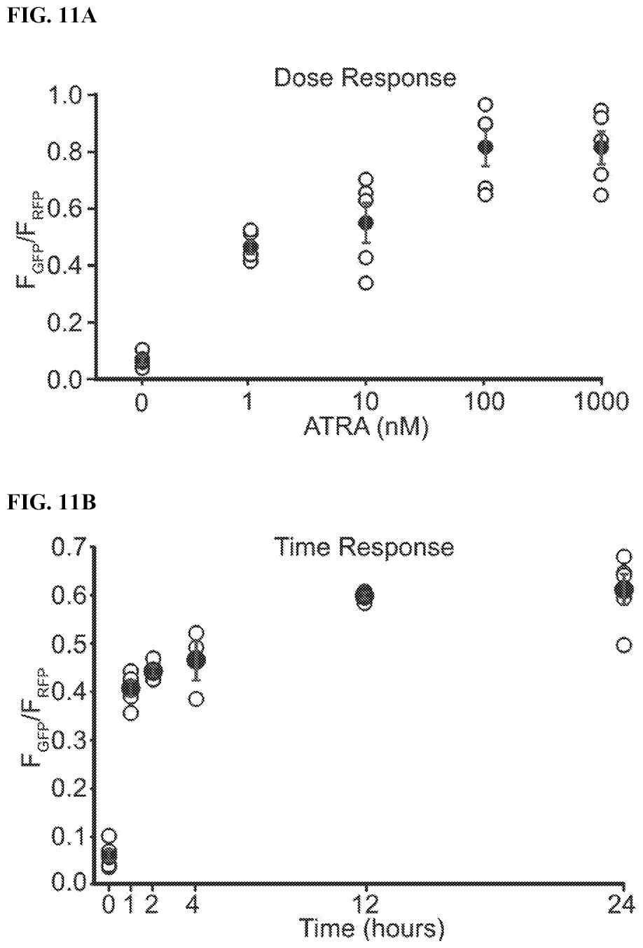

We first tested the RAR reporter in a human cell line (HEK293). Transfected cells expressed RFP, but very little GFP (FIG. 6B, FIG. 6C). Treatment with ATRA (1 μM, 48 hrs) induced GFP expression. The increase in GFP to RFP ratio was dose- and time-dependent (FIGS. 11A-11B). Next, an AAV-packaged RAR reporter was used for intravitreal injections in vivo. In WT retina, many RGCs were transduced by the virus and therefore expressed RFP, but very few expressed GFP. In contrast, many rd1-RGCs expressed both RFP and GFP (FIG. 7A).

To quantify these observations, we compared the distribution of GFP fluorescence values across RFP-expressing RGCs from rd1 and WT retina. In rd1, ˜70% of cells had GFP values above threshold (>2 SD above background), in contrast to only ˜20% of cells in WT. The mean GFP fluorescence intensity in rd1 RGCs was 4-fold higher than the mean GFP value measured in WT retinas (851.3±66.7 vs. 213.4±11.1; n=8, p<0.001).

We also observed increased RA signaling in transgenic s334ter rats, in which photoreceptor degeneration is caused by a rhodopsin mutation identical to a genetic subtype of human RP (31). The RAR reporter virus showed that all of the RFP-expressing cells expressed GFP above threshold in s334ter retina, whereas only about half expressed GFP in WT retina (FIG. 7B). The mean GFP fluorescence in s334ter RGCs was almost 3-fold higher than the WT value (1336.1±9.4 vs. 476.8±7.5; n=10, p<0.001). These findings show that in two different animal models, photoreceptor degeneration is correlated with an increase in RA signaling in RGCs.

P2X and HCN up-regulation in RGCs is limited to presumptive OFF-RGCs (11), whose dendrites ramify in the outer sublamina of the IPL. The RPE, which is the main source of retinoids for the retina, is located beneath the photoreceptors, raising the possibility of an RA gradient, with the dendrites of OFF-RGCs exposed to a higher concentration than the dendrites of ON-RGCs. We used a GFP-specific antibody to amplify the signal produced by the RAR reporter. However, we found no difference in GFP immunolabeling across the ON-vs. OFF-sublamina of the IPL (FIG. 7C), inconsistent with an RA gradient. This finding implies that cell-type specific differences in pathophysiological remodeling of RGCs are mediated downstream of the RAR, consistent with distinct genetic and/or epigenetic programs intrinsic to OFF- vs. ON-RGCs.

Our results show that RA signaling is necessary and sufficient for pathophysiological remodeling of RGCs during degenerative blindness. Moreover, we can actually detect elevated RA signaling in RGCs during rod and cone degeneration. Although the time course of degeneration differs between mouse and man, retinal remodeling follows a stereotyped progression across species (32-35). These parallels suggest remodeling is initiated by a common signal, namely RA. Data mining of human transcriptomes (36) shows heightened expression of RA-induced genes in RP (Table 1).

The immediate precursor to RA is retinaldehyde, the vitamin A derivative that serves as the chromophore (15). Retinaldehyde is produced in RPE cells and shuttled across the subretinal space by the extracellular carrier Interphotoreceptor Retinoid Binding Protein (IRBP) where it binds opsins in photoreceptor outer segments. Since the retina contains millions of rods and cones each with millions of opsins, loss of the outer segments removes an enormous molecular sink that would normally sequester retinaldehyde. Moreover, loss of the inner segments creates a breach in the outer limiting membrane (OLM), which may allow retinaldehyde to diffuse through the remaining layers. Several retinal cell types, including amacrine cells and Müller glial cells, express the enzyme RALDH (27), which can convert retinaldehyde to RA. In WT mice, the low concentration of free retinaldehyde limits production of RA. However, when we experimentally bypassed the outer segment retinoid sink by injecting retinaldehyde into the vitreous, we observed the same physiological remodeling of RGCs as when we injected ATRA (FIGS. 4A-4C). This implies enzymatic conversion of retinaldehyde to RA. Supporting this idea, co-injection of retinaldehyde with a RALDH inhibitor prevented remodeling.

RA can signal in two distinct ways. First, it binds to and activates nuclear receptors (RARs) that control gene transcription (37,38). However, there is also evidence that RA can regulate protein kinases and phosphatases in the cytoplasm, altering protein phosphorylation (39). Retinal remodeling is associated with changes in gene transcription (37), including P2X receptors and HCN channels (11). We found that BMS-493, a drug that specifically blocks RARs, nearly eliminates degeneration-dependent remodeling in rd1 and P2X activity caused by injecting ATRA. This strongly suggests that RA signals through RAR to change the transcriptional program of RGCs during photoreceptor degeneration.

Biophysical studies show that upon chronic activation, the pore of P2X receptors dilates, allowing large molecules (up to ˜14 Å) to pass (40-42). We find that treatments that increase RA in wild-type retina allow YO-PRO-1 to enter RGCs, and this is prevented by blocking P2X receptors (FIGS. 4A-4C). Dilated P2X receptors also allow photoswitches into RGCs, enabling light to control action potential firing (11). We found that RA treatment enabled QAQ to photosensitize WT RGCs, which are otherwise resistant to photosensitization (FIGS. 5A-5E). RGC photosensitization by azobenzene photoswitches might be facilitated by co-administering drugs that activate RA signaling, such as ATRA (tretinoin, Allergan, NDA #090098) or its isomer, 13-cis retinoic acid (Accutane, Hoffmann-La Roche Inc., NDA #018662), both of which are already FDA-approved for unrelated indications.

Why did RA enable QAQ to photosensitize WT RGCs, but not BENAQ? QAQ acts primarily on voltage-gated Na⁺ channels (43) that are in all RGCs, both in healthy and degenerating retina. However, HCN channels, the primary target for BENAQ, are sparse in RGCs of healthy retina, but up-regulated in degenerated retina (11). While a single injection of ATRA into the eye of an adult WT mouse triggered P2X-dependent membrane permeabilization, ATRA is rapidly degraded and therefore exposure to the drug may have been too brief to up-regulate HCN channels, rendering BENAQ ineffective. Likewise, while blocking RA signaling reduced the heightened firing of RGCs in darkness, a single injection of ATRA in WT retina was insufficient to increase spontaneous firing. While transient enhancement of RA signaling was not able to recapitulate all aspects of remodeling (44), apparently chronic exposure to RA, as it occurs in a degenerated retina, is able to up-regulate HCN and induce hyperexcitability.

We have shown that blocking RAR leads to an enhancement of light responses in RGCs, suggesting a therapeutic strategy for improving low-level vision before photoreceptor degeneration is complete. Signals from the few remaining rods and cones can be maximized by blocking RA signaling, reducing noisy background firing in RGCs. An analogous decrease in signal-to-noise occurs with tinnitus in hearing loss, where degeneration of a subset of cochlear hair cells leads to hyperactivity of auditory neurons (45), corrupting the remaining sound-elicited neural signals that are transmitted to the brain. By reducing the background noise, treatment with RA blockers lowered light detection threshold and increased the maximal response, enabling RGCs to more effectively encode visual information.

There are several targets along the RA-signaling pathway for possible pharmacological intervention. RA synthesis can be inhibited with an ALDH inhibitor such as disulfiram (Antabuse, Odyssey Pharms, NDA #088482). However, we found that directly antagonizing RARs was most effective for mitigating remodeling. RAR antagonists have been developed as cancer therapeutics (46) but may be repurposed for enhancing visual function after death of rods and cones, which is incomplete in the vast majority of patients with retinal degenerative diseases.

Artificial light responses evoked with electrical, optogenetic, or optopharmacological stimulation will be superimposed on the heightened background activity of remodeled RGCs, limiting the ability to imitate the natural neural code for vision (47). The only vision restoration technology currently approved by the FDA, the Argus II multi-electrode retinal prosthetic, provides spatial acuity that is 3-4 fold lower than predicted by the spacing of the electrodes (48). Reducing the spontaneous activity of RGCs by blocking RA signaling might boost the performance of this and other vision-restoration technologies (49).

Materials and Methods

Reagents.

Photoswitch compounds were synthesized and prepared as formate salts as previously described (43,50,51). All other chemicals were purchased from Sigma-Aldrich, Tocris Bioscience, Life Technologies, or Santa Cruz Biotech. Those chemicals that are insoluble in water were first dissolved in DMSO and diluted in ACSF to a final concentration containing <1% DMSO.

Animals.

Retinas were isolated from WT mice (C₅₇BL/6J strain, Jackson Laboratory or Charles River), homozygous rd1/rd1 mice (C₃H/HeJ strain, Charles River Laboratories), WT rats (Long Evans strain, Charles River Laboratories) and S334-ter rats (line 3). All animal use procedures were approved by the UC Berkeley Institutional Animal Care and Use Committee.

Intravitreal Injections.

Before injection, mice were anesthetized with isoflurane (2%) and their pupils were dilated with tropicamide (1%) and phenylephrine (2.5%). Proparacaine (0.5%) was used as a topical analgesic. Genteal was applied under a glass coverslip to keep the cornea lubricated. An incision was made through the sclera below the ora serrata with a 30 G needle and ˜1 μl of solution was injected into the vitreous with a blunt-ended 33 G Hamilton syringe. After injection, the antibiotic tobramycin (0.3%) was applied to the eye. Final drug concentrations after intravitreal injection were: all-trans retinoic acid (ATRA, 100 nM), liarozole (100 μM), diethylaminobenzaldehyde (DEAB, 20 μM), citral (50 μM), BMS-493 (500 nM), and retinaldehyde (1 μM). The above values correspond to the final concentration in the eye after injecting 1 μl of drug accounting for a 5-fold dilution. An injection of PBS including 1% DMSO was used as vehicle control.

Tissue Preparation.

Eyes were enucleated immediately following euthanasia. Corneas were punctured and globes were placed into oxygenated artificial cerebral spinal fluid (ACSF) containing (in mM) 119 NaCl, 2.5 KCl, 1 KH 2 PO 4, 1.3 MgCl₂, 2.5 CaCl₂, 26.2 NaHCO₃, and 20 D-glucose, aerated with 95% O₂/5% CO₂. Retinas were dissected and kept in ACSF at room temperature until recording.

Multi Electrode Array Recordings.

For extracellular recordings, a flat-mounted retina was placed ganglion cell layer down onto a 60-electrode Multi-Electrode Array (MEA 1060-2-BC, Multi-Channel Systems). After mounting the retina, photoswitches were applied for 30 min, followed by a 15 min wash. BENAQ was applied at a concentration of 100 μM and QAQ at 300 μM. A solution containing a mixture of neurotransmitter receptor blockers isolated RGCs from synaptic inputs: (in μM) 10 AP4, 40 DNQX, 30 APS, 10 SR-95531, 50 TPMPA, 10 strychnine, and 50 tubocurarine. Extracellular spikes were high-pass filtered at 200 Hz and digitized at 20 kHz and were counted when exceeding 4 SD from the mean background voltage signal. Typically, each electrode recorded spikes from one to three individual RGCs. Principal component analysis of the spike waveforms was used for sorting spikes generated by individual cells (Offline Sorter, Plexon). Stimulation light was generated from a mercury arc lamp. Unfiltered broad spectrum light was used for BENAQ-treated retinas. Narrow band optical filters (Chroma) were used to deliver alternating intervals of 380 nm and 500 nm for stimulation of QAQ-treated as described previously (5,12). The Photoswitch Index (5) (PI) was established for individual retinas in light/darkness for treatment with BENAQ or in 380 nm/500 nm light for treatment with QAQ (11,12).

Yo-PRO-1 Loading Assay.

After dissection, retinas were cut into thirds and mounted on a windowed nitrocellulose filter paper. Retinas were treated with 200 nM Yo-PRO-1 (Life Technologies) in oxygenated ACSF for 15 minutes, followed by treatment and then treated with nuclear ID (Enzo Life Sciences) at a 1:500 dilution for 3 minutes. ACSF was perfused continuously at 3 ml/min for a period of 5 minutes to wash away excess dye. In experiments employing TNP-ATP (200 μm), the retinas was pretreated with the compound for ten minutes before beginning YO-PRO-1 treatment.

RAR Reporter Virus Assay.

A plasmid was designed and synthesized (Vigene Biosci., Maryland, USA) to include a cytomegalovirus promoter (CMV) upstream to the coding sequence for RFP, followed by poly-A and a stop sequence. A fragment containing three repetitions of the retinoic acid response element (RARE) sequence followed by the weak promoter SV40 was sub-cloned from an original plasmid containing RARE-SV40-LacZ (52). The final construct was packaged in an AAV viral backbone, and viral yields were purified from HEK293T cells (10¹³-10¹⁴ particles/μl). An AAV9 serotype was used for intravenous infection of P0-P2 WT and rd1 mice(53), while an AAV2 serotype was used for intravitreal infection of P90-P120 WT and s334ter rats. Mice were sacrificed and analyzed at P60-P90 and rats ˜15 days after infection. Retinas were isolated and imaged in flat-mount configuration using transparent PDFA membranes (Millipore). During imaging, retinas were continuously perfused with oxygenated ACSF. In every retina, at least 6 fields were imaged.

Immunolabeling and TUNEL Assay.

S334ter rat retinas infected with the RARE double reporter were dissected, fixed and frozen, as previously described (54). The tissue was cut in 14 μm thick cross-sections using a Leica cryostat. Immunocytochemical solution (ICC) was composed of standard 1×PBS (Gibco), 2.5% BSA (Sigma) and 0.1% Triton (Sigma). The tissue was blocked using ICC solution supplemented with unconjugated rabbit anti-mouse secondary antibody (Life Technologies) at RT for 1 hr. Slides were incubated with primary mouse anti-GFP antibody (#JL8, Clontech), with no RFP cross-reactivity, at 4° C. ON. An Alexa Fluor-488 conjugated goat anti-mouse secondary antibody (Life Technologies) was used at RT for 1 hr in the dark. The same procedure was carried out for detection of RARβ in mouse retinas following injection of vehicle or ATRA. Slides were incubated with a primary rabbit anti-RARβ antibody (ab53161, Abcam) and detected using an Alexa Fluor-488 conjugated goat anti-rabbit secondary antibody (Life Technologies). TUNEL assay (In Situ Cell Death Detection Kit, Roche) was carried out per manufacturer's instructions. Retinas were collected 5-6 days following injection with vehicle, ATRA, or ATRA and Liarozole.

Imaging and Analysis. Confocal Microscopy:

Yo-PRO-1 loading assays and RARE double reporter virus assay in mice were imaged using using a spinning disk confocal microscope (Olympus BX61WI). The excitation source was a mercury lamp and fluorescence was collected by a 40× water immersion imaging objective. Standard GFP and RFP filter cubes (Olympus, U-URA) with excitation and emission spectral peaks at ex: 488 nm, 561 nm, em: 519 nm, >575 nm, respectively. 1.5 μm section Z-stacks were acquired using a Hamamatsu ImageEM CCD C9100-13. RARE double reporter virus in rats, RARb immunolabeling and TUNEL assay were imaged using a Laser Scanning Confocal Microscope (LSM 780 NLO, Zeiss), using Zen software and default configuration for RFP, GFP, AF488 and nuclear-ID detection.

Image Analysis.

All image analysis was performed using ImageJ or Fiji software (55,56). Yo-PRO-1 loading assay, ROIs were manually selected using the nuclear-ID channel based upon morphological characteristics. This was performed after computationally flattening the retina by performing a maximum Z projection onto a single plane. Following this, a background subtraction was performed with a rolling radius of 50 pixels. Cells identified as vascular endothelial cells or pericytes were not included in the analysis. This was confirmed using transillumination. A threshold for YO-PRO-1 loading was established by measuring the level of autofluorescence of untreated retinas in each channel and finding a baseline value with +2SD being the threshold for a YO-PRO-1 positive cell. Nuclear-ID was used to count the total number of cells within a field of view. The percentage of cells above the threshold was then calculated for comparison. For analysis of RARE double reporter virus assay, ROIs were manually selected on the RFP channel first and then superimposed to the GFP channel. Single cell values for both RFP and GFP were filtered by using a RFP minimum threshold established in naive unlabeled retinas.

Data Analysis and Statistics.

Unless otherwise stated, all statistical significance (p-value) calculations were performed using the two-tailed unpaired Student's t test. Results with p<0.05 were considered significant. P-values are: *<0.05, **<0.01, ***<0.001. Pairwise comparisons for non-parametric data employed the Wilcoxon Rank Sum Test or the Mann-Whitney U-test. In the case where ANOVA was employed, bootstrapping was used to account for unequal group sizes and a Tukey HSD test was employed as a post-hoc test to define which comparisons and interactions produced statistically significant changes. A modified Thompson-Tau method was employed for the detection of outliers. Non-normal data distributions were analyzed using the Kruskal-Wallis ANOVA with a Dunn's post hoc test. Sigmoidal curves were fit using OriginPro™ and the output curves for vehicle and BMS-493 are y=START+(END−START)*x\hat{ }n/(k\hat{ }n+x\hat{ }n).

Transcriptomic Analysis.

The publicly available dataset accompanying Mullins RF et al 2012 (“Dataset S1; http://iovs.arvojournals.org/data/Journals/IOVS/933465/IOVS-12-9477-1883-s01.xls) (36) was mined for retinoic-acid associated genes. Only probes that were present (“P”) in both samples, healthy (“Ctl”) and retinitis pigmentosa (“RP”) were included in the analysis.

TABLE 1
Analysis of Retinoic-Acid related genes in human retinitis pigmentosa transcriptome
data. Data mining of the human transcriptome dataset published by Mullins R F et al.,
IOVS 2012 (36). Each probe is accompanied by the gene name and title, and its relative
expression value each sample. The ratio was obtained by dividing the control (Ctl)
value by the RP value. A) List of representative photoreceptor-associated genes, strongly
upregulated in the control sample and downregulated to absent in the RP sample. B)
List of retinoic acid-associated genes and their relative expression levels in human
RP as compared to healthy retinas. Genes are ordered increasing expression ratios.
Genes induced by RA show the greatest levels of up-regulation.

| | Probe ID | Gene symbol | Gene title | RP value | Ctl value | Ratio |
|---|---|---|---|---|---|---|
| A | 206455_s_at | RHO | rhodopsin (opsin 2, rod | 5.27 | 6653.68 | 1262.33 |
| | | | pigment) (retinitis | | | |
| | | | pigmentosa 4, autosomal | | | |
| | | | dominant) | | | |
| | 206623_at | PDE6A | phosphodiesterase 6A, | 7.27 | 7075.39 | 972.13 |
| | | | cGMP-specific, rod, alpha | | | |
| | 206417_at | CNGA1 | cyclic nucleotide gated | 4.85 | 4000.59 | 824.19 |
| | | | channel alpha 1 | | | |
| | 210060_at | PDE6G | phosphodiesterase 6G, | 17.08 | 11841.48 | 693.26 |
| | | | cGMP-specific, rod, | | | |
| | | | gamma | | | |
| | 207514_s_at | GNAT1 | guanine nucleotide binding | 34.29 | 10843.19 | 316.15 |
| | | | protein (G protein), alpha | | | |
| | | | transducing activity | | | |
| | | | polypeptide 1 | | | |
| B | 206392_s_at | RARRES1 | retinoic acid receptor | 238.91 | 17.86 | 0.07 |
| | | | responder (tazarotene | | | |
| | | | induced) 1 | | | |
| | 206391_at | RARRES1 | retinoic acid receptor | 171.95 | 14.47 | 0.08 |
| | | | responder (tazarotene | | | |
| | | | induced) 1 | | | |
| | 206424_at | CYP26A1 | cytochrome P450, family | 75.33 | 8.03 | 0.10 |
| | | | 26, subfamily A, | | | |
| | | | polypeptide 1 | | | |
| | 221872_at | RARRES1 | retinoic acid receptor | 1440.23 | 184.66 | 0.12 |
| | | | responder (tazarotene | | | |
| | | | induced) 1 | | | |
| | 204070_at | RARRES3 | retinoic acid receptor | 3506.99 | 567.23 | 0.16 |
| | | | responder (tazarotene | | | |
| | | | induced) 3 | | | |
| | 202449_s_at | RXRA | retinoid X receptor, alpha | 799.80 | 186.41 | 0.23 |
| | 209496_at | RARRES2 | retinoic acid receptor | 4748.54 | 1746.00 | 0.36 |
| | | | responder (tazarotene | | | |
| | | | induced) 2 | | | |
| | 219440_at | RAI2 | retinoic acid induced 2 | 579.20 | 233.92 | 0.40 |
| | 1552378_s_at | RDH10 | retinol dehydrogenase 10 | 71.11 | 32.61 | 0.45 |
| | | | (all-trans) | | | |
| | 202052_s_at | RAI14 | retinoic acid induced 14 | 744.23 | 362.75 | 0.48 |
| | 1566472_s_at | RETSAT | retinol saturase (all-trans- | 45.23 | 22.16 | 0.48 |
| | | | retinol 13, 14-reductase) | | | |
| | 230217_at | RLBP1L1 | retinaldehyde binding | 17.60 | 9.13 | 0.51 |
| | | | protein 1-like 1 | | | |
| | 230217_at | RLBP1L1 | retinaldehyde binding | 17.60 | 9.13 | 0.51 |
| | | | protein 1-like 1 | | | |
| | 203749_s_at | RARA | retinoic acid receptor, alpha | 31.44 | 16.43 | 0.52 |
| | 226143_at | RAI1 | retinoic acid induced 1 | 149.23 | 82.43 | 0.55 |
| | 219140_s_at | RBP4 | retinol binding protein 4, | 160.54 | 94.22 | 0.58 |
| | | | plasma | | | |
| | 225467_s_at | RDH13 | retinol dehydrogenase 13 | 37.47 | 23.16 | 0.61 |
| | | | (all-trans/9-cis) | | | |
| | 222049_s_at | RBP4 | Retinol binding protein 4, | 140.31 | 88.48 | 0.63 |
| | | | plasma | | | |
| | 209148_at | RXRB | retinoid X receptor, beta | 36.13 | 25.18 | 0.69 |
| | 227360_at | RDH13 | Retinol dehydrogenase 13 | 46.30 | 32.68 | 0.70 |
| | | | (all-trans/9-cis) | | | |
| | 209478_at | STRA13 | stimulated by retinoic acid | 174.24 | 128.05 | 0.73 |
| | | | 13 homolog (mouse) | | | |
| | 222203_s_at | RDH14 | retinol dehydrogenase 14 | 1029.22 | 791.73 | 0.76 |
| | | | (all-trans/9-cis/11-cis) | | | |
| | 1559190_s_at | RDH13 | MRNA; cDNA | 616.06 | 495.08 | 0.80 |
| | | | DKFZp313H0740 (from | | | |
| | | | clone DKFZp313H0740) /// | | | |
| | | | Retinol dehydrogenase 13 | | | |
| | | | (all-trans/9-cis) | | | |
| | 202575_at | CRABP2 | cellular retinoic acid | 36.00 | 31.16 | 0.86 |
| | | | binding protein 2 | | | |
| | 205350_at | CRABP1 | cellular retinoic acid | 12074.17 | 10583.63 | 0.87 |
| | | | binding protein 1 | | | |
| | 219825_at | CYP26B1 | cytochrome P450, family | 17.84 | 16.21 | 0.90 |
| | | | 26, subfamily B, | | | |
| | | | polypeptide 1 | | | |
| | 218337_at | RAI16 | retinoic acid induced 16 | 23.71 | 23.81 | 1.00 |
| | 226021_at | RDH10 | retinol dehydrogenase 10 | 3003.88 | 3020.59 | 1.00 |
| | | | (all-trans) | | | |
| | 227467_at | RDH10 | retinol dehydrogenase 10 | 3315.81 | 3339.23 | 1.00 |
| | | | (all-trans) | | | |
| | 215099_s_at | RXRB | retinoid X receptor, beta | 8.19 | 8.42 | 1.02 |
| | 238017_at | RDHE2 | epidermal retinal | 14.48 | 15.22 | 1.05 |
| | | | dehydrogenase 2 | | | |
| | 238017_at | RDHE2 | epidermal retinal | 14.48 | 15.22 | 1.05 |
| | | | dehydrogenase 2 | | | |
| | 205080_at | RARB | retinoic acid receptor, beta | 271.37 | 321.84 | 1.18 |
| | 238066_at | RBP7 | retinol binding protein 7, | 199.44 | 243.93 | 1.22 |
| | | | cellular | | | |
| | 205954_at | RXRG | retinoid X receptor, gamma | 170.24 | 214.16 | 1.25 |
| | 217775_s_at | RDH11 | retinol dehydrogenase 11 | 1205.90 | 1559.68 | 1.29 |
| | | | (all-trans/9-cis/11-cis) | | | |
| | 206154_at | RLBP1 | retinaldehyde binding | 3790.88 | 5330.12 | 1.40 |
| | | | protein 1 | | | |
| | 206154_at | RLBP1 | retinaldehyde binding | 3790.88 | 5330.12 | 1.40 |
| | | | protein 1 | | | |
| | 217776_at | RDH11 | retinol dehydrogenase 11 | 1762.30 | 2572.59 | 1.45 |
| | | | (all-trans/9-cis/11-cis) | | | |
| | 203750_s_at | RARA | retinoic acid receptor, alpha | 37.85 | 70.09 | 1.85 |
| | 221701_s_at | STRA6 | stimulated by retinoic acid | 98.40 | 204.99 | 2.08 |
| | | | gene 6 homolog (mouse) | | | |
| | 203423_at | RBP1 | retinol binding protein 1, | 3028.05 | 7068.31 | 2.33 |
| | | | cellular | | | |
| | 208530_s_at | RARB | retinoic acid receptor, beta | 21.66 | 52.60 | 2.42 |
| | 210106_at | RDH5 | retinol dehydrogenase 5 | 68.75 | 595.74 | 8.66 |
| | | | (11-cis/9-cis) | | | |
| | 236291_at | RDH5 | retinol dehydrogenase 5 | 9.83 | 97.06 | 9.87 |
| | | | (11-cis/9-cis) | | | |
| | 220683_at | RDH8 | retinol dehydrogenase 8 | 53.69 | 1151.37 | 21.44 |
| | | | (all-trans) | | | |
| | 210318_at | RBP3 | retinol binding protein 3, | 75.63 | 4464.43 | 59.02 |
| | | | interstitial | | | |
| | 242998_at | RDH12 | retinol dehydrogenase 12 | 7.09 | 2559.60 | 360.64 |
| | | | (all-trans/9-cis/11-cis) | | | |

### Example 2. Genetic Manipulation of RAR—Gene Therapy in Retina to Reduce RA-Signaling and Improve Low-Level Vision

An alternative approach to treating vision degeneration is to genetically eliminate RARs (e.g., RARα, RARβ, or RARγ), individually or as a group. This can be achieved through various techniques, including but not limited to: genomic editing (1), viral delivery of dominant negative forms of these receptors (2), and RNA interference approaches (3).

(1) Through genomic editing, including but not limited to CRISPR, TALEN, zinc-finger nuclease, and similar, a part or all of the sequence of RARs is eliminated from the genome, rendering its expression null. CRISPR, TALEN, and zinc-finger nuclease genome editing systems are useful tools for generating double-strand breaks at specific genomic regions of interest (e.g., exons, introns, genes associated a particular function).

(2) Through viral or otherwise delivery of DNA sequences into the retina, it is possible to introduce a dominant negative form of the retinoic acid receptor under strong expressing promoters. A dominant negative RAR is a retinoic acid receptor wherein the function is disrupted, for example wherein retinoic acid-mediated release from suppression is prevented. For example, in the human RARα, truncating the protein at amino acid 403 leads to a dominant negative form that competes against the endogenous unaltered receptor (e.g., wild-type RAR), resulting in the suppression of RAR-induced genes (see additional details in Damm K et al, PNAS Apr. 1, 1993 vol. 90 no. 7 2989-29933; Novitch B G et al, Neuron. 2003 Sep. 25; 40(1):81-95).

(3) Through viral or otherwise delivery of siRNA, shRNA, miRNA or any RNA-interference method, by which RAR mRNA transcripts are bound by the antisense and degraded by the cell, before the translation process results in production of the protein.

Expression of all types of sequences described in the enumerated methods 1, 2 and 3 above, can be also made drug-inducible, through the use of drug-inducible promoters (e.g., Tetracycline-Controlled Transcriptional Activation (TET), including but not limited to, TET-ON, TET-OFF, etc. Note, the difference between TET-ON and TET-OFF is not whether the transactivator turns a gene on or off, as the name might suggest; rather, both proteins activate expression. The difference relates to their respective response to tetracycline or doxycycline (Dox, a more stable tetracycline analogue); TET-OFF activates expression in the absence of Dox, whereas TET-ON activates in the presence of Dox.

In addition, through the techniques mentioned above, it is possible to eliminate RALDH, the enzyme that converts retinaldehyde to retinoic acid, therefore eliminating the binding of retinoic acid to its receptors in retinal neurons.

Finally, the serotype of the virus used to deliver the construct, as well as the promoter(s) used for expression, need to be chosen for cell-specific delivery and expression. For example, AAV2 infects mostly RGCs in the retina with reduced delivery in the INL. For example, the promoter Thy1 drives expression that is restricted to RGCs, while hSynapsin promoter can be used to express in RGCS and in bipolar cells, etc.

### Example 3: Retinoic Acid Triggers Pathophysiological Retinal Hyperactivity in Degenerative Blindness

Light responses are initiated in photoreceptors, processed by interneurons, and synaptically transmitted to retinal ganglion cells (RGCs), which send information to the brain. Retinitis pigmentosa (RP) is a blinding disease caused by photoreceptor degeneration, depriving downstream neurons of light-sensitive input. In addition, photoreceptor degeneration triggers hyperactive firing of RGCs that obscures light responses initiated by surviving photoreceptors. Here we show that retinoic acid (RA), signaling through the RA receptor (RAR), is the trigger for hyperactivity. A genetically-encoded fluorescent reporter shows elevated RAR signaling in degenerated retinas from murine models of RP. Enhancing RAR signaling in healthy retinas mimics the pathophysiology of degenerating retinas. Drug inhibition of RAR reduces hyperactivity in degenerating retinas and unmasks light responses in RGCs. Gene therapy inhibition of RAR increases innate and learned light-elicited behaviors in vision-impaired mice. Identification of RAR as the trigger for hyperactivity presents a degeneration-dependent therapeutic target for enhancing low-level vision in RP and other blinding disorders.

Retinitis Pigmentosa (RP) is an inherited blinding disease caused by the loss of rod and cone photoreceptors. RP progresses slowly, with retinal light responses and visual acuity declining over years or decades after the initial diagnosis. Retinal ganglion cells (RGCs) maintain synaptic connectivity with the brain (1,2), making them a potential substrate for artificial vision restoration by optoelectronics (3), optogenetics (4), or optopharmacology (5,12). However, because these technologies supplant light responses initiated by any residual rods and cones, they are only appropriate for end-stage degenerative disease. Hence there is an unmet need for treatment strategies that enhance, rather than replace, retinal light responses.

Even though downstream retinal neurons survive, their physiology and morphology gradually change (33,34). Months after the photoreceptors die, new dendritic branches appear in several types of retinal neurons and even later, cell body position begins to change in mouse, rat, and rabbit models of RP, mirroring events that occur over years in advanced human RP (6-8,57). A critical part of this process is that RGCs become hyperactive. Since visual stimuli are encoded by the spike patterns of RGCs, increased background firing reduces information transfer to the brain, degrading visual sensitivity. RGC hyperactivity has been attributed to increased excitatory synaptic drive (9,58), but a large component remains after blocking chemical synaptic transmission (44,59-61). Therefore, hyperactivity of RGCs must be the result of a change in voltage-gated channels intrinsic to RGCs (11,12) and/or increased electrical coupling between inner retinal neurons and RGCs (62-64).

While stereotypical pathophysiological events occur across mammalian species (6,32,34), the signal that tells downstream neurons that the photoreceptors are degenerating is unknown. Our goal in this study was to identify this signal and ask whether blocking it can reverse pathophysiological changes, thereby improving visual sensitivity. In principle, several types of signals might induce remodeling. Perhaps death of photoreceptors leads to a decrease in a light-dependent synaptic signal, such as glutamate-induced Ca′ influx, which might act as a suppressor of remodeling in healthy retina (33). Inconsistent with this idea, mice with mutations that eliminate phototransduction without causing degeneration, show no pathophysiology (12). Perhaps photoreceptor death increases an inducer of remodeling. Retinoic acid (RA) has been implicated in triggering new dendritic growth in the outer retina after light-induced damage (20,21), leading us to ask whether it might also serve as the trigger for pathophysiological remodeling in RP.

RA is a transcriptional regulator that plays crucial roles in early embryonic development and differentiation (16,22,26). RA can also serve as a neural signal in adulthood, mediating synaptic plasticity in cortex and hippocampus (17-19). RA is derived from retinaldehyde (RAL), the chromophore for opsins. The loss of outer segments eliminates most opsin from the retina, perhaps allowing increased biosynthesis of RA. If RA is the trigger for RGC hyperactivity, treatments that interfere with RA synthesis or signal transduction should prevent or reverse modeling, confirming that RA is necessary. Treatments that enhance RA signaling should mimic pathophysiological changes, confirming that RA is sufficient. A reporter of RA-induced transcription should reveal whether RA signal transduction is heightened during retinal degeneration. Finally, interventions that prevent RA signaling should reverse hyperactivity, thereby improving impaired vision.

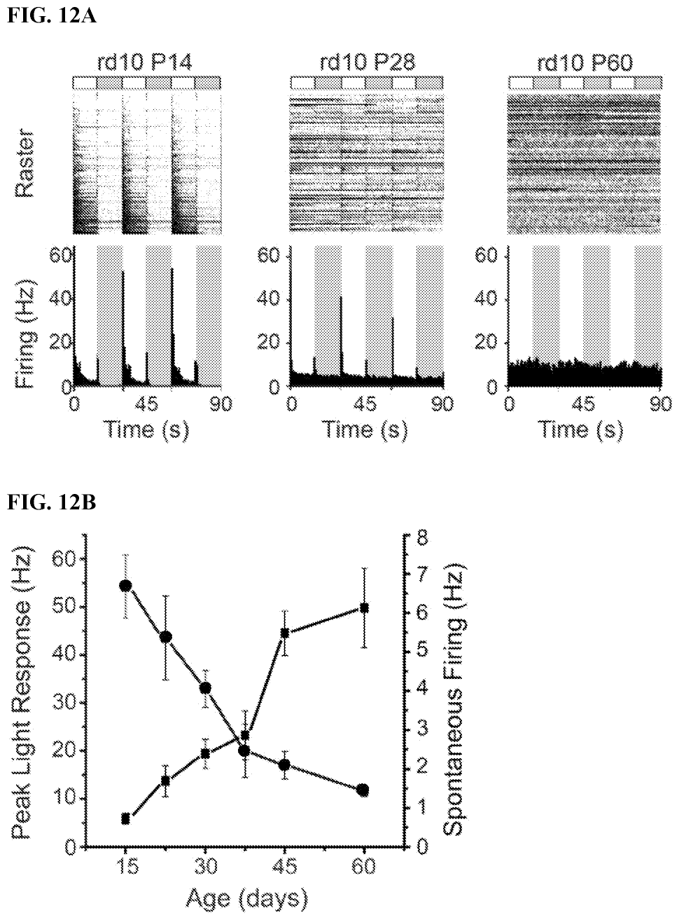

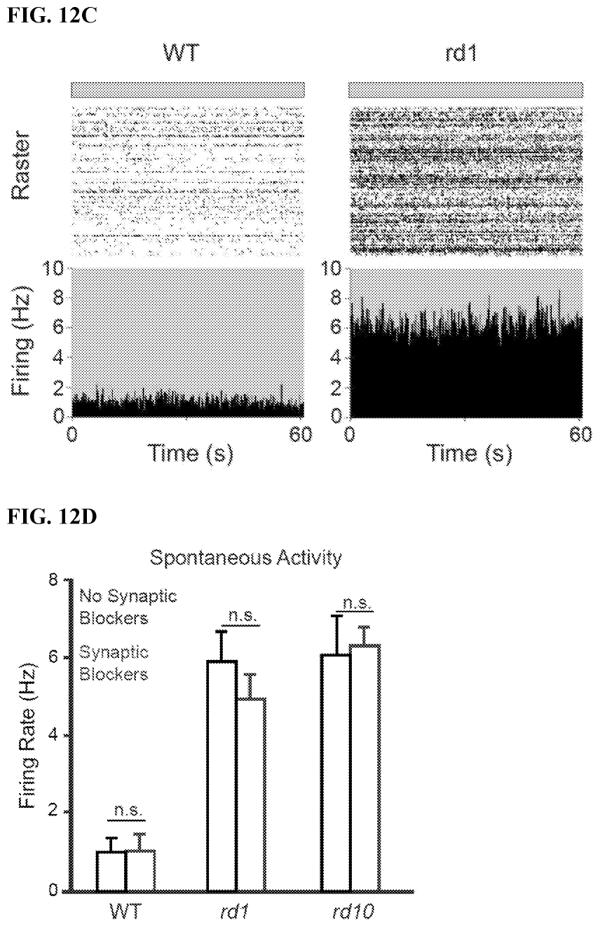

Photoreceptor degeneration leads to RGC hyperactivity. As a starting point, we measured spontaneous RGC firing in healthy retinas from wild-type (WT) mice and degenerated retina from slowly degenerating rd10 mice and rapidly degenerating rd1 mice. Multielectrode array (MEA) recordings from isolated rd10 retinas show that the rise of spontaneous RGC activity correlates with the loss of light responses (FIGS. 12A-12B), as shown previously (10). Before the onset of photoreceptor degeneration (P14), spontaneous activity in darkness was low (<1 Hz) and light-elicited firing was robust. Partway through the progression of degeneration (P28), spontaneous activity increased to 3 Hz while light responses were reduced by ˜50%. After degeneration was complete (P60), spontaneous activity increased by 6-fold, whereas light responses were undetectable. In the rd1 mouse, photoreceptors death occurs early (P10-14), and RGCs become hyperactive as compared to WT (FIG. 12C). By P60, rd1-RGCs fire at ˜6 Hz (FIG. 12D), similar to rd10-RGCs, but 6-fold-faster than WT-RGCs

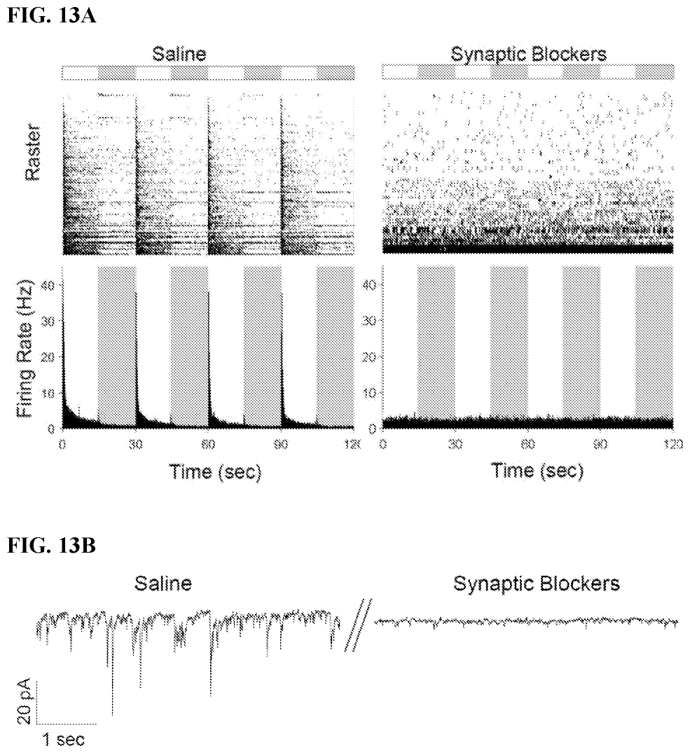

To assess the component of hyperactivity that is independent of chemical synaptic input, we blocked synaptic transmission with a mixture of neurotransmitter receptor blockers. Light-responses in RGCs, as well as spontaneous excitatory postsynaptic currents (FIGS. 13A-13B) were eliminated in this solution. However, the spontaneous firing of RGCs was unaffected in all three strains (FIG. 12D), and remained 5-6-fold faster in rd1 and rd10 as compared to WT, indicating that none of the hyperactivity is a consequence of chemical synaptic transmission (44,59-61). We have found that photoreceptor degeneration leads to an increase in the activity of excitatory ion channels intrinsic to RGCs (12). Degeneration leads to activation of gap junctional proteins that couple RGCs to amacrine cells (62-64). Gap junction uncoupling reduces but does not eliminate hyperactivity (62,65,66). Hence, RGC hyperactivity is a collective property of the electrically coupled network of neurons in the inner retina, but what initiates the hyperactivity is unknown.

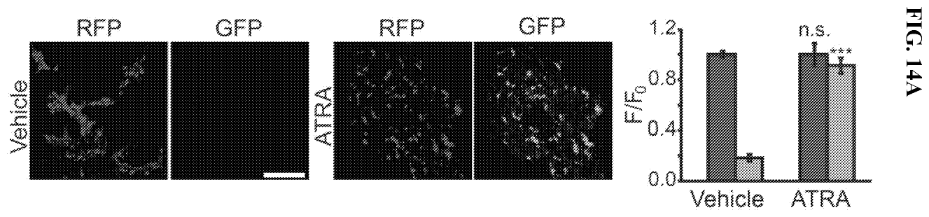

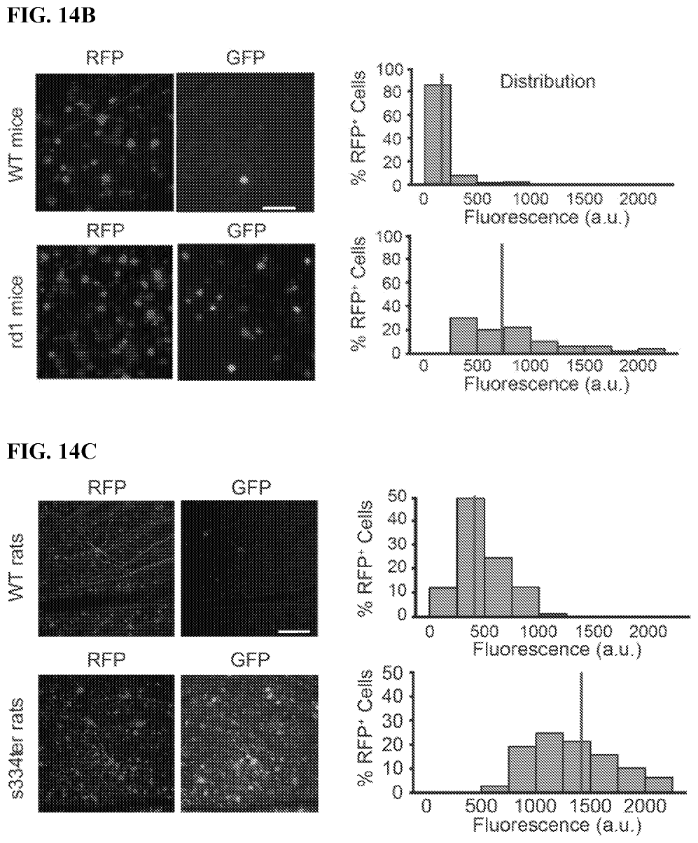

Detecting heightened RA-induced signaling in degenerated retina with a RAR-reporter. RA signaling is mediated by nuclear retinoic acid receptors (RAR). Three different RAR isoforms exist (α, β and γ), all of them are expressed in the mammalian retina (67-69). Upon RA binding, activated RAR binds the DNA at RA-response element (RARE) sequences, driving transcription of downstream target genes (70). If RGCs are exposed to increased levels of RA in degenerated retina, RAR-dependent transcription should be increased. To test this, we developed a genetically-encoded double-fluorescent RAR-reporter for measurement of RAR-dependent transcription (FIG. 6A). Multiple RARE sequences were inserted upstream of the SV40 weak promoter, driving GFP expression, while CMV was used to drive the expression of RFP (viral infection control). In HEK293 cells, transfected cells expressed RFP, but very little GFP (FIG. 14A). Treatment with all-trans retinoic acid (ATRA) induced GFP expression. The increase in GFP to RFP ratio was dose- and time-dependent (FIGS. 11A-11B).

Using an AAV viral vector, we injected the RAR reporter into the vitreous of rd1 or WT mice. Retinas were isolated 45-90 days later for imaging-based single-cell RFP and GFP fluorescence quantification in the ganglion cell layer (GCL). We compared the distribution of GFP fluorescence values across all RFP-expressing cells from WT and rd1 retinas (FIG. 14B). Median and mean GFP fluorescence values in rd1 retina were ˜4-fold higher than in WT (p<0.001, Mann-Whitney). We also employed the RAR-reporter in s334ter transgenic rats, in which photoreceptor death is caused by a rhodopsin mutation identical to that found in a subtype of human RP (31) and in WT Long-Evans rats with healthy retinas. As in mice, RAR signaling was higher in degenerated rat retina than in WT retina (FIG. 14C). The median and the mean GFP fluorescence values in s334ter-RGCs were ˜3-fold higher than the WT values (p<0.001, Mann-Whitney).

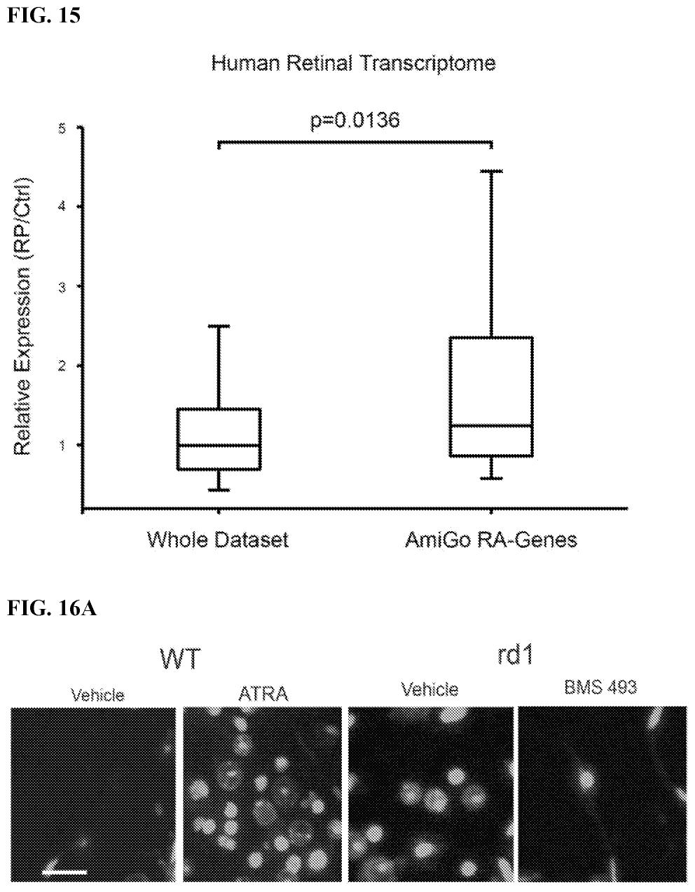

These results indicate that RA-induced gene transcription is enhanced in rodent models of RP. Is RA signaling also enhanced in human RP? We compared published human transcriptome data (36) from a sample of RP retina with a sample of non-diseased retina (FIG. 15). Of all the genes represented in the retinal transcriptome dataset, 120 sequences were validated to be from RA-responsive genes, as categorized by the NIH Gene Ontology database (71). For each of these, we calculated the relative expression between the RP sample and the non-diseased sample (RP/control). Transcript levels probed by the 120 RA-responsive sequences were more than twice as abundant as the transcript levels probed by entire population of 31,108 sequences (RP/control RA-responsive genes=3.03±0.71; RP/control for entire population=1.44±0.014; p=0.0136, Mann-Whitney), consistent with increased RAR-transcription in human RP.

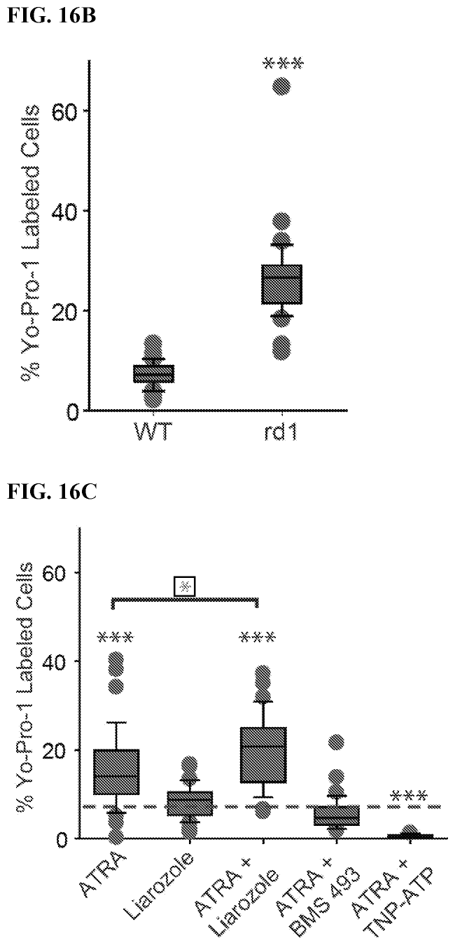

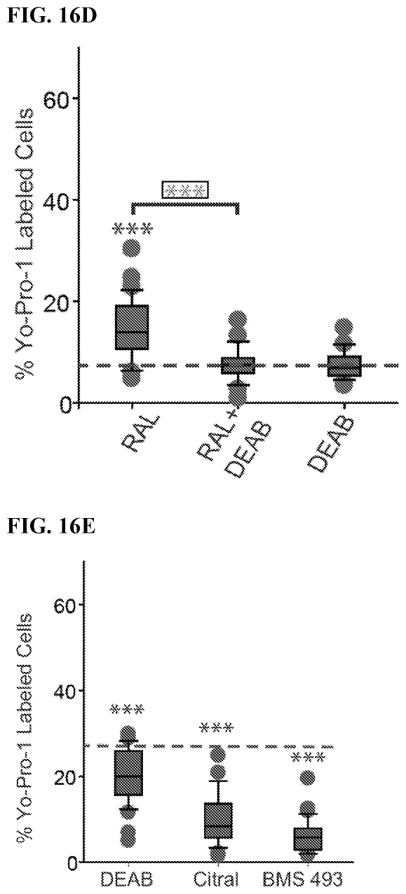

RA increases dye-permeability of RGCs. In healthy retinas, RGCs are impermeant to organic cations. In degenerated retinas, RGCs show increased membrane permeation and cytoplasmic accumulation of large molecules, including cationic fluorescent dyes and charged azobenzene photoswitches (11), as a result of up-regulation and activation of P2X receptors (40,41). To test whether RA triggers hyperpermeability, we used Yo-Pro-1, a P2X-permeant fluorescent nuclear dye. As shown previously (11), Yo-Pro-1 labels a much greater percentage of rd1-RGCs than WT-RGCs (FIGS. 16A-16B). However, intravitreal injection of ATRA in WT retinas significantly increased the fraction of cells incorporating Yo-Pro-1 (FIG. 16C). Liarozole, an inhibitor of CYP26 (cytochrome P450 for RA degradation), had no effect by itself, but potentiated the action of ATRA.

Retinaldehyde dehydrogenase (RALDH) converts RAL into RA (27,72,73). RALDH is expressed in the retinal pigmented epithelium (RPE) and retinal neurons. Injecting RAL into WT retina induced hyperpermeability, significantly increasing Yo-Pro-1 labeling above baseline and by the same amount as ATRA (FIG. 16D). Injection with the RALDH inhibitor diaethylaminobenzaldehyde (DEAB), or co-injection of RAL and DEAB, caused no change in Yo-Pro-1 labeling in WT, indicating that the hyperpermeability of RGCs is dependent on enzymatic synthesis of RA.

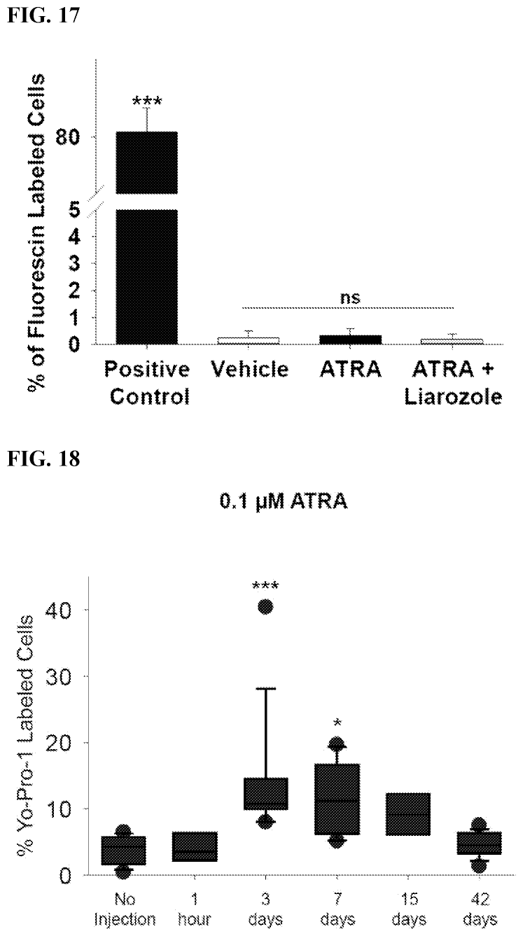

If the hyperpermeability induced by RA is a consequence of RAR-mediated upregulation of P2X receptors, blocking RAR should reduce Yo-Pro-1 labeling. Indeed, co-injecting ATRA with BMS 493, a pan-RAR inverse agonist, eliminated the effect of ATRA on WT-RGCs permeability (FIG. 16C). Block of P2X receptors with its antagonist TNP-ATP also prevented ATRA-induced hyperpermeability. We ruled out the possibility that ATRA indirectly causes RGC hyperpermeability by killing photoreceptors, using a TUNEL assay (FIG. 8A, FIG. 17). Photoreceptors do not regenerate, so the reversibility of hyperpermeability after ATRA injection is inconsistent with a cytotoxic effect (FIG. 18).

Having shown that RGC hyperpermeability can be mimicked in WT with treatments that elevate RA and activate RAR, we next asked whether hyperpermeability can be blocked in rd1-RGCs with treatments that interfere with RA synthesis or block RAR (FIG. 16E). Intravitreal injection of DEAB or citral, both RALDH inhibitors, significantly reduced the percentage of rd1-RGCs labeled with Yo-Pro-1, as compared to vehicle-injected. Injection of BMS 493 reduced Yo-Pro-1 labeling in rd1 to WT levels. These results indicate that blocking RAR is more effective than blocking RA synthesis, which would spare signaling by RA that was present before drug treatment.

These results show that RA is both necessary and sufficient for inducing hyperpermeability. Increasing RA synthesis or preventing degradation in WT retinas was sufficient to induce hyperpermeability through P2X receptors, similar to that observed in rd1 mice. Blocking RA synthesis or RAR signal transduction in rd1 retinas reduced permeability to a level comparable to that in WT-RGCs, demonstrating that RA and RAR-activation are necessary for pathophysiological hyperpermeability.

RA-signaling enables chemical photosensitization of RGCs. Azobenzene photoswitches are synthetic photoisomerizable molecules that can bestow light-sensitivity on neurons that express no native photoreceptor proteins and ordinarily have no intrinsic light response (43,51). Remarkably, photoswitch compounds affect RGCs from blind retinas, but have no effect on RGCs from healthy retinas (12) across mammalian species, suggesting a common mechanism. We have found that photoswitches, like Yo-Pro-1, enter RGCs through up-regulated P2X receptors (11). If RA is the initiator of both processes, increasing or decreasing RA-signaling should have effects on photoswitching that parallel dye-labeling.

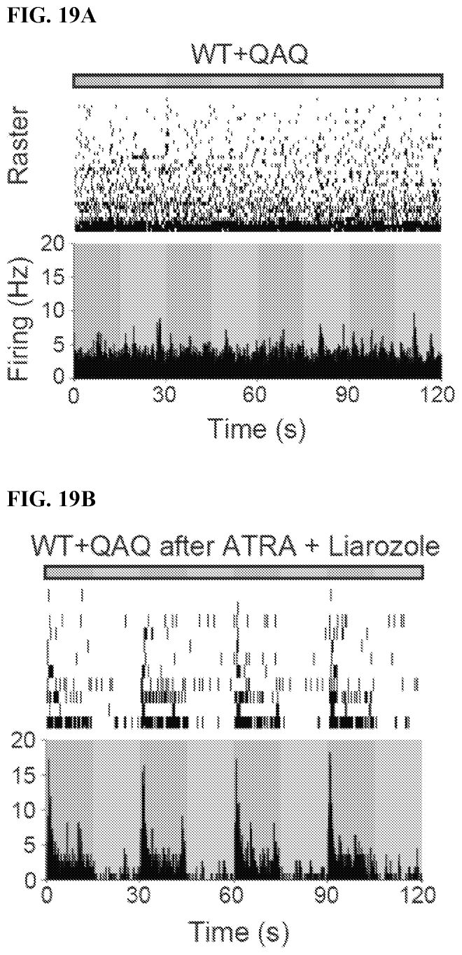

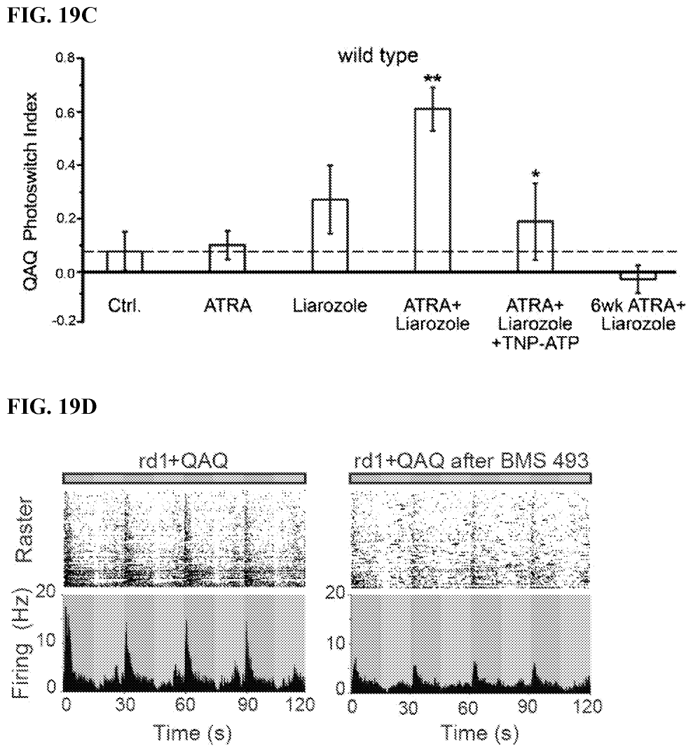

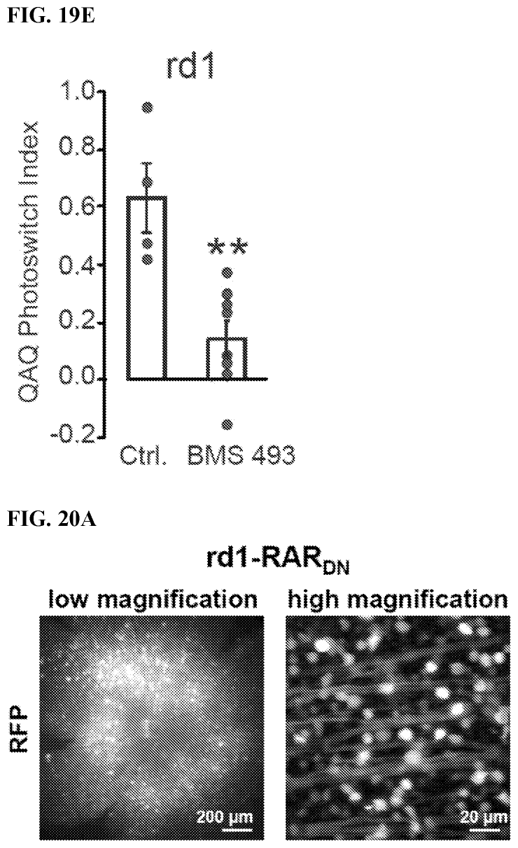

To test this, we carried out intravitreal injections with drugs that alter RA-signaling and, 3 to 7 days later, measured light responses imparted by QAQ, a photoswitch that acts on voltage-gated Na⁺, K⁺, and Ca²⁺ channels found in all neurons. Light-dependent firing was quantified by calculating Photoswitch Index (PI) (5). We first asked whether increasing RA signaling in WT-RGCs could mimic the degeneration-dependent photosensitization observed in rd1-RGCs. Injection of WT retina with ATRA plus liarozole enabled QAQ to elicit light-dependent firing (FIGS. 19A-19B), significantly higher than its control (FIG. 19C) and similar to rd1 retina. Neither ATRA nor liarozole alone enabled significant QAQ photosensitization. The effect of ATRA plus liarozole was blocked by TNP-ATP, and wore off within 6 weeks after injection, consistent with reversible enhancement of RA-signaling. These features, including synergy between ATRA and liarozole, block by P2X receptor antagonists, and reversibility weeks after injection, mirror the effects of RA-signaling on Yo-Pro-1 labeling, consistent with a common mechanism. Consistent with this, inhibition of RAR in rd1 mice injected intravitreally with BMS-493, prevented QAQ photosensitization and reduced the PI to a value similar to that observed in WT (FIGS. 19D-19E).

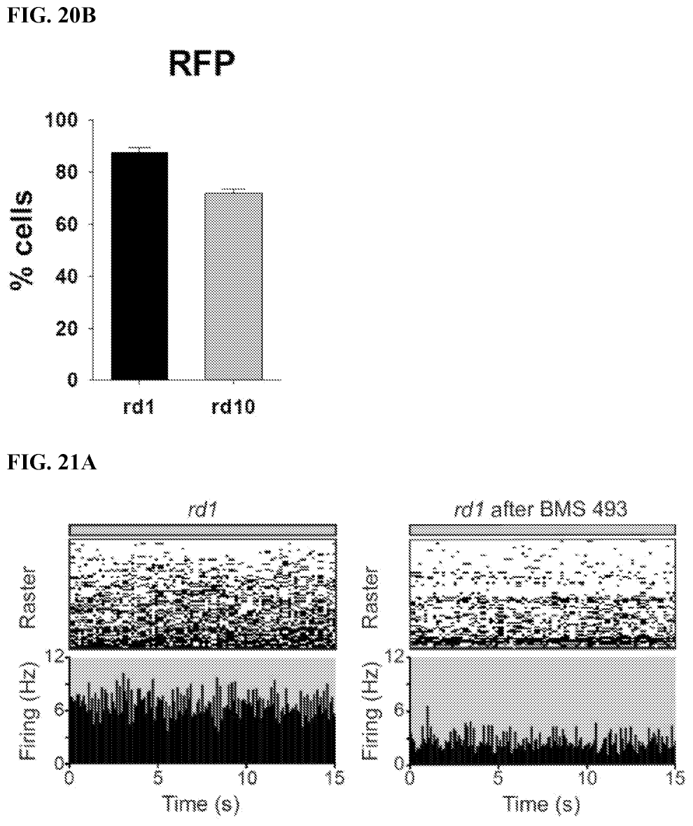

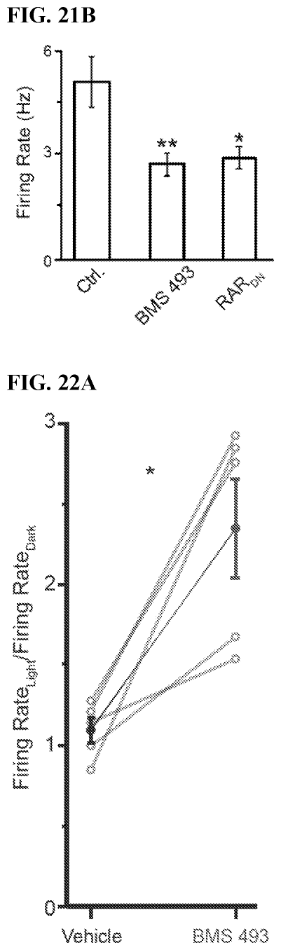

RAR inhibitors reduce hyperactivity and enhance light sensitivity in degenerating retinas. Our results indicate that RA signal transduction is necessary and sufficient for inducing degeneration-dependent hyperpermeability of RGCs. However, the effects of RA could potentially expand to all other cell types in the surviving inner retina of blind mice. If RA is necessary and sufficient for inducing enhanced spontaneous activity in the inner retina, including RGCs but also potentially amacrine and bipolar cells, then blocking RA signaling should reduce pathologically-enhanced spontaneous firing in the degenerated retina. To test this, we obtained MEA recordings in saline (FIG. 21A) from isolated rd1 retina injected either with BMS-493 or with viral vector containing a dominant-negative form of RARα (RAR_{DN}) which represses RA-induced gene transcription (74). Intravitreal infection of rd1 eyes with RAR_{DN} resulted in widespread viral transduction across the retina (>80% RFP-expressing cells in GCL, FIGS. 20A-20B). Spontaneous firing rates were significantly lower in BMS-493- or RAR_{DN}-treated retina as compared to controls (FIG. 21B). Hence RAR activity is necessary for hyperactivity in the inner retina.

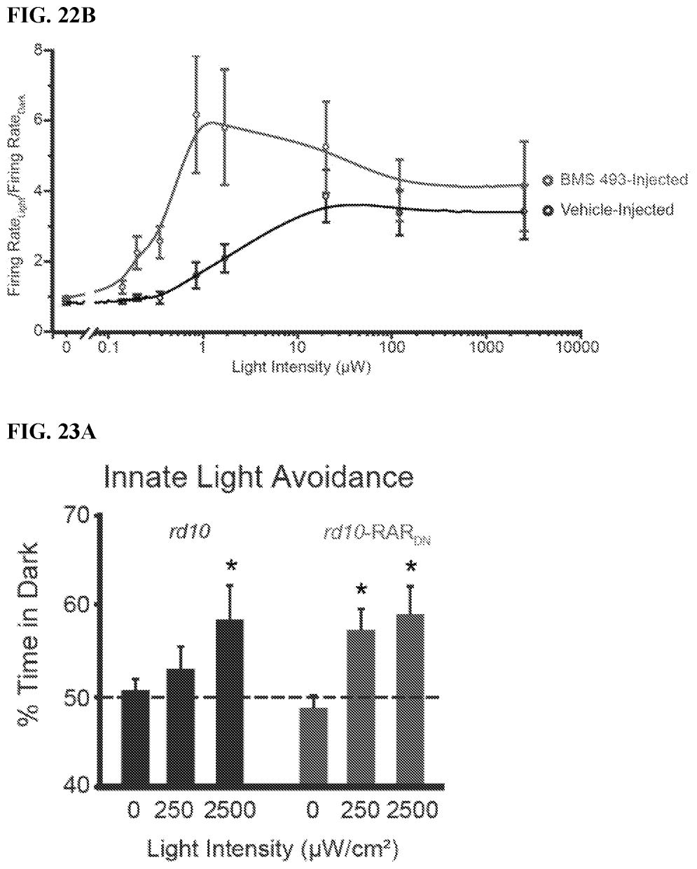

In human RP, photoreceptors gradually degenerate. The death of cones is secondary to the death of rods, and the cell bodies of some cones can persist for years, particularly in the fovea (75). Remnant cones can generate light-responses that are reduced in sensitivity and amplitude, but not completely eliminated (76). However, high background firing of RGCs obscures light responses, particularly to low-intensity stimuli. Blockers of RA signaling might augment light-responses and enhance visual performance. To test this idea, we used the rd10 mice at 6 weeks of age, when their retinas were incompletely degenerated. We injected one eye with BMS-493 and the other with vehicle, and evaluated retinal sensitivity with light flashes of varying intensity. BMS-493-treated retinas showed a transient increase in RGC firing in response to a brief flash (50 ms) of dim light, whereas vehicle-treated retinas showed no RGC response to the same flash (FIGS. 3A-3B, Kruskal-Wallis ANOVA, Dunn's post-hoc p=0.0263). The emergence of the light response was associated with a decrease in the background firing rate in darkness. BMS-493-enhanced the light response in all mice tested (FIG. 22A). Measuring over a variety of intensities revealed a shift in the intensity-response curve (FIG. 22B) reflecting an increase in sensitivity and peak response to light. The response threshold was lower for BMS-493-injected than for vehicle-injected eyes (0.15 mW vs 0.85 mW). Hence, inhibiting RA-signaling dramatically boosts the light-response of RGCs in partially degenerated retinas.

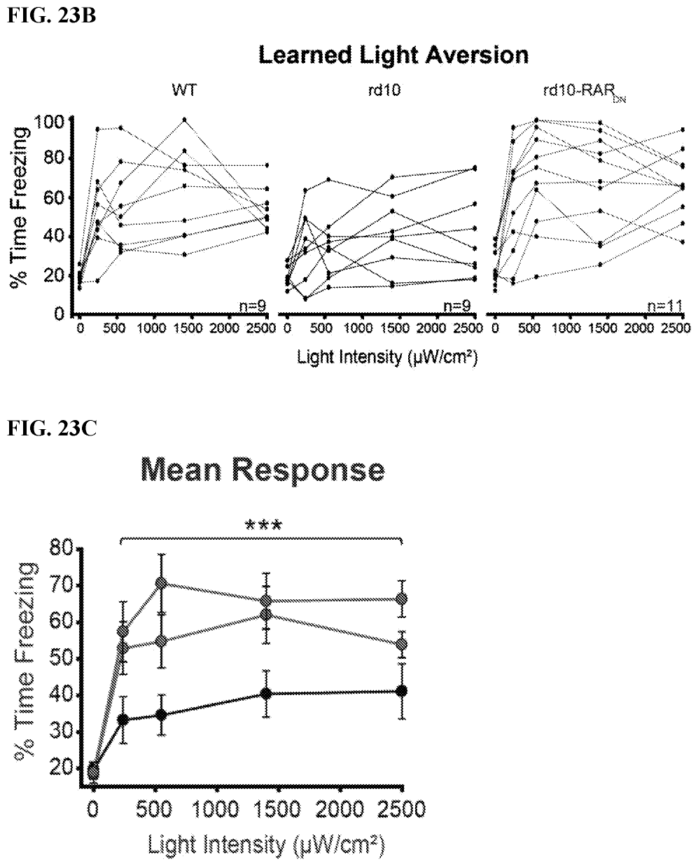

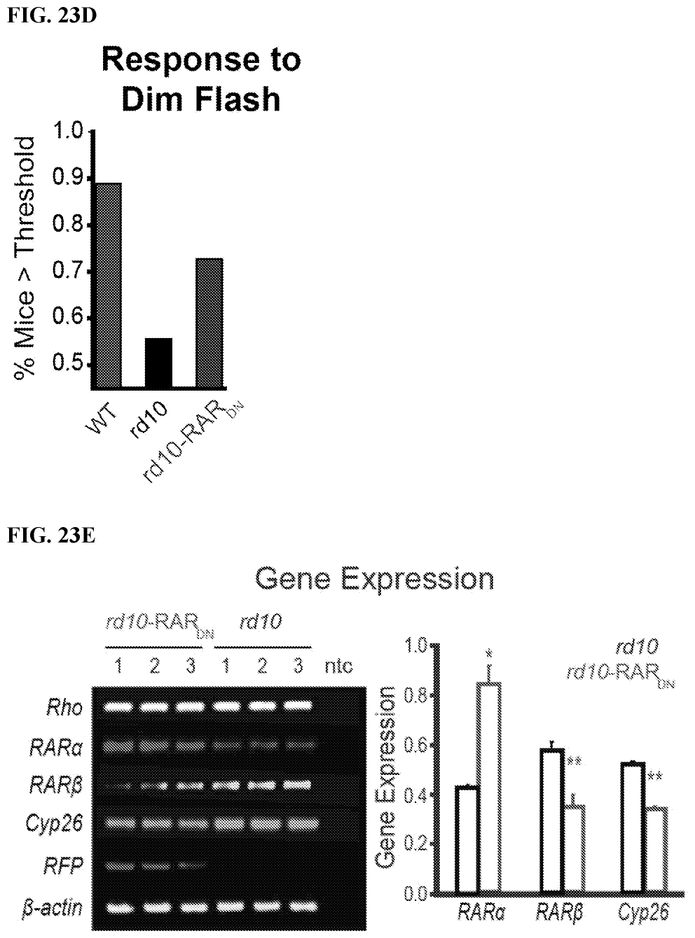

Inhibiting RAR with gene therapy enhances behavioral light sensitivity in vision-impaired mice. We next asked whether the increase in light sensitivity bestowed by RAR inhibition translates into increased behavioral sensitivity to light in-vivo (FIGS. 23A-23E). We injected neonatal rd10 mice with RAR_{DN} (FIGS. 20A-20B, FIGS. 21A-21B, and Materials and Methods), with the goal of inhibiting RAR before degeneration is complete, such that visual function is impaired but not entirely lost. Since intraocular injections are damaging in neonates, we used the AAV9 serotype, which can be injected into the vasculature to efficiently transduce retinal neurons early in postnatal life (77). Light-elicited behavior was evaluated at P30-40.

First, we tested innate light-aversion in P37-39 mice (FIG. 23A) using an automated double-chamber light/dark box (Materials and Methods) and quantified the time spent in each side. In darkness, mice had no preference. Illuminating one side with dim light had no effect on untreated rd10 mice, but rd10-RAR_{DN} mice preferred the dark side significantly more. In 10-fold brighter light, both treated and untreated mice preferred the dark chamber. Hence RAR_{DN} increased the light sensitivity of innate behavior in vision-impaired mice.

We next examined learned light-aversion in P33-35 rd10 and WT mice (FIGS. 23B-23D). Employing a shock-box (Materials and Methods), we tested whether RAR_{DN} could enhance learned aversion to visual cues of different intensities. This test was used previously to measure light-sensitivity in rd1 mice treated with photoswitches for vision restoration (12). Light-adapted rd10, rd10-RAR_{DN} and WT mice learned to associate 10-seconds-long/6000 μW/cm² light-flashes with 2-seconds-long/0.7 mA electric shocks. Their freezing behavior was the assessed before and after light-stimuli with increasing intensities. Individual traces for each mouse show the variability of the response (FIG. 23B). As compared to WT, rd10 mice show poor responses, which seem to be rescued by using RAR_{DN}-gene therapy. WT and rd10-RAR_{DN} mice were able to recall the behavior starting at the second light-flash, and maintained a consistent response for higher intensities, in spite of behavioral extinction (FIG. 23C). Analysis of the slope of the response to the first and dimmest light flash was used to establish a threshold, equally applied to all mice (FIG. 23D). We found that 8 out of 9 WT mice responded to the first light flash, 5/9 in rd10 and 8/11 in rd10-RAR_{DN}.

The same mice were used for PCR confirmation of RAR-signaling manipulation by RAR_{DN}. At P40, mice were sacrificed, and mRNA was extracted and purified from their retinas (FIG. 23E). Semi-quantitative reverse-transcriptase assay shows no change, associated with RAR_{DN}, in the transcription of rhodopsin and β-actin. RARα expression was significantly increased in rd10-RAR_{DN}, reflecting overexpression of the virus, and RFP (mStrawberry) expression was detected only in treated retinas. RAR_{DN} significantly reduced the expression of RAR-regulated genes, such as RAM and Cyp26, demonstrating an effective downregulation of RAR-signaling.

Taking together the results in FIGS. 3A-3B, FIGS. 21A-21D, and FIGS. 23A-23E, we demonstrate that inhibition of RAR-activation effectively reduces pathophysiological hyperactivity in degenerating retinas, rescuing electrophysiological light responses ex-vivo and behavioral light responses in-vivo. These results open the possibility of using RAR as a drug and gene therapy target for improving light responses in patients suffering from slow-progressing degenerative blindness.

RA is the photoreceptor degeneration-dependent trigger for pathophysiological remodeling. Our evidence shows that RA is necessary and sufficient for inducing pathophysiological remodeling of the retina during the progression of photoreceptor degenerative disease. The P2X receptor-dependent hyperpermeability of RGCs, characteristic of degenerated retina, is greatly reduced with drugs that block RALDH, and eliminated with a drug that inhibits RAR. Likewise, pathophysiological changes can be induced in WT retina with agents that either directly or indirectly increase RA. Providing the retina with RA itself or with excess RAL, induces hyperpermeability, mimicking the pathophysiological state. Further supporting the hypothesis that RA is the trigger, our RAR-reporter detected enhanced RA-signaling in mouse and rat degenerated retina. RA has also been implicated in morphological remodeling of dendrites in the outer retina in a light-induced model of blindness (20). These results suggest that both fast functional changes and slower structural remodeling are triggered by RA.

The loss of photoreceptors in RP deprives downstream retinal neurons of sensory information. Sensory deprivation leads to homeostatic plasticity in neural circuits in the brain, including changes in excitatory and inhibitory synaptic strengths and in the intrinsic membrane properties of postsynaptic neurons (78). In principle, homeostatic plasticity could contribute to degeneration-dependent RGC hyperactivity. However, studies on blind mice that have dysfunctional but intact photoreceptors, indicate that the loss of light-dependent synaptic signaling alone is insufficient to trigger remodeling (12), the physical loss of photoreceptors is required. Photoreceptor death in RP, Usher's syndrome, retinal detachment and AMD trigger similar remodeling events (33,79), consistent with a common triggering mechanism perhaps involving RA.

Source and mechanism of action of RA in the degenerating retina. RA is synthesized from RAL by the RALDH, which is expressed ubiquitously in the retina (27,72,73). RAL is produced by isoforms of retinol dehydrogenase (RDH) that are expressed only in cells exposed to the subretinal space, including photoreceptors, RPE and Muller glial cells (80). Delivering excess RAL to WT retina induced the same changes as observed in WT retinas treated with ATRA or in untreated rd1 (FIGS. 16A-16E, FIGS. 19A-19E). The effect of RAL was blocked with a RALDH inhibitor, confirming that conversion to RA is required. These results suggest that availability of RAL is limiting for the initiation of subsequent pathophysiological events. The retina contains millions of photoreceptors each with millions of opsins. Loss of photoreceptors removes an enormous molecular sink for RAL, breaching the outer limiting membrane, and compromising the compartmentalization of RAL to the subretinal space. Several cell types in the degenerated retina could convert some of the excess RAL into RA. Our RAR-reporter shows that RA-induced transcription is heightened in RGCs, demonstrating that RA reaches RGCs and activates gene transcription.

RA can signal through activation of nuclear RARs that regulate gene transcription (68,69). Alternatively, RA can signal through a non-canonical mechanism that is RAR-independent, in which RA directly binds to protein kinases and phosphatases (39). However, we found that blocking the activity of RAR with BMS 493 or RAR_{DN} was sufficient to reduce hyperactivity and hyperpermeability in degenerated retina. Previously, we implicated up-regulation and activation of P2X7 receptors in hyperpermeability, and up-regulation of HCN channels in hyperactivity (11,12). Alas, the genes encoding P2X7 and HCN isoforms do not possess the RARE sequence in their promoter. However, RAR can trigger a cascade of events that ultimately lead to activation of RA-independent genes (68). At the post-transcriptional level, RAR can bind directly to RNA granules, regulating local dendritic protein synthesis in neurons, an event that is important for homeostatic synaptic plasticity (18,81).

RGCs can be categorized by their light-response properties, and dendritic stratification pattern into ON, OFF, or ON/OFF types. Only the OFF-RGCs exhibit hyperpermeability (11) and hyperactivity (44,58) as a consequence of photoreceptor degeneration. If RA is the trigger for remodeling, then OFF-RGCs must either exposed to higher RA or they must respond more vigorously to RA. The dendrites of OFF-RGCs ramify in the sublamina of the inner plexiform layer that is closest to the outer retina, which includes the degenerating photoreceptors, providing a possible basis for selective exposure to RA. Alternatively, OFF-RGCs may be selectively responsive to RA by expressing a transcriptional program that is absent or different than other RGCs. Bipolar and amacrine cells also show physiological and morphological remodeling after photoreceptor degeneration. Bipolar cells sprout new dendritic branches in response to elevated RA (20). A-II amacrine cells show increased phosphorylation of connexin-36 during degeneration (63) enhancing electrical coupling behind spontaneous oscillations. The trigger for remodeling in these cells might also be RA, but this has not yet been investigated.

Improving low-level vision by blocking RA signal transduction. Retinal remodeling in RP is preserved across mammalian species, with stereotypical changes in physiology and morphology (6,32-34). The fundamental defect in RP is loss of photoreceptors, but downstream retinal remodeling might greatly exacerbate vision impairment during disease progression. Hyperactive firing of RGCs masks light-elicited signals initiated by surviving photoreceptors, potentially corrupting visual information sent to the brain. An analogous situation occurs with tinnitus in hearing loss, where degeneration of cochlear hair cells leads to hyperactivity of auditory neurons (45), interfering with the remaining sound-elicited neural signals. Separating the component of the visual deficit resulting directly from photoreceptor death from the component imposed by RGC hyperactivity is not straightforward in humans. While non-invasive recording methods such as pattern electroretinogram can detect changes in RGC firing with rapidly changing visual stimuli (82), spontaneous RGC firing in the absence of visual stimuli cannot be detected. Unfortunately, there are no models of inherited retinal degeneration in non-human primates. However, positron emission tomography scans show increased glucose metabolism in the visual cortex of early-blind patients, attributed to elevated spontaneous neural activity (83). Human subjects with RP have a heightened threshold for electrically-induced phosphenes, consistent with interference by spontaneous retinal activity (84). Directly linking human RP with enhanced RA-induced transcription is limited by the availability of retinal tissue samples with non-degraded mRNA, but the very limited data that have been collected suggest an increase in RA-responsive gene transcription (FIG. 15, (36)).

We have shown that inhibition of RAR reduces retinal hyperactivity, increases light-sensitivity, and boosts behavioral light responses in vision-impaired mice. Meclofenamic acid, an uncoupler of gap junctions, also augments RGC light responses by reducing hyperactivity (62,63), but micromolar concentrations are required and gap junctions are essential for normal retinal functioning. In contrast, RAR inhibitors act at nanomolar concentrations, and they interfere with RA-dependent transcription, a process that is largely absent in RGCs in the healthy retina. Thus, RAR inhibitory drugs should be efficacious in degenerating retinal tissue, while having minimal effects on healthy retinal tissue. Our findings open the door to the possible use of pharmacological inhibitors of RARs as a first-in-class vision-enhancing drug. A rich pharmacopoeia of RAR inhibitors has already been developed (46,85). In this study we used BMS-493, a pan-RAR inverse agonist, but RAR antagonists are also available, either with broad or narrow subunit-specificity. Our findings also suggest a gene therapy approach, utilizing AAV to deliver RAR_{DN}. By selecting a specific AAV serotype or a specific promoter, or a combination of both, a particular type of retinal neuron may be targeted for blockade of RA signal transduction (86,87).

Even after all the photoreceptors have degenerated and light perception is absent, reducing RGC hyperactivity could still be beneficial in blind patients. Responses evoked by optoelectric (48,88), optogenetic (4,65), or optopharmacological (89) stimulation of the degenerated retina are superimposed on the heightened background activity of RGCs, curtailing the encoding of visual images. The combination of a light-sensitive actuator with an RAR inhibitor could have a synergistic effect, boosting neural signals to more effectively restore visual function to blind patients.

Materials and Methods

Animals.

Animals used included WT mice (C57BL/6J strain, Jackson Laboratory or Charles River), homozygous rd1 mice (strain 000659, Jackson Laboratory), homozygous rd10 mice (strain 004297, Jackson Laboratory), WT rats (Long Evans strain, Charles River Laboratories) and s334-ter rats (line #3, Matthew LaVail, UCSF). All animal use procedures were approved by the UC Berkeley Institutional Animal Care and Use Committee.

Cell Lines.

HEK293T cells were routinely grown on polystyrene flasks (Nunc). Media used was Dulbecco's Modified Eagle Medium (DMEM, Thermo-Fisher), containing 10% Fetal Bovine Serum (Thermo-Fisher), 1% GlutaMAX (Gibco) and 1% penicillin-streptomycin (Sigma-Aldrich).

Chemicals.

All chemicals were obtained from Sigma-Aldrich, Tocris Bioscience, Life Technologies, or Santa Cruz Biotech.

Viruses. Retinoic Acid Receptor (RAR) Reporter:

A custom-designed and synthesized AAV vector (Vigene Biosci., Maryland, USA) included a cytomegalovirus promoter (CMV) upstream to the coding sequence for the red fluorescent protein (RFP, ‘mStrawberry’), followed by poly-A tail and a stop sequence. A fragment containing three repetitions of the retinoic acid response element (RARE) sequence followed by the weak promoter SV40 was sub-cloned from pGL3-RARE-luciferase (Addgene Plasmid #13458), a kind gift of the Underhill Lab (90). Finally, a green fluorescent protein (GFP) sequence was sub-cloned downstream to SV40, for a final construct of pAAV-CMV-RFP-stop-RARE(×3)-SV40-GFP. The presence of inverted terminal repeat sequences was confirmed by enzymatic digestion. Final titer was ˜10″ particles/μl.

RAR_{DN}.:

The Dominant-Negative form of RARα, pCIG-RARα403-myc (Addgene Plasmid #16286) was a kind gift of the Jessell Lab (91). The coding region for the truncated RARα subunit was sub-cloned into a pAAV backbone under the expression of human synapsin 1 (hSyn1). The final construct was pAAV-hSyn1-RARα403-myc-RFP-WPRE. The presence of inverted terminal repeat sequences was confirmed by enzymatic digestion. Final titer was ˜10¹⁴ particles/μL.

Serotype:

Both viruses were produced as AAV2 and AAV9 serotypes. AAV2 (92) was used for intravitreal injections in adult mice and rats, while AAV9 (77) was used for tail-vein injection of P2-3 mouse neonates.

Injections. Intravitreal Injections:

Adult mice and rats were intravitreally injected with drugs or viruses. Before injection, animals were anesthetized with isoflurane (2%) and their pupils were dilated with tropicamide (1%) and phenylephrine (2.5%). Proparacaine (0.5%) was used as a topical analgesic. Genteal was applied under a glass coverslip to keep the cornea lubricated. An incision was made through the sclera below the ora serrata with a 30 G needle. Solutions were injected into the vitreous with a blunt-ended 33 G Hamilton syringe. After injection, the antibiotic tobramycin (0.3%) was applied to the eye. Final drug concentrations (after 5-fold dilution in the vitreous) were: 100 nM all-trans retinoic acid (ATRA), 100 μM liarozole (24), 20 μM diethylaminobenzaldehyde (DEAB) (28,93), 50 μM citral, 500 nM BMS 493 (85), 1 μM retinaldehyde, and vehicle comprising of 1× phosphate buffered saline (PBS) containing 1% dimethyl sulfoxide (DMSO). Intravitreal injections were performed using AAV2 serotype only. WT and rd1 mice were injected with the RAR-reporter virus or the RAR_{DN} virus, at age 1-1.5 month-old. WT and s334ter rats were injected with the RAR-reporter virus at age 3-4 month-old. Final volume of injections was 1-1.5 μL for mice and 5 μL for rats.

Tail Vein Injections:

Neonatal mice were injected via one or both tail veins at ages P2-3 with an AAV9 (77) virus to achieve expression of the vector in the central retina. Prior to each injection, neonates were cryo-anesthetized on a latex glove placed on wet ice for 45-60 seconds, immobilized and treated with topical analgesia. All tail vein injections were performed with a final volume of ˜7-10 μL. WT and rd1 mice were injected with the RAR-reporter virus. rd10 mice were injected with the RAR_{DN} virus.

Tissue Preparation.

Eyes were obtained from mice and rats immediately following euthanasia. Retinas were removed and kept in saline (artificial cerebrospinal fluid) containing (in mM) 119 NaCl, 2.5 KCl, 1 KH₂PO₄, 1.3 MgCl₂, 2.5 CaCl₂, 26.2 NaHCO₃, and 20 D-glucose, aerated with 95% O₂/5% CO₂ and at room temperature until recording. For imaging (Yo-Pro-1, RAR-reporter), retinal pieces were flat-mounted on filter papers with GCL side up.

Multi Electrode Array Recordings.

Flat-mounted retina was placed ganglion cell layer down onto a 60-electrode Multi-Electrode Array (MEA 1060-2-BC, Multi-Channel Systems). After mounting the retina was left to dark adapt for 20 minutes under constant perfusion with oxygenated saline at 34° C. 300 μM QAQ at was applied for 30 min, followed by a 5 min wash. A solution containing a mixture of neurotransmitter receptor blockers isolated RGCs from synaptic inputs: (in μM) 10 AP4, 40 DNQX, 30 APS, 10 SR-95531, 50 TPMPA, 10 strychnine, and 50 tubocurarine. In experiments where P2X channels were blocked, the retina in the MEA chamber, was pretreated with 100 μM TNP-ATP (23). Extracellular spikes were high-pass filtered at 200 Hz and digitized at 20 kHz and were counted when exceeding 4 SD from the mean background voltage signal. Typically, each electrode recorded spikes from one to three individual RGCs. Principal component analysis of the spike waveforms was used for sorting spikes generated by individual cells (Offline Sorter, Plexon). Stimulation light was generated from a 100 W mercury arc lamp. Neutral density filters were used to alter the light intensity of the 50 ms light flashes. Narrow band optical filters (Chroma) were used to deliver alternating intervals of 380 nm and 500 nm for stimulation of QAQ-treated as described previously (5,12). A typical MEA protocol consisted of ten cycles of alternating 15 s light and dark intervals. Native light sensitivity was measured with ten cycles of alternating 50 ms light and 15 s dark. Spontaneous firing rate in the dark was measured as the average firing of the dark intervals. The Photoswitch Index (PI) was established for individual retinas in 380 nm/500 nm. PI=(mean firing rate in 380 nm light−mean firing rate in 500 nm light)/(mean firing rate in 380 nm light+mean firing rate in 500 nm light).

Yo-Pro-1 Labeling Assay.

After dissection, retinas were cut into thirds and flat-mounted on a windowed nitrocellulose filter paper. Retinas were treated with 200 nM Yo-Pro-1 (Life Technologies) in oxygenated saline for 15 minutes, followed by staining with nuclear ID (Enzo Life Sciences) at a 1:500 dilution for 3 minutes. Saline was perfused continuously at 3 ml/min for a period of 5 minutes to wash away excess dye. In experiments employing TNP-ATP (100 μM), the retinas were pretreated with the compound for 10 minutes before beginning Yo-Pro-1 treatment.

RAR Reporter Virus Assay. In Vitro:

HEK293T cells were grown on poly-lysine (Sigma)-coated glass coverslips in 24-well plates (Nunc), in serum-free media to avoid vitamin A. When cultures reached ˜70% confluency, they were transfected with the RAR-reporter plasmid using Lipofectamine 2000 (Thermo Fisher). 48 hrs post-transfection, cells were checked for RFP expression, and then treated with ATRA or vehicle (1% DMSO) for 48 hrs. Cells were then fixed using 4% paraformaldehyde and mounted on glass slides using DAPI-Fluoromont G (Southern Biotech) for imaging.

In Vivo:

RAR-reporter injected mice and rats, were sacrificed and enucleated.

Retinas were isolated as previously described. Whole retinas were partially sectioned on their periphery, making cuts with a scalpel on four symmetrical sides radial to the optic nerve. Whole retinas were flat-mounted onto transparent PDFA membranes (Millipore) and placed on a saline bath. During imaging, retinas were continuously perfused with oxygenated saline.

Tunel Assay.

TUNEL assay (In Situ Cell Death Detection Kit, Roche) was carried out per manufacturer's instructions. Retinas were collected 5-6 days following injection with vehicle, ATRA, or ATRA and liarozole. Retinas were fixed inside the cup using 4% paraformaldehyde and embedded in tissue freezing medium. The tissue was cut in 14 μm thick cross-sections using a Leica cryostat.

Imaging and Analysis. Confocal Microscopy:

Yo-Pro-1 loading and RAR-reporter virus were imaged using a spinning disk confocal microscope (Olympus BX61WI). The excitation source was a mercury lamp, and fluorescence was collected by a 40× water-immersion objective and standard GFP (488/519 nm) and RFP (561/575 nm) filter cubes (Olympus, U-URA). 1.5 μm section Z-stacks were acquired using a Hamamatsu ImageEM CCD C9100-13.

Image Analysis:

All image analysis was performed using ImageJ or Fiji software (NIH) (55,56). For Yo-Pro-1 loading assay, regions of interests (ROIs) were manually selected using the nuclear-ID channel based upon morphological characteristics. Vascular-associated cells were excluded from ROI selection based on their elongated aspect ratio and spatial relation to each other. ROI selection was performed after computationally flattening the retina by employing a maximum Z projection onto a single plane. Background was subtracted using a rolling radius of 50 pixels. A threshold for Yo-Pro-1 loading was established by measuring the level of autofluorescence of untreated retinas in each channel and finding a baseline value with +2 SD being the threshold for a Yo-Pro-1 positive cell. Nuclear-ID was used to count the total number of cells within a field of view. The percentage of cells above the threshold was then calculated for comparison. For analysis of RAR-reporter virus assay, ROIs were manually selected on the RFP channel first and then superimposed to the GFP channel. Single cell values for both RFP and GFP were filtered by using a RFP minimum threshold established in naive unlabeled retinas.

Behavioral Assays. Innate Light Aversion:

Innate light aversion was tested using a light/dark box (Harvard Apparatus, Coulbourn Instruments, H10-24). For the first two days, mice were habituated in the dark to the test room for 2 hrs/day, and on the third day they were habituated in the dark to the box for ˜20 minutes. The day of the test, mice were dark adapted for at least 1 hour, then placed in the box in the dark and their activity recorded for 10 minutes. Light was delivered to one side of the box using a single blue LED lamp. Aversion to light was tested at an intensity of ˜250 μW/cm² for 10 minutes, and ˜2500 μW/cm² for another 10 minutes (intensity measured ˜25 cm from light source). Automated data was generated by infrared sensors on both chambers, recorded and analyzed using Graphic State software (Coulbourn Instruments). The test box was thoroughly cleaned in between mice using 10% bleach.

Learned Light Aversion:

Learned light aversion was tested using a shock-box (Harvard Apparatus, Habitest), containing a shock-delivery grid, a recording camera, and a custom-built panel of three individual LED white lamps coupled to a manual dimmer. Two days prior to the test, light-adapted mice were individually habituated to the box for 10 minutes. A day before the test, mice were introduced into the chamber for 10 minutes, and were exposed to three consecutive conditioning stimuli, each of them consisting of a 10 seconds-long light pulse (˜6000 μW/cm²) coupled with a 2 seconds-long electric shock (0.5-0.8 mA). The third day, mice were placed in the box for 11 minutes, during which they were exposed to 4 light stimuli, each 30 seconds-long interspaced by 2 minutes of darkness. The light intensity was incremented between each stimulus. Recordings and analysis was performed by FreezeFrame (Coulbourn Instruments).

Semi-Quantitative RT-PCR.

Semi-quantitative reverse transcription PCR was employed to assess relative transcription of target genes. Retinas were dissected and immediately homogenized (for each mouse, both retinas were pulled together). RNA was extracted and purified using RNAeasy Kit (Qiagen). cDNA was obtained by reverse transcribing 500 ng of RNA, using SuperScript III Kit (Thermo-Fisher). Semi-quantitative PCR reactions were carried out using AccuPower PCR Pre-Mix tubes (Bioneer), including 0.5 μM primer mix. Analysis of gene expression was conducted using ImageJ to determine mean gray value of gel bands (densitometry).

Quantification and Statistical Analysis.

If not stated otherwise, the central tendency is shown as the mean. Variability was calculated as standard error of the mean (SEM). Unless otherwise specified, error bars represent SEM. In all cases, measurements were taken from distinct samples. The specific statistical test for significance performed for each experiment is stated in its corresponding result description and figure legend. If not specified otherwise, Student's t-tests and ANOVA tests were 1-tailed. Pairwise comparisons for non-parametric data employed the Wilcoxon Rank Sum Test. In the case where ANOVA was employed, bootstrapping was used to account for unequal group sizes, a Tukey HSD test was employed as a post-hoc test to define which comparisons and interactions produced statistically significant changes. The Thompson-Tau method was employed for the detection of outliers. Results with p<0.05 were considered significant. Symbols for p values were used as follows: *<0.05, **<0.01, ***<0.001.

### REFERENCES

1. Medeiros, N. E., Curcio, C. A. (2001). Preservation of ganglion cell layer neurons in age-related macular degeneration. Investigative Ophthalmology & Visual Science, 42(3), 795-803. 2. Mazzoni, F., Novelli, E., Strettoi, E. (2008). Retinal ganglion cells survive and maintain normal dendritic morphology in a mouse model of inherited photoreceptor degeneration. Journal of Neuroscience, 28(52), 14282-14292. 3. Humayun, M. S., Dorn, J. D., Da Cruz, L., Dagnelie, G., Sahel, J. A., Stanga, P. E., Ho, A. C. (2012). Interim results from the international trial of Second Sight's visual prosthesis. Ophthalmology, 119(4), 779-788. 4. Bi, A., Cui, J., Ma, Y. P., Olshevskaya, E., Pu, M., Dizhoor, A. M., Pan, Z. H. (2006). Ectopic expression of a microbial-type rhodopsin restores visual responses in mice with photoreceptor degeneration. Neuron, 50(1), 23-33. 5. Polosukhina, A., Litt, J., Tochitsky, I., Nemargut, J., Sychev, Y., De Kouchkovsky, I., Kramer, R. H. (2012). Photochemical restoration of visual responses in blind mice. Neuron, 75(2), 271-282. 6. Jones, B. W., Pfeiffer, R. L., Ferrell, W. D., Watt, C. B., Marmor, M., Marc, R. E. (2016). Retinal remodeling in human retinitis pigmentosa. Experimental eye research, 150, 149-165. 7. Phillips, M. J., Otteson, D. C., Sherry, D. M. (2010). Progression of neuronal and synaptic remodeling in the rd10 mouse model of retinitis pigmentosa. Journal of Comparative Neurology, 518(11), 2071-2089. 8. Anderson, E. E., Greferath, U., Fletcher, E. L. (2016). Changes in morphology of retinal ganglion cells with eccentricity in retinal degeneration. Cell and tissue research, 364(2), 263-271. 9. Stasheff, S. F. (2008). Emergence of sustained spontaneous hyperactivity and temporary preservation of OFF responses in ganglion cells of the retinal degeneration (rd1) mouse. Journal of neurophysiology, 99(3), 1408-1421. 10. Stasheff, S. F., Shankar, M., Andrews, M. P. (2011). Developmental time course distinguishes changes in spontaneous and light-evoked retinal ganglion cell activity in rd1 and rd10 mice. Journal of neurophysiology, 105(6), 3002-3009. 11. Tochitsky, I., Helft, Z., Meseguer, V., Fletcher, R. B., Vessey, K. A., Telias, M., Kramer, R. H. (2016). How Azobenzene Photoswitches Restore Visual Responses to the Blind Retina. Neuron, 92(1), 100-113. 12. Tochitsky, I., Polosukhina, A., Degtyar, V. E., Gallerani, N., Smith, C. M., Friedman, A. Kramer, R. H. (2014). Restoring visual function to blind mice with a photoswitch that exploits electrophysiological remodeling of retinal ganglion cells. Neuron, 81(4), 800-813. 13. Leveillard, T., Mohand-Said, S., Lorentz, O., Hicks, D., Fintz, A. C., Clerin, E., Dolle, P. (2004). Identification and characterization of rod-derived cone viability factor. Nature genetics, 36(7), 755-759. 14. Aft-Ali, N., Fridlich, R., Millet-Puel, G., Clerin, E., Delalande, F., Jaillard, C., Olivier-Bandini, A. (2015). Rod-derived cone viability factor promotes cone survival by stimulating aerobic glycolysis. Cell, 161(4), 817-832. 15. Palczewski, K. (2012). Chemistry and biology of vision. Journal of Biological Chemistry, 287(3), 1612-1619. 16. Duester, G. (2008). Retinoic acid synthesis and signaling during early organogenesis. Cell, 134(6), 921-931. 17. Mey, J., Mccaffery, P. (2004). Retinoic acid signaling in the nervous system of adult vertebrates. The Neuroscientist: a review journal bringing neurobiology, neurology and psychiatry, 10(5), 409-421. 18. Chen, L., Lau, A. G., Sarti, F. (2014). Synaptic retinoic acid signaling and homeostatic synaptic plasticity. Neuropharmacology, 78, 3-12. 19. McCaffery, P., Zhang, J., Crandall, J. E. (2006). Retinoic acid signaling and function in the adult hippocampus. Journal of Neurobiology, 66(7), 780-791. 20. Lin, Y., Jones, B. W., Liu, A., Tucker, J. F., Rapp, K., Luo, L., Shaw, M. V. (2012). Retinoid receptors trigger neuritogenesis in retinal degenerations. FASEB journal: official publication of the Federation of American Societies for Experimental Biology, 26(1), 81-92. 21. Innocenti, B., Pfeiffer, S., Zrenner, E., Kohler, K., Guenther, E. (2004). ATP-induced non-neuronal cell permeabilization in the rat inner retina. Journal of Neuroscience, 24(39), 8577-8583. 22. Conlon, R. A., Rossant, J. (1992). Exogenous retinoic acid rapidly induces anterior ectopic expression of murine Hox-2 genes in vivo. Development, 116(2), 357-368. 23. Virginio, C., Robertson, G., Surprenant, A., North, R. A. (1998). Trinitrophenyl-substituted nucleotides are potent antagonists selective for P2X1, P2X3, and heteromeric P2X2/3 receptors. Molecular Pharmacology, 53(6), 969-973. 24. Van Wauwe, J., Van Nyen, G., Coene, M. C., Stoppie, P., Cools, W., Goossens, J., Janssen, P. A. (1992). Liarozole, an inhibitor of retinoic acid metabolism, exerts retinoid-mimetic effects in vivo. Journal of Pharmacology and Experimental Therapeutics, 261(2), 773-779. 25. del Mar Vivanco-Ruiz, M., Tiollais, P., Stunnenberg, H., Dejean, A. (1990). Identification of a Retinoic Acid Responsive Element in the Retinoic Acid Receptor (Beta) Gene. Nature, 343(6254), 177. 26. Kam, R. K. T., Deng, Y., Chen, Y., Zhao, H. (2012). Retinoic acid synthesis and functions in early embryonic development. Cell & bioscience, 2(1), 11. 27. Fischer, A. J., Wallman, J., Mertz, J. R., Stell, W. K. (1999). Localization of retinoid binding proteins, retinoid receptors, and retinaldehyde dehydrogenase in the chick eye. Journal of neurocytology, 28(7), 597-609. 28. Chute, J. P., Muramoto, G. G., Whitesides, J., Colvin, M., Safi, R., Chao, N. J., McDonnell, D. P. (2006). Inhibition of aldehyde dehydrogenase and retinoid signaling induces the expansion of human hematopoietic stem cells. Proceedings of the National Academy of Sciences, 103(31), 11707-11712. 29. Shimozono, S., Iimura, T., Kitaguchi, T., Higashijima, S. I., Miyawaki, A. (2013). Visualization of an endogenous retinoic acid gradient across embryonic development. Nature, 496(7445), 363-366. 30. Heyman, R. A., Mangelsdorf, D. J., Dyck, J. A., Stein, R. B., Eichele, G., Evans, R. M., Thaller, C. (1992). 9-cis retinoic acid is a high affinity ligand for the retinoid X receptor. Cell, 68(2), 397-406. 31. Green, E. S., Menz, M. D., LaVail, M. M., Flannery, J. G. (2000). Characterization of rhodopsin mis-sorting and constitutive activation in a transgenic rat model of retinitis pigmentosa. Investigative Ophthalmology & Visual Science, 41(6), 1546-1553. 32. Humphries, M. M., Rancourt, D., Farrar, G. J., Kenna, P., Hazel, M., Bush, R. A., Erven, A. (1997). Retinopathy induced in mice by targeted disruption of the rhodopsin gene. Nature genetics, 15(2), 216-219. 33. Marc, R. E., Jones, B. W., Watt, C. B., Strettoi, E. (2003). Neural remodeling in retinal degeneration. Progress in retinal and eye research, 22(5), 607-655. 34. Marc, R. E., Jones, B. W. (2003). Retinal remodeling in inherited photoreceptor degenerations. Molecular neurobiology, 28(2), 139-147. 35. Jones, B. W., Pfeiffer, R. L., Ferrell, W. D., Watt, C. B., Marmor, M., Marc, R. E. (2016). Retinal remodeling in human retinitis pigmentosa. Experimental eye research, 150, 149-165. 36. Mullins, R. F., Kuehn, M. H., Radu, R. A., Enriquez, G. S., East, J. S., Schindler, E. I., Stone, E. M. (2012). Autosomal Recessive Retinitis Pigmentosa Due To ABCA4 Mutations: Clinical, Pathologic, and Molecular Characterization ABCA4-Associated Retinitis Pigmentosa. Investigative ophthalmology & visual science, 53(4), 1883-1894. 37. Hackam, A. S., Strom, R., Liu, D., Qian, J., Wang, C., Otteson, D., Zack, D. J., Leveillard, T. (2004). Identification of gene expression changes associated with the progression of retinal degeneration in the rd1 mouse. Investigative ophthalmology & visual science, 45(9), 2929-2942. 38. Marletaz, F., Holland, L. Z., Laudet, V., Schubert, M. (2006). Retinoic acid signaling and the evolution of chordates. Int. J. Biol. Sci., 2(2), 38-47. 39. Aggarwal, S., Kim, S. W., Cheon, K., Tabassam, F. H., Yoon, J. H., Koo, J. S. (2006). Nonclassical action of retinoic acid on the activation of the cAMP response element-binding protein in normal human bronchial epithelial cells. Molecular biology of the cell, 17(2), 566-575. 40. Virginio, C., MacKenzie, A., Rassendren, F. A., North, R. A., Surprenant, A. (1999). Pore dilation of neuronal P2X receptor channels. Nature neuroscience, 2(4), 315-321. 41. Browne, L. E., Compan, V., Bragg, L., North, R. A. (2013). P2X7 receptor channels allow direct permeation of nanometer-sized dyes. Journal of Neuroscience: the official journal of the Society for Neuroscience, 33(8), 3557-3566. 42. Li, M., Toombes, G. E., Silberberg, S. D., Swartz, K. J. (2015). Physical basis of apparent pore dilation of ATP-activated P2X receptor channels. Nature neuroscience, 18(11), 1577-1583. 43. Mourot, A., Fehrentz, T., Le Feuvre, Y., Smith, C. M., Herold, C., Dalkara, D., Kramer, R. H. (2012). Rapid optical control of nociception with an ion-channel photoswitch. Nature methods, 9(4), 396-402. 44. Sekirnjak, C., Jepson, L. H., Hottowy, P., Sher, A., Dabrowski, W., Litke, A. M., Chichilnisky, E. J. (2011). Changes in physiological properties of rat ganglion cells during retinal degeneration. Journal of neurophysiology, 105(5), 2560-2571. 45. Middleton, J. W., Kiritani, T., Pedersen, C., Turner, J. G., Shepherd, G. M., Tzounopoulos, T. (2011). Mice with behavioral evidence of tinnitus exhibit dorsal cochlear nucleus hyperactivity because of decreased GABAergic inhibition. Proceedings of the National Academy of Sciences, 108(18), 7601-7606. 46. Germain, P., Chambon, P., Eichele, G., Evans, R. M., Lazar, M. A., Leid, M., Gronemeyer, H. (2006). International union of pharmacology. LX. Retinoic acid receptors. Pharmacological reviews, 58(4), 712-725. 47. Nirenberg, S., Pandarinath, C. (2012). Retinal prosthetic strategy with the capacity to restore normal vision. Proceedings of the National Academy of Sciences, 109(37), 15012-15017. 48. Stronks, H. C., Dagnelie, G. (2014). The functional performance of the Argus II retinal prosthesis. Expert review of medical devices, 11(1), 23-30. 49. Barrett, J. M., Hilgen, G., Sernagor, E. (2016). Dampening Spontaneous Activity Improves the Light Sensitivity and Spatial Acuity of Optogenetic Retinal Prosthetic Responses. Scientific Reports, 6. 50. Fortin, D. L., Banghart, M. R., Dunn, T. W., Borges, K., Wagenaar, D. A., Gaudry, Q., Kramer, R. H. (2008). Photochemical control of endogenous ion channels and cellular excitability. Nature methods, 5(4), 331-338. 51. Mourot, A., Kienzler, M. A., Banghart, M. R., Fehrentz, T., Huber, F. M., Stein, M., Trauner, D. (2011). Tuning photochromic ion channel blockers. ACS chemical neuroscience, 2(9), 536-543. 52. Karttunen, J., Sanderson, S., Shastri, N. (1992) Detection of rare antigen-presenting cells by the lacZ T-cell activation assay suggests an expression cloning strategy for T-cell antigens. Proc. Natl. Acad. Sci. U.S.A 89(13): 6020-6024 53. Bostick, B., Ghosh, A., Yue, Y., Long, C., Duan, D. (2007). Systemic AAV-9 transduction in mice is influenced by animal age but not by the route of administration. Gene therapy, 14(22), 1605-1609. 54. Ray, A., Sun, G. J., Chan, L., Grzywacz, N. M., Weiland, J., Lee, E. J. (2010). Morphological alterations in retinal neurons in the S334ter-line3 transgenic rat. Cell and tissue research, 339(3), 481-491. 55. Schneider, C. A., Rasband, W. S. Eliceiri, K. W. (2012), NIH Image to ImageJ:25 years of image analysis, Nature methods 9(7): 671-675. 56. Schindelin, J., Arganda-Carreras, I., Frise, E., et al. (2012), Fiji: an open-source platform for biological-image analysis, Nature methods 9(7): 676-682. 57. O'Brien, E. E., Greferath, U., Fletcher, E. L. (2014). The effect of photoreceptor degeneration on ganglion cell morphology. The Journal of comparative neurology, 522(5), 1155-1170. 58. Margolis, D. J., Newkirk, G., Euler, T., Detwiler, P. B. (2008). Functional stability of retinal ganglion cells after degeneration-induced changes in synaptic input. The Journal of neuroscience: the official journal of the Society for Neuroscience, 28, 6526-6536. 59. Borowska, J., Trenholm, S., Awatramani, G. B. (2011). An intrinsic neural oscillator in the degenerating mouse retina. The Journal of neuroscience: the official journal of the Society for Neuroscience, 31, 5000-5012. 60. Trenholm, S., et al. (2012). Intrinsic oscillatory activity arising within the electrically coupled AII amacrine-ON cone bipolar cell network is driven by voltage-gated Na+ channels. The Journal of physiology, 590, 2501-2517. 61. Yee, C. W., Toychiev, A. H., Sagdullaev, B. T. (2012). Network deficiency exacerbates impairment in a mouse model of retinal degeneration. Frontiers in systems neuroscience, 6, 8. 62. Toychiev, A. H., Ivanova, E., Yee, C. W., Sagdullaev, B. T. (2013). Block of gap junctions eliminates aberrant activity and restores light responses during retinal degeneration. The Journal of neuroscience: the official journal of the Society for Neuroscience, 33, 13972-13977. 63. Ivanova, E., Yee, C. W., Baldoni, R., Jr., Sagdullaev, B. T. (2016). Aberrant activity in retinal degeneration impairs central visual processing and relies on Cx36-containing gap junctions. Experimental eye research, 150, 81-89. 64. Choi, H., et al. (2014). Intrinsic bursting of All amacrine cells underlies oscillations in the rd1 mouse retina. Journal of neurophysiology 112, 1491-1504. 65. Barrett, J. M., Degenaar, P., Sernagor, E. (2015). Blockade of pathological retinal ganglion cell hyperactivity improves optogenetically evoked light responses in rd1 mice. Frontiers in cellular neuroscience, 9, 330. 66. Eleftheriou, C. G., et al. (2017) Meclofenamic acid improves the signal to noise ratio for visual responses produced by ectopic expression of human rod opsin. Molecular vision, 23, 334-345. 67. Janssen, J. J., et al. (1999). Retinoic acid receptors and retinoid X receptors in the mature retina: subtype determination and cellular distribution. Current eye research, 19, 338-347. 68. Balmer, J. E., Blomhoff, R. (2002). Gene expression regulation by retinoic acid. Journal of lipid research, 43, 1773-1808. 69. Benbrook, D. M., Chambon, P., Rochette-Egly, C., Asson-Batres, M. A. (2014). History of retinoic acid receptors. Sub-cellular biochemistry, 70, 1-20. 70. de The, H., Vivanco-Ruiz, M. M., Tiollais, P., Stunnenberg, H., Dejean, A. (1990). Identification of a retinoic acid responsive element in the retinoic acid receptor beta gene. Nature, 343, 177-180. 71. Harris, M. A., et al. (2004). The Gene Ontology (GO) database and informatics resource. Nucleic acids research, 32, D258-261. 72. McCaffery, P., Lee, M. O., Wagner, M. A., Sladek, N. E., Drager, U. C. (1992). Asymmetrical retinoic acid synthesis in the dorsoventral axis of the retina. Development, 115, 371-382. 73. Harper, A. R., Wiechmann, A. F., Moiseyev, G., Ma, J. X., Summers, J. A. (2015) Identification of active retinaldehyde dehydrogenase isoforms in the postnatal human eye. PloS one, 10, e0122008. 74. Damm, K., Heyman, R. A., Umesono, K., Evans, R. M. (1993). Functional inhibition of retinoic acid response by dominant negative retinoic acid receptor mutants. Proceedings of the National Academy of Sciences of the United States of America, 90, 2989-2993. 75. Milam, A. H., Li, Z. Y., Fariss, R. N. (1998). Histopathology of the human retina in retinitis pigmentosa. Progress in retinal and eye research, 17, 175-205. 76. Busskamp, V., et al. (2010). Genetic reactivation of cone photoreceptors restores visual responses in retinitis pigmentosa. Science, 329, 413-417. 77. Byrne, L. C., Lin, Y. J., Lee, T., Schaffer, D. V., Flannery, J. G. (2015). The expression pattern of systemically injected AAV9 in the developing mouse retina is determined by age. Molecular therapy: the journal of the American Society of Gene Therapy, 23, 290-296. 78. Turrigiano, G. G. (1999). Homeostatic plasticity in neuronal networks: the more things change, the more they stay the same. Trends in neurosciences, 22, 221-227. 79. Coblentz, F. E., Radeke, M. J., Lewis, G. P., Fisher, S. K. (2003). Evidence that ganglion cells react to retinal detachment. Experimental eye research, 76, 333-342. 80. Parker, R. O., Crouch, R. K. (2010). Retinol dehydrogenases (RDHs) in the visual cycle. Experimental eye research, 91, 788-792. 81. Maghsoodi, B., et al. (2008). Retinoic acid regulates RARalpha-mediated control of translation in dendritic RNA granules during homeostatic synaptic plasticity. Proceedings of the National Academy of Sciences of the United States of America, 105, 16015-16020. 82. Porciatti, V. (2015). Electrophysiological assessment of retinal ganglion cell function. Experimental eye research, 141, 164-170. 83. De Volder, A. G., et al. (1997). Brain energy metabolism in early blind subjects: neural activity in the visual cortex. Brain research, 750, 235-244. 84. Delbeke, J., et al. (2001). Electrical stimulation of anterior visual pathways in retinitis pigmentosa. Investigative ophthalmology & visual science, 42, 291-297. 85. Germain, P., et al. (2009). Differential action on coregulator interaction defines inverse retinoid agonists and neutral antagonists. Chemistry & biology, 16, 479-489. 86. Martin, K. R., Klein, R. L., Quigley, H. A. (2002). Gene delivery to the eye using adeno-associated viral vectors. Methods, 28, 267-275. 87. Trapani, I., Auricchio, A. (2018). Seeing the Light after 25 Years of Retinal Gene Therapy. Trends in molecular medicine, 24, 669-681. 88. Dagnelie, G., et al. (2017). Performance of real-world functional vision tasks by blind subjects improves after implantation with the Argus(R) II retinal prosthesis system. Clinical & experimental ophthalmology, 45, 152-159. 89. Tochitsky, I., Kienzler, M. A., Isacoff, E., Kramer, R. H. (2018). Restoring Vision to the Blind with Chemical Photoswitches. Chemical reviews, 118, 10748-10773. 90. Hoffman, L. M., et al. (2006). BMP action in skeletogenesis involves attenuation of retinoid signaling. The Journal of cell biology, 174, 101-113. 91. Novitch, B. G., Wichterle, H., Jessell, T. M., Sockanathan, S. (2003). A requirement for retinoic acid-mediated transcriptional activation in ventral neural patterning and motor neuron specification. Neuron, 40, 81-95. 92. Cehajic-Kapetanovic, J., Le Goff, M. M., Allen, A., Lucas, R. J., Bishop, P. N. (2011). Glycosidic enzymes enhance retinal transduction following intravitreal delivery of AAV2. Molecular vision, 17, 1771-1783. 93. Russo, J. E., Hauguitz, D., Hilton, J. (1988). Inhibition of mouse cytosolic aldehyde dehydrogenase by 4-(diethylamino)benzaldehyde. Biochemical pharmacology, 37, 1639-1642.

What is claimed is:

1. A method of treating vision degeneration, said method comprising administering to a subject in need thereof an effective amount of a retinoic acid receptor inhibitor.

2. The method of claim 1, wherein the retinoic acid receptor inhibitor is an RAR antagonist.

3. The method of claim 2, wherein the RAR antagonist inhibits the binding of a nuclear receptor coactivator to the retinoic acid receptor.

4. The method of claim 1, wherein the retinoic acid receptor inhibitor is an RAR inverse agonist.

5. The method of claim 4, wherein the RAR inverse agonist increases the binding of a nuclear receptor corepressor to the retinoic acid receptor.

6. The method of claim 1, wherein light sensitivity of retinal ganglion cells in the subject is increased.

7. The method of claim 1, wherein hyperexcitability of retinal ganglion cells in the subject is inhibited.

8. The method of claim 1, wherein increases in the number, activity, or cellular distribution of hyperpolarization-activated cyclic nucleotide-gated channel in retinal ganglion cells are reduced.

9. The method of claim 1, wherein the vision degeneration is associated with retinitis pigmentosa, age-related macular degeneration, cone dystrophy, rod-cone dystrophy, Leber's congenital amarurosis, Usher's syndrome, Bardet-Biedl-syndrome, or Stargardt disease.

10. A method of inhibiting the activity of a retinoic acid receptor in a subject in need thereof, comprising contacting the retinoic acid receptor with a retinoic acid receptor inhibitor.

11. The method of claim 10, wherein the retinoic acid receptor inhibitor is an RAR antagonist.

12. The method of claim 11, wherein the RAR antagonist inhibits the binding of a nuclear receptor coactivator to the retinoic acid receptor.

13. The method of claim 10, wherein the retinoic acid receptor inhibitor is an RAR inverse agonist.

14. The method of claim 13, wherein the RAR inverse agonist increases the binding of a nuclear receptor corepressor to the retinoic acid receptor.

15. The method of claim 10, wherein the retinoic acid receptor contacts a retinoid x receptor.

16. The method of claim 15, wherein the retinoic acid receptor inhibitor contacts the retinoid x receptor.

17. The method of claim 1, wherein the retinoic acid receptor is RARα.

18. The method of claim 1, wherein the retinoic acid receptor inhibitor has the formula: 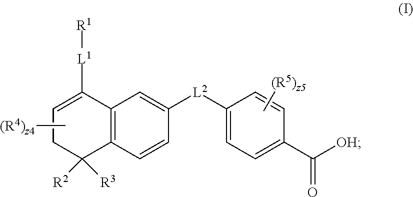
wherein
L¹ is a bond, —S(O)₂—, —NH—, —O—, —S—, —C(O)—, —C(O)NH—, —NHC(O)—, —NHC(O)NH—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —S(O)₂—, —NH—, —O—, —S—, —C(O)—, —C(O)NH—, —NHC(O)—, —NHC(O)NH—, —NHC(O)NH—, —C(O)O—, —OC(O)—, —C(S)—, —C(S)NH—, —NHC(S)—, —NHC(S)NH—, —NHC(S)NH—, —C(S)O—, —OC(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are each independently hydrogen, or substituted or unsubstituted alkyl, or substituted or unsubstituted heteroalkyl;
R⁴ and R⁵ are each independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z4 is an integer from 0 to 3; and
z5 is an integer from 0 to 4.

19. The method of claim 18, wherein L² is

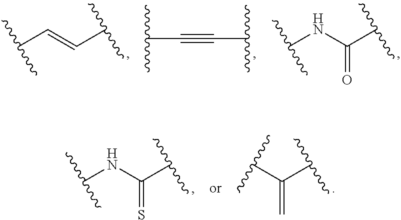

20. The method of claim 18, wherein -L¹-R¹— has the formula:

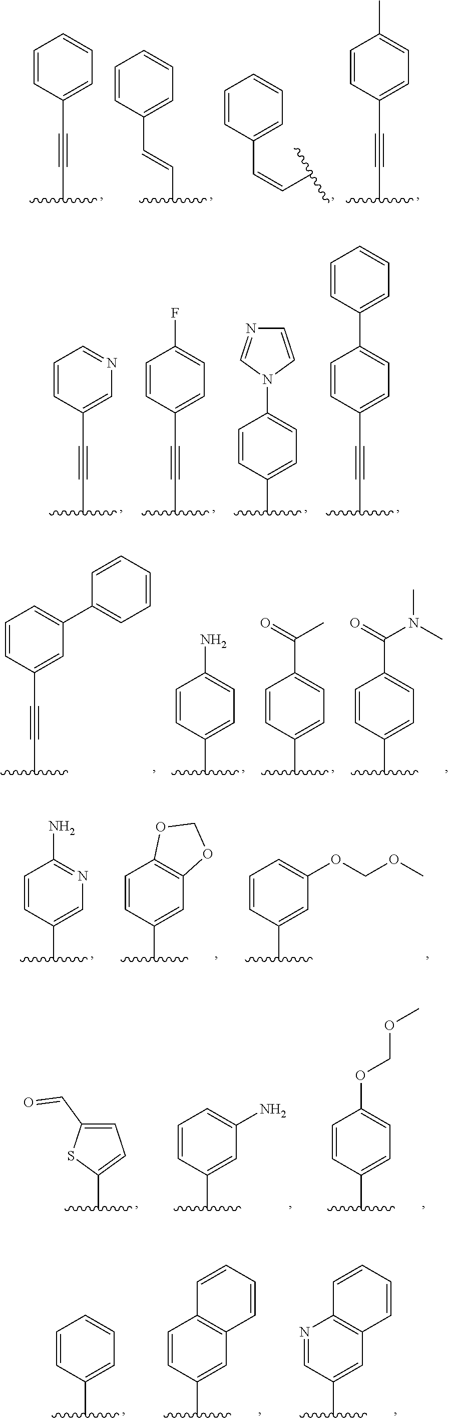

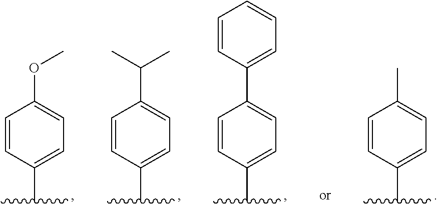

21. The method of claim 1, wherein the retinoic acid receptor inhibitor is

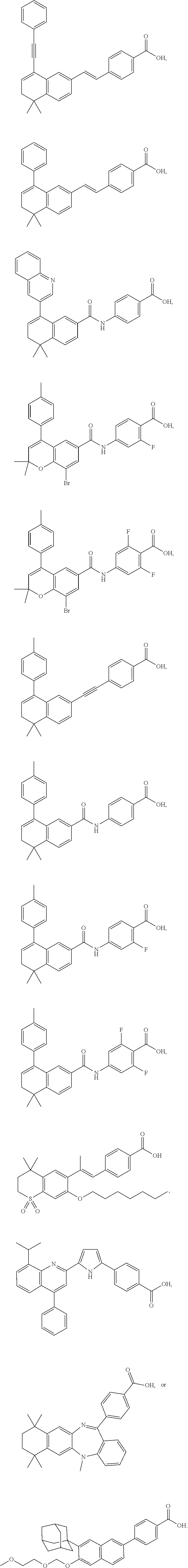

22. The method of claim 21, wherein the retinoic acid receptor inhibitor is

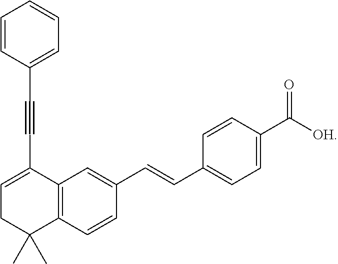 [SMILES: CC1(C)CC=C(C#Cc2ccccc2)c2cc(/C=C/c3ccc(C(=O)O)cc3)ccc21]

23. The method of claim 1, wherein the retinoic acid receptor inhibitor comprises a nucleic acid.

24. The method of claim 23, wherein the retinoic acid receptor inhibitor is a nucleic acid.

25. The method of claim 1, wherein the retinoic acid receptor inhibitor comprises a gene modulating reagent.

26. The method of claim 25, wherein the gene modulating reagent is a gene editing reagent or a gene modulating nucleic acid.

27. The method of claim 26, wherein the gene editing reagent is a CRISPR complex, a TAL effector nuclease, a zinc-finger nuclease, a meganuclease, or a homing endonuclease.

28. The method of claim 27, wherein the CRISPR complex comprises a guide RNA and a Cas9 nuclease.

29. The method of claim 28, wherein the guide RNA comprises a nucleic acid sequence at least 80% identical to an RNA sequence of a retinoic acid receptor or a fragment thereof or a complement thereof.

30. The method of claim 28, wherein the guide RNA comprises a nucleic acid sequence identical to an RNA sequence of a retinoic acid receptor or a fragment thereof, or a complement thereof.

31. The method of claim 29, wherein the guide RNA comprises a nucleic acid sequence from 10 to 30 nucleotides in length.

32. The method of claim 28, wherein the guide RNA comprises a nucleic acid sequence at least 80% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site.

33. The method of claim 28, wherein the guide RNA comprises a nucleic acid sequence from 10 to 30 nucleotides in length and at least 80% identical to an RNA sequence of a retinoic acid receptor or a fragment thereof, or a complement thereof, or an RNA sequence or a fragment thereof, or a complement thereof corresponding to a nucleic acid sequence upstream or downstream of the retinoic acid receptor transcription start site.

34. The method of claim 26, wherein the gene modulating nucleic acid is an antisense nucleic acid or an siRNA.

35. The method of claim 34, wherein the antisense nucleic acid comprises a nucleic acid sequence at least 80% identical to a nucleic acid sequence complementary to an RNA sequence of a retinoic acid receptor or a fragment thereof.

36. The method of claim 34, wherein the antisense nucleic acid comprises a nucleic acid sequence complementary to an RNA sequence of a retinoic acid receptor or a fragment thereof.

37. The method of claim 34, wherein the antisense nucleic acid comprises a nucleic acid sequence at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

38. The method of claim 35, wherein the antisense nucleic acid comprises a nucleic acid sequence from 10 to 50 nucleotides in length.

39. The method of claim 34, wherein the antisense nucleic acid comprises a nucleic acid sequence from 10 to 50 nucleotides in length and at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

40. The method of claim 34, wherein the siRNA comprises a nucleic acid sequence at least 80% identical to a nucleic acid sequence complementary to an RNA sequence of a retinoic acid receptor or a fragment thereof.

41. The method of claim 34, wherein the siRNA comprises a nucleic acid sequence identical to an RNA sequence complementary to an RNA sequence of a retinoic acid receptor or a fragment thereof.

42. The method of claim 34, wherein the siRNA comprises a nucleic acid sequence at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

43. The method of claim 40, wherein the siRNA comprises a nucleic acid sequence from 20 to 30 nucleotides in length.

44. The method of claim 34, wherein the siRNA comprises a nucleic acid sequence from 20 to 30 nucleotides in length and at least 80% identical to a nucleic acid sequence complementary to an RNA sequence of a retinoic acid receptor or a fragment thereof, or a nucleic acid sequence or a fragment thereof upstream or downstream of the retinoic acid receptor transcription start site.

45. The method of claim 23, wherein the retinoic acid receptor inhibitor comprises an expression vector.

46. The method of claim 45, wherein the expression vector is a viral vector.

47. The method of claim 45, wherein the expression vector is an adenovirus vector, adeno-associated virus vector, or a lentiviral vector.

48. The method of claim 45, wherein the expression vector is capable of expressing a dominant negative retinoic acid receptor protein.

49. The method of claim 48, wherein the dominant negative retinoic acid receptor protein is a truncated retinoic acid receptor compared to the wildtype retinoic acid receptor protein.

50. A method of treating vision degeneration, said method comprising administering to a subject in need thereof an effective amount of an inhibitor of the level of retinoic acid in the subject.

51. The method of claim 50, wherein the inhibitor is a retinaldehyde dehydrogenase inhibitor.

52. The method of claim 51, wherein the retinaldehyde dehydrogenase inhibitor is diethylaminobenzaldehyde, citral, or disulfiram.

53. The method of one of claims 1 to 52, wherein the retinoic acid receptor inhibitor is administered topically to the eye.

54. The method of one of claims 1 to 52, wherein the retinoic acid receptor inhibitor is administered by intraocular, subconjunctival, intravitreal, retrobulbar, or intracameral administration.

55. The method of one of claims 1 to 52, wherein the retinoic acid receptor inhibitor is administered by intravitreal or intravenous administration.

56. The method of one of claims 1 to 52, wherein the retinoic acid receptor inhibitor is administered by intravitreal administration.

57. The method of one of claims 1 to 52, wherein the vision degeneration is associated with a reduction in cone cells.

58. The method of one of claims 1 to 52, wherein the vision degeneration is associated with a reduction in rod cells.
